SEQUENCE LISTING (471 sequences)

SEQ ID NO: 1 - DNA, 297 bp, Canis lupus familiaris
gaggtccagctggtgcagtctggggctgaggtgaggaaaccagtttcatctgtgaaggtctcctggaaggcatctggatacacctacatggatgcttatatgcactggttatgacaagcttcaggaataaggtttgggtgtatgggatggattggtcccaaagatggtgccacaagatattcacagaagttccacagcagagtctccctgatggcagacatgtccaaagcacagcctacatgctgctgagcagtcagaggcctgaggacacacctgcatattactgtgtgggacact

SEQ ID NO: 2 - DNA, 294 bp, Canis lupus familiaris
gaggtccagctggtgcagtctggggctgaggtgaagaagccaggtacatccgtgaaggtctcatgcaagacatctggatacaccttcactgactactatatgtactgggtacgacaggcttcaggagcagggcttgattggatgggacagattggtccctaagatggtgccacaaggtatgcacagaagtttcagggcagagtcaccctgtcaacagacacatccacaagcacagcctacatggagctgagcagtctgagagctgaggacacagccatgtactactctgtgaga

SEQ ID NO: 3 - DNA, 291 bp, Canis lupus familiaris
gaggtccagctggtgcagtctggggctgaggtgaagaagctaagggcatcagtgatagtcccctgcaagacatctggatacagcttcactgactacattttggaatgggtatgacaggctccaggaccagggcttgagtggatgggatggattggtcctgaagatggtgagacaaagtatgtgcagaagttccaggcagagtcaccctgatggcagacacaaccacaagcacagccaacatggagctgaccagtctgagagctgaggacacagccatgtactactgtgtga

SEQ ID NO: 4 - DNA, 294 bp, Canis lupus familiaris
gaggtccagctggtgcagtctggggctgaggtgaagaagccaggggcatctgtgaaggtctcctgcaagacatctggatacaccttcattaactactatatgatctgggtacgacaggctccaggagcagggcttgattggatgggacagattgatcctgaagatggtgccacaagttatgcacagaagttccagggcagagtcaccctgacagcagacacatccacaagcacagcctacatggagctgagcagtctgagagctggggacatagctgtgtactactgtgcgaga

SEQ ID NO: 5 - DNA, 296 bp, Canis lupus familiaris
gaggtgcagctggtggagtctgggggagacctggtgaagcctggggggtccctgagactctcctgtgtggcctctggattcaccttcagtagcaactacatgagctggatccgccaggctccagggaaggggctgcagtgggtctcacaaattagcagtgatggaagtagcacaagctacgcagacgctgtgaagggccgattcaccatctccagagacaatgccaagaacacgctgtatctgcagatgaacagcctgagagatgaggacacggcagtgtattactgtgcaaggga

SEQ ID NO: 6 - DNA, 296 bp, Canis lupus familiaris
gaggtgcagctggtggagtctgggggagacatggtgaagcctggggggtccctgagactctcctgtgtggcctctggatttaccttcagtagttactacatgtattgggcccgccaggctccagggaaggggcttcagtgggtctcacacattaacaaagatggaagtagcacaagctatgcagacgctgtgaagggccgattcaccatctccagagacaacgcaaagaatacgctgtatctgcagatgaacagcctgagagctgaggacacagcggtgtattactgtgcaaagga

SEQ ID NO: 7 - DNA, 297 bp, Canis lupus familiaris
gaggtgcagctggtggagtctgggggagacctgatgaagcctgggggggtccctgagactctcctgtgtggcctctgaattcatcttcagtggctactggaagtactggatccaccaagctccagggaaggggctgcagtgggtcacatggattagcaatgatggaagtagcaaaagctatgcagacgctgtgaagggccaattcaccatctccaaagacaatgccaaatacacgctgtatctgcagatgaacagcctgagagccgaggacatggccgtgtattactgtatgatgca

SEQ ID NO: 8 - DNA, 296 bp, Canis lupus familiaris
gaggtgcagctggtggagtctgggggagacctggtgaagcctggggggtccctgagactttcctgtgtggcctctggattcaccttcagtagctaccacatgagctgggtccgccaggctccagggaaggggcttcagtgggtcgcatacattaacagtggtggaagtagcacaagctatgcagacgctgtgaagggccgattcaccatctccagagacaacgccaagaacacgctgtatcttcagatgaacagcctgagagccgaggacacggccgtgtattactgtgcgagtga

SEQ ID NO: 9 - DNA, 297 bp, Canis lupus familiaris
gaggtgcagctggtggagtctgggggagccctggtgaagcctgggggggtccctgagactctcctatgtggcctctggattcaccttcagtagctaccacatgagctgggtccgccaggctccagggaaggggctgcagtgggtcgcatacattaacagtggtggaagtagggatccctgggtggcgcagtggtttggcgcctgcctttggcccagggcacgatcctggagacccgggatcgaatcccacgtcgggctccctgcatggagcctgcttctccctctgcctgtgtctct

SEQ ID NO: 10 - DNA, 293 bp, Canis lupus familiaris
gaggtgcagctggtggagtctgggggagacctggtgaagcctggggggtccctgagactctcctgtgtagcctctggattcaccttcagtagctccgacatgagctggatccgccaggctccaggaaaggggcttcagtgggtcgcatacattagcaatgatggaagtagcacaagctacgcagacgctgtgaagggccgattcaccatctccagagacaacgccaagaacacgctctatctgcagatgaacagcctcagagccgaggacacggccgtgtattactgtgcaga

SEQ ID NO: 11 - DNA, 296 bp, Canis lupus familiaris
gaggagcaactggtggagtttggaggacacatggtgaatcctgggggttccctgggtctctcctgtcaggcctctggattcaccttcagtagctatggcatgagctgggtccgccaggctcaaaagaaggggctgcagtgggtcggacatattagctatgatggaagtagtacatactacgcagacactttgagggacagattcaccatctccagagacaacaccaagaacatgctgtatctgcagatgaacagcctgagagccgaggacacagccgtgtattactgcatgaggaa

SEQ ID NO: 12 - DNA, 296 bp, Canis lupus familiaris
gaggtgcagctggtggagtctgggggagacctggtgaagcctggggggtccctgagactctcctgtgtggcctctggattcaccttcagtaactacgaaatgtactgggtccgccaggctccagggaaagggctggagtgggtcgcaaggatttatgagagtggaagtaccacatactatgcagaagctgtaaagggccgattcaccatctccagagacaacgccaagaacatggcgtatctgcagatgaacagcctgagagccgaggacacggccgtgtattactgtgcgagtga

SEQ ID NO: 13 - DNA, 296 bp, Canis lupus familiaris
gaggtgcagctggtggagtctggaggagacctggtgaagcctggggggtccctgagactttcctgtgtggcctctggattcaccttcagtagctatgacatggactgggtccgccaggctccagggaaggggctgcagtggctctcagaaattagcagtagtggaagtagcacatactacgcagacgctgtgaagggccgattcaccatctccagagacaacgccaagaacacgctgtatctgcagatgaacagcctgagagccgaggacacggccgtgtattactgtgcaaggga

SEQ ID NO: 14 - DNA, 296 bp, Canis lupus familiaris
gaggtgcagctggtggagactgagggagacctggtgaagcctgggggatccctgagactttcctgtgtggcctctggattcaccttcagtagctacgacatggactgggtctaccaggctccagggaaagggttacagtgggtcacatacattagcaatggtggaagtagcacaaggtatgcagacgctgtgaagggccaattcaccatctccagagacaacgccaggaacacgctctatctgcagatgaacagcctgagagacaaggacatggccgtgtattactgtgtgagtga

SEQ ID NO: 15 - DNA, 293 bp, Canis lupus familiaris
gaggtgcagctggtggagtctaggggagacgtggtgaagcctggggaggtccctctcctgtgtggcctctagattcaccttcagtagctactacatgggctgggtccactaggctccagggaaggggctgcagtgggtcgcaggtattaccaatgatagaagtagcacaagctatgcagacgctgtgaagggccgattcaccatctccagagacaatgccaagaacacgctgtatctgcagatgaacagcctgggagccgaggacacggctgtgtattattgtgtgaaacaga

SEQ ID NO: 16 - DNA, 296 bp, Canis lupus familiaris
gaggtgcagctggtggagtctggggagacctggtgaagcctggggggtctctgagactctcctgtgtggcctctggattcaccttcagtagctactacatgagctgggtccgccaggctccagggaaggggctgcagtgggtcggatacattaacagtggtggaagtagcacatactatgcagacgctgtgaagggccgattcaccatctccagagacaatgccaagaacacgctgtatctgcagatgaacagcctgagagccgaggacacagctgtgtattactgtgggaaggga

SEQ ID NO: 17 - DNA, 296 bp, Canis lupus familiaris
gaggagcaactggtggagtttggaggacacatggtgaatcctgggggttccctgggtctctcctgtcaggcctctggattcaccttcagtagctatggcatgagctgggtccgccaggctcaaaagaaggggctgcagtgggtcggacatattagctatgatggaagtagcacatactacacagacactgtgagggacagattcaccatctccagagacaacaccaagaacatgctgtatctgcagatgaacagcctgagagccgaggacacagccgtgtattactgcatgaggaa

SEQ ID NO: 18 - DNA, 296 bp, Canis lupus familiaris
gaggtgcagatggtggagtctgggggagacctggtgaagcctgggggatccctgagactctcctgtgtggcctctggattcaccttcagtaactacaaaatgtactgggtccaccaggctccagggaaagggctggagtgggtcgcaaggatttatgagagtggaagtaccacatactacgcagaagctgtaaagggccgattcaccatctccagagacaacgccaagaacatggtgtatctgcagatgaacagcctgagagcctaggacacggccgtgtattactgtgtgagtga

SEQ ID NO: 19 - DNA, 296 bp, Canis lupus familiaris
gaggtacagctggtggagtctggaggagacctggtgaagcctggggggtccctgagactctcctgtgtggcctctggattcacctttagtagttactacatgttttggatccgccaggcaccagggaagggcaatcagtgggtcggatatattaacaaagatggaagtagcacatactacccagacgctgtgaagggccgattcaccatctccagagacaacgccaagaacacactgtatctgcagatgaacagcctgacagtggaggacacagccctttattactgtgcgagaga

SEQ ID NO: 20 - DNA, 296 bp, Canis lupus familiaris
gaggtgcagctggtggagtctgggggagaccttgtgaaacctgaggggtccctgagactctcctgtgtggtctctggcttcaccttcagtagctacgacatgagctgggtccgccaggctccagggaaggggctgcagtgggtcgcatacattagcagtgatggaaggagcacaagttacacagacgctgtgaagggccgattcaccatctccagagacaatgccaagaacacgctgtatctgcagatgaacagcctgagaactgaggacacagccgtgtattactgtgcgaagga

SEQ ID NO: 21 - DNA, 296 bp, Canis lupus familiaris
gaggtgcagctggtggagtctgggggagacctggtgaagcctgcggggtccctgagactgtcctgtgtggcctctggattcaccttcagtagctacagcatgagctgggtccgccaggctcctgagaaggggctgcagttggtcgcaggtattaacagcggtggaagtagcacatactacacagacgctgtgaagggccgattcaccatctccagagacaacgccaagaacacagtgtatctgcagatgaacagcctgagagccgaggacacggccatgtattactgtgcaaagga

SEQ ID NO: 22 - DNA, 294 bp, Canis lupus familiaris
gaggtgcagctggtggagtctgggggatacctggtgaagcctggagggtcctgagactctcctctgtgtcctctggattcaccttcagtatctactgcatgtgatgggtctgccaggctccaggaaaggggctgcagtgagtcgcatacagtaacagtggtggaagtagcactaggtacacagacgctgtgaagggctgattcaccacctccagagacaatgccaagaacacactgtatctgcagatgaacagcctgagagtgaggacacagcggtgtattactgtgcaggtga

SEQ ID NO: 23 - DNA, 296 bp, Canis lupus familiaris
gaggtgcagctgttggagtctgggggagacctggtgaagcctggggggtccctgagactgtcctgtgtggtctctggattcaccttcagtaagtatggcatgagctgggtctgccaggctttggggaaggggctacagttggtcgcagctattagctaagatggaaggagcacatactacacagacactgtgaagggccgattcaccatctccagagacaatgccaagaacacgctgtacctgcagatgaacagcttgagagctgaggacacggccgtgtattactgtgagagtga

SEQ ID NO: 24 - DNA, 284 bp, Canis lupus familiaris
gaggtgaagctagtggagtctgggggagacctggtgaagcctgggggatcaattagactctcctatgtgacctctggattcaccttcaggagctactggatgagctgggtcagccaggctccagggaaggggctgcagtgggtcatatgggttaatactggtggaagcagaaaaagctatgcagatgctgtgaaggggtgattcaccatctccagagacaatgccaagaacacgctgtatctgcatatgaacagcctgagagccctgtattattatgtgagtga

SEQ ID NO: 25 - DNA, 292 bp, Canis lupus familiaris
gaggtgcagatgatggagtctgggggagaactgatgaagcctgcaggatccctgagacctcctgtgtggcctctggattcaccttcagtagctactggatgtactggatccaccaaactccggggaaggggctgcagtgggtcgcaggtattagcacagatggaagtagcacaagctacgtagacgctctgaagggctgattcaccatctccagagacaacgccaagaacacgctctatctgcagatgaacagcctgagagccgaggacatggccatgtattactgtgcaga

SEQ ID NO: 26 - DNA, 296 bp, Canis lupus familiaris
gaggtgcagctggtggagtctgggggagacctggagaagcctgggggatccctgagactgtcctgtgtggcctctggattcaccttcagtagctacggcatgagctgggtccgccaggctccagggaaggggctgcagggggtctcattgattaggtatgatggaagtagcacaaggtatgcagacgctgtgaagggccgattcaccatctccagagacaatgccaagaacacgctgtatctgcagatgaacagcctgagagccgaggacacagccgtgtattcctgtgcgaagga

SEQ ID NO: 27 - DNA, 296 bp, Canis lupus familiaris
gaggtgcagctggtggagtctgggggagaccttgtgaagcctgaggggtccctgagactctcctgtgtggcctctggattcaccttcagtagcttctacatgagctggttctgccaggctccaaggaaggggctacagtgggttgcagaaattagcagtagtggaagtagcacaagctacgcagacattgtgaagggccgattcaccatctccagagacaatgccaagaacatgctgtatctgcagatgaacagcctgagagccgaggacatggccgtatattattgtgcaaggta

SEQ ID NO: 28 - DNA, 296 bp, Canis lupus familiaris
gaggtgcagctggtggagcctgggggagaactggtgaagcctggggcgtccctgagactctcctgtgtggtccctggattcaccttcagtagctacaacatgggctgggctcaccagcctccagggaaggggatgcagtgggtcgcaggttttaacagcggtggaagtagcacaagctacacagatgctgtgaagggtgaattcaccatctccagagacaatgtcaagaacacgctgtatctgcagatgaacagcctgagatccgaggacacggccgtgtattactgtgtgaagga

SEQ ID NO: 29 - DNA, 296 bp, Canis lupus familiaris
gaggtgtagctggtggagtctgggggagacctggtgaagcctggggggtccctgagactctcctgtgtgggctctggattcaccttcagtagctactggatgagctgggtccgccaggctccagggaaggggctacagtgggttgcagaaattagcggtagtggaagtagcacaaactatgcagacgctgtgaagggccgattcatcatctccagagacaatgccaagaacacgctgtatctgcagatgaacagcctgagagccgaggacacggccatgtattactgtgcaaggga

SEQ ID NO: 30 - DNA, 300 bp, Canis lupus familiaris
aaggtgcatctggtggagtctgcgggagacgtggtgaagcctaggaggtccctgagactctcctgtgtgggctctggattcaccttcagtagctacagcatgtggtgggcccgtgaggctcccgggatggggctacagggggtcgcaggtattagatatgatggaagtagcacaagctacgcagacgctctgaagggccgattcaccatctccagagacaatgccaaaaacacactgtatctgtagaagaacagcctgagagccgagggaggacacggccgtgtattactgtgcgaggga

SEQ ID NO: 31 - DNA, 296 bp, Canis lupus familiaris
gaggtgcagctagtggagtctgggggagacctggtgaagtctgggggggtccctgagagtctcctgtgtgggctctggattcaccttcagtagctactggatgtactgggtccaccaggctccagggaaggggctccatgggtcgcatggattaggtatgatggaagtagcacaagctacgcagaagctgtgaaaggccgattcactgtttctagagacaacgccaagaacacgctgtatctgcagatgaacagcctgagagccgaggacacggccgtgtattactgtgtgaggga

SEQ ID NO: 32 - DNA, 303 bp, Canis lupus familiaris
gaggtgcagctggtggagtcctggggagacctggtgaagactggaggtttcctgagactctcctgtctcctgtgtggcttccggattcaccttcagtaactacagcatgatctgggtccgccaggctccaaggaaggggctgcagtggatcacaactattagcaatagtggaagtagcacaaatcacgcagacacagtaaagggccgatttaccatctccagagacaacaccaagaacacgctgtatctacagatgagcagcctgggagccgatgacacggccctgtattactgtgtgaggga

SEQ ID NO: 33 - DNA, 296 bp, Canis lupus familiaris
gaggtgcagctggtggagtctgggggagaactggtgaagcctggggggtccctgagactctcctgtgtggcctctggattcaccttcagtagctactacatgagctggatccgccaggctcctgggaaggggctgcagtgggtcgcagatattagtgacagtggaggtagcacatactacactgacgctgtgaagggccgattcaccatctccagagacaacgtcaagaactcgctgtatttgcagatgaacagcctgagagccgaggacacggccgtgtattactgtgcgaagga

SEQ ID NO: 34 - DNA, 296 bp, Canis lupus familiaris
ggggtgcagctggtggagtctgggggagacctggtgaagcctggggggtccctgacactctcctgtgtggcctatggattcaccttcagtagctacagcatgcaatgggtctgtcaggctccagggaagggggtgcagtgggtcgcatacattaacagtggtggaagtagcacaagctccgcagatgctgtgaagggtcgattcatcatctccagagacaacgtcaagaacacgctatatctgcagatgaacagcctgagagccgaggacaccgccgtgtattactgtgcgggtga

SEQ ID NO: 35 - DNA, 294 bp, Canis lupus familiaris
gagatgcagctggtggaggctgggggagacctggtgaagcttggggggtccctgagactcttctgtgtggcctctggatttaccttcagtagctattggatgagctgggtcggccaggctccagggaaagggttgcagtgggttgcatacattaacagtggtggaagtagcacatactatgcagacgctgtgaagggccgattcaccatctccagagacaatgccaagaacacgctgtatctgcagatgaactgcctgagagccgaggacacggccgtatattactgtgtggga

SEQ ID NO: 36 - DNA, 115 bp, Canis lupus familiaris
cagacactgtgaagggccgattcaccatctccagagacaacgccaagaacacgctctatctgcagatgaacagcctgagagctgaggacacggccgtgtattactgtgcgaagga

SEQ ID NO: 37 - DNA, 296 bp, Canis lupus familiaris
gaggtgcagctggtggagtctgggggagacctggtgaagcctgtgggatccctgagactctcctgtgtggcctctggattcaccttcagtagctatgacatgaactgggtccgccaggctccagggaaggggctgcagtgggtcgcatacattagcagtggtggaagtagcacatactatgcagatgctgtgaagggccggttcaccatctccagagacaacgccaagaacacgctgtatcttcagatgaacagcctgagagccgaggacacggccatgtattactgtgcgggtga

SEQ ID NO: 38 - DNA, 296 bp, Canis lupus familiaris
gaggggcagctggcggagtctgggggagacctggtgaagcctgagaggtccctgagactcgcccgtgtggcctctggattcaccttcatttcctataccatgagctgggtccacaaggctcctgggaaggggctgccgtgagtcgcatgaatttattctagtggaagtaacatgagctatgcagacgctgtgaagggccgattcaccatctccagagacaatgccaagaacatgctgtatctgcagatgaacagcctgagagctgaggacatggccatgtattactgtgtgaatga

SEQ ID NO: 39 - DNA, 293 bp, Canis lupus familiaris
gaggtacagctggtggagtctggggaagatttggtgaagcctggagggtccctgagactctcctgtgtggcctctggattcaccttcagtagcagtgaaatgagctgggtccaccaggctccagggcaggggctgcagtgggtctcatggattaggtatgatggaagtatctcaaggtatgcagacactgtgaagggccgattcaccatctccagagacaatgtcaagaacacgctgtatctgcagatgaacagcctgagagccgaggacacggccatatattactgtgcaga

SEQ ID NO: 40 - DNA, 296 bp, Canis lupus familiaris
gaggtgcagctggtggagtctgggggagacctggtgaagcctggggggaccttgagactgtcctgtgtggcctctggattcacctttagtagctatgacatgagctgggtccgtcagtctccagggaaggggctgcagtgggtcgcagttatttggaatgatggaagtagcacatactacgcagacgctgtgaagggccgattcaccatctccagagacaacgccaagaacacgctgtatctgcagatgaacagcctgagagccgaggacacggccgtgtattactgtgcgaagga

SEQ ID NO: 41 - DNA, 296 bp, Canis lupus familiaris
gaggtacagctggtggaatctgggggagacctcgtgaagcctgggggttccctgagactctcctgtgtggcctcgggattcaccttcagtagctactacatgagctggatccgccaggctcctgggaaggggctgcagtgggtcgcagatattagtgatagtggaggtagcacaggctacgcagacgctgtgaagggccggttcaccatctccagagagaacgccaagaacaagctgtatcttcagatgaacagcctgagagccgaggacacagccgtgtattactgtgcgaagga

SEQ ID NO: 42 - DNA, 296 bp, Canis lupus familiaris
atgcaatgggtccgtcaggctcctgggaagggggtgcagtgggtcgcatacattaacagtggtggaagtagcacaagcttcgcagatgctgtgaagggcatgagctggtttcgccaggctccagggaaggggctgcaatgggttacatggattgggtatgatggaagtagcacatactacacagacactgtaaagggccgattcactatctccatagacaacgccaagaacatgctgtatctgcagatgaacagcctgagagccgaggacatagccctgtattactgtgcgaggga

SEQ ID NO: 43 - DNA, 296 bp, Canis lupus familiaris
gaggtgcagctggtggagtctgggggagacctggtgaagcctggggggtccctgagactctcctgtgtagcctctggattcaccttcagtaactacgacatgagctgggtccgccaggctcctgggaaggggctgcagtgggtcgcagctattagctatgatggaagtagcacatactacactgacgctgtgaagggccgattcaccatctccagagacaacgccaggaacacagtgtatctgcagatgaacagcctgagagccgaggacacggctgtgtattactgtgcgaagga

SEQ ID NO: 44 - DNA, 297 bp, Canis lupus familiaris
gaagtgcagctggtggagtctgggggaagacctggtgaagccaggggggtccctgagactctcctgtgtgacctctggattcaccttcagtaggtatgccatgagctgggtcggccaggctccagggaagggcctgcagtgggttgcagctattagcagtagtggaagtagcacatactacgtagatgctgtgaagggccgattcaccatctccatagacaacgccaagaacatggtgtatctgcagatgaacagcctgagagctgaggatattgctgtgtattactgtgggaagga

SEQ ID NO: 45 - DNA, 296 bp, Canis lupus familiaris
aaggtgtagctggtggagtctgggggagacctgatgaagcctgggggttccctgagactgtcctgtgtggcctctggattcaccttcaggagctatggcatgagctgggtctgccaggcttcagggaaggggctgcagtgggtcgcagctattagctatgatggaaggagcacatactacacagacactgtgaagggccgattcaccatctccagagacaatggcaagaacacgctgtacctgcagatgaacagcttgagagctgaggacacggccgtgtattactgtgcgagtga

SEQ ID NO: 46 - DNA, 296 bp, Canis lupus familiaris
gaggtgcagctggtggagtctgggggagacctggtgaagcctgggggttccctgagactctcatgtgtgacttctggattcaccttcagtagctattggatgagctgtgtccgccaggctccagggaaggagctgcagtgggtcgcgtacattaacagtggtggaagtagcacatggtacacagacgctgtgaagggtcgattcaccatctccagagacaacgccaagaacacgctgtatctgcagatgaacaacctgagagccgaagacacggccgtgtattactgtgcgaggga

SEQ ID NO: 47 - DNA, 295 bp, Canis lupus familiaris
gaagtacagctgctggagtctgggggagaccgagtgaaacctggggggtcccagagactctcctgtgtggcctcaaggttcaccttcagtagctacagcatgcattgtctccgtcagtctcctgggatggggctacagtgggtcacatacattagcagtaatggaagcagcacatactatgcagacgctgtgaagggtcgattcaccatctccagagacaaagccaagaacatgctttatctacagatgaacagcctgagagctcaggacatagccctgtattactgtgcagatg

SEQ ID NO: 48 - DNA, 293 bp, Canis lupus familiaris
gaggtacagctggtggagtctggggaagatttggtgaagcctggagggtccctgagactctcctgtgtggcctctggattcaccttcagtagcagtgaaatgagctgggtccaccaggctccagggcaggggctgcagtgggtctcatggattaggtatgatggaagtagctcaaggtatgcagacactgtgaagggccgattcaccatctccagagacaatgccaagaacacgctgtatctgcagatgaacagcctgagagccgaggacacggccatatattactgtgcaga

SEQ ID NO: 49 - DNA, 293 bp, Canis lupus familiaris
gaggtgcagctggtggagtctgggggagacctggcgaagcctggggggtccctgagactctcctgtgtggcctctggattaaccttcagtagctacagcatgagctgggtccgccaggctcctgggaaggggctgcagtgggtcacagctattagctatgatggaagtagcacatactacactgacgctgtgaagggccgattcaccatctccagagacaacgccaggaacacagtgtatctgcagatgaacagcctgagagccgaggacacagctgtgtattactgtgtgga

SEQ ID NO: 50 - DNA, 293 bp, Canis lupus familiaris
gaggtgccactggtggaatctgggggagagctggtgaagcctggggggtccctgagactctcctttgtagcctctgcattcactttcagtagttactggataagctgggtccgccaagctccagggaaagggctgcactgagtctcagtaattaacaaagatggaagtaccacataccacgcagatgctgtgaagggccgattcaccatctccagagacaatgccaagaacacgctgtatctgcagatgaacagcctgagagctgaggacacggctgtgtattactgtgcaca

SEQ ID NO: 51 - DNA, 295 bp, Canis lupus familiaris
gaggagcagttggtgaaatctaggggagacctggtgaagcctggcgggtccctgagactcttctgtgagtcctctacattcacctttcatagcaacagcatacattggctccaccagtctcccggtagtggctacagtgggtcatatccaatagcagtaatggaagtagcatgtactatgcagacgctgtaaagggctgattcaccatctccagagacagcaccaggaacatgctgtatctgcagatgaacagcctgagagctgaggacacagccgtgcattgctgtgcgaggga

SEQ ID NO: 52 - DNA, 294 bp, Canis lupus familiaris
gaggtgcagctggtggagtctgggggagacctcatgaagcctggggggtccctgagactctcctgtgtggccgctggattcaccttcagtagctacagcatgagctgggtccgccaggctcccgggaaggggattcagtgggtcgcatggatttaagctagtggaaatagcacaagctacacagatgctgtgaagggccgattcaccatctccagagaacgccaagaacacagtgtttctgcagatgaacagcctgagagctgaggacaaggccatgtattactgtgcgaggga

SEQ ID NO: 53 - DNA, 296 bp, Canis lupus familiaris
gaggtgcagctggtggagtctgggggagacctggtgaagcctggggggtccttgagactctcctgtgtggcctctggtttcaccttcagtagcaacgacatggactgggtccgccaggctccagggaaggggctgcagtggctcacacggattagcaatgatggaaggagcacaggctacgcagatgctgtgaagggccgattcaccatctccagagacaacgccaagaacacgctgtatctgcagatgaacagcctgagagctgaggacacagccgtgtattactgtgcgaagga

SEQ ID NO: 54 - DNA, 295 bp, Canis lupus familiaris
gaggtgcagctggaggagtctgggggagacctggtgaagcctggggttccctaagactgtcctgtgtgacctccggattcactttcagtagctatgccatgcactgggtccgccaggctccagggaaggggctgcagtgggtcgcagttattagcagggatggaagtagcacaaactacgcagacgctgtgaagggccgattcaccatctccagagacaacgccaagaacatgctgtatctacagatgaacagcctgagagctgaggacacggccatgtattactgtgcgaagga

SEQ ID NO: 55 - DNA, 296 bp, Canis lupus familiaris
gaagtgcagctggtggagtatgggggagagctggtgaagcctggggggtccctgagactgtcctgtgtggcctccggattcaccttcagtatctactacatgcactgggtccaccaggctccagggaaggggctgcagtggttcgcatgaattaggagtgatggaagtagcacatactacactgatgctgtgaagggccgattcaccatctccagagacaattccaagaacactctgtatctgcagatgaccagcctgagagccgaggacacggccctatattactgtgcgatgga

SEQ ID NO: 56 - DNA, 295 bp, Canis lupus familiaris
gagatgcagctggtggagtctagggaggcctggtgaagcctggggggtccctgagactctcctgtgtggaccctggattcaccttcagtagctactggatgtactgggtccaccaggctccagggatggggctgcagtggcttgcagaaattagcagtactggaagtagcacaaactatgcagacgctgtgaggggcccattcaccatctccagagacaatgccaagaacacgctgtacctgcaggtgaacagcctgagagccgaagacacggccgtgtattactgtgtgagtga

SEQ ID NO: 57 - DNA, 297 bp, Canis lupus familiaris
gaggtgcagctggtggagtctgggggagacctgatgaagcctggggggtccctgagactctcctgtgtggcctccggattcactatcagtagcaactacatgaactgggtccgccaggctccagggaaggggctgcagtgggtcggatacattagcagtgatggaagtagcacaagctatgcagacgctgtgaagggccgattcaccatctccagagacaatgccaagaacacgctgtatctgcagatgaacagcctgagagccgaggacacggccgtgtattactgtgtgaaggga

SEQ ID NO: 58 - DNA, 295 bp, Canis lupus familiaris
gaggtgcagctggtggagtctggggaaacctggtgaagcctggggagtctctgagactctcttgtgtggcctctggattcaccttcagtagctactggatgcattgggtctgccaggctccagggaaagggttggggtgggttgcaattattaacagtggtggaggtagcacatactatgcagacacagtgaagggccaattcaccatcttcagagacaatgccaagaacatgctgtatctgcagatgaacagcctgagagcccaggacatgaccgcgtattactgtgtgagtga

SEQ ID NO: 59 - DNA, 253 bp, Canis lupus familiaris
gaggtgcagctggtggaatctgggggagacctggtgaagcctgggggatccctgagactctcctgtgtggcctctggattcaccttcagtagctactatatggaatgggtctgccaggctccagggaggggctgaagtgggtcgcacggattagcagtgacggaagtagcacatactacacagacgctgtgaagggccgattcaccatctccagagacaatgccaagacggccgtgtattactgtgcgaagga

SEQ ID NO: 60 - DNA, 295 bp, Canis lupus familiaris
gaagtgcagcttgtggagtctgggggagagctggtgaagcctgggggttccctgagactgtcctgtgtggcctctggattcaccttcagtagctactacatgcactgggtctgcaggctccagggaaggggctgcagtgggttgcaagaattaggagtgatggaagtagcacaagctacccagacgctgtgaagggcagattcaccatctccagagacaattccaagaacactctgtatctgcagatgaacagcctgagagctgatgatacggccctatattactgtgcaaggga

SEQ ID NO: 61 - DNA, 296 bp, Canis lupus familiaris
gaggtgcagctggtggagtctgggggagacctggtgaagcctgggggatccctgagactctcttgtgtggcctccggattcaccttcagtagccatgccaagagctgggtccgccaggctccagggaaggggctgaagtgggtagcagttattagcagtagtggaagtagcacaggctccgcagacactgtgaagggccgattcaccatctccagagacaatgccaagaacacgctgtatctgcagatgaacagcctgagagctgaggacacagccgtgtattactgtgcgaagga

SEQ ID NO: 62 - DNA, 294 bp, Canis lupus familiaris
gaggtacagctggtggagtctggaggagaccttgtgaagactgagcggtccctgagactctcctgtgtggcctctggattcaccttcagtagcttctacatgaggtgtctgccagactccagggaagggactacagtgggttgcagaaattagcagtagtggaagtagcacaagctacacagatgctctgaagggctgattctccatctccaaaaacaatgccaagaacacgctgtatctgcagatgaacagcctgagagccgaggtcacagccgtatattactgtgcaaggta

SEQ ID NO: 63 - DNA, 284 bp, Canis lupus familiaris
gaggtgaagctggtggagtctgggggagacctgttgaagcctgggggatcaattaaactctcctatgtgacctctggattcaccttcaggagctactggatgagctgggtcagccaggctccagggaaggggctgcagtgggtcacatgggttaatactggtggaagcagcaaaagctatgcagatgctgtgaaggggcaattcaccatctccagagacaatgccaagaacacgctgtatctgcatatgaacagcctgatagccctgtattattgtgtgagtga

SEQ ID NO: 64 - DNA, 296 bp, Canis lupus familiaris
gaggtgcagctggtggagtctggtggaaacctggtgaagcctgggggttccctgagactgtcctgtgtggcctctggattaaccttctatagctatgccatttactgggtccacgaggctcctgggaaggggctgcagtgggtcgcagctattaccactgatggaagtagcacatactacactgacgctgtgaagggccgattcaccatctccagagacaatgccaagaacacgctgtatctgcagatgaacagcctgagagctgaggacatgcccgtgtattactgtgcgaggga

SEQ ID NO: 65 - DNA, 292 bp, Canis lupus familiaris
gaggagcagctggtggagtctcggggagatctggtgaagtctggggggtccctgagactctcctgtgtggccccttgattcaccttcagtaactgtgacatgagctgggtccattaggctccaggaaagggctgcagtgtgttgcatacattagctatgatggaagtagcacaggttacaaagacgctgtgaagggccgattcaccatctccagagacaacgccaagaacatgctgtatcttcagatgaacagcctgagagctgaggacacggctctgtattactgtgcaga

SEQ ID NO: 66 - DNA, 295 bp, Canis lupus familiaris
gaggagcagttggtgaaatctaggggagacctggtgaagcctggcgggtccctgagactcttctgtgagtcctctacgttcacctttcatagctacagcatgcattggctccaccagtctcccggtagtggctacagtgggtcatatccaatagcagtaatggaagtagcatgtactatgcagacgctgtaaagggctgatacaccatctccagagacaacaccaggaacatgctgtatctgcagatgaataacctgagagctgaggacacagccgtgcattgctgtgcgaggga

SEQ ID NO: 67 - DNA, 291 bp, Canis lupus familiaris
gaggtgcagctggtggagtctgcgggagaccccgtgaagcctggggggtccctgagactctcctgtgtggccgctggattcaccttcagtagctacagcatgagctgggtccgccaggctcccgggaaggggatgcagtgggtcgcatggatatatgctagcggaagtagcacaagctacgcagacgctgtgaagggccgattcaccatctccagagacaacgccaagaacacactgtttctgcagatgcctgagagctgaggacacggccatgtattcctgtgcagggga

SEQ ID NO: 68 - DNA, 295 bp, Canis lupus familiaris
gatgtacagctggtggagtctgggggagacctggtgaagcctggggggtccctgagactgtcctgtgtggcctctggattcacctgcagtagctactacatgtactagacccaccaaattccagggaaggggatgcagggggttgcacggattagctatgatggaagtagcacaagctacaccgacgcaatgaaaggccgattcaccatctccagagacaacgccaagaacatgctgtatctgcaatgaacagcctgagagccgaggacacagccgtgtattactgtgtgaagga

SEQ ID NO: 69 - DNA, 291 bp, Canis lupus familiaris
gaggtgcagctggtggagtctggcggagacctggtgaagcctgggcggtccctgagactgtcctgtatggcctctggattcacttcagtagctacagcatgagctgtgtccgccaggctcctgggaagggctgcagtgggtcgcaaaaattagcaatagtggaagtagcacatactacacagatgctgtgaagggccgattcaccatctccagagacaatgccaagaacacgctctatctgcagatgaacagcctgagagccgaggacacggccttgtattactgtgcaga

SEQ ID NO: 70 - DNA, 296 bp, Canis lupus familiaris
gaggtgcagctggtggagtctgggggagacctggtgaagcctggggggtccctgagactgtcctgtgtggcctctggattcaccttcagtagctactacatgtactgggtccgccaggctccagggaaggggcttcagtgggtcgcacggattagcagtgatggaagtagcacatactacgcagacgctgtgaagggccgattcaccatctccagagacaatgccaagaacacgctgtatctgcagatgaacagcctgagagccgaggacacggctatgtattactgtgcaaagga

SEQ ID NO: 71 - DNA, 295 bp, Canis lupus familiaris
gaagtgcagctggtggagtctgggggagagctggtgaagcctggggggtccctgagactctcctgtgtggcctctggattcaccttcagtagctactacatgtactgggtccgccaggctccagggaaatggctgctgtgggtcacatgaattaggagtgatggaagtagcacatatacactgatgctgtgaaggaccgatacaccatctccaaagacaattccaagaacattctgtatctgcagatgaacagcctgagagccaaggacacggccctatatccctgtgcaatgga

SEQ ID NO: 72 - DNA, 296 bp, Canis lupus familiaris
gaggtacagctggtggagtctgggggagacctggtgaagcctgggggatccctgagactgtcctgtgtggcctctggattcaccttcagtagctatgccatgagctgggtccgccaggctccagggaaggggctgcagtgggtcgcatacattaacagtggtggaagtagcacatactacgcagatgctgtgaagggccggttcaccatctccagagacaatgccaggaacacactgtatctgcagatgaacagcctgagatccgaggacacagccgtgtattactgtccgaagga

SEQ ID NO: 73 - DNA, 296 bp, Canis lupus familiaris
gaggtgcagctggtggagtctggaggagaccttgtgaagcctgagcggtccctgagactctcctgtgtggcctctggattcaccttcagtagcttctacatgagctggttctgccaggctccagggaaggggctacagtgtgttgcagaaattagcagtagtggaaatagcacaagctacgcagacgctgtgaagggccgattcaccatctccagagacaacgccaagaacacgctgtatctacggatgcacagcctgagagccgaggacacggctgtatattactgtgcaaggta

SEQ ID NO: 74 - DNA, 282 bp, Canis lupus familiaris
gaggtgaagctggtggagtgtgggggagacctggtgaagcccgggggatcgattagactctcctttgtgacctctggattcaccttcaggagctattggatgggctgtgtcagccaggctccagggaaggggctgcagtgggtcacatgggttaatactggtggaagcagcaaaagctatgcagatgctatgaaggggcgatttaccatctccaggcacaaagccaagaacacactatctgcatatgaacagcctgagagccgtgtattattgtgtgagtga

SEQ ID NO: 75 - DNA, 296 bp, Canis lupus familiaris
gaggtgcagctggtggagtctggcggagacctggtgaagcctggggattccctgagactgtcctgtgtggcctctggattcaccttcagtagctatgccatgagctgggtccgccaggctcctaggaaggggctgcagtgggtcggatacattagcagtgatggaagtagcacataatacgcagacgctgtgaagggccgattcaccatttccagagacaatgccaagaacacgctgtatctgcagatgaacagcctgagagctgaggatacggccctgtataactgtgcaaggga

SEQ ID NO: 76 - DNA, 292 bp, Canis lupus familiaris
gaggtgcagctgatggagtctgggggagacctggtgaagcctggggggtccctgagactctcctgtgtggcccctggattcaccttcagtaactatgacatgagctcggtccattagactccaggaaagggctgcagtgtattgcatatattagctatgatggaagtagcacaggttacaaagacgctgtgcagggccgattcaccatctccagagacaacgccaagaacacgctgtatcttcagatgaacagcctgagagctgagcacacggccctgtattactgtgcaga

SEQ ID NO: 77 - DNA, 295 bp, Canis lupus familiaris
gaggtgcagctggtggagtctgggggagacttggtgaagccttgtgggctcctgagactctcctgtgtggcttctggattcaccttcagtagctacatcatgagctgggtccgccaggctccagggaagtggctgcagtgggtcgcatacattaacagtggtggaagtagcacaaggtacacagatgctgtgaagggccgattcacctctccagagacaacgccaagaacatgctgtatctgcagttgaacagcctgagagccgaggacaccgctgtgtattactgtgcgaggga

SEQ ID NO: 78 - DNA, 293 bp, Canis lupus familiaris
gaattgcagctggtggagcttgggggagatctggtgaagccaggggggtccctgagactctcctgtgtggcctctggattcaccttcagtagctatgccatgagttgggtctgccaggctccagggaaggggctgcagtgggttgcagctattagcagtagtggaagtagcacataccatgtagacgctgtgaagggccgattcaccatctccagagacaacgccaagaacacagtgtatctgcagatgaacagcctgagagccgaggacacggccgtgtattactgtgcaga

SEQ ID NO: 79 - DNA, 293 bp, Canis lupus familiaris
gaggtgccactggtggaatctgggggagagctggtgaagcctgaggggtccctgagattctcctgtgtagcctctggattcactttcagtagttactggataagctgggtccgccaagctccagggaaagggctgcactgggtctcagtaattaacaaagatggaagtaccacataccacgcagatgctgtgaagggccgattcaccatctccagagacaatgccaagaacacgctgtatctgcagatgaacagcctgagagctgagggcacgactgtgtattactgtgcaca

SEQ ID NO: 80 - DNA, 282 bp, Canis lupus familiaris
gaggagcagttggtgaagtctgggggagacctggtgaagcttggcaggtccctgagtcctctacattcacctttcatagctacagcatgcattggctccaccagtctcccggtagtggctacagtgggtcatatccaatagcagtaatggaagtagcatgtactatgcagacgctgtaaagggttgattcaccatctccagagacaacaccaggaacacgctgtatctgcagatgaacagcctgagagccgacgacacggccgtgtgttgctgtgcgaggga

SEQ ID NO: 81 - DNA, 296 bp, Canis lupus familiaris
gaggtgcagctggtggagtctgggggagaccttgtgaagccggaggggtccctgagactctcctgtgtggccgctggattcacctttagtagctacagcatgagctgggtccgccaggctcccgggaagggggtgcagtgggtcacatagatttatgctagtggaagtagcacaagctacacagatgctgtgaagggccgattcaccatctccagagacaacgccaagaacacagtgtttctgcagatgaacagcctgagagctgagaacacggccatgtattcctgtgcaaggga

SEQ ID NO: 82 - DNA, 296 bp, Canis lupus familiaris
tggggaattccctctggtgtggcctctggattcacctgcagtagctccctcacctccctctcctgtgtggcctctagattcaccttcagtagctactacatatactgtatccaccaagctccagggaaggggctgcaggtggtcgcatggattagctatgatggaagtagaacaagctacgccgacgctatgtagggccaattcatcatctccagagaaaacaccaagaacacgctgtatctgtagatgaacagcctgagtgccaaggacacggcactatatccctgtgcgaggaa

SEQ ID NO: 83 - DNA, 296 bp, Canis lupus familiaris
gaggtgcagctggtggagtctgggggagatctggtgaagcctgggggatccctgagactctcttgtgtggcctctggattcaccttcagtagctactacatggaatgggtccgccaggctccagggaaggggctgcagtgggtcgcacagattagcagtgatggaagtagcacatactacccagacgctgtgaagggtcaattcaccatctccagagacaatgccaagaacacgctgtatctgcagatgaacagcctgggagccgaggacacggccgtgtattactgtgcaaagga

SEQ ID NO: 84 - DNA, 296 bp, Canis lupus familiaris
gaggtgcagctggtggagtctggaggaaacctggtgaagcctggggggtccctgagactctcttgtgtggcctctggattcaccttcagtagctactacatggactgggtccgccaggctccagggaagaggctgcagtgggtcgcagggattagcagtgatggaagtagcacatactacccacaggctgtgaagggccgattcaccatctccagagacaacgccaagaacacgctctatctgcagatgaacagcctgagagccgaggactctgctgtgtattactgtgcgatgga

SEQ ID NO: 85 - DNA, 296 bp, Canis lupus familiaris
gaggtgcagctggtggagtctggaggagacctggtgaagtctggggggtccctgagactctcttgtgtggcctctggattcaccttcagtagctactacatgcactgggtccgccaggctacagggaaggggctgcagtgggtcacaaggattagcaatgatggaagtagcacaaggtacgcagacgccatgaagggccaatttaccatctccagagacaattccaagaatacgctgtatctgcagatgaacagccagagagccgaggacatggccctatattactgtgcaaggga

SEQ ID NO: 86 - DNA, 297 bp, Canis lupus familiaris
gagttgcagctggtagagtctgggggagacctggtgaagcctggggggtctctgagactttcttgtgtgtcctctggattcaccttcagtagctactggatgcactgggtcctccaggctccagggaaagggctggagtgggtcgcaattattaacagtggtggaggtagcatatactacgcagacacagtgaagggccgattcaccatctccagagaaaacgccaagaacacgctctatctgcagatgaacagcctgagagctgaggacagggccatgcattactgtgcgaaggga

SEQ ID NO: 87 - DNA, 290 bp, Canis lupus familiaris
gaactcacactgcaggagtcagggccaggactggtgaagccctcacagaccctctctctcacctgtgttgtgtccggaggctccgtcaccagcagttactactggaactggatccgccagcgccctgggaggggactggaatggatggggtactggacaggtagcacaaactacaacccggcattccagggacgcatctccatcactgctgacacggccaagaaccagttctccctgcagctgagctccatgaccaccgaggacacggccgtgtattactgtgcaagaga

SEQ ID NO: 88 - DNA, 295 bp, Canis lupus familiaris
ctggcacccctgcaggagtctgtttctgggctggggaaacccaggcagatccttacactcacctgctccttctctgggttcttattgagcatgtcagtatgggtgtcacatgggtcctttacccaccaggggaaggcactggagtcaatgccacatctggtgggagaacgctaagtaccacagcctgtctctgaacagcagcaagatgtatagaaagtccaacacttggaaagataaaggattatgtttcacaccagaagcacatctattcaacctgatgaacagccagcctgat

SEQ ID NO: 89 - DNA, 299 bp, Canis lupus familiaris
ctggcacccctgcaggagtctgtttctgggctggggaaacccaggcagacccttacactcacctgctccttctctgggttcttattgagcatgtcagtgtgggtgtcacatgggtcctttacccaccaggggaaggcactggagtcaatgccacgtctggtgggagaacactaagtaccacagcctgtctctgaacagcagcaagatgtatagaaagtccaacacttggaaagataaaggattatgtttcacaccagaagcacatctattcaacctgatgaacaatcagcctgatgaga

SEQ ID NO: 90 - DNA, 31 bp, Canis lupus familiaris
gtactactgtactgatgattactgtttcaac

SEQ ID NO: 91 - DNA, 19 bp, Canis lupus familiaris
ctactacggtagctactac

SEQ ID NO: 92 - DNA, 17 bp, Canis lupus familiaris
tatatatatatggatac

SEQ ID NO: 93 - DNA, 19 bp, Canis lupus familiaris
gtatagtagcagctggtac

SEQ ID NO: 94 - DNA, 19 bp, Canis lupus familiaris
agttctagtagttggggct

SEQ ID NO: 95 - DNA, 11 bp, Canis lupus familiaris
ctaactggggc

SEQ ID NO: 96 - DNA, 53 bp, Canis lupus familiaris
tgacatttactttgacctctggggcccgggcaccctggtcaccatctcctcag

SEQ ID NO: 97 - DNA, 54 bp, Canis lupus familiaris
aacatgattacttagacctctggggccagggcaccctggtcaccgtctcctcag

SEQ ID NO: 98 - DNA, 50 bp, Canis lupus familiaris
caatgcttttggttactggggccagggcaccctggtcactgtctcctcag

SEQ ID NO: 99 - DNA, 48 bp, Canis lupus familiaris
ataattttgactactggggccagggaaccctggtcaccgtctcctcag

SEQ ID NO: 100 - DNA, 51 bp, Canis lupus familiaris
acaactggttctactactggggccaagggaccctggtcactgtgtcctcag

SEQ ID NO: 101 - DNA, 54 bp, Canis lupus familiaris
attactatggtatggactactggggccatggcacctcactcttcgtgtcctcag

SEQ ID NO: 102 - DNA, 302 bp, Canis lupus familiaris
gatattgtcatgacacagacgccaccgtccctgtctgtcagccctagagagacggcctccatctcctgcaaggccagtcagagcctcctgcacagtgatggaaacacctatttggattggtacctgcaaaagccaggccagtctccacagcttctgatctacttggtttccaaccgcttcactggcgtgtcagacaggttcagtggcagcgggtcagggacagatttcaccctgagaatcagcagagtggaggctaacgatactggagtttattactgcgggcaaggtacacagcttcctcc

SEQ ID NO: 103 - DNA, 302 bp, Canis lupus familiaris
gatattgtcatgacacagaccccactgtccctgtccgtcagccctggagagccggcctccatctcctgcaaggccagtcagagcctcctgcacagtaatgggaacacctatttgtattggttccgacagaagccaggccagtctccacagcgtttgatctataaggtctccaacagagaccctggggtcccagacaggttcagtggcagcgggtcagggacagatttcaccctgagaatcagcagagtggaggctgatgatgctggagtttattactgcgggcaaggtatacaagatcctcc

SEQ ID NO: 104 - DNA, 302 bp, Canis lupus familiaris
gatattgtcatgacacagaccccactgtccctgtctgtcagccctggagagactgcctccatctcctgcaaggccagtcagagcctcctgcacagtgatggaaacacgtatttgaactggttccgacagaagccaggccagtctccacagcgtttaatctataaggtctccaacagagaccctggggtcccagacaggttcagtggcagcgggtcagggacagatttcaccctgagaatcagcagagtggaggctgacgatactggagtttattactgcgggcaaggtatacaagatcctcc

SEQ ID NO: 105 - DNA, 302 bp, Canis lupus familiaris
gatattgtcatgacacagaacccactgtccctgtccgtcagccctggagagacggcctccatctcctgcaaggccagtcagagcctcctgcacagtaacgggaacacctatttgaattggttccgacagaagccaggccagtctccacagggcctgatctataaggtctccaacagagaccctggggtcccagacaggttcagtggcagcgggtcagggacagatttcaccctgagaatcagcagagtggaggctgacgatgctggagtttattactgcatgcaaggtatacaagctcctcc

SEQ ID NO: 106 - DNA, 302 bp, Canis lupus familiaris
gatattgtcatgacacagaccccaccgtccctgtccgtcagccctggagagccggcctccatctcctgcaaggccagtcagagcctcctgcacagtaacgggaacacctatttgaattggttccgacagaagccaggccagtctccacagggcctgatctatagggtgtccaaccgctccactggcgtgtcagacaggttcagtggcagcgggtcagggacagatttcaccctgagaatcagcagagtggaggctgacgatgctggagtttattactgcgggcaaggtatacaagatcctcc

SEQ ID NO: 107 - DNA, 302 bp, Canis lupus familiaris
gatattgtcatgacacagaccccactgtccctgtctgtcagccctggagagactgcctccatctcttgcaaggccagtcagagcctcctgcacagtgatggaaacacgtatttgaattggttccgacagaagccaggccagtctccacagcgtttgatctataaggtctccaacagagaccctggggtcccagacaggttcagtggcagcgggtcagggacagatttcaccctgagaatcagcagagtggaggctgacgatactggagtttattactgcgggcaagttatacaagatcctcc

SEQ ID NO: 108 - DNA, 302 bp, Canis lupus familiaris
gatattgtcatgacacagaccccactgtccctgtccgtcagccctggagagactgcctccatctcctgcaaggccagtcagagcctcctgcacagtgatggaaacacgtatttgaattggttccgacagaagccaggccagtctccacagcgtttgatctataaggtctccaacagagaccctggggtcccagacaggttcagtggcagcgggtcagggacagatttcaccctgagaatcagcagagtggaggctgacgatactggagtttattactgcatgcaaggtacacagtttcctcg

SEQ ID NO: 109 - DNA, 302 bp, Canis lupus familiaris
gatatcgtcatgacacagaccccactgtccctgtccgtcagccctggagagactgcctccatctcctgcaaggccagtcagagcctcctgcacagtaacgggaacacctatttgttttggttccgacagaagccaggccagtctccacagcgcctgatcaacttggtttccaacagagaccctggggtcccacacaggttcagtggcagcgggtcagggacagatttcaccctgagaatcagcagagtggaggctgacgatgctggagtttattactgcgggcaaggtatacaagctcctcc

SEQ ID NO: 110 - DNA, 303 bp, Canis lupus familiaris
gatatcgtgatgacccagaccccattgtccttgcctgtcacccctggagagctagcctcatcactgtgcaggaggccagtcagagcctcctgcacagtgatggatatatttatttgaattggtactttcagaaatcaggccagtctccatactcttgatctatatgctttacaaccagacttctggagtcccaggctggttcattggcagtggatcagggacagatttcaccctgaggatcagcagggtggaggctgaagatgctggagtttattattgccaacaaactctacaaaatcctcc

SEQ ID NO: 111 - DNA, 302 bp, Canis lupus familiaris
gatatcgtcatgacgcagaccccactgtccctgtctgtcagccctggagagccggcctccatctcctgcagggccagtcagagcctcctgcacagtaatgggaacacctatttgtattggttccgacagaagccaggccagtctccacagggcctgatctacttggtttccaaccgtttctcttgggtcccagacaggttcagtggcagcgggtcagggacagatttcaccctgagaatcagcagagtggaggctgacgatgctggagtttattactgcgggcaaaatttacagtttccttc

SEQ ID NO: 112 - DNA, 302 bp, Canis lupus familiaris
gaggttgtgatgatacagaccccactgtccctgtctgtcagccctggagagccggcctccatctcctgcagggccagtcagagtctccggcacagtaatggaaacacctatttgtattggtacctgcaaaagccaggccagtctccacagcttctgatcgacttggtttccaaccatttcactggggtgtcagacaggttcagtggcagcgggtctggcacagattttaccctgaggatcagcagggtggaggctgaggatgttggagtttattactgcatgcaaagtacacatgatcctcc

SEQ ID NO: 113 - DNA, 298 bp, Canis lupus familiaris
gatatcatgatgacacagaccccactctccctgcctgccacccctggggaattggctgccatcttctgcagggccagagtctcctgcacaataatggaaacacttatttacactggttcctgcagacatcaggccaggttccaaggcatctgaaccatttggcttccagctgttactctggggtctcagacaggttcagtggcaacgggtcagggacagatttcacactgaaaatcagcagagtggaggctgaggatgttagtgtttattagtgcctgcaagtacacaaccttccatc

SEQ ID NO: 114 - DNA, 302 bp, Canis lupus familiaris
gaggccgtgatgacgcagaccccactgtccctggccgtcacccctggagagctggccactatctcctgcagggccagtcagagtctcctgcgcagtgatggaaaatcctatttgaattggtacctgcagaagccaggccagactcctcggccgctgatttatgaggcttccaagcgtttctctggggtctcagacaggttcagtggcagcgggtcagggacagatttcacccttaaaatcagcagggtggaggctgaggatgttggagtttattactgccagcaaagtctacattttcctcc

SEQ ID NO: 115 - DNA, 302 bp, Canis lupus familiaris
gatatcgtcatgacacagaccccactgtccgtgtctgtcagccctggagagacggcctccatctcctgcagggccagtcagagcctcctgcacagtgatggaaacacctatttggattggtacctgcagaagccaggccagattccaaaggacctgatctatagggtgtccaactgcttcactggggtgtcagacaggttcagtggcagcgggtcagggacagatttcaccctgagaatcagcagagtggaggctgacaacgctggagtttattactgcatgcaaggtatacaagatcctcc

SEQ ID NO: 116 - DNA, 302 bp, Canis lupus familiaris
gatatcgtcatgacacagactccactgtccctgtctgtcagccctggagagacggcctccatctcctgcagggccaatcagagcctcctgcacagtaatgggaacacctatttggattggtacatgcagaagccaggccagtctccacagggcctgatctatagggtgtccaaccacttcactggcgtgtcagacaggttcagtggcagcgggtcagggacagatttcaccctgaagatcagcagagtggaggctgacgatgctggagtttattactgcgggcaaggtacacactctcctcc

SEQ ID NO: 117 - DNA, 291 bp, Canis lupus familiaris
gaaatagtcttgacctagtctccagcctccctggctatttcccaaggggacagagtcaaccatcacctatgggaccagcaccagtaaaagctccagcaacttaacctggtaccaacagaactctggagcttcttctaagctccttgtttacagcacagcaagcctggcttctgggatcccagctggcttcattggcagtggatgtgggaactcttcctctctcacaatcaatggcatggaggctgaaggtgctgcctactattactaccagcagtagggtagctatctgct

SEQ ID NO: 118 - DNA, 286 bp, Canis lupus familiaris
gaaatcgtgatgacacagtctccagcctccctctccttgtctcaggaggaaaaagtcaccatcacctgccgggccagtcagagtgttagcagctacttagcctggtaccagcaaaaacctgggcaggctcccaagctcctcatctatggtacatccaacagggccactggtgtcccatcccggttcagtggcagtgggtctgggacagacttcagcttcaccatcagcagcctggagcctgaagatgttgcagtttattactgtcagcagtataatagcggatata

SEQ ID NO: 119 - DNA, 285 bp, Canis lupus familiaris
gagattgtgccaacctagtctctagccttctaagactccagaagaaaaagtcaccatcagctgctgggcagtcagagtgttagcagctacttagcctggtaccagcaaaaacctggacaggctcccaggctcttcatctatggtgcatccaacagggccactggtgtcccagtccgcttcagcggcagtgggtgtgggacagatttcaccctcatcagcagcagtctggagtcagtctgaagatgttgcaacatattactgccagcagtataatagctacccacc

SEQ ID NO: 120 - DNA, 305 bp, Canis lupus familiaris
gaaatcgtgatgacccagtctccaggctctctggctgggtctgcaggagagagcgtctccatcaactgcaagtccagccagagtcttctgtacagcttcaaccagaagaactacttagcctggtaccagcagaaaccaggagagcgtcctaagctgctcatctacttagcctccagctgggcatctggggtccctgcccgattcagcagcagtggatctgggacagatttcaccctcaccatcaacaacctccaggctgaagatgtgggggattattactgtcagcagcattatagttctcctcc

SEQ ID NO: 121 - DNA, 287 bp, Canis lupus familiaris
gacatcacgatgactcagtgtccaggctccctggctgtgtctccaggtcagcaggtcaccacgaactgcagggccagtcaaagcgttagtggctacttagcctggtacctgcagaaaccaggacagcgtcctaagctgctcatctacttagcctccagctgggcatctggggtccctgcccgattcagcagcagtggatctgggacagatttcaccctcaccgtcaacaacctcgaggctgaagatgtgagggattattactgtcagcagcattatagttctcctct

SEQ ID NO: 122 - DNA, 305 bp, Canis lupus familiaris
gacattatgctgacccagtctccagcctccttgaccatgtgtctccaggagagagggccaccatctcttgcagggccagtcagaaagccagtgatatttggggcattacccaccatattaccttgtaccaacagaaatcagaacagcatcctaaagtcctgattaatgaagcctccagttgggtctggggtcctaggcaggttcagtggctgtgggtctgggactgatttcagcctcacaattgatcctgtggaggctggcgatgctgtcaactattactgccagcagagtaaggagtctcctcc

SEQ ID NO: 123 - DNA, 289 bp, Canis lupus familiaris
gaaattgcagattgtcaaatggataataccaggatgcggtctctagcctccctgactcccaggggagagaaccatcattacccataaaataaatcctgatgacataataagtttgcttggtatcaatagaaaccaggtgagattcctcgagtcctggtatacgacacttccatccttacaggtcccaaactggttcagtggcagtgtctccaagtcagatcttactctcatcatcagcaatgtgggcacacctgatgctgctacttattactgttatgagcattcagga

SEQ ID NO: 124 - DNA, 38 bp, Canis lupus familiaris
gtggacgttcggagcaggaaccaaggtggagctcaaac

SEQ ID NO: 125 - DNA, 39 bp, Canis lupus familiaris
tttatactttcagccagggaaccaagctggagataaaac

SEQ ID NO: 126 - DNA, 38 bp, Canis lupus familiaris
gttcacttttggccaagggaccaaactggagatcaaac

SEQ ID NO: 127 - DNA, 38 bp, Canis lupus familiaris
gcttacgttcggccaagggaccaaggtggagatcaaac

SEQ ID NO: 128 - DNA, 38 bp, Canis lupus familiaris
gatcacctttggcaaagggacacatctggagattaaac

SEQ ID NO: 129 - DNA, 306 bp, Canis lupus familiaris
cagtctgtgctgactcagctggcctcggtgtctggggccctgggccacagggtcagcatctcctggactggaagcagctccaacataagggttgattatcctttgagctgataccaacagctcccagaatgaagaacgaacccaaactcctcatctatggtaacagcaattggctctcaggggttccagatccattctctagaggctccaagtctggcacctcaggctccctgaccaactctggcctccaggctgaggacgaggctgattgttactgcgcagcgtgggacatggatctcagtgctc

SEQ ID NO: 130 - DNA, 294 bp, Canis lupus familiaris
caatctgtgctgactcagctggcctcagtgtctgggtccttgggccagagggtcaccatctcctgctctggaagcacaaatgacattggtattattggtgtgaactggtaccagcagctcccagggaaggcccctaaactcctcatatacgataatgagaagcgaccctcaggtatccccgatcgattctctggctccaagtctggcaactcaggcaccctgaccatcactgggctccaggctgaggacgaggctgattattactgccagtccatggatttcagcctcggtggt

SEQ ID NO: 131 - DNA, 299 bp, Canis lupus familiaris
cagtctgtgctgactcagccagcctccgtgtctgggtccctgggccagagggtcaccatttcctgcactggaagcagctccaacgttggttatagcagtagtgtgggctggtaccagcagttcccaggaacaggccccagaaccatcatctattatgatagtagccgaccctcgggggtccccgatcgattctctggctccaagtctggcagcacagccaccctgaccatctctgggctccaggctgaggatgaggctgattattactgctcatcttgggacaacagtctcaaagctcc

SEQ ID NO: 132 - DNA, 296 bp, Canis lupus familiaris
caggctgtgctgaatcagccggcctcagtgtctggggccctgggccagaaggtcaccatctcctgctctggaagcacaaatgacattgatatatttggtgtgagctggtaccaacagctcccaggaaaggcccctaaactcctcgtggacagtgatggggatcgaccctcaggggtccctgacagattttctggctccagctctggcaactcaggcaccctgaccatcactgggctccaggctgaggacgaggctgattattactgtcagtctgttgattccacgcttggtgctca

SEQ ID NO: 133 - DNA, 294 bp, Canis lupus familiaris
cagtctgtactgactcaatcagcctcagcgtctgggtccttgggccagagggtctccgtctcctgctctagcagcacaaacaacattggtattattggtgtgaagtggtaccagcagatcccaagaaaggcccctaaactcctcatatatgataatgagaagagaccctcaggtgtccccaattgattctctggctccaagtctggcaacttaggcaccctaaccatcaatgggcttcaggctgagggcgaggctgattattactgccagtccatggatttcagcctcggtggt

SEQ ID NO: 134 - DNA, 296 bp, Canis lupus familiaris
cagtctgtgctgactcaaccagcctcagtgtccgggtctctgggccagagggtcaccatctcctgcactggaagcagctccaacattggtagagattatgtgggctggtaccaacagctcccgggaacacgccccagaaccctcatctatggtaatagtaaccgaccctcgggggtccccgatcgattctctggctccaagtcaggcagcacagccaccctgaccatctctgggctccaggctgaggacgaggctgattattactgctctacatgggacaacagtctcactgttcc

SEQ ID NO: 135 - DNA, 296 bp, Canis lupus familiaris
cagtctatgctgactcagccagcctcagtgtctgggtccctgggccagaaggtcaccatctcctgcactggaagcagctccaacatcggtggtaattatgtgggctggtaccaacagctcccaggaataggccctagaaccgtcatctatggtaataattaccgaccttcaggggtccccgatcgattctctggctccaagtcaggcagttcagccaccctgaccatctctgggctccaggctgaggacgaggctgagtattactgctcatcatgggatgatagtctcagaggtca

SEQ ID NO: 136 - DNA, 299 bp, Canis lupus familiaris
caggctgtgctgactcagccgccctcagtgtctgcggtcctgggacagagggtcaccatctcctgcactggaagcagcaccaacattggcagtggttatgatgtacaatggtaccagcagctcccaggaaagtcccctaaaactatcatctatggtaatagcaatcgaccctcaggggtcccggatcgcttctctggctccaagtcaggcagcacagcctctctgaccatcactgggctccaggctgaggacgaggctgattattactgccagtcctctgatgacaacctcgatgatca

SEQ ID NO: 137 - DNA, 296 bp, Canis lupus familiaris
cagtctgtgctgactcagccggcctcagtgtccgggtctctgggccagagagtcaccatctcctgcactggaagcagctccaacatcgatagaaaatatgttggctggtaccaacagctcccgggaacaggccccagaaccgtcatctatgataatagtaaccgaccctcgggggtccctgatcgattctctggctccaagtcaggcagcacagccaccctgaccatctctgggctccaggctgaggacgaggctgattattactgctcaacatacgacagcagtctcagtagtgg

SEQ ID NO: 138 - DNA, 296 bp, Canis lupus familiaris
cagtctgtgctgactcagccggcctcagtgtctgggtccctgggccagagggtcaccatctcctgcactggaagcagctccaacatcagtagatataatgtgaactggtaccaacagctcctgggaacaggccccagaaccctcatctatggtagtagtaaccgaccctcgggggtccccgattgattctctggctccaagtcaggcagcccagctaccctgaccatctctgggctccaggctgaggatgaggctgattattactgctcaacatacgacaggggtctcagtgctcg

SEQ ID NO: 139 - DNA, 296 bp, Canis lupus familiaris
cagcctgtgctgactcagccgccctcagggtctgggggcctgggccagaggttcagcatctcctgttctggaagcacaaacaacatcagtgattattatgtgaactggtactaacagctcccagggacagcccctaaaaccattatctatttggatgataccagaccccctggggtcccggattgattctctgtctccaagtctagcagctcagctaccctgaccatctctgggctccaggctgaggatgaagctgattattactgctcatcctggggtgatagtctcaatgctcc

SEQ ID NO: 140 - DNA, 296 bp, Canis lupus familiaris
cagtctgtgctgactcagccggcctcagtgtctgggtccctgggccagaggatcaccatctcctgcactggaagcagctccaacattggaggtaataatgtgggttggtaccagcagctcccaggaagaggccccagaactgtcatctatagtacaaatagtcgaccctcgggggtgcccgatcgattctctggctccaagtctggcagcacagccaccctgaccatctctgggctccaggctgaggatgaggctgattattactgctcaacgtgggatgatagtctcagtgctcc

SEQ ID NO: 141 - DNA, 296 bp, Canis lupus familiaris
cggtctgtgctgactcagccgccctcagtgtcgggatctgtgggccagagaatcaccatctcccgctctggaagcacaaacagcattggtatacttggtgtgaactggtaccaagagctcccaggaaaggcccctaaactcctcgtagatggtactgggaatagaccctcaggggtccctgaccgattttctggctccaaatctggcaactcaggcactctgaccatcactgggcttcagcctgaggacgaggctgattattattgtcagtccattgaacccatgcttggtgctcc

SEQ ID NO: 142 - DNA, 299 bp, Canis lupus familiaris
caggctgtgctgactccgctgccctcagtgtctgcggccctgggacagacggtcaccatctcttgtactggaaatagcacccaaatcagcagtggttatgctgtacaatggtaccagcagctcccaggaaagtcccctgaaactatcatctatggtgatagcaatcgaccctcgggggtcccagatcgattctctggcttcagctctggcaattcagccacactggccatcactgggctccaggatgaggacgaggctgattattactgccagtccttagatgacaacctcaatggtca

SEQ ID NO: 143 - DNA, 296 bp, Canis lupus familiaris
cagtctgtgctgactcagccggcctcagtgtctgggtccctgggccagagggtcaccatctcctgcactggaagcagctccaacatcggtagatatagtgttggctggttccagcagctcccgggaaaaggccccagaaccgtcatctatagtagtagtaaccgaccctcaggggtccctgatcgattctctggctccaagtcaggcagcacagccaccctgaccatctctgggctccaggctgaggacgaggctgattattactgctcaacatacgacagcagtctcagtagtag

SEQ ID NO: 144 - DNA, 295 bp, Canis lupus familiaris
cagtctgtgctgacatagccaccctcagtgtctggggccctgggccagagggtcaccatctcctgcactggaagcagctcaagcatgggtagttattatgtgagctggcacaagcagctcccaggaacaggccccagaaccatcatgtgttgtaaaaacatcgaccttcgggaatctccaatcaagtctctggctcccattctggcaacacagccaccctgaccatcactgggctcctggctgaggatgaggctgattattactgttcaacatgggatgacaatctcaatgcacc

SEQ ID NO: 145 - DNA, 294 bp, Canis lupus familiaris
cagtctgtgctgactcagctgccctcagtgtctggggccctgggccagagggtcaccatctcctgctctggaagcagctctaaacttggggcttatgctctgaactagaaccaacaattcccaggaacagattccaatttcctcatctatgatgatagtaattgatctttctggatgcctgattaattctgtggctccacatccagcagttcaggctccctgaccatcactgggctctgggatgaggacaaggctgattattactgccagtgccattaccatagcctccgtgct

SEQ ID NO: 146 - DNA, 298 bp, Canis lupus familiaris
cagtctgtgctgactcagccagcctcagtgtctggatccctgggccaaagggtcaccatctcctgcactggaagcacaaacaacatcggtggtgataattatgtgcactggtaccaacagctcccaggaaaggcacccagtctcctcatctatggtgatgataacagagaatctggggtcccggaacgattctctggctccaagtcaggcagctcagccactctgaccatcactgggctccatgctgaggacgaggctgatattattgccagtcctacgatgacagcctcaatactca

SEQ ID NO: 147 - DNA, 296 bp, Canis lupus familiaris
cagtctgtgctgactcagccgccctcagtgtcaggatctgtgggccagagaatcaccatctcctgctctggaagcacaaacagcattggtatacttggtgtgaactggtaccaactgctctcaggaaaggcccctaaactcctcgtagatggtactggaaatcgaccctcaggggtccctgaccgattttctggctccaaatctggcaactcaggcactctgaccatcactgggcttcagcctgaggacgaggctgattattattgtcagtccattgaacccatgcttggtgctcc

SEQ ID NO: 148 - DNA, 296 bp, Canis lupus familiaris
cagtctgtcctgactcagccggcctcagtgtctggggttctgggccagagggtcaccatctcctgcactggaagcagctccaacattggtggaaattatgtgagctggcaccagcaggtcccagaaacaggccccagaaacatcatctatgctgataactaccgagcctcgggggtccctgatcgattctctggctccaagtcaggcagcacagccaccctgaccatctctgtgctccaggctgaggatgaggctgattattactgctcagtgggggatgatagtctcaaagcacc

SEQ ID NO: 149 - DNA, 311 bp, Canis lupus familiaris
cagtccatcctgactcagcagccctcagtctctgggtcactgggccagagggtcaccatctcttgcactggattccctagcaacaatgattatgatgcaatgaaaattcatacttaagtgggctggtaccaacagtccccaggaaagtcacccagtctcctcatttatgatgaaaccagaaactctggggtccctgatcgattctctggctccagaactggtagctcagcctccctgcccatctctggactccaggctgaggacaagactgagtattactgctcagcatgggatgatcgtcttgatgctca

SEQ ID NO: 150 - DNA, 292 bp, Canis lupus familiaris
cagtctgtgctaactcagccaccctcagtgtcggggtcgctgggccagagggtcaccatctcctgctctggaagcacaaacaacatcagtattgttggtgcgagctggtaccaacagctcccaggaaaggcccctaaactcctcgtggacagtgatggggatcgaccgtcaggggtccctgaccgattttctggctctaagtctggcaaatcagccaccctgaccatcactgggcttcaggctgaggacgaggctgattattactgtatattggtcccacgctttgtgctca

SEQ ID NO: 151 - DNA, 285 bp, Canis lupus familiaris
cagtctgtgctgactcagccactgttagggcctggggccctgggcagagggtcaccctctcctgacctggaagagtcccagtattggtgattatggtatgaaatggtacaagcagcttgcaaggacagaccccagactcgtcatctatggcaatagcaattgatcctcgggtccccaatcaattttctggctctggttttggcatcactggctccttgaccacctctgggctccagactgaaaaataggctgattactagtgcttctccagtgatccaggcctgt

SEQ ID NO: 152 - DNA, 299 bp, Canis lupus familiaris
cagtctgtgctgactcaaccggcctccgtgtctgggtccctgggccagagagtcaccatctcttgcactagaagcagctcgaacgttggctatggcaatgatgtgggatggtaccagcagctcccaggaacaggccccagaaccatcatctataataccaatactcgaccctctggggttcctgatcgattctctggctccaaatcaggcagcacagccaccctgaccatctctggactccaggctgaggacgaggctgattattactgctcttcctatgacagcagtctcaatgctca

SEQ ID NO: 153 - DNA, 293 bp, Canis lupus familiaris
cagtctgtgctaactcagccggcctcagtgtctggttccctgggtcagagggtcaccatctgcactggaagcagctccaacattggtacatatagtgtaggctggtaccaacagctcccaggatcaggccccagaaccatcatctatggtagtagtaaccgaccgttgggggtccctgatcgattctctggctccaggtcaggcagcacagccaccctgaccatctctgggctccaggctgaggacgaagctgattattactgcttcacatacgacagtagtctcaaagctcc

SEQ ID NO: 154 - DNA, 296 bp, Canis lupus familiaris
cagtctgtgctgaatcagccaccttcagtgtctggatccctgggccagagaatcaccatctcctgctctggaagcacgaatgacatcggtatgcttggtgtgaactggtaccaacagctcccaggaaatgcccctaaactccttgtagatggtactgggaatcgaccctcaggggtccctgaccaattttctggctccaaatctggcaattcaggcactctgaccatcactgggctccaggctgaggacgaggctgattattattgtcagtcctatgatctcacgcttggtgctcc

SEQ ID NO: 155 - DNA, 280 bp, Canis lupus familiaris
cagtccatgatgactcagccaccctcagtgtctgggtcactgggccagagggtcaccatctactgcactggaatccctagcaacactgattatagtggattggaaatttatacttatgtgagctggtaccaacagtataaggaaaggcacccagtctcctcatctatggggatgataccggaaactctgaggtccctgatcaattctctggctccaggtctggtagctcaacctccctgaccatctctggactccaggctgaggatagtcttaatgctca

SEQ ID NO: 156 - DNA, 296 bp, Canis lupus familiaris
cagtctgtgctgactcagccggcctcagtgactgggtccctgggccagagggtcaccatctcctgcactggaagcagctccaacatcggtggatataatgttggctggttccagcagctcccgggaacaggccccagaaccgtcatctatagtagtagtaaccgaccctcgggggtcccggatcgattctctggctccaggtcaggcagcacagccaccctgaccatctctgggctccaggctgaggacgaggctgagtattactgctcaacatgggacagcagtctcaaagctcc

SEQ ID NO: 157 - DNA, 296 bp, Canis lupus familiaris
cagtctgtgctgactcaaccggcctcagtgtccaggtccctgggccagatagtcaccatctcttgcgctggaagcagctccaacatccgtacaaaatatgtgggctggtactaacagctcccgagaacaggccccagaaccgtcatctatggtaatagtaactgaccctcgggggtcctcgatcaattctctggctccaagtcaggcagcatagccaccctgaccatctctgtgctccaggctgaggacgaggcttattattactgctcaacatatgacagcagtctcagtgctct

SEQ ID NO: 158 - DNA, 298 bp, Canis lupus familiaris
cagtctgtgctgactcaaccggcctctgtgtctggggccctgggccagaggtcaccatctcctgcactaggagcagctccaatgttggttatagcagttatgtgggctggtaccagcagctcccaggaacaggccccaaaaccatcatctataataccaatactcgaccctctggggttcctgatcgattctctggctccaaatcaggcagcacagccacccttaccattgctggactccaggctgaggacgaggctgattattactgctcatcctatgacagcagtctcaaagctcc

SEQ ID NO: 159 - DNA, 272 bp, Canis lupus familiaris
cagtctatgctgactcaccctggccagaggatcaccctctcctgacctggaagagtcccagtattggtgattatggtgtgaaatggtacaggcagctagcaagaacagaccccagactcctcatttatagcaatagcaatcgatccttgagtccccaatcaattttccgcctctggttttgacattactggctccttgaccacctccaggctccagactgaaaaataggctgattactagtgcttatacagtgatccaggcttgtggggctg

SEQ ID NO: 160 - DNA, 296 bp, Canis lupus familiaris
cagtctgtgctgactcagccgacctcagtgtcgtggtccctgggccagagggtcacaatctcatgctctagaagcacgaataacatcggtattgtcggggcgagctggtaccaacagctcccaggaaaggcccctaaactcctcgtggacagtgatggggatcaactgtcaggggtccctgaccgattttctggctccaagtctggcaactcagccaacctgaccatcactgggctccaggctgaggacaaggctgattattactgccagtcctttgatcacacgcttggtgctcg

SEQ ID NO: 161 - DNA, 296 bp, Canis lupus familiaris
cagtctgtgttgagtcagccagcctcagtgtctggggttctgggccagagggtcaccatctcctgcactggaagcagctccaacatcggtggaaattacgtgagctggcaccagcaggtcccagaaacaggccccagaaacatcatctatgctgataactactgagcctcgggggtccctgatggattctctggctccaagtaaggcagcacagccaccccgaccatctctgtgctccaggctgaggatgaggctgattattactgctcagtgggggataatagtctcaaagcacc

SEQ ID NO: 162 - DNA, 293 bp, Canis lupus familiaris
cagtctgtgctgactcagccagcctcagtgtcggggtccctgggccagagagtcaccatctcctgctctggaaggacaaacatcggtaggtttggtgctagctggtaccaacagctcccaggaaaggcccctaaactcctcgtggacagtgatggggatcgaccgtcaggggtccctgaccgattttccggctccaagtctggcaactcggccactctgaccatcactggtctccatgctgaggacgaggctgattattactgtctgtctattggtcccacgcttggtgctca

SEQ ID NO: 163 - DNA, 279 bp, Canis lupus familiaris
cagtctgtgctgactcagccactgttagggcctggggccctggccagaggctcactctctcctgccctggaagagtcccagtattggtgattatgatgtgaagtggtacaggcagctcacaagaacagaccctagactcctcatccatggtgatagcaattgatcctcgggtccccaatcacttttctggctctgtttttggcatcactggctgcttgaccacctctgggctccagactgaaaaataggctgattactagtgcttatccagtgatccag

SEQ ID NO: 164 - DNA, 298 bp, Canis lupus familiaris
cagtctgtgctgactcaaccggcctctgtgtctggggccctgggccagaggtcaccatctcctgcactaggagcagctccaatgttggttatagcagttatgtgggctggtaccagcagctcccaggaacaggccccaaaaccatcatctataataccaatactcgaccctctggggtccctgatcgattctctggctccaaatcaggcaggacagccacccttaccattgctggactccaggctgaggacgaggctgattattactgctcatcctatgacagcagtctcaaagctcc

SEQ ID NO: 165 - DNA, 299 bp, Canis lupus familiaris
caggctgtgctgactcagccggcctcagtgtctgggtccctgggccagagggtcaccatctcctgcactggaagcagctccaatgttggttatggcaattatgtgggctggtaccagcagctcccaggaacaggccccagaaccctcatctatggtagtagttaccgaccctcgggggtccctgatcgattctctggctccagttcaggcagctcagccacactgaccatctctgggctccaggctgaggatgaagctgattattactgctcatcctatgacagcagtctcagtggtgg

SEQ ID NO: 166 - DNA, 296 bp, Canis lupus familiaris
cagtctgtgctgactcagccagcctcagcgtctgggtccttgggccagagggtcactgtctcctgctctagcagcacaaacaacatcggtattattggtgtgaagtggtaccagcagatcccaggaaaggcccataaactcctcatatatgataatgagaagcgaccctcaggtgtccccaatcgattctctggctccaagtctggcgacttaagcaccctgaccatcaatgggcttcagggtgaggacgaggctgattattattgccagtccatggatttcagcctcggtggtca

SEQ ID NO: 167 - DNA, 314 bp, Canis lupus familiaris
cagtctgtgctgactcagccagcctcagtgtctgggtccctgggccagagggtcaccatctcctgcactggaatccccagcaacacagattttgatggaatagaatttgatacttctgtgagctggtaccaacagctcccagaaaagccccctaaaaccatcatctatggtagtactctttcattctcgggggtccccgatcgattctctggctccaggtctggcagcacagccaccctgaccatctctgggctccaggctgaggacgaggctgattattactgctcatcctgggatgatagtctcaaatcata

SEQ ID NO: 168 - DNA, 299 bp, Canis lupus familiaris
cagtctgtgctgactcagccagcctcagtgtctggatccctgggccaaagggtcaccatctcctgcactggaagcacaaacaacatcggtggtgataattatgtgcactggtaccaacagctcccaggaaaggcacccagtctcctcatctatggtgatgataacagagaatctggggtccctgaacgattctctggctccaagtcaggcagctcagccactctgaccatcactgggctccaggctgaggacgaggctgattattattgccagtcctacgatgacagcctcaatactca

SEQ ID NO: 169 - DNA, 289 bp, Canis lupus familiaris
cagtctgtgctgactcagccgccctcagtgtcgggatctgtgggccagagaatcaccatctcctgctctggaagcacaaacagctaccaacagctctcaggaaaggcctctaaactcctcgtagatggtactgggaaccgaccctcaggggtccccgaccgattttctggctccaaatctggcaactcaggcactctgaccatcactgggcttgggacgaggctgaggacgaggctgaggacgaggctgattattattgttagtccactgatctcacgcttggtgctcc

SEQ ID NO: 170 - DNA, 292 bp, Canis lupus familiaris
caggccgccctgggcaatgagttcgtgcaggtcaaggctgagacagacctgcagaattcaggtttgtctgagacacagctcatcagatgtgtgcagtgtgtgtcctggtaccaacggctcccatgaatgggtcctaaatccttatctagaaataacatttagatcactttgtggcccggatccattctctggctccatgtctggcaactctggcctcatgaacatcactgggctatggtctgaagatggagctgctcttcacaggccctcttgggacaaaattcttggggct

SEQ ID NO: 171 - DNA, 309 bp, Canis lupus familiaris
cagtccatcctgactcagccgccctcagtctctgggtcactgggccagagggtcaccatctcctgcaatggaatccctgacagcaatgattatgatgcatgaaaattcatacttacgtgagctggtaccaacagttcccaagaaagtcaccagtctcctcatctacgatgataccagaaactctggggaccctgatcaattctctggctccagatctggtaactcagcctccctgcccatctctggactccaggctgaggacgaggctgagtattactgctcagcatgggatgatcgtcttgatgctca

SEQ ID NO: 172 - DNA, 296 bp, Canis lupus familiaris
cagtctgtactgactcagccggcctcagtgtctgggtccctgggccagagggtcaccatctcctgcactggaagcagctccaacatcggtggatattatgtgagctggctctagcagctcccgggaacaggccccagaaccatcatctatagtagtagtaaccgaccttcaggggtccctgatcgattctctggctccaggtcaggcagcacagccaccctgaccatctctgggctccaggctgaggatgaggctgattattactgttcaacatacgacagcagtctcaaagctcc

SEQ ID NO: 173 - DNA, 296 bp, Canis lupus familiaris
cttcctgtgctgacccagccaccctcaaggtctgggggtctggttcagaagatcaccatcttctgttctggaagcacaaacaacatgggtgataattatgttaactggtacaaacagcttccaggaacggcccctaaaaccatcatctaagtggatcatatcagaccctcaggggtcctggagagattctctgtctccaattctggcagctcagccaacctgaccatctctgggctccaggatgaggactaggctgattattattgctcatcctggcatgatagtctcagtgctcc

SEQ ID NO: 174 - DNA, 295 bp, Canis lupus familiaris
caggctgtgctgactcagctgccctcagtgtctgcagccctgggacagagggtcaccatctgcactggaagcagcaccaacatcggcagtggttattatacactatggtaccagcagctgcaggaaagtcccctaaaactatcatctatggtaatagcaatcgacccttgagggtcccggatcgattctctggctccaagtatggcaattcagccacgctgaccatcactgggctccaggctgaggacgaggatgattattactgccagtcctctgatgacaacctcgatggtca

SEQ ID NO: 175 - DNA, 299 bp, Canis lupus familiaris
cagtctgtgctgactcagccggcctcggtgtctgggtccctgggccagagggtcaccatctcctgcactggaagcagctccaatgttggttatggcaattatgtgggctggtaccagcagcttccaggaacaggccccagaaccattatctgttataccaatactcgaccctctggggttcctgatcgatactctggctccaagtcaggcagcacagccaccctgaccatctctgggctccaggctgaagacgagactgattattactgtactacgtgtgacagcagtctcaatgctag

SEQ ID NO: 176 - DNA, 296 bp, Canis lupus familiaris
cagtctgtgctgactcagcctccctcagtgtccgggttcctgggccagagggtcaccatctcctgcactggaagcagctccaacatcggtagaggttatgtgcactggtaccaacagctcccaggaacaggccccagaaccctcatctatggtattagtaaccgaccctcaggggtccccgatcgattctctggctccaggtcaggcagcacagccactctgacaatctctgggctccaggctgaggatgaggctgattattactgctcatcctgggacagcagtctcagtgctct

SEQ ID NO: 177 - DNA, 285 bp, Canis lupus familiaris
cagtctgtgctgactcagccactgttagggcctgggttcctggccagagggtcaccctctcctgccctggaagagtctcagttttggtgattatggtgtgaaacggtacaggaagctcgcatggacagaccccagactcctcatctatggcaatagcaattgattctcgggtccccagtctattttctggctctggttttggcatcactggctccttgaccacctccgggctccagactgaaaaataggctgatttctagtgcttctccagtgatccaggccttt

SEQ ID NO: 178 - DNA, 299 bp, Canis lupus familiaris
cagtctgcgctgactcaaacggcctccatgtctgggtctctgggccagagggtcaccgtctcctgcactggaagcagttccaacgttggttatagaagttatgtgggctggtaccagcagctcccaggaacaggccccagaaccatcatctataataccaatactcgaccctctggggttcctgatcgattctctggctccatatcaggcagcacagccaccctgactattgctggactccaggctgaggacgaggctgattattactgctcatcctatgacagcagtctcaaagctcc

SEQ ID NO: 179 - DNA, 294 bp, Canis lupus familiaris
cagtctgtgctgaatcagctgccttcagtgttaggatccctgggccagagaatcaccatctcctgctctggaagcacgaatgacatcggtatgcttggtgtgaactggtaccaagagccgccaggaaaggcccctaaactcctcgtagatggtactgggaatcgaccctcagggtccctgccgattttctggctccaaatctggcaactcaggcactctgaccatcactgggctccaggctgaggacgaggctgattattattgtcagtccactgatctcacgcttggtgctcc

SEQ ID NO: 180 - DNA, 296 bp, Canis lupus familiaris
cagtctgtgctgactcagcctccctcagtgttcaggtccctgggccagagggtcactatatcctgcactggaagcagctccaacgtcggtagaggttatgtgatctggtaccaacagctcctgggaacacgcccaagaaccctcatatatggtagtagtaaccaaccctcaggggtccccaatcaattctctggctccaggtcaggcagcacagacactctgacaatctctgggttccaggctgaggatgaggctgattattactgctcatcctgggacagcagtctcagtgctct

SEQ ID NO: 181 - DNA, 299 bp, Canis lupus familiaris
cagtctgtgctgactcaaccagtctcagtgtctggggccctgtgccagagggtcaccatctcctgcactggaaacagctccaacattggttatagcagttgtgtgagctgatatcagcagctcccaggaacaggccccagaaccatcatctatagtatgaatactcaaccctctggggttcctgatcgattctctggctccaggtcaggcaactcagccaccctaaccatctctgggctccaggctgaggacaaggctgactattactgctcaacatatgacagcagtctcagtgctca

SEQ ID NO: 182 - DNA, 296 bp, Canis lupus familiaris
cagtctgtgctgactcagccgacctcagtgtcggggtcccttggccagagggtcaccatctcctgctctggaagcacgaacaacatcggtattgttggtgcgagctggtaccaacagctcccaggaaaggcccctaaactcctcgtggacagtgatggggatcgaccgtcaggggtccctgaccggttttccggctccaagtctggcaactcagccaccctgaccatcactgggcttcaggctgaggacgaggctgattattactgccagtcctttgataccacgcttgatgctca

SEQ ID NO: 183 - DNA, 290 bp, Canis lupus familiaris
cagtctgtactgactcagcagccgttagtgcttggggccctggccagagggtcagcttctcctgccttggaagagtcccagtattggtaattatggtgtgaaatggtacaagcagctcaaaaggacagaccccagacttctcatctatggcaatagcaattgatcctcgggtccccaatcaattttctggctctggttttggcatcactggctccttgaccacctatgggctccagactgaaaaataggctgattactagtgcttttccagtgatccagtcctgaggggc

SEQ ID NO: 184 - DNA, 298 bp, Canis lupus familiaris
cagtctgtgctgactcaaccggcctccgtgtctggggccttgggccagagggtcaccatctcctgcactggaagcagctccaatgttggttatagcagctatgtgggcttgtaccagcagctcccaggaacaggcctcaaaaccatcatctataataccaatactcgaccctctggggttcctgatcaattctctggctccaaatcaggcagcacagccacctgaccattgctggacttcaggctgaggacgaggctgattattactgctcatcctatgacagcagtctcaaagctcc

SEQ ID NO: 185 - DNA, 299 bp, Canis lupus familiaris
caggctgtgctgactcagccaccctctgtgtctgcagccctggggcagagggtcaccatctcctgcactggaagtaacaccaacatcggcagtggttatgatgtacaatggtaccagcagctcccaggaaagtcccctaaaactatcatttatggtaatagcaatcgaccctcgggggtcccggttcgattctctggctccaagtcaggcagcacagccaccctgaccatcactgggatccaggctgaggatgaggctgattattactgccagtcctatgatgacaacctcgatggtca

SEQ ID NO: 186 - DNA, 293 bp, Canis lupus familiaris
cagtctgtgctgactcagccagcttcagtgtctgggtccctgggccagaggatcaccatctcctgcactaaaagcagctccaacatcggtaggtattatgtgagctgacaacagctcccaggaacaggccccagaaccgtcatctatgataataataactgaccctcgggggtccctgatcaattttctggctctaaatcaggcagcacagccaccctgaccatctctaggctccaggctgaggacgatgctgattattactgctcgccatatgccagcagtctcagtgctgg

SEQ ID NO: 187 - DNA, 296 bp, Canis lupus familiaris
cagtctgtgttgactcaaccggcctcagtgtctgggtccctgggccagagggtcatcatctcctgcactggaagcagctccagcattggcagaggttatgtgggctggtaccaacagctcccaggaacaggccccagaaccctcatctatggtattagtaacctacccccgggagtccccaatagattctctggttcgaggtcaggcagcacagccaccctgaccatcgctgagctccaggctgaggacgaggctgattattactgctcatcgtgggacagaagtctcagtgctcc

SEQ ID NO: 188 - DNA, 297 bp, Canis lupus familiaris
caggctgtgctgactcagcccgccctcagtgtctgcggccttgggacagagggtcaccatctcctgcactggaagcagcaccaacatcagcagtggttacgttgtacaatggtaccagcagctcccaggaaagtcccctaaaacaatctatggtactagcaagtgacccttggggatcccggttcaattctctggctccaagtcaggcagcacagccaccctgaccatcactggtatctaggctgaggacgaggctgattattactgccaatcctatgatgacaacctcgatggtca

SEQ ID NO: 189 - DNA, 300 bp, Canis lupus familiaris
caggctgtacggaatcaaccgccctcagagtctgcagccctgggacagagagtcaccatctcctgcacgggaagcagatccaacattggcagtggttatgctgtacaatggtaccaacggctcacaggaaagtctccttaaaactatcatctatggtaatagcaatcaaccctcgggggtcctggatcaattctctggctccaagtgaggcagcacagccaccctgaccatcactgggatccagtctgaggacgaggctgattattactgccagtcctatgatagaagtctctgtgctca

SEQ ID NO: 190 - DNA, 296 bp, Canis lupus familiaris
cagtctgtgctgactcagccggcctcagtgtctgggtccctgggcctgagggtcaccatctgctgcactggaagcagctccaacatcagtagttattatgtgggctggtaccaaccactcgcgggaacaggccccagaactgtcatctatgataatagtaaccgtccctcgggggtccctgatcaattctctggctccaagtcaggcagcacagccaccctgaccatctctcggctccaggctgaggacgaggctgattattacggctcatcatatgacagcagtctcaatgctgg

SEQ ID NO: 191 - DNA, 299 bp, Canis lupus familiaris
cagtctgtgctgactcagccagcctcagtgtctcagtccctgggtcagagggtcaccatctcctgtactggaagcagctccaatgttggttataacagttatgtgagctggtaccagcagctcccaggaacagtccccagaaccatcatctattataccaatactcgaccctatggggttcctgatcgattctctggctccaaatcaggcaactcagccaccctgaccattgctggactccaggctgaggacgaggctgattattattgctcaacatatgacagcagtctcagtggtgc

SEQ ID NO: 192 - DNA, 293 bp, Canis lupus familiaris
cagtctgtgctgaatcagacgccctcagtgtcggggtccctgggccagagagtcgccatctcctgctctggaagcacaaacatcagtaggtttggtgcgagctggtaacaacagctcctgggaaaggcttcaaaactcctcctagacagtgatggggatcaaccatcagtggtccctgactgattttccggctccaagtctggcaactcaggtgccctgaccatcactgggctccaggctgaggacgaggctgattattactgccagtcctttgatcccacacttggtgctca

SEQ ID NO: 193 - DNA, 293 bp, Canis lupus familiaris
caggctttgctgactcagccaccctcagtgtctgaggccctgggacagagggtcaccatctcctgcactggaagcagcaccaacatcggcagtggttatgatgtacaatggtaccagcagctcccaggaaagtcccctcaaactatcgtatacggtaatagcaattgaccctcgggggtcccagatcaattctctggctccaagtctcacaattcagccaccctgaccatcactgggctccagactgaggacgaggctgattattactgccagtcctctgatgacaacctcga

SEQ ID NO: 194 - DNA, 296 bp, Canis lupus familiaris
cagtctgtgctgactcagccagcctcagtgtctgggtccctgggccagagggtcaccatctcctgcactggaagcagctccaacatcggtagatatagtgtaggctgataccagcagctcccgggaacaggccccagaactgtcatctatggtagtagtagccgaccctcgggggtccccgatcgattctctggctccaagtcaggcagcacagccaccctgaccatctcagggctccaggctgaggacgaggctgattattactgttcaacatacgacagcagtctcaaagctcc

SEQ ID NO: 195 - DNA, 296 bp, Canis lupus familiaris
cagcctgtgctcactcagccgccctcagtgtctgggttcctgggacagagggtcactatctcctgcactggaagcagctccaacatccttggtaattctgtgaactggtaccagcagctcacaggaagaggccccagaaccgtcatctattatgataacaaccgaccctctggggtccctgatcaattctctggctccaagtcaggcaactcagccaccctgaccatctctgggctccaggctgaggacgagactgattattactgctcaacgtgggacagcaggctcagagctcc

SEQ ID NO: 196 - DNA, 296 bp, Canis lupus familiaris
cagtctgtgctgactcagccggcctcagtgtctgggtccctgggccagagggtcaccatctcctgcactgaaagcagctccaacatcggtggatattatgtgggctggtaccaacagctcccaggaacaggccccagaaccatcatctatagtagtagtaaccgaccctcaggggtccctgattgattctctggctccaggtcaggcagcacagccaccctgaccatctctgggctccaggctgaggacgaggctgattattactgctctacatgggacagcagtctcaaagctcc

SEQ ID NO: 197 - DNA, 296 bp, Canis lupus familiaris
ctgcctgtgctgacccagccgccctcaaggtctgggggtctggttcagaggttcaccatcttctgttctggaagcacaaacaacataggtgataattattttaactggtacaaacagcttccaggaacggcccctaaaaccatcatctaagtggatcatatcagaccctcaggggtcctggagagattctctgtctccaattctggcagctcagccaacctgaccatctctgggctccaggctgaggactaggctgattattattgctcatcctgggatgatagtctcaatgctcc

SEQ ID NO: 198 - DNA, 295 bp, Canis lupus familiaris
caggctgtgctgactcagctgccctcagtgtctgcagccctgggacagagggtcaccatctgcactggaagcagcaccaacatcggcagtggttattatacactatggtaccagtagctgcaggaaagtcccctaaaactatcatctatggtaatagcaatcgacccttgagggtcccggatcgattctctggctccaagtatggcaattcagccacgctgaccatcactgggctccaggctgaggacgaggatgattattactgccagtcctctgatgacaacctcgatggtca

SEQ ID NO: 199 - DNA, 292 bp, Canis lupus familiaris
cagtctgtgctgactcagccggcctcagtgtctgggtccctgggtcagagggtcaccatctcctgcactggaagcagctccaacatcggtgaatattatgtgagttggctccagcagctcccgggaacacgccccagaaccgtcatctatagtagtagtaaccgaccctcaggggtccctgatcgattctctggctccaagtcaggtagcatagccaccctatctctgggctccaggctgaagacgaggctgattattactgtactacgtgggacagcagtctcaatgctgg

SEQ ID NO: 200 - DNA, 296 bp, Canis lupus familiaris
cagtctgtgctgactcagccggcctcagtgtccgggtccctgggccagagggtcaccatctcctgcactggaagcagctccaacatcggtagaggttatgtgggctggtaccaacagctcccgggaacaggccccagaaccctcatctatggtaatagtaaccgaccctcaggggtccccgatcggttctctggctccaggtcaggcagcacagccaccctgaccatctctgggctccaggctgaggatgaggctgattattactgctcatcgtgggacagcagtctcagtgctct

SEQ ID NO: 201 - DNA, 295 bp, Canis lupus familiaris
cagtctgtgctgactcagcctccctcagtgtctgggtccctgggccagaggtcaccgtctcctgcactggaagctgcttcaacattggtagatatagtgtgagctggctccagcagctcccgggaacaggccccagaaccatcatctattatgatcgtagccgaccctcaggggttcccgatcgattctctggctccaagtcaggcagcacagccaccctgaccatctctgggctccaggctgaggacgaggctgattattactgctcatcctatgacagcagtctcaaaggtca

SEQ ID NO: 202 - DNA, 299 bp, Canis lupus familiaris
cagtctgtgctgactcaaccagtctcagtgtctggggccctgtgccagagggtcaccatctcctgcactggaagcagctccaacattggttatagcagctgtgtgagctgatatcagcagctcccaggaacaggccccagaaccatcatctatagtatgaatactctaccctctggggttcctgatcgattgtctggctccaggtcaggcaactcagccaccctaaccatctctgggctccaggctgaggacaaggctgactattactgctcaacatatgacagcagtctcaatgctca

SEQ ID NO: 203 - DNA, 296 bp, Canis lupus familiaris
cagtctgtgctgacccagctggcctcagtgtctgggtccctgggccagagggtcaccatcacctgcactggaagcagctccaacattggtagtgattatgtgggctggttccaacagctcccaggaacaggccctagaaccctcatctaaggcaatagtaaccgaccctcgggggtccctgatcaattctctggctccaagtctggcagtacagccaccctgaccatctctgggctccaggctgaggatgatgctgattattactgcacatcatgggatagcagtctcaaggctcc

SEQ ID NO: 204 - DNA, 312 bp, Canis lupus familiaris
cagtctgtgctgactcagcctccctcagtgtctgggaccctggggcaaagggtcatcatctcctgcactggaatccccagcaacataaatttagaagaattgggaatcgctactaaggtgaactggtaccaacagctcccaggaaaggcacccagtctcctcatctatgatgatgatagcagaggttctgggattcctgatcgattctctggctccaagtctggcaactcaggcaccctgaccatcactgggctccaggctgaggatgaggctgattattattgccaatcctatgatgaaagccttggtgtt

SEQ ID NO: 205 - DNA, 295 bp, Canis lupus familiaris
cagtctgtgctgactcagcctccctcagtgttcaggtccctgggccagagggtcaccatctcctgcactggaagcagctccaacgtcggtagaggttatgtgatctggtaccaaagctcctgggaacacgcccaagaaccctcatatatggtagtagtaaccaaccctcaggggtccccaatcgattctctggctccaggtcaggcagcacagacactctgacaatctctgtgttccaggctgaggatgaggctgattattactgctcatcctgggacagcagtctcagtgctct

SEQ ID NO: 206 - DNA, 296 bp, Canis lupus familiaris
cagtctgtgctgaatcagctgccttcagtgttaggatccctgggccagagaatcaccatctcctgctctggaagcacgaatgacatcggtatgcttggtgtgaactggtaccaagagctcccaggaaaggcccctaaactcctcgtagatggtactgggaatcgaccctcaggggtccctgaccgattttctggctccaaatctggcaactcaggcactctgaccatcactgggctccaggctgaggacgaggctgattattattgtcagtccactgatctcacgcttggtgctcc

SEQ ID NO: 207 - DNA, 296 bp, Canis lupus familiaris
cagtctgtgctgactcagccggcctcagtgtctgggtccctgggccagagggtcaccatctcctgcactggaagcagctccaacatcggtagaggttatgtgggctggtaccagcagctcccaggaacaggccccagaaccctcatctatgatagtagtagccgaccctcgggggtccctgatcgattctctggctccaggtcaggcagcacagcaaccctgaccatctctgggctccaggctgaggacgaggctgattattactgctcagcatatgacagcagtctcagtggtgg

SEQ ID NO: 208 - DNA, 299 bp, Canis lupus familiaris
cagtctgtgctgactcagccggcctcagtgtctgggtccctgggccagagggtcaccatctcctgcactggaagcagctccaatgttggttatggcaattatgtgggctggtaccagcagctcccaggaacaagccccagaaccctcatctatgatagtagtagccgaccctcgggggtccctgatcgattctctggctccaggtcaggcagcacagcaaccctgaccatctctgggctccaggctgaggatgaagccgattattactgctcatcctatgacagcagtctcagtggtgg

SEQ ID NO: 209 - DNA, 299 bp, Canis lupus familiaris; identical to SEQ ID NO: 142

SEQ ID NO: 210 - DNA, 298 bp, Canis lupus familiaris
cagtctgtgctgactcaaccggcctccgtgtctggggacttgggccagagggtcaccatctcctgcactggaagcagctccaattttggttatagcagctatgtgggcttgtaccagcagctcccaggaacaggccccagaaccatcatctataataccaatactcgaccctctggggttcctgatcgattctctggctccaaatcaggcagcacagccacctgaccattgctggacttcaagctgaggacgaggctgattattactgctcatcctatgacagcagtctcaaagctcc

SEQ ID NO: 211 - DNA, 279 bp, Canis lupus familiaris
cagtctgtactgactcagccgccattagtgcttggggccctggccagagggtcaccttctcctgccttggaagagtcccagtattggtgattatggtgtgaaatggtacaagcagctcaaaaggacagaccccagacttctcatctatggcaatagcaattgatcctcgggtccccaatcaattttctggctctggttttggcatcactggctccttgaccacctatgggctccagactgaaaaataggctgattactagtgcttctccggtgatccag

SEQ ID NO: 212 - DNA, 296 bp, Canis lupus familiaris
cagtctgtgctgactcagccgacctcagtgtcggggtcccttggccagagggtcaccatctcctgctctggaagcacgaacaacatcggtattgttggtgcgagctggtaccaacagctcccaggaaaggcccctaaactcctcgtgtacagtgttggggatcgaccgtcaggggtccctgaccggttttccggctccaactctggcaactcagccaccctgaccatcactgggcttcaggctgaggacgaggctgattattactgccagtcctttgataccacgcttggtgctca

SEQ ID NO: 213 - DNA, 299 bp, Canis lupus familiaris; identical to SEQ ID NO: 202

SEQ ID NO: 214 - DNA, 296 bp, Canis lupus familiaris
cagtctgtgctgactcagcctccctcagtgttcaggtccctgggccagagggtcaccatctcctgcactggaagcagctgcaacgtcggtagaggttatgtgatctggtaccaacagctcctgggaacacgcccaagaaccctcatatatggtagtagtaaccaaccctcaggggtccccaatcgattctctggctccaggtcaggcagcacagccactctgacaatctctgggttccaggctgaggatgaggctgattattactgctcatcctgggacagcagtctcagtgctct

SEQ ID NO: 215 - DNA, 296 bp, Canis lupus familiaris
cagtctgtgctgaatcagctgccttcagtgttaggatccctgggccagagaatcaccatctcctgctctggaagcacgaatgacatcggtatgcttggtgtgaactggtaccaagagctcccaggaaaggcccctaaactcctcgtagatggtactgggaatcgaccctcaggggtccctgactgattttctggctccaaatctggcaactcaggcactctgaccatcactgggctccaggctgaggacgaggctgattattattgtcagtccactgatctcacgcttggtgctcc

SEQ ID NO: 216 - DNA, 296 bp, Canis lupus familiaris
cagtctgtgctgactcagccggcctcagtgtctgggtccctgggccagagggtcaccatctcctgcactggaagcagctccaacatcggtagaggttatgtgggctggtaccagcagctcccaggaacaggccccagaaccctcatctatgataatagtaaccgaccctcgggggtccctgatcgattctctggctccaagtcaggcagcacagccaccctgaccatctctgggctccaggctgaggacgaggctgattattactgctcaacatacgacagcagtctcagtggtgg

SEQ ID NO: 217 - DNA, 299 bp, Canis lupus familiaris
cagtctgtgctgactcagccggcctcagtgtctgggtccctgggccagagggtcaccatctcctgcactggaagcagctccaatgttggttatggcaattatgtgggctggtaccagcagctcccaggaacaggccccagaaccctcatctatcgtagtagtagccgaccctcgggggtccctgatcgattctctggctccaggtcaggcagcacagcaaccctgaccatctctgggctccaggctgaggatgaagccgattattactgctcatcctatgacagcagtctcagtggtgg

SEQ ID NO: 218 - DNA, 299 bp, Canis lupus familiaris
caggctgtgctgactccgctgccctcagtgtctgcggccctgggacagacggtcaccatctcttgtactggaaatagcacccaaatcggcagtggttatgctgtacaatggtaccagcagctcccaggaaagtcccctgaaactatcatctatggtgatagcaatcgaccctcgggggtcccagatcgattctctggcttcagctctggcaattcagccacactggccatcactgggctccaggatgaggacgaggctgattattactgccagtccttagatgacaacctcgatggtca

SEQ ID NO: 219 - DNA, 299 bp, Canis lupus familiaris; identical to SEQ ID NO: 178

SEQ ID NO: 220 - DNA, 266 bp, Canis lupus familiaris
cagtctgtgctgactcagccactgttagggcctgggttcctggccagagggtcaccctctcctgccctggaagagtctcagttttggtgattatggtgtgaaacggtacaggaagctcgcatggacagaccccagactcctcatctatggcaatagcaattgattctcgggtccccagtctattttctggctctggttttggcatcactggctccttgaccacctccgggctccagactgaaaaataggctgatttctagtgcttc

SEQ ID NO: 221 - DNA, 296 bp, Canis lupus familiaris
caatctgtgctgatccagccggcctcagtgtcgggatccctgggccagagagtcaccatctcctgctctggaaggacaaacaacatcggtaggtttggtgcgagctggtaccaacagctcccaggaaaggcccctaaactcctcgtggacagtgatggggattgaccgtcaggggtccctgaccggttttccggctccaggtctggcagctcagccaccctgaccatcactggggtccaggctgaggatgaggctgattattactgccagtcctttgatcccacgcttggtgctca

SEQ ID NO: 222 - DNA, 309 bp, Canis lupus familiaris
cagtctgtgctgactcaaccgtcctcagtgtccgggtccctgggccagagggtcactgtcccctgcactggaagcagctccaacattggtagatatagtgtgagctggctatatctgctggctccagcagctcccgggaacaggccccagaaccatcatctattatgattgtagccgaccctcaggggttcccgatcgattctctggctccaagtcaggcagcacagccaccctgaccatctctgggctccaggctgaggacgaggctgattattactgctcatcctatgacagcagtctcaaaggtca

SEQ ID NO: 223 - DNA, 296 bp, Canis lupus familiaris; identical to SEQ ID NO: 176

SEQ ID NO: 224 - DNA, 296 bp, Canis lupus familiaris; identical to SEQ ID NO: 216

SEQ ID NO: 225 - DNA, 296 bp, Canis lupus familiaris; identical to SEQ ID NO: 206

SEQ ID NO: 226 - DNA, 296 bp, Canis lupus familiaris; identical to SEQ ID NO: 214

SEQ ID NO: 227 - DNA, 299 bp, Canis lupus familiaris; identical to SEQ ID NO: 202

SEQ ID NO: 228 - DNA, 292 bp, Canis lupus familiaris
caaggtcagctgccctgaggacagagtccatgacaggtcagggcagaaacagggactctgaatccagctctgagtcaggacacatcaggagtgtccaatatgtgtcctgctaccaacagctccatgagtgggcagtcaaatcctcatgtattatgatggcttgaccttctgtggaccctggtccattctctgcctccatgtctggcagctctggctctctggccattgctgggctgagccaggaggatgaggtcatgcttcactgcccctccagtgacagcatttcaaggat

SEQ ID NO: 229 - DNA, 296 bp, Canis lupus familiaris
cagtctgtgctgactcagccgacctcagtgtcggggtcccttggccagagggtcaccatctcctgctctggaagcacgaacaacatcggtattgttggtgcgagctggtaccaacagctcccaggaaaggcccctaaactcctcgtgtacagtgatggggatcgaccgtcaggggtccctgaccggttttccggctccaactctggcaactcagacaccctgaccatcactgggcttcaggctgaggacgaggctgattattactgccagtcctttgataccacgcttgatgctca

SEQ ID NO: 230 - DNA, 297 bp, Canis lupus familiaris
cagtctgccctgactcaaccttcctcggtgtctgggactttgggccagactgtcaccatctcctgtgatggaagcagcagtaacattggcagtagtaattatatcgaatggtaccaacagttcccaggcacctcccccaaactcctgatttactataccaataatcggccatcagggatccctgctcgcttctctggctccaagtctgggaacacggcctccttgaccatctctgggctccaggctgaagatgaggctgattattactgcagcgcatatactggtagtaatactttc

SEQ ID NO: 231 - DNA, 297 bp, Canis lupus familiaris
cagtctaacctaattgagcccccctttttgtccaggattctaggatggactgtcactgtctcctgtgttttaagcagctgtgacatcaggagtgataatgaaatatcctggtaccaatagcacccgagcatgactcagaaattcctgatttactataccagttcttgggcatcagatatccctgattgctttcctggctcccagtctggaaacatggcctgtctgaccatttccaggctccaggctaatgatgacgctgattatcattgttacttatatgatggtagtggcgctttt

SEQ ID NO: 232 - DNA, 296 bp, Canis lupus familiaris
cagtctgccctgactcagcctccctcgatgtctgggacactgggacagaccatcatcatttcctgtactggaagcggcagtgacattgggaggtatagttatgtctcctggtaccaagagctcccaagcacgtcccccacactcctgatttatggtaccaataatcggccattagagatccctgctcgcttctctggctccaagtctggaaacacagcccccatgaccatctctgggcttcaggctgaagatgaggctaattattactgttgctcatatacaaccagtggcacaca

SEQ ID NO: 233 - DNA, 286 bp, Canis lupus familiaris
cagtctgccttgacccaacctccctttgtgtctgggactttgagacaaactgtcacatctcttgcaatggaagcagcagccacactggaacttataaccctacctctggcaccagcaatgtctggaaaggcccccacactccagatagatgctgtgagttctttgccttcagggcttccagctctgtcctcaggctctgagtctagcaacacagcctccagtccatttttggactgcaccctgaggacaaggctgattattactgattgtccagggacagccagag

SEQ ID NO: 234 - DNA, 284 bp, Canis lupus familiaris
gccaacaagctgactcaatccctgtttatgtcagtggccctgggacagatggccaggatcacctgtgggagagacaactctggaagaaaaagtgctcactggtaccagcagaagccaagccaggctcccgtgatgcttatcgatgatgattgcttccagccctcaggattctctgagcaattctcaggcactaactcggggaacacagccaccctgaccattagtgggcccccagcgaggacgcggctattactgtgccaccagccatggcagttggagcacct

SEQ ID NO: 235 - DNA, 284 bp, Canis lupus familiaris
tccaatgtactgacacagccacccttggtgtcagtgaacctgggacagaaggccagcctcacctgtggaagaaacagcattgaagataaatatgtttcatggtcccagcaggagccaggccaggcccccatgctggtcatctattatagtacacaagaaaccctgagcgattttctgcctccagctctagctcggggtacatgatcaccctgaccaacagtggggcctaggacaaggacgaggatggctattactgtcagtcctatgacagtagtggtactcct

SEQ ID NO: 236 - DNA, 288 bp, Canis lupus familiaris
tcctatgtgctgactcagtcaccctcagtgtcagtgaccctgggacagacggccagcatcacctgtaggggaaacagcattggaaggaaagatgttcattggtaccagcagaagccgggccaagcccccctgctgattatctataatgataacagccagccctcagggatccctgagcgattctctgggaccaactcagggagcacggccaccctgaccatcagtgaggcccaaaccaacgatgaggctgactattactgccaggtgtgggaaagtagcgctgatgct

SEQ ID NO: 237 - DNA, 288 bp, Canis lupus familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggct

SEQ ID NO: 238 - DNA, 288 bp, Canis lupus familiaris
tccactgggttgaatcaggctccctccatgttggtggccctgggacagatggaaacaatcacctgctccggagatatcttagggaaaagatatgcatattggtaccagcataagccaagccaagcccctgtgctcctaatcaataaaaataatgagcgggcttctgggatccctcactggttctctggttccaactcgggcaacatggccaccctgaccatcagtggggcccgggctgaggacgaggctgactattactgccagtcctatgacagcagtggaaatgct

SEQ ID NO: 239 - DNA, 288 bp, Canis lupus familiaris
tcctatgtgctgactctgctgctatcagtgaccgtgaacctgggacagaccaccagcatcacctgtggtggagacagcattggagggagaactgtttactggtaccagcagaagcctggccagcgccccctgctgattatctataatgatagcaattgaccctcagggatccctgcctgattctctggctccaactcagggaacagggcctccctaaccatcattggggcctgggcctaagacgagtctgagtattacggagaggtgtgggacagcagtgctaaggct

SEQ ID NO: 240 - DNA, 283 bp, Canis lupus familiaris
tcctatatgctgactcagcagccattggcaagtgtaaacctcagccagtgggccagcaccacctgtggtggagataacattggagaaaaaaccgtccaatggaaccagcagaagcctggctaagctcccattacggctatctataaaggtagtgatctgccctcagggatccctgagcaattccctggccccaatttggggaacggggcctccctgaacatcagcggggctaagccgacgacgaggctattactgccagtcagcagacattagtggtaaggct

SEQ ID NO: 241 - DNA, 288 bp, Canis lupus familiaris
tcctatgtgctgacacagctgccatccgtgagtgtgaccctgaggcagacggcccgcatcacctgtgggggagacagcattggaagtaaaagtgtttactggtaccagcagaagctgggccaggcccctgtactgattatctatagagatagcaacaggccgacagggatccctgagcgattctctggcgccaactcggggaacacggccaccctgaccatcagcggggccctggccgaggacgaggctgactattactgccaggtgtgggacagcagtactaaggct

SEQ ID NO: 242 - DNA, 287 bp, Canis lupus familiaris
tccactgggttgaatcaggctccctccgtgttgctggcactgggacagatggcaacaatcacctgatccagagatgtctttgggaaaaatatgcatattggtaccagcagaagccaagccaagcccctgtgctcctaatcaataaaaataatgagcaggattctgggatccctgaccggttctctggctccaactcgggcaacacggccaccctgaccatcagtggggcccgggccgaggacgaggctgactattactgccagtcctatgacagcagtggaaatgtt

SEQ ID NO: 243 - DNA, 288 bp, Canis lupus familiaris
tcctatgtgctgtctcagccgccatcagcgactgtgactctgaggcagacggcccgcctcacctgtgggggagacagcattggaagtaaaagtgttgaatggtaccagcagaagccgggccagccccccgtgctcattatctatggtgatagcagcaggccgtcagggatccctgagcgattctccggcgccaactcggggaacacggccaccctgaccatcagcggggccctggccgaggacgaggctgactattactgccaggtgtgggacagcagtactaaggct

SEQ ID NO: 244 - DNA, 288 bp, Canis lupus familiaris
tcctatgtactgactcagctgccatcagtgactgtgaacctgggacagaccaccagcatcacctgtggtggagacagcattggagggagaactgtttactggtaccagcagaagcctggccagcgccccctgctgattatctataatgatagcaattggccctcagagatccctgcctgattctctggctccaactcagggaacagggcctccctaaccatcattggggcctgggcctaagatgagtctgagtattacggagaggtgtgggacagcagtgctaaggct

SEQ ID NO: 245 - DNA, 281 bp, Canis lupus familiaris
tcctatatgctgactcagcagccattggcaagtgtaaacctcagccagtgggccagcaccacctgtggtggagataacattggagagaaaactgtccaatggaaccagcagaagcctggctaagctctcattatggctatctataaaggtagtgatctaccctcagggatccctgagcaattccctggccccaactcgggtcggggcctccctgaacatcagcggggctacgccgacgactaggctattactgccagtcagcagacattagtggtaaggct

SEQ ID NO: 246 - DNA, 288 bp, Canis lupus familiaris
tcctatgtgctgacacagctgccatccatgagtgtgaccctgaggcagacggcccgcatcacctgtgagggagacagcattggaagtaaaagagtttactggtaccagcagaagctgggccaggtccctgtactgattatctatgatgatagcagcaggccgtcagggatccctgagcgattctccggcgccaactcggggaacacagccaccctgaccatcagcggggccctggccgaggacgaggctgactattactgccaggtgtgggacagcagtactaaggct

SEQ ID NO: 247 - DNA, 288 bp, Canis lupus familiaris
tccactgggttgaatcaggctccctccgtgttggtggccctgggacagatggaaacaatcacctgctcgagagatgtcttagggaaaagatatgcatataggtaccagcataagccaagccaagcccctgtgctcctaatcaataaaaataatgagcaggattctgggatccctgaccggttctctggctccaactcgggcaacacggccaccctgaccatcagtggggcccgggctgaggacgaggctgagtattactgccagtcctatgacagcagtggaaatgtt

SEQ ID NO: 248 - DNA, 286 bp, Canis lupus familiaris
tcctatgtgctgacacagctgccatccgtgaatgtgacccagaggcagacggcccgcatcacctgtgggggagacagcattggaagtaaaagtgtttactggtaccagcagaagctgggccaggcccctgttgattatctatagagacagcaacaggccgacagggatccctgagcgattctctggcgccaacacggggaacatggccaccctgactatcagcggggccctggccgtggacgaggctgactattactgccaggtgtgggacagcagtgctaaggct

SEQ ID NO: 249 - DNA, 288 bp, Canis lupus familiaris
tcccctgggctgaatcagcctccctccgtgttggtggccctgggacagatggcaacaaacacctgctccggagatgtcttagggaaaagatatgcatattggtaccagcataagccaagccaagcccctgtgctcctaatcaataaaaataatgagctgggttctgggatccctgaccgattctctggctccaactcgggcaacacggccaccctgaccatcagtggggcccgggccgaggacgaggctgactattactgccagtcctatgacagcagtggaaatgct

SEQ ID NO: 250 - DNA, 288 bp, Canis lupus familiaris
tcctatgagctgactcagccaccatccgtgaatgtgaccctgagggagacggcccacatcacctgtgggggagacagcattggaagtaaatatgttcaatggatccagcagaatccaggccaggcccccgtggtgattatctataaagatagcaacaggccgacagggatccctgagcgattctctggcgccaactcagggaacacggctaccctgaccatcagtggggccctggccgaagacgaggctgactattactgccaggtgggggacagtggtactaaggct

SEQ ID NO: 251 - DNA, 288 bp, Canis lupus familiaris
tcctatgtactgactcagctgccatcagtgactgtgaacctgggacagaccaccagcatcacctgtggtggagacagcattggagggagaactgtttactggtaccagcagaagcctggccagcgccccctgctgattatctataatgatagcaattggccctcagagatccctgcctgattctctggctccaactcagggaacagggcctccctaaccatcattggggcctgggcctaagacgagtctgagtattacggagaggtgtgggacagcagtgctaaggct

SEQ ID NO: 252 - DNA, 283 bp, Canis lupus familiaris
tcctatatgctgactcagcagccattggcaagtgtaaacctcagccagtgggccagcaccacctgtggtggagataacattggagaaaaaactgtccaatggaaccagcagaagcctggctaagctcccattacggctatctataaaggtagtgatctgccctcagggattcctgagcaattccctggccccaactcgggaaacggggcctccctgaacatcagcggggctaagccgacgactaggctattactgccagtcagcagacattagtggtaaggct

SEQ ID NO: 253 - DNA, 288 bp, Canis lupus familiaris
tcctatgtgctgacacagctgccatccgtgagtgtgaccctgaggcagacggcccgcatcacctgtgggggagacagcattggaagtaaaaatgtttactggtaccagcagaagctgggccaggcccctgtactgattatctatgatgatagcagcaggccgtcagggatccctgagcgattctccggcgccaactcggggaacacggccaccctgaccatcagcggggccctggccgaggatgaggctgactattactgccaggtgtgggacagcagtactaagcct

SEQ ID NO: 254 - DNA, 288 bp, Canis lupus familiaris
tccactgggttgaatcaggcttcctccgtgttggtggccctgggacagatggaaacaatcacctgctcgagagatgtcttagggaaaagatatgcatataggtaccagcataagccaagccaagcccctgtgctcctaatcaataaaaataatgagcaggattctgggatccctgaccggttctctggctccaactcgggcaacacggccaccctgaccatcagtggggcccgggctgaggacgaggctgagtattactgccagtcctatgacagcagtggaaatgtt

SEQ ID NO: 255 - DNA, 288 bp, Canis lupus familiaris
tcctatgtgctgacacagctgccatccgtgaatgtgaccctgaggcagccggcccacatcacctgtgggggagacagcattggaagtaaaagtgttcactggtaccaacagaagctgggccaggcccctgtactgattatctatggtgatagcaacaggccgtcagggatccctgagcgattctctggtgacaactcggggaacacggccaccctgaccatcagtggggccctggccgaggacgaggcttactattactgccaggtgtgggacagcagtgctcaggct

SEQ ID NO: 256 - DNA, 288 bp, Canis lupus familiaris
tccagtgtgctgactcagcctccttcagtatcagtgtctctgggacagacagcaaccatctcctgctctggagagagtctgagtaaatattatgcacaatggttccagcagaaggcaggccaagtccctgtgttggtcatatataaggacactgagcggccctctgggatccctgaccgattctccggctccagttcagggaacacacacaccctgaccatcagcggggctcgggccgaggacgaggctgactattactgcgagtcagaagtcagtactggtactgct

SEQ ID NO: 257 - DNA, 288 bp, Canis lupus familiaris
tcctatgtgttgactcagctgccttcagtgtcagtgaacctgggaaagacagccagcatcacctgtgagggaaataacataggagataaatatgcttattggtaccagcagaagcctggccaggcccccgtgctgattatttatgaggatagcaagcggccctcagggatccctgagcgattctctggctccaactcggggaacacggccaccctgaccatcagcggggccagggccgaggatgaggctgactattactgtcaggtgtgggacaacagtgctaaggct

SEQ ID NO: 258 - DNA, 288 bp, Canis lupus familiaris
tccagtgtgctgactcagcctccctcggtgtcagtgtccctgggacagacggcgaccatcacctgctctggagagagtctgagcagatactatgcacaatggtatcagcagaagccaggccaagcccccatgacagtcatatatggggacagagagcgaccctcagggatccctgaccgattctccagctccagttcagagaacacacacaccttgacaatcagtggagcccaggctgaggatgaggctgaatattactgtgagatatgggacgccagtgctgatgat

SEQ ID NO: 259 - DNA, 288 bp, Canis lupus familiaris
tcctacgtggtgacccagccaccctcagtgtcagtgaacctgggacagacggccagcatcacctgtgggggagacaacattgcaagcacatatgtttcctggcagcagcagaagtcgggtcaagcccctgtgacgattatctatcgtgatagcaaccggccctcagggatccctgagcgattctctggctccaactcggggaacacggccaccctgaccatcagcagggcccaggccgaggatgaggctgactattactgccaggtgtggaagagtggtaataaggct

SEQ ID NO: 260 - DNA, 317 bp, Canis lupus familiaris
ttgcccgtgctgacccagcctacaaatgcatctgcctccctggaagagtcggtcaagctgacctgcactttgagcagtgagcacagcaattacattgttcagtggtatcaacaacaaccagggaaggcccctcggtatctgatgtatgtcaggagtgatggaagctacaaaaggggggacgggatccccagtcgcttctcaggctccagctctggggctgaccgctatttaaccatctccaacatcaagtctgaagatgaggatgactattattactgtggtgcagactatacaatcagtggccaatacggttaagc

SEQ ID NO: 261 - DNA, 303 bp, Canis lupus familiaris
ttgcccgtgctgacccagcctccaagtgcatctgcctccctggaagcctcggtcaagctcacatgcactctgagcagtgagcacagcagttactatatttactggtatgaacaacaacaaccagggaaggcccctcggtatctgatgagggttaacagtgatggaagccacagcaggggggacgggatccccagtcgcttctcaggctccagctctggggctgaccgctatttaaccatctccaacatccagtctgaggatgaggcagattattactgtggtgcacccgctggtagcagtagc

SEQ ID NO: 262 - DNA, 317 bp, Canis lupus familiaris
ttgcccgtgctgacccagcctacaaatgcatctgcctccctggaagagtcggtcaagctgacctgcactttgagcagtgagcacagcaattacattgttcattggtatcaacaacaaccagggaaggcccctcggtatctgatgtatgtcaggagtgatggaagctacaaaaggggggacgggatccccagtcgcttctcaggctccagctctggggctgaccgctatttaaccatctccaacatcaagtctgaagatgaggatgactattattactgtggtgcagactatacaatcagtggccaatacggttaagc

SEQ ID NO: 263 - DNA, 299 bp, Canis lupus familiaris
ttgcccgtgctgacccagcctccaagtgcatctgcctccctggaagcctcggtcaagctcacatgcactctgagcagtgagcacagcagttactatatttactggtatcaacaacaaccagggaaggcccctcggtatctgatgaaggttaacagtgatggaagccacagcaggggggacgggatccccagtcgcttctcaggctccagctctggggctgaccgctatttaaccatctccaacatccagtctgaggatgaggcaggttattactatggtgtacccctggtagcagtagc

SEQ ID NO: 264 - DNA, 317 bp, Canis lupus familiaris
ttgcccatgctgacccagcctacaaatgcatctgcctccctggaagagtcggtcaagctcacatgcactttgagcagtgagcacagcaattacattgttcaatggtatcaacaacaaccagggaaggcccctcggtatctgatgcatgtcaggagtgatggaagctacaacaggggggacgggatccccagtcgcttctcaggctccagctctggggctgaccgctatttaaccatctccaacatcaagtctgaagatgaggatgactattattacagtggtgcatactatacaatcagtggccaatacggttaagc

SEQ ID NO: 265 - DNA, 302 bp, Canis lupus familiaris
ttgcccatgctgacccagcctccaagtgcatctgcctccctggaagcctcggtcaagctcacatgcactctgagcagtgagcaaagcagttactatatttactggtatcaacaacaacaaccagggaaggcccctcggtatctgatgaaggttaacagtgatggaagccacagcagggcgtcgggatccccagtcgcttctcaggctccagctctggggctgaccgctatttaaccatctccaacatccagtctgaggatgaggcagattattactgtggtgtacccactggtagcagtagc

SEQ ID NO: 266 - DNA, 317 bp, Canis lupus familiaris
ttgcccatgctgaccgagcctacaaatgcatctgcctccctggaagagtcagtcaagctcacctgcactttgagcagtgagcacagcaattacattgttcgatggtatcaacaacaaccagggaaggcccctcggtatctgatgtatgtcaggagtgatggaagctacaacaggggggacgggatccccagtcgcttttcaggctccagctctggggctgaccgctatttaaccatctccaacatcaagtctgaagatgaggctgagtattattacggtggtgcagactataaaatcagtgaccaatatggttaaga

SEQ ID NO: 267 - DNA, 303 bp, Canis lupus familiaris
ttgcccgtgctgacccagcctccaagtgcatctgcctgcctggaaacctcggtcaagctcacatgcactctgagcagtgagcacagcagttactatatttactggtatcaacaacaacaaccagggaaggcccctcggtatctgatgaaggttaacagtgatggaagccacagcaggggggacgggatccccagtcgcttctcaggctccagctctggggctgaccgctatttaaccatctccaacatccagtctgaagatgaggcagattattactgtggtgtacccgctggtagcagtagc

SEQ ID NO: 268 - DNA, 323 bp, Canis lupus familiaris
caggctgtgctgacccagccgccctccctctctgcatccctgggatcaacagccagactcacctgcaccctgagcagtggcttcagtgttggcagctactacatatactggtaccagtagaagccagggagccctccccggtatctcctgtactaactactactcaagtacacagctgggccccggggtccccagccatttctctggatccaaagacaactcggccaatgcagggctcctgctcacctctgggctgcagcctgaggacgaggctgactactactgtgctacaggttattgggatgggagcaactatgcttacc

SEQ ID NO: 269 - DNA, 304 bp, Canis lupus familiaris
cagcctgtgctgacccagccgccctccctctctgcatccctgggaacagcggccagaaatacctgcactctgagcagtgacctcagtgttggcagctgtgctataagctgatcccagcagaagccagggagccctccctggtatctcctgaactactaaacacacccatgcaagcaccaggactcacatctgtagccgcttctctggatttgaggatgcctctgccagtgcagggctctgctcatctctggaggctgaccatcactgtgctaagatcatggcagtgggggcagctagtgttaca

SEQ ID NO: 270 - DNA, 277 bp, Canis lupus familiaris
cagcctgtgctgacccagccgccgtcctctctgcatccctgggaacaacagccagactcacctgcaccctgagcagtggcttcaatatgtggggctaccatatattctggtaccagcagaagccagggagccctccccggtatctgctgaacttctactcagataagcaccagggctccaaggacacctcggccaatgcagggatcctgctcatctctgggctccagcctgaggacgaggctgactactactgtaaaatctggtacagtggtctggt

SEQ ID NO: 271 - DNA, 218 bp, Canis lupus familiaris
cagcctctgctacccagccacccccttctctgcgtctccaggtactacagccagacccacctgcaccctgagcagtggcaacagtgttggcagctgttccttataacggctcccacaaagacagagggccctccctggtatctgctgaggttcccctctaatagacaccatgtctctggatccacacataccttggccaatgcagggctcctgctcat

SEQ ID NO: 272 - DNA, 319 bp, Canis lupus familiaris
cagcctgtgctgaccaagtgccctctctttctgcatctcctggaacaacagtcagactcacttgcacctggagcagtggctccagcactggcagctactatatacactggttccagagccacagagccagagccacagagctctccctggtatctcctgtactactactcagactcagataagcaccagggctctggggttctcagctctgtctcctgatccaaggatgcctcagttattggagggctctctcatctctgggctgcagcctgaggattagactgaccttcactgtctaatcagaaacaataatgcttct

SEQ ID NO: 273 - DNA, 314 bp, Canis lupus familiaris
cagcctgtgctgacccagctgccctccctctctgcataccggggaacaaactccagatgtacctacaccctgagcagtgtcgccaactactaaacatacttctcaaagagaatacagggcaccttccacagtacatcctgtactactactcagactcaagtgcatgattgggatttggggtcccaggcacttctctggatccaaagatgcctcagccaatgcagggatcctgctgatctctgggctgcagccagaggacaagtctgactgtcactgtgctacagatcatggcagtgggagcagcttccgatact

SEQ ID NO: 274 - DNA, 314 bp, Canis lupus familiaris
cagccagggctgacccagccacactccctctctgcatatcagggagaaacagccacacatacctgcaccctgagcggtggcttcagtgttggcagctgccatatatactggatccagaagaagccagagagccctccctgatgtctcctgaactactactaagactcagataaggcctcgacgtccccagccctactctgaatccaaagacaccttgcccaaggtgggaatcctgctcatctctgggctgcagccggaggacaaggctgtctcttactgtataatatggcacagtggttctggtcacagggaca

SEQ ID NO: 275 - DNA, 307 bp, Canis lupus familiaris
caccctgtgctgacccagctgccctccctctctgcatccctgggaacaacagccagactcatgtgcaccctgagcagtggctgcagtggtggccatacgctggttccagcagccaggaggcctcctgagtacctgctgatggtctactgagactcaccagggccccggtggccccagccgcttctctggctccaaggacacctcggccaatgcagggctcctgctcatctctaggctgcagcctgaggacgaggctgactgtcactgtgttacagaccatggcagtgggagcagctcccgaaactca

SEQ ID NO: 276 - DNA, 326 bp, Canis lupus familiaris
cagccagggctggcccagcttccccccacctccctctgcatctccaggaacaacagccagactcacatgaaccatgagcagtggcttcatcgttggcgctgctacatatactggttccaacagaagccagggagcaccgccccagtatctcctgaggttctactcagactcagataagcactagggctcaacgaccccagccctgttctggatctgaagacacctccgccgaagcagggcctctgctcatctctgggctgcagcgtgaggacaaggctgactcttatgggacaatctggcacagtggtcctggtcacagggacaca

SEQ ID NO: 277 - DNA, 305 bp, Canis lupus familiaris
cagcctgtgctgacccagctgccctccctttctgcatccctgggaacaacagccagactcacatgcaccctgagcagcggctgcagcggtggccacacattggttccagcagccaggaggcctcctgagtacctgctgatggtctactgagactcaccagggccccggtgttgccagcctcttctctggctccaaggacacctcggccaatgcaggactcctgctcatctctgggctgcagcctgaggatgaggctgactgtcactgtgctacagaccatggcagtgggagcagctccggatact

SEQ ID NO: 278 - DNA, 305 bp, Canis lupus familiaris
cagcctgtgctgacccagctgccctccctttctgcatccctgagaacaacagccagactcacctgcaccctgagcagtggctgcagtggtggccatatgctggttccagcagccaggaagcctcctgagtatctgctgacggtcttctgagactcaccagggccccgaggtccccagcctcttctctggctccaaggacacctcagccaatgcaggactcctgctcatctctgggctgcagcctgaggatgaggctgactgtcactgtgctacagaccatggcagtgggagcagctcccgatact

SEQ ID NO: 279 - DNA, 300 bp, Canis lupus familiaris
caccctgggctgacccagtcgtcctccctctctgcatccctgggaacaacagccagactcacctgcaccctgagcagtggcttcagaaatgacaggtatgtaataagttggttccagcagaaatcagggagcccttcctggtgtctcctgtattattactcgaactcaagtacacatttgggctctgaggttcccagctgcttctctggatccaagacaaggccacacccacactgagtagacccctctctgggtgggtctagagctccagctccacctgaggctgatgcacaattgcag

SEQ ID NO: 280 - DNA, 321 bp, Canis lupus familiaris
cagccagggctggcccagctgccctccctctctgcatctccaggaacaacagccagactcacatgaaccatgagcagtggcttcattgttggtggctgctacatatactggttccaacagaagccagggagcatgccccccagtatctcctgaggttctactcagactcagataagcaccaggtctcaacatccccagcccggctctggatctgaagacactcagccgaagcagggcctctgctcatctctgggctgcagcatgaggacaaggctgactcttactgtacaatctggcacagtggtcctggtcacagggaca

SEQ ID NO: 281 - DNA, 220 bp, Canis lupus familiaris
cagcctgtgctgacccattgccctccctctctgcatcctgggaaataacaaccagactcacctgcactctgagcagcggctgcagcggtggccatacagtggttccagcagcaaggaagcctcctgagtacctgctgacgttctactgagactcaccagggctctagggtccccagccacttctctggtttcaaggacaccacggccaatgcagggcact

SEQ ID NO: 282 - DNA, 309 bp, Canis lupus familiaris
cagcctgtgctgacccagtcgccctccctctcggcatctttggaacaacagtcagactcacctgtaccctgatcagtggctccagtgttggcagctattacatcaactggttccagaagaagccacggagccctccccagtatctcctgtactactacttagactcagataagcaccagggctctggggtccccagctgcttctcctgatccaaggatgcctcagtcattggaggacaccctcatctctgaactgcagcctgaggactagactgaccttcgctgtctaatcagaaacaataatgcttct

SEQ ID NO: 283 - DNA, 315 bp, Canis lupus familiaris
cagcctgtgctgacccagcctccctctctctctgcatctctgggaacaatagccagacaaacatgcagcctgagcaggggctacagtatggggacttatgtcatacgctggttccagcagtagcaagaaactctcctgagtatctgctgaggttatactgagcctcagcaggtctctggggaccccagctgagtctttagatccaagatgcctcagccaattcagggctcctgcttatctctgtgctgcagcctgaggacaagggttactattactgttctgtacatcatggaattgtgagcagctatacttacc

SEQ ID NO: 284 - DNA, 325 bp, Canis lupus familiaris
cagcttgtggtgacccagccgccctccctctctgcatccctgggatcatccgccagactcacctgcaccctgagcagtggcttcagtgttggcagttattctgtaacttggttccagcagaagccagggagccctctctggtacctcctgtactaccactcagactcagataagcaccagggctccagggtccccagccgcttctctggatccaaggacacctcggccaatgcagggctcctgctcatctctgggctgcagcctgaggatgaggctgactactactgtgcctccgctcatggcagtgggagcaactaccattact

SEQ ID NO: 285 - DNA, 318 bp, Canis lupus familiaris
cagccagtgctgacccagctgccctccttctctgtatctctgggaacaacagtcagactcacctgcaccctgagcagtgttggcagctactaaacatccttttcaaggagaaaccaaggagccccccaccccggtatctcctatactactattcagactcagataaaccccaggtctctggggtccccagccacttctctgcatccaaagactcctaggccaatgcagggctcctgctcgcctctgggctgcagcctgaggacgaggctgactatcactgtgctataaatcatgacagtgggagtagttcctgatact

SEQ ID NO: 286 - DNA, 309 bp, Canis lupus familiaris
cagcctttggtgacccagcgccctccctctctgcatctcctgaaacaacagtcagactcacatgcaccctgagcagtggccccagtgctggcagctactacatacactggttccagtggaagccacggtgcccgccccggtatctcctgtactactactcagactcagatgagcaccagggctctggggtccccagccgcttctcctgatccaaggatgcctcagccagggcagggctccctcatctctgggctacagtctgaggtctacactgaccttcactgtctaatcggaaacaataatgtttct

SEQ ID NO: 287 - DNA, 303 bp, Canis lupus familiaris
cagcctgtgctgacccagcgacctccctctctgcatccctgggaacaacagccagactcacctgcaccctgagcagcggctgaagcggtggccatacgctggttccagcagccaggaagcctcctgagtacctgctgatggtctactgagactcaccaggctatggggtccccagcatcttctctggctccaaggacacctcggccaatgcagggctcctgctcatctctgggctgcagcctgaggtcgaggctgactgtcactgtgctacagaccatggcagtgggagcagctcccgatact

SEQ ID NO: 288 - DNA, 309 bp, Canis lupus familiaris
cagcctgtgctgacccagtcgccctccctctcagcatctttggaacaacagtcagactcacctgtaccctgatcagtggctccagtgttggcagctattacatcaactggttccagaagaagccacggagccctccccagtatctcctatactactacttagactcagataagcaccagggctctggggtccccagctgcttctcctgatccaaggatgcctcagtcattggagggcaccctcatctctgagctgcagcctgaggactagactgaccttcgctgtctaatcggaaacaataatgcttct

SEQ ID NO: 289 - DNA, 327 bp, Canis lupus familiaris
cagcctgtgctgacccagccaccctccctctctgcatccccgggaacaacagccagactcacctgcaccctgagcagtggcttcagtgttggtgactatgacatgtactggtaccagaagaagccaggaagcccccaccccgggatctcctgtactactactcagactcatataaacaccagggctccggggtctccagcagcttctctggatccaaggatacctcagccaatacagggctcctgctcatctctgggccacagcctgaggacgaggctgactactactgtgctacagatcatggcagtgagagcaggtactcttacc

SEQ ID NO: 290 - DNA, 292 bp, Canis lupus familiaris
cagcctctgctacccagcacccccttcgctgcgtttccaggtactacagccagaatcacctgcaccctgagcaggggcatcagtgttgggagctgttccttataacggctcccgcagaggcagggagccctgcctggtatctgctgaggttcccctctaatagacaccacatctctggatccaaagaaacctcggccaatgcagggctcctgctcattgttgtgctgccacctgacaactagtctatcagtggtggttgaggactaggactattactgggatgctttggttt

SEQ ID NO: 291 - DNA, 307 bp, Canis lupus familiaris
cagcctttgctgatccagcgccctccctctctgcatctcctggaacaacagtcagactcacctgcacccagagcagtggcccctgtgttggcagctactacatacactggttccagtggaagccatggagccctccctggtatcttctgtactactaatcagactcagatgagcaccagggctctggggtccccagccgcttctcctgatccaaggatgcctcagccagagcagggctccctcatctctggactgcagcctgaggactagactgaccttcactgtctaatcagaaacaataatgttt

SEQ ID NO: 292 - DNA, 312 bp, Canis lupus familiaris
tgcaggtccctgtcccagcctttgccctccctctttgcatctcctggaagaacagtcagatccacctgcacccagagcagtggcccctgtgttggcagctactacatacaccggttccagtggaagccacggagccgtctccatatctcctgtactactactcagactcagatgagcaccagagctctggagtccccaactgcttctcctgatccaaggatgcctcagggaaggcagggctccctcatctctgggctacaggctgaggacaagactgacctttactgtctaatccaaaacaataatgtttct

SEQ ID NO: 293 - DNA, 325 bp, Canis lupus familiaris
cagcctgtgctgacccagccaccctccctctctgcatccctgggatcaacagccagacccacctgcaccctgagcagtggcttcagtgttggaagctaccatatactctggttccagcagaagtcagagagccctccccggtatctcctgaggttctactcagattctaatgaacaccagggtcccggggtccccagccgcttctctggatccaaggacacctcaacctatgcagggctcttgctcatctctgggctgcagcctgaggacgaggctgactactactgtgctacagaccatggcagtgggagcagctacacttacc

SEQ ID NO: 294 - DNA, 287 bp, Canis lupus familiaris
cagcctttgctgacccagcgccctccctctctgcatctcctggaacaaaagtcagactcacctgcatccagagcagtggatccagcgttggcagctactacatacactggttccagtagaagccatggagccctccccagtatctcctgtactactacttagactcagataagcactaggcctatggggaacccagatccttcccctgatccaaggatgcctcagtcaatgcagggtcaaagagaggggattatttagagtggacaattggggcctttggccaggag

SEQ ID NO: 295 - DNA, 313 bp, Canis lupus familiaris
cagccagtgcagacccagctgccctccttctctgtacctctgggaacaacagccagactcacctgcaccctgagcagtgttggcggccagtaaacatccttttcaaggagaaaccaaggagccccccagtctctcctgtactattacccagactcagataaaccccaggtctctggggtccccagccacttctctgaatccaaagactcctaggccaatgcagggctcctgctcgcctctgggctgcagcctgaggacgaggctgactatcactgtgctgtaaatcatgacagtgggagcagctccggatact

SEQ ID NO: 296 - DNA, 298 bp, Canis lupus familiaris
caggctgtggtgacccagcttccttctctgcatccctgggaacaacagccagactcacatgcaccctgagctgtggcttcagtattgatagatatgctataaactggttccagcagaaggcagagagccttccctggtacctactgtgctattactggtactcaagtacacagttgggcttcagcgtccccagctgcatctctggatccaagacaaggccacattcacaaacgagtagacccatctctggttgggtctagagctccagccccacctgagactgatgcacaattgcagc

SEQ ID NO: 297 - DNA, 306 bp, Canis lupus familiaris
cagcctgtatagacccagtcaccctccctttctgcatctttggaacaacagtcagagtcacctgtaccctgagcagtggctccagtgttggcagctactacatatactggttccaggagaagccatggagcaatccccggtatctcctgtactactcaggctcagatgagcaccagggctctgggatccgtagctgcttctcctgatacaatgatgcctcagccaaggcagagctccctaatctctgggctgcagcctgaggactatactgaccttcactgtctaatcagaaacaataatcctttt

SEQ ID NO: 298 - DNA, 227 bp, Canis lupus familiaris
tagcctgtgctgacccagcgccctcccactctgcatccctgggaacaacagccagactcacctgcgccctgagcagcggctgcagcagtgaccatacgctggttccagcagccagaaggcctcctgagtacctgctgacggtctactgagactcaccagcgccccggggtcctcagcctcttctctggctccaaggacacctcggccaatgcagggcactcagatgg

SEQ ID NO: 299 - DNA, 301 bp, Canis lupus familiaris
cagcctgtgatgacccagctgtcctccctctctgcatccctggaaacaacaaccagacacacctgcaccctgagcagtggcttcagaaataacagctgtgtaataagttgattccagcagaagtcagggagccctccctggtgtctcctgtactattactcagactcaagtatacatttgggctctgaggttcccagctgcttctctggatccaagacaaggccacacccacactgagtagacccatccctgggtgggtctagagctccagccccactggaggctgatgcacaattgcagc

SEQ ID NO: 300 - DNA, 307 bp, Canis lupus familiaris
caacctttgcggacccagcgcactccctctgcatctcctggaacaacagttagactcatctgcacccagagcagtggccccagtgttggcagctactacaaacactggttccagcagaagccacggagccctccccggtacttcctgtactacttctcagactcagatgagcaccagggctctggggaccgcagccacttctcctgatccaaggatgactcaggaaaggcagggctccctcatctctgggctacagcctgaggactagactgaccttcactgtctaatcagaaacaataatgcttct

SEQ ID NO: 301 - DNA, 323 bp, Canis lupus familiaris
ttaaaaccaaccaaaccaaaccaaaccaaaacaaaacaaaacaaaataacagccagattcacctgctccctgagcagtggcttcagtgttggtggctataacacactggtaccagcagaagccagggagccctccctgttacctcctgtactactactcagaatcagataaacaccatggctccgggatcaccagctgcttccctggccctatggacacctcggccaatgcagggctcctgctcatctcagggctgcagcctgaggacgaggctgactactactgcggtatactccacagcagtgggagcagctactcttacc

SEQ ID NO: 302 - DNA, 307 bp, Canis lupus familiaris
caggctgtgaggacacactcctccttcctctctgcacctttgggatcatcaaccagactcacctgcatccttcccagggcctgaatgttggcaggtactgaacatactggacaaggagaatcaaggagacatcaggagttccctcagatccagataagtgccagggcacggggttctcagccacttctatggatctaatgatgcctcaggcaatgcaggtctcctgctcatgtctgggctgcagcctgaggacgaggctgactatgactatgctgcacattgtggggtgggagcagctcccgatact

SEQ ID NO: 303 - DNA, 305 bp, Canis lupus familiaris
cagcctgtgctgacccagccgccctccctctctgcatccctgggaacaacagccagactcacctgcaccctgagcagcagctgcagcggtggccatatgctggttccagcatgcaagaggcctcctgagtacctgctgatggtctactgagactcaccagggccctggggtccccagcctcttctctggctccaaggaagcctcggccaatgcagggctcctgctcatctctgggctgcagcctgagaatgaggctgactgtcactgtgctacagaccatggcagtgggaacagctcccaatact

SEQ ID NO: 304 - DNA, 307 bp, Canis lupus familiaris
cagcctttgctgacccagcgtcctccctctctgcatctcctggaacaacagtcagactcacatgtaccctgagcagtggccccggtgctggcagctactacacacactggttccagcagaggccacagagtcctccccggtatctcctgtactactactcagactcagatgatctccagggctccgggttccccagccactcctcctgatccaaggatgcctcagccagggcagggctcccatctctggggtacagcctgaggactacactgaccttcactgtctaatcggaaacaataatgtttct

SEQ ID NO: 305 - DNA, 303 bp, Canis lupus familiaris
cagccagggctggcccagctgccccccacctccctctgcatctccaggaacaacagccagactcacatgaaccatgagcagtggcttcattgttggcagctgctacatatactggttccaacagaagccagggagcccccctcccccaatatctcttgaggttgtattcagaatcagataaacaccagggctcaatgtccccagccctgctctggatctgaagacacctccgccgaagcagggcctctgctcatctctgggctgcagcgtgaggacaaggctgactcttactgtacaatctgg

SEQ ID NO: 306 - DNA, 305 bp, Canis lupus familiaris
cagcctgtgctgacccagccgccctccctctctgcatccctgggaacaacagccagactcacctgcaccatgagcagcagctacagtggtggccatacactggttccagcagccaggaggcctcctgagtacctgctgatggtctactgagatttaccagggccccggggtccccagccgcttctctggctccaaggacatctcggccaatgcagggctcctgctcatctctgggctgtagcctgaggacgaggctgactgtcactgtgctacagaacatggcagcgggagcagctcccaatact

SEQ ID NO: 307 - DNA, 215 bp, Canis lupus familiaris
ctgcctctgctacccagccaccgccttctctgcatctccaggtactacagccagacccacctgcaccctgaacagtggcatcagtattcgcagctgttccttataatggctcccgcaaaggcagggagccctgcctggtatctgctaaggttgtactctaataaataccatggctctagggtcccaagccacatctctggatccaaagaaacctc

SEQ ID NO: 308 - DNA, 309 bp, Canis lupus familiaris
cagcctttgctgacccagcgtcctccctctctgcatctcctggaacaacagtcagactcacctgtatccagagcagtggccccagtgttggcagctactacatacaccggttccagcggaaaccacggagccctcccctgtatctcctgtactactactcagactcagataagcactaggcctacagggtccccagctgcttctcctgatccatggatgcctcagccagtgcagtgctccctcatctctgggctacagcctgaggactagactgaccttcactgtctaatcggaaacaataatgcttct

SEQ ID NO: 309 - DNA, 319 bp, Canis lupus familiaris
cagcttgtgctgacccagccgccctccctctctgcatccctgggatcaacaaccagactcacctgcaccctgagcagtggcttcagtgttggtggctatagcatatactggcaccagcagaagccagggagcactccctggtacctcctgtactactactcaagtacagagttgggacctggggtccccagctgcttctctggatccaaagacacctcagccaatgtagggctcctgctcatctcagggctgcagcctgaggatgagactgactactactgtgctataggtcacggcagtgggagcagctacacttacc

SEQ ID NO: 310 - DNA, 303 bp, Canis lupus familiaris
cagccagggctggcccagctgcccccccacctccctctgcatctccaggaataacagccagactcacatgaaccatgagcagtggcttcattgttggccgctgctacatatactgattccaacagaagccaaggagcccccgctccaccagtatctcctgatattctactcagactcagataagcaccagggctcaacgtccccagccctgctctgaatctgaagacacctccgcgaagcagggcttctgctcatctctgggctcagcgtgaggacaaggctgactcttactgtacaatctgg

SEQ ID NO: 311 - DNA, 304 bp, Canis lupus familiaris
tagcctgtgctgacccagtgctctccctctctgcatccctgggaacaacagccagactcccctgcaccctgagcagcggctgcagcggtgtccatacgcaggttccagcagccaggaggcctcctgaatacctgctgatggtctacggtgactcaccagggccccggggtccccagccgcttctctggctccgaggacacctcggccaatgcagggctcctgctcatctctgggctgcagcctgaggacaagactgactgtcactgtgctacagaccatggcagtaggagcagttcccaatact

SEQ ID NO: 312 - DNA, 319 bp, Canis lupus familiaris
cagcctgtgctgacccagctgcccttcctctctgcatccctggagacaacaagcagatgtacctacacccagagcggtgtcggcagctactacacatactcatcaaggacaatccagggagacctccctggtatttcctgtactactactcagactcaactacatggttgggatttggtgtccccaaccacttctctgtatccaaagatgcctcagccaatgcagggctcctgctcatctctgggctgcagccagaggacaaggatgactgtcactgtgctgcattcagatcatggcagtgggagcagctcccgatact

SEQ ID NO: 313 - DNA, 309 bp, Canis lupus familiaris
cagcctttgctgatccagtgccctccctctctgcatctcctggaacaagagtcagactcacctgcacccagagcagtggccccagggttggcagctactacatacactggttgcagcggaaaccacggagccctcctcagtatctcctgtactactactcagaatcagatgagcaccagggctctggggtccccagccacttctcctgatccaaggatgcctcaggcaaggcagggctccctcatccctgggctacagcctgagggctagactgaccttcactgtctaatccgaaacaataatgtttct

SEQ ID NO: 314 - DNA, 314 bp, Canis lupus familiaris
cagccagggctggcccagctgccctccctctctgcatctccaggaacaacagccagactcacatgaaccatgaacagtggcttcattcttggcggctgatacatatacttgttccaacagaaaccagggaacccccgctccccgtattgcctgaggttctactcagactcagataagcaccagggctcaacatccccagccctgctctggatctgaagacacctcaactgaagcagggcctctgctcatctctggatgtccagcgtgaggacaaggttgattcttactgtacaatctggcacagtggtcctggt

SEQ ID NO: 315 - DNA, 305 bp, Canis lupus familiaris
cagcctctgctgacccagccaccctccctctctgcatccctgggaacaagacccagagtcacctgcaccctgagcaacaactgcagtggtggccatacgctggttccagcagccaggaagcctcctgaatacctattgatggtttactgagacttaccagggcccccggggccccagctgcttctctggctccaaggacaccttggccaatgcaggactcctgctcatctctgggctgtagcctgaggatgaggctgactgtcactgtgctacagaccatggcagtgggagcagctcccgatact

SEQ ID NO: 316 - DNA, 320 bp, Canis lupus familiaris
caggctgtggtgacccagcttccttctctgcatccctgggaacaacagccagattcacatgcaccctgagctatggcttcagtattgatagatatgttataagctggttccagcagaaggcagagagccttccctggtacctactgtactattactgatactcaagtacacagttgggcttcggcattcccagctgcgtctctggatccaagacaaggccacattcacaaatgagtagacccatctctggttgggtctagagctccagccccacctgagactgatgcacaattgcagccacattgtcttgatatcggaaa

SEQ ID NO: 317 - DNA, 306 bp, Canis lupus familiaris
cagcctgtatagacccagtcaccctccctttctgcatctttggaacaacagtcagactcacctgtaccctgagcagtggctccagtgttggcagctactacatatactggttccaggagaagccatggagcaatccccggtatctcctgtactattcaggctcagatgagcaccagggctctgggatccctagctgcttctcctgatccaaggatgcctcagccaaggcagagctccctcatctctgggctgcagcctgaggactagactgaccttcactgtctaatcagaaacaataatgcttct

SEQ ID NO: 318 - DNA, 283 bp, Canis lupus familiaris
cagcctgtgctgacccagcgccctcccactctgcatccctgggaacaacagccagactcacctgcaccctgagcagcggctgcagcggtggccatatgctggttccagcagccagaaggcctcctgagtacctgctgacggtctactgagactcaccagggcccctgggtcctcagcctcttctctgactccaaagacacctcggccaatgcagggcactcagatggctgtgaagttcatacaacagggtcctcatgggggctcatggtaccacttcacgttt

SEQ ID NO: 319 - DNA, 301 bp, Canis lupus familiaris
cagcctgtgatgacccagctgtcctccctctcagcatccctggaaacaacaacaagactcacctgaaccctgagcagtggcttcagaaatgacagatgtgtaataagttggttccagcagaagtcagggagccctccctggtgtctcctgtactattactcggactcaagtacacatttgggctctgaggttcccagctgcttctctggatccaagacaaggccacacccacactgagtagacccatccccgggtgggtctagagctccagccccactggaggctgatgcacaattgcagc

SEQ ID NO: 320 - DNA, 309 bp, Canis lupus familiaris
caacctttgcggacccagcgccctccctctctgcatctcctggaacaacagttagactcatctgcacccagagcagtggccccagtgttggcagctactacaaacactggttccagcagaagccacggagccctccccggtacctcctgtactactactcagactcagatgagcaccagggctctggggaccacagccacttctcctgatccaaggatgcctcaggaaaggcagggctccctcatctctgggctacagcctgaggactagactgaccttcactgtctaatcagaaacaataatgcttct

SEQ ID NO: 321 - DNA, 314 bp, Canis lupus familiaris
cagcctgtgctgaccagctgccctctctgcatccctgggaacaacaggcagatgtacttacaccctgagcagttttggcagctactacacatactcgtcaaggagaatacagggagacctccctggtatttcctgtactactactcagactcaactacatggttgggatttggggtccccaaccacttctctggatccaaagatgcctcagccaatgcagggctcctgctcatctctgggctgcagccagaggacaaggatgactgtcactgtgctgcatacatatcaaggcagtggaagcagctcccaatact

SEQ ID NO: 322 - DNA, 304 bp, Canis lupus familiaris
ctgcctgtgctgacccagtgccctccctctctgcatccctgggaacaacagccagactcacctgcaccctgagcagtggctgcagcggtggccatatgctggttccagcagccaggaggcctcctaagtacctgctgatggtctactgagactcatcacggtcctggggtccctagcctcttctctggctccaaggacacctcggccaatgcagggctcctgctcatctctgggctgcagcctgaggacgaggctgactgtcattgtgctacagaccatggcagtgggagcagctcctgatact

SEQ ID NO: 323 - DNA, 325 bp, Canis lupus familiaris
cagcctgtgctgacccagccaccctccctctctgcatccctgggaacaacagccagactcacctgcaccctgagcagtggcttcagtgttggtgactatgacatgtactggtaccagcagaagccagggagccctccccgggatctcctgtactactactcggactcatataaaaaccagggctctggggtctccaaaagcttctctggatccaaggatacctcagccaatgcagggctcctgctcatctctgggctgcagcctgaggacgaggctgactactactgtgctacagatcatggcagtgagagcagctactcttacc

SEQ ID NO: 324 - DNA, 306 bp, Canis lupus familiaris
cagcctgtatagacccagtcaccctccctttctgcatctttggaacaacagtcagactcacctgtaccctgagcagtggctccagtgttggcagctactacatatactggttccaggagaagccatggagcaatccccggtatctcctgtactactcaggctcagatgagcaccagggctctgggatccctagctgtttctcctgatccaaggatgcctcagccaaggcagagctccctcatctctgggctgcagcctgaggactatactgaccttcactgtctaatcagaaacaataatgcttct

SEQ ID NO: 325 - DNA, 305 bp, Canis lupus familiaris
cagcctgtgctgacccagccgccctccctctctgcatccctgggaacaacagccagactcacctgcaccatgagcagcagctgcagcggtggccatatgctggtaccagcatgcaagaggcctcctgagtacctgctgatggtctactgagactcaccagggccctggggtccccagcctcttctctggctccaaggacaccttggccaatgcagggctcctgctcatctctgggctgcagcctgagaatgaggctgactgtcactgtgctacagaccatggcagtgggaacagctcccaatact

SEQ ID NO: 326 - DNA, 323 bp, Canis lupus familiaris
taaaaccaaaccaaaccaaaccaaaccaaaacaaaacaaaacaaaataacagccagattcacctgctccctgagcagtggcttcagtgttggtggctataacacactggtaccagcagaagccagggagccctccctgttacctcctgtactactactcagaatcagataaacaccatggctccgggatcaccagctgcttccctggccctatggacacctcggccaatgcagggctcctgctcatccttgggctgcagcctgaggacgaggctgactactactgcggtatactccacagcagtgggagcagctactcttacc

SEQ ID NO: 327 - DNA, 302 bp, Canis lupus familiaris
aagcctgtgctgacccagcgccctccctctctgcatccctgggaacaacagccagactcacctgcaccctgagcagcggctggagtggtggctataggctggttccagcagccaggaagcctcctgagtacctgctgatggtctactgagactcaccaggctatggggtccccagcatcttctctggctccaaggaagcctcggccaatgcagggctcctgctcatctctggcctgcagcctgaggtcgaggctgactgtcactgtgctacagaccatggcagtgggagcagctcccgatac

SEQ ID NO: 328 - DNA, 308 bp, Canis lupus familiaris
cagcctgtgctaacccagtcgctctccctcttgacatctttggaacaacagtcagactcacctgtaccgtgaacagtggctccagtgttggcagctattacatcaactggttccagtataagccatggagctctccctagtatcacctgtactactacttagactcagataagcaccagggctctggggtccccagctgcttctcctgatccaaggatgcctcagtcattggagggcaccctcatctctgggctgcagcctgaggactagactgaccttcacgtctaatcagaaacaataatgcttct

SEQ ID NO: 329 - DNA, 304 bp, Canis lupus familiaris
ctgcctgtgctgacccagccgccctccctctctgcatccctgggatcaacagccagactcacctgcacactgagcagtggctgcagcggtggccatatgctggttccagcagccaggaggcctcctgtgtacctgctgatggtctactgagactcaccagggccccagtgtccccagccactactctggtttcaaagacacctcggccaatgcaggtcactcagatagctgcgaaattcatacaacaagggtcctcatggggactcatgggcaccccttcagattttcctgcctgcatgaacag

SEQ ID NO: 330 - DNA, 300 bp, Canis lupus familiaris
cagggatggcccagctgttcccccacctccctctgcatctccaggaacaacagccagactcacatgaaccatgagcagtggcttcattgttggcggctgctacatatactggttccaacagaagccagggagtccccttccccccatatctcctgagtttctactcagactcagataagcaccagggctcaaaatccccagccctgttctggatctgaagacacctcagccaaagcagcgcctctgctcatctctgggctgcagggtgaggataagaatgactcttactctacaatctgg

SEQ ID NO: 331 - DNA, 314 bp, Canis lupus familiaris
caacctttgcggacccagtgccctccctctctgcatctcctggaacaacagttagactcatctgcacccagagcagtggccccagtgttggcagctactacaaacactggttccagcagaagccacggagccctccccagtacctcctgtactacttctcagactcagatgagcaccagggctctggggactgcagccacttcccctgatccaaggatgcctcaggaaagcagggctccctcatctctgggctacagcctgaggactagactgaccttcactgtctaatcagaaacaataatgcttcttacagt

SEQ ID NO: 332 - DNA, 305 bp, Canis lupus familiaris
cagcctgtgctgacccagccgccctccctctctgcatccctgggaacaacattcagactcacctgcaccctgagcagcagctgcagcggtggccatatgctggttccagcatgcaagaggcctcctgagtacctactgatggtctactgagactcaccagggccctggggtccccagcctcttctccggctccaaggacaccttggccaatgcagggctcctgctcatctctgggctgcagcctgagaatgaggctgactgtcactgtgctacagaccatggcagtgggaacagctcccaatact

SEQ ID NO: 333 - DNA, 307 bp, Canis lupus familiaris
caggctgtgacgacacactcctccttcctctctgcacctttgggatcatcaaccagactcacctgcatccttcccagggcctgaatgttggcaggtactgaacatactggacaaggagaatcaaggaggcatcaggagttccctcagatccagataagtgccagggcacggggttctcagccacttctatggatctaatgatgcctcaggcaatgcaggtctcctgctcatgtctgggctgcagcctgaggacgaggctgactatgactatgctgcacattgtggggtgggagcagctcccgatact

SEQ ID NO: 334 - DNA, 303 bp, Canis lupus familiaris
aagcctgtgctgacccagcgccctttctctctgcatccctgggaacaacagccagactcacctgcaccctgagcagcggctggagtggtggctataggctggttccagcagccaggaagcctcctgagtacctgctgatggtctactgagactcaccaggctatggggtccccagcatattctctggctccaaggaagcctcggccaatgcagggctcctgctcatctctgggctgcagcctgaggtcgaggctgactgtcactgtgctacagaccatggcagtgggagcagctcccgatact

SEQ ID NO: 335 - DNA, 291 bp, Canis lupus familiaris
cagactgtggtcaccaaggatccatcactctcagtgtttccaggagggacagtcacattcacatgtggcctcagctctgggtcagtctttacaagtaactaccccagctggtaccagcagacccatggccgggctcctcacatgcttatctacagcacaagcagctgcccccccggggtccctgatcgcttctctggatccatctctgggaacaaagttgccctcaccatcacaggagcccagcctgaggatgagactattattgttcactgcgtatgggtagtacattta

SEQ ID NO: 336 - DNA, 309 bp, Canis lupus familiaris
cagcctgtgctaacccagtcgccctccctcttgacatctttggaacaacagtcagactcacctgtaccgtgaacagtggctccagtattggcagctattacatcaactggttccaggagaagccatggagctctccctggtatcacctatactacttcttagactcagataagcaccagggctctggggtccccagctgcttctcctgatccaaggatgcctcagtcattggagggcaccctcatctctgggctgcagcctgaggactagactgaccttcactgtctaatcagaaacaataatgcttct

SEQ ID NO: 337 - DNA, 217 bp, Canis lupus familiaris
ctgcctgtgctgacccagccgccctccctctctgcatccctgggatcaacagccagactcacctgcacactgagcagtggctgcagcggtagccatatgctggttccagcagccaggaggcctcctgggtacctgctgatggtctactgagactcaccagggccccagtgtccccagccactactctggatgcaaagacacctcggccaatgcaggt

SEQ ID NO: 338 - DNA, 300 bp, Canis lupus familiaris; identical to SEQ ID NO: 330

SEQ ID NO: 339 - DNA, 307 bp, Canis lupus familiaris; identical to SEQ ID NO: 300

SEQ ID NO: 340 - DNA, 301 bp, Canis lupus familiaris; identical to SEQ ID NO: 299

SEQ ID NO: 341 - DNA, 317 bp, Canis lupus familiaris
cagcctgtgctaacccagtcgctctccctcttgacatctttggaacaacagtcagactcacctgtaccgtgaacagtggctccagtgttggcagctattacatcaactggttccagtataagccatggagctctccctagtatcacctgtactactacttagactcagataagcaccagggctctggggtccccagctgcttctcctgatccaaggatgcctcagtcattggagggcaccctcatctcggggctgcagcctgaggactagactgaccttcactgtctaatcagaaacaataatgcttctaacagtga

SEQ ID NO: 342 - DNA, 288 bp, Canis lupus familiaris
cccagcgccctttctctctgcatccctgggaacaacagccagactcacctgcaccctgagcagcggctagagtggtggctataggctggttccagcagccaggaagcctcctgagtacctgctgatggtctactgagactcaccaggctatggggtccccagcatcttctctggctccaaggacacctcggccaatgcagggctcctgctcatctctgggctgcagcctgaggtcgaggctgactgtcactgtgctacagaccatggcagtgggagcagctcccgata

SEQ ID NO: 343 - DNA, 278 bp, Canis lupus familiaris
ataacagccagattcacctgctccctgagcagtggcttcagtgttggtggctataacacactggtaccagcagaagccagggagccctccctgttacctcctgtactactactcagaatcagataaacaccatggctccgggatcaccagctgcttccctggccctatggacacctcggccaatgcagggctcctgctcatctcagggctgcagcctgaggacgaggctgactactactgcggtatactccacagcagtgggagcagctactcttacc

SEQ ID NO: 344 - DNA, 307 bp, Canis lupus familiaris
caggctgtgacgacacactcctccttcctctctgcacctttgggatcatcaaccagactcacctgcatccttcccagggcctgaatgttggcaggtactgaacatactggacaaggagaatcaaggaggcatcaggagttccctcagatccagataagtgccagggcacggggttctcagccacttctatggatctaatgatgcctcaggcaatgcaggtttcctgctcatgtctgggctgcagcctgaggacgaggctgactatgactatgctgcacattgtggggtgggagcagctcccgatact

SEQ ID NO: 345 - DNA, 305 bp, Canis lupus familiaris
cagcctgtgctgacccagccgccctccctctctgcatccctgggaacaacattcagactcacctgcaccctgagcagcagctgcagcggtggccatatgctggttccagcatgcaagaggcctcctgagtacctactgatggtctactgagactcaccagggccctggggtccccagcctcttctctggctccaaggacaccttggccaatgcagggctcctgctcatctctgggctgcagcctgagaatgaggctgactgtcactgtgctacagaccatggcagtgggaacagctcccaatact

SEQ ID NO: 346 - DNA, 275 bp, Canis lupus familiaris
caggctgtggtgactccagagcccttctgaccatccccaggagtgacagtcacttttacctgtgactccagcactggagagtcattaatagtgactatccacgttagttccagcagaagcctagacaaactcgcaccacacacacaacaaacactcacggactcccacccagttctcaggctccctccaggctcaaaactgccctcacctttttggggtcccagcctgagaaagaaggtgagtactaccatatgctggtctatcttggttcttgg

SEQ ID NO: 347 - DNA, 290 bp, Canis lupus familiaris
caggctgtggtgactcaggaaccctcactgaccgtgtccctggagggacagtcactctcacctgtgcctccagcactggcgaggtcaccaatggacactatccatactggttccagcagaagcctggccaagtccccaggacattgatttataatacacacataatactcctggacccctacccggttctcaggctgcctctttgggggcaaagctgccttgaccatcacaggggcccagcccgaggatgaagctgaggactactgctggctagtatatatggtaatagg

SEQ ID NO: 348 - DNA, 289 bp, Canis lupus familiaris
caggctgtggtgattcaggaatcctcactaacagtgcccccaggaggaacactctcacctgtgcctcgaacactggcacagtcaccaatgtcagtatccttactggtttcagcagaaccctagtcaagtccccagggcattgacttaggatacaagcaataaacacttctggatccctaccaagctttcagtttccctccttggatgtaaaactcccctgaccttctctggttccctagcctgaggccaaggctgattaccactggtgggtactcatagtggtgctgca

SEQ ID NO: 349 - DNA, 263 bp, Canis lupus familiaris
caggtcatggtgactcaggagccttcatggccatgtccccaggagggacagtcactctcacctatgcctccagcacaggacactatccatactggatccaagaaaatattggccaagtcagggccatttatttataataaaaacaacaaatactgatttctcatgctcccttcttgggagcaaatctgacatgaccatctcctagtgcccagcctgaggacgaggatgagtacccatgggggctacactatagtggtgctggg

SEQ ID NO: 350 - DNA, 246 bp, Canis lupus familiaris
cagattgtggtgactcaggagccttcatggtcgtgtccccaggagggacagtcactctcactatgcctccagcacagaacactatccatactggatccaggaaaatattggccaagtctagagcatttatttataaaagaaacaataaatactgatttctaggctcccttcttgggaataaatctgacttgaccatctgctagtgcgcagcctgaggacgaggctgagtacccctaggggttacac

SEQ ID NO: 351 - DNA, 296 bp, Canis lupus familiaris
caggctgtgatgactcaggagtcctcactaacagtgtccccaggagggacattcactctcacctgtgcctccagccactggcatagtaacaatgctcagtatccttcctggttttaccagaagcctggccaagttcccagggcattgatttaggatacaagcaatgaaaattcctggacccccaccaagtgctcaggttccctttgtggagcaatattctcctgaccctctacagtgccttggtgagaacatagctgagtggcactggtggctgcttttattgtgatgctgggtgc

SEQ ID NO: 352 - DNA, 288 bp, Canis lupus familiaris
caggctgtgatgactcaagagtcctcactaacagtgtccccaggagggacattcactctcacctgcgcctccagctactggcatagtaacaatgctcagtatccttactggttttagcagaatcctggccaagtccccagggcattgatttaggatacaagcaatgaacacacctggacccccaccatgtgctcaggttccctttgtggagcaatattctcctgaccctctacagtgccttggtgagaacatagctgagtggcactggtggctgcttttattgtgatg

SEQ ID NO: 353 - DNA, 275 bp, Canis lupus familiaris
cagactgtggtggcataggagccttcatggccatatccccaggagggacagtcactctcacctatccctccagcacaggacactatctatactggatctagtagcatactggccaagtctaggtcatttatttataataaaaacaataaatactcatagacctccactcatttctcaggctcccatcttgggggcaaatctgactggattgtcccctagtgcccagcctgaggatgaggctgagtaccgctggggctacactatggtggtgtggg

SEQ ID NO: 354 - DNA, 275 bp, Canis lupus familiaris; identical to SEQ ID NO: 353

SEQ ID NO: 355 - DNA, 299 bp, Canis lupus familiaris
cagactgtggtgacccaggagccatcactctcagtgtctctgggagggacagtcaccctcacatgtggcctcagctccgggtcagtctctacaagtaactaccccaactggtcccagcagaccccagggcaggctcctcgcacgattatctacaacacaaacagccgcccctctggggtccctaatcgcttcactggatccatctctgggaacaaagccgccctcaccatcacaggagcccagcctgaggacgaggctgactactactgtgctctgggattaagtagtagtagtagtta

SEQ ID NO: 356 - DNA, 296 bp, Canis lupus familiaris
cagactgtggtaacccaggagccatcactctcagtgtctccaggagggacagtcacactcacatgtggcctcagctctgggtcagtctctacaagtaaccaccctagctggtaccagcagacccaagggaaggctcctcgcatgcttatctacaacacaaacaaccgcccctctgggatccctaattgcttctctggatccatctctgggaacaaagcctccctcaccatcacaggagcccagcctgaggacgagactgactattactgtttattgtatatgggtagtaacattta

SEQ ID NO: 357 - DNA, 307 bp, Canis lupus familiaris
cagattgtggtgacccaggagccatcactctaagtttctccaggagggacagtcacactcacatgtggcctcagctctgggtcagtccctacaagtaactaccccagctggtttcagcagaccccaggccgggctcctagaacagttatctacaacacaaacagctgcccctctggggtccctaatcgcttcactggatccatctctggcaacaaagccgccctcaccatcacaagagcccagcctgaggatgaggctgactcctgctgtgctgaatatcaaagcagtgggagcagctacacttacc

SEQ ID NO: 358 - DNA, 296 bp, Canis lupus familiaris
cagactgtggtaacccaggaaccatcactctcagtgtctccatgagggacagtcacactcacatgtggcctcagctctgggtcagtctctacaagtaactaccccaactggtaccagcagacccaaggccgggctcctcacagggttatctacaacacaaacaaccgcccctctggggtccctgatcgcttctctggatccatctctgggaacaaagccgccctcaccatcacagctgcccagcctgaggacgaggctgactattactgttcattgtatatgggtagtaacatttg

SEQ ID NO: 359 - DNA, 291 bp, Canis lupus familiaris
cagactgtgatcacccaagatacatcactctcagtgtctccaggagggacagtcacactcacatgtggcctcagctctgggtcagtctctacaagtaactaccccagctggtaccagcagacccaaggccgggatcctcgcatgcttatctacagcacaaacagccacccctctggggtccctaattgcttcactagatccatctctgggaagaaagctgccctcaccatcacaggagcccagcctgaggatgagactattattgttcactaaatatgggtagtacatgta

SEQ ID NO: 360 - DNA, 306 bp, Canis lupus familiaris
cagattgtggtgacccaggacccatcactgtcagtgtctagaggagggacagtcacactcacttgtggcctcagctctgggtcagtcactacaataaataccccagctggtcccagcagaccccagggcaggctcctcgcatgattatctatgacacaaacagccgcccctctggggtccctgatcgcttctctggatccatctgtgggaacaaagctgccctcaccatcacaggagcccatcctgaggatgagactgactactactgtggtatacaacatggcagtgggagcagcctcacttacc

SEQ ID NO: 361 - DNA, 307 bp, Canis lupus familiaris
cagattgtggtgacccaggagccatcactgtcagtgtctccaggaggaacagttacactcacatgtggcctaagctctgggtcagtcactataagtaactaccctgattggtaccagcagactccaggcaggtctcctcgcatgcttatctacaacacaaacaaccgcccctctggggtccctaatcacttctctggatccatctctgggaacaaagccgccctcaccatcacaggagcccagcctgaggatgaggcttactactactgtgctgtgtatcaaggcagtgggagcagctacacttacc

SEQ ID NO: 362 - DNA, 291 bp, Canis lupus familiaris
cagactgtggtcacccaggatccatcactctcagtgtctccaggaggaacagtcacactcacatgtggcctcagctctgggtcagtctctacaagtaactaccccggctggtaccagcagacccaagtgaaagctccttgcatgcttatctacagcacaaacagctacccctctggggttcctaattgcttcactggatccatctctgggaagaaagctgccctcaccatcacaggagaccagcctgaggatgagactattattgttcactgcatatgggtagtacactta

SEQ ID NO: 363 - DNA, 306 bp, Canis lupus familiaris
cagactgtggtggctcaggagtcatcagtctcagtgtctccaggagggacagtcacactcacttgtggcctcagctctgggtcagtgactacaagtaactaccacagctggtaccagcggacccaaggccggtctcctcacatgcttatctatgacacaagcagccgtccttctgaggtcctgatcgcttccctggttccatctctgggaacaaagctgccctcactgtcagaggagcccagcctgaggacgaggctgactactactgtggcatgcatgatgtcagtgggaggaattacaattacc

SEQ ID NO: 364 - DNA, 302 bp, Canis lupus familiaris
cagattgtggtggccaggaggcattgttgtcagtgtctccaggagggagagtcacactcacttgtggcctcagctctgggtcagtcactacaagtaactaccccaactggttccagcagaccccagggcgggctcctggcacgattatctacagcacaaaagactgcccctctggggtccctgactgcttctctagatccatctctgggaacaaagccgccctcaccatcacaggagcccagtctgaggacgaggctattactgttttacacgacatggtagtgggagctgctacacttacc

SEQ ID NO: 365 - DNA, 288 bp, Canis lupus familiaris
cagactgtggtaacccaggagccatcactctcagtgtctccaggagggacagtcacactcacttgtggcctcagctctgggtcagtctctacaggtaacaaacctggctggtaccagcacaccccaggccaggctcctcgcaggattatctatgacacaagcagccgcccttctggggtccctgatcgcttctctggatccatctctgagaacaaaactgccctcaccatcacagaagcccaacctgaggatgaggctgactacatcatatatgagtggtggtgctta

SEQ ID NO: 366 - DNA, 305 bp, Canis lupus familiaris
cagattgtggtgacccaggaggcatcgttgttagtgtctcctggagggatagtcacactcacttgtggcctcagctctggatcaatcactacaagtaactaccccaactggctccagcagaccccagggcgggctcctcgcagatgatctatggcacaaaaagccgcccctctggggtccctgatcgcttctgtagatccatctctgggaacaaagccgccctcaccatcacaggagcccagtctgaggatgaggctgactattactgttttacacgacatggcagtgggagcagctacaattac

SEQ ID NO: 367 - DNA, 309 bp, Canis lupus familiaris
cagactgtggtgacccaggagtcatcagtctcagtgtctccaggaggaacagtcacactcccttgtggcctcagctctgggtcactgactacaagtaacactacaccagctggtaccagcagacccaaggccagtctcctcgcatgcttgtctatgacacaagcagctgtccctctgaggttcctgatcacttctctggatccatttctgggaacaaagccaccctcaccatcacaggagcccagcctgaggacgaggctgactactactgtggcatgcatgatgtcagtgggagcagctaaaattacc

SEQ ID NO: 368 - DNA, 311 bp, Canis lupus familiaris
catattttggtgactcaggagccatcactgtcagtgtctccatgagggacagtcacactcacttgtggcctcagctctgggtcagtcactacaagtaactaccccaggtataccagcagaacccaggcaaggctcctagcacagttatctacaacaaaaacagctgcccctctggggtccatggtcgattctctggatccatctctggaagcaaagccgccttcacaatcacaggagcccagcctgaggttgaggctgactactactgtgttacagaacatggctcctcacatgggaacagcctcactcac

SEQ ID NO: 369 - DNA, 291 bp, Canis lupus familiaris
cagactgtggtcacccaggatccgtcactctcagtgtctccaggagggacagtcacattcacatgtggcctcagctctgggtaagtctctacaagaaactaccccagctggtaccagcagacccaaggccaggctccttgcatgcttatctacagcacaagcagacacccttctggggtccctgatcgcttctctggatccatctctgggaacaaagtcgccctcaccatcacaggagcccagcctgaggataagactattattgttcactgcatatgggtagtacattta

SEQ ID NO: 370 - DNA, 296 bp, Canis lupus familiaris
cagactgtggtaacccaggagccatcactctcagtgtctccaggagggacagtcacactcacatgtggcctcagctctgggtcagtctctacaagtaattaccctggctggtaccagcagacccaaggccgggctcctcgcacgattatctacaacacaagcagccgcccctctggggtccctaatcgcttctctggatccatctctggaaacaaagccgccctcaccatcacaggagcccagcccgaggatgaggctgactattactgttccttgtatacgggtagttacactga

SEQ ID NO: 371 - DNA, 296 bp, Canis lupus familiaris
cagactgtggtcacccagaagccatcactctcagtgtctccaggagggacagtcacactcatatgtggcttcagctctgggtcagtctctacaagtaattaccctggctggtaccagcagacccaaggccgggcttctcgcacaattatctacagcacaagcagccgcccctctggggtccctaatcgcttccctggatccatctctgggaacaaagccgccctcaccatcacaggagcccagcctgaggacgaggctgactattactgttccttgtatatgggtagttacactga

SEQ ID NO: 372 - DNA, 300 bp, Canis lupus familiaris
cagattgtagtgacccaggaaccatcactgtctccaggagggacagtcctactcacttgtggcctcagctctgggtcagtcactacaagtaactactccagctggtaccagcagaccccagggcgggctcctcgcacgattatctacaacactaacagccacccctctggagtccctgatcgcttctctggatccatctctgggaacaaagcggcgctcaccatcacaggagcccagcctgaggacgaggctgactactactgtgttacagaacatggtagtgggagcagcttcacttac

SEQ ID NO: 373 - DNA, 307 bp, Canis lupus familiaris
cagactgtggtgactcaggagtcatcagtctcagtgtctccaggagggacagtcacactcacgtgtgacctcagctctgggtcagtgactacaagtaacaaccccagctggtaccagcagacccaaggccgatctcctcgcatgcttatctatgacacaagcagctgtccctcggaggtccctgatcgcttctctggatccatttctgggaacacagctgccctcaccatcacaggagcccagcctgaggacaaggctgactactactgtagtatgcatgatgtcagtgggagcagctacaattacc

SEQ ID NO: 374 - DNA, 296 bp, Canis lupus familiaris
cagactgtggtcacccaggagccatcactctcagtgtctccaggagggacagtcacactcacatgtggcctcagttctgggtcagtcactataagtaactaccccagctggtcccagcagaccccagggcaggctcctcacacaataatctacaggacaaacagctgaccctctggggtccctgatcgcttctctggatccatctctgggaacaacgccgccctcagcatcacagtcgcccagcctgaggacgaggctgactattactgttcattgtatatgggtagtaacattta

SEQ ID NO: 375 - DNA, 303 bp, Canis lupus familiaris
cagattgtggtgacccaggagccatcactctcagtgtctagaggagggacagtcacactcacttgtggcctcagctctgagtcaatcactacaactaccccagctgatcccagcagaccccagggcaggctcctcacacaattatctatgacaaaaacagccgcccctctggggtccctgatcacttctcaggatccatctgtgggaacaaagccaccctcaccatcacaggaacccagcctgaggacaaggctgactactactgtggtatccaacatggcagtaggaggagcctcattaacc

SEQ ID NO: 376 - DNA, 306 bp, Canis lupus familiaris
cagactgttgtgactcaggagtcatcagtctcagtgtctccaggagggacagtaacactcacgtgtagcctcagctctgggtcagtgactacaagtaagtactccagctggaccagtagacccaaggccgatctcctcgcatgcttatctatgacacaagcagccgtccctctgaggtccctgatcgcttctctggatccatctccgggaacaaagctgccctcaccatcacaggagcccagcctgaggacgaggctgactactactgtggtatgcatgatgtcagtgggaggagttacaattacc

SEQ ID NO: 377 - DNA, 302 bp, Canis lupus familiaris
cagattgtggtggccaggaggcattgttgtcagtgtcctctggagggagagtcacactcacttgtggcctcagctctgggtcagtcactacaagtaactaccccaactggttccagcagaccccagggcgggctcctggcacgattatgtacagcacaaaagactgcccctctggggtccctgattgcttctctagatccatctctgggaacaaagccgccctcaccatcacaggagcccagtctgaggacgaggttattactgttttacacgacatggtagtgggagctgctacacttacc

SEQ ID NO: 378 - DNA, 296 bp, Canis lupus familiaris
cagactgtggtaacccaggagccatcactctcagtgtctccaggagggacagtcacactcacttgtggcctcagctctgggtcagtctctacaggtaacaaacctggctggtaccagcacaccccaggccaggctcctcgcaggattatctatgacacaagcagccgcccttctggggtccctgatcgcttctctggatccatctctgagaacaaagctgccctcaccatcacagaagcccagcctgaggatgaggctgcctaccactgttcgctgtatatgagtggtggtgctta

SEQ ID NO: 379 - DNA, 306 bp, Canis lupus familiaris
cagattgtggtgacccaggaggcatcgttgtcagtgtctcctggagggatagtcacactcacttgtggcctcagctctggatcaatcactacaagtaactaccccaactggttccagcagaccccagggcgggctcctcgcagatgatctatggcacaaaaagccgcccctctggggtccctgatcgcttctgtagatccatctctgggaacaaagccgccctcaccatcacaggagcccagtctgaggatgaggctgactattactgttttacacgacatggcagtgggagcagctacaattacc

SEQ ID NO: 380 - DNA, 307 bp, Canis lupus familiaris
cagactgtggtgacccaggagtcatcagtctcagtgtctccagtcggaacagtcacactcacttgtggcctcagctctgggtcactgactacaagtaactacaccagctggtaccagcagacccaaggccagtctcctcgcatgcttgtctatgacacaagcagctgtccctctgaagttcctgatcacttctctggatccatttctgggaacaaagccgccctcaccatcacaggagcccagcctgaggacgaggctgactactactgtggtatgcatgatgtcagtgggagcagctaaaattacc

SEQ ID NO: 381 - DNA, 288 bp, Canis lupus familiaris
catattttggtgactcaggagccatcactgtcagtgtctccatgagggacagtcacactcacttgtggcctcagctctgggtcagtcactacaagtaactaccccaggtataccagcagaacccaggcaaggctcctagcacagttatctacaacaaaaacagctgcccctctggggtccatggtcgattctctggatccatctctggaagcaaagccgccttcacaatcacaggagcccagcctgaggttgaggctgactactactgtgttacagaacatggctcct

SEQ ID NO: 382 - DNA, 291 bp, Canis lupus familiaris
cagactgtggtcaaccaggatccgtcactctcagtgtctccaggagggacagtcacattcacatgtggcctcagctctgggtaagtctctgcaagaaactaccccagctggtaccagcagacccaaggccaggctccttgcatgcttatctacagcacaagcagccgcccttctggggtccctgatcgcttctctggatccatctctgggaacaaagtcgccctcaccatcacaggagcccagcctgaggatgagactattattgttcactgcatatgggtagtacattta

SEQ ID NO: 383 - DNA, 296 bp, Canis lupus familiaris
cagactgtggtaacccaggagccatcactctcagtgtctccaggagggacagtcacactcacatgtggcctcagctctgggtcagtctctacaagtaattaccctggctggtaccagcagaccctaggccgggctcctcgcacgattatctacagaacaagcagccgcccctctggggtccctaatcgcttctctggatccatctctgggaacaaagccgccctcaccatcacaggagcccagcctgaggacgaggctgactattactgttccttgtatatgggtagttacactga

SEQ ID NO: 384 - DNA, 291 bp, Canis lupus familiaris
cagactgtggtcaccaaggatccatcactctcagtgtctccaggagggacagtcacattcacatgtggcctcagctctgggtcagtctttacaagtaactaccccagctggtaccagcagacccatggccgggctcctcgcatgcttatctacagcacaaggagctgcccccccggggtccctgatcgcttctctggatccatctctgggaacaaagttgccctcaccatcacaggagcccagcctgaggatgagactattattgttcactgtgtatgggtagtacattta

SEQ ID NO: 385 - DNA, 296 bp, Canis lupus familiaris
cagactgtggtcacccagaagccatcactctcagtgtctccaggagggacagtcacactcatatgtggcctcagctctgggtcagtctctacaagtaattaccctggctggtaccagcagacccaaggccgggcttctcgcacaattatctacagcacaagcagccgcccctctggggtccctaatcgcttcactggatccatctctgggaacaaagccgccctcaccatcacaggagcccagcctgaggacgaggctgactattactgttccttgtatatgggtagttacactga

SEQ ID NO: 386 - DNA, 307 bp, Canis lupus familiaris
cagattgtggtgacccaggaaccatcactgtcagtgtctccaggagggacactcacactcacttgtggcctcagctctgggtcagtcactacaagtaactaccccagctggtaccagcagaccccaggccaggctcctagcacagttatctacaacacaaacagccgcccctctggtgtccctgatcacttctctggatccgtctctgggaacaaagccgccctcatcatcacaggagcccagcctgaggacgaggctgatgactactctgttgcagaacatgtcagtgggagcagcttcacttacc

SEQ ID NO: 387 - DNA, 296 bp, Canis lupus familiaris; identical to SEQ ID NO: 370

SEQ ID NO: 388 - DNA, 291 bp, Canis lupus familiaris
cagactgtggtcaccaaggatccatcactctcagtgtttccaggagggacagtcacattcacatgtggcctcagctctgggtcagtctttacaagtaactaccccagctggtaccagcagacccatggccgggctcctcgcatgcttatctacagcacaagcagctgcccccccggggtccctgatcgcttctctggatccatctctgggaacaaagttgccctcaccatcacaggagcccagcctgaggatgagactattattgttcactgtgtatgggtagtacattta

SEQ ID NO: 389 - DNA, 296 bp, Canis lupus familiaris
cagactgtggtcacccagaagccatcactctcagtgtctccaggagggacagtcacactcatatgtggcctcagctctgggtcagtctctacaagtaattaccctggctggtaccagcagacccaaggccgggcttctcgcacaattatctacagcacaagcagccgcccctctggggtccctaatcgcttccctggatccatctctgggaacaaagccgccctcatcatcacaggagcccagcctgaggacgaggctgactattactgttccttgtatatgggtagttacactga

SEQ ID NO: 390 - DNA, 38 bp, Canis lupus familiaris
ttgggtattcggtgaagggacccagctgaccgtcctcg

SEQ ID NO: 391 - DNA, 38 bp, Canis lupus familiaris
tatggtattcggcagagggacccagctgaccatcctcg

SEQ ID NO: 392 - DNA, 38 bp, Canis lupus familiaris
tagtgtgttcggcggaggcacccatctgaccgtcctcg

SEQ ID NO: 393 - DNA, 38 bp, Canis lupus familiaris
ttacgtgttcggctcaggaacccaactgaccgtccttg

SEQ ID NO: 394 - DNA, 38 bp, Canis lupus familiaris
tattgtgttcggcggaggcacccatctgaccgtcctcg

SEQ ID NO: 395 - DNA, 38 bp, Canis lupus familiaris
tggtgtgttcggcggaggcacccacctgaccgtcctcg

SEQ ID NO: 396 - DNA, 38 bp, Canis lupus familiaris
tgctgtgttcggcggaggcacccacctgaccgtcctcg

SEQ ID NO: 397 - DNA, 38 bp, Canis lupus familiaris; identical to SEQ ID NO: 396

SEQ ID NO: 398 - DNA, 38 bp, Canis lupus familiaris; identical to SEQ ID NO: 393

SEQ ID NO: 399 - DNA, 306 bp, Canis lupus familiaris
  modified_base (1)..(1): a, c, t, g, unknown or other
nagtccaaaaccagccccagtgtgttcccgctgagcctctgccaccaggagtcagaagggtacgtggtcatcggctgcctggtgcagggattcttcccaccggagcctgtgaacgtgacctggaatgccggcaaggacagcacatctgtcaagaacttcccccccatgaaggctgctaccggaagcctatacaccatgagcagccagttgaccctgccagccgcccagtgccctgatgactcgtctgtgaaatgccaagtgcagcatgcttccagccccagcaaggcagtgtctgtgccctgcaaa

SEQ ID NO: 400 - DNA, 342 bp, Canis lupus familiaris
gataactgtcatccgtgtcctcatccaagtccctcgtgcaatgagccccgcctgtcactacagaagccagccctcgaggatctgcttttaggctccaatgccagcctcacatgcacactgagtggcctgaaagaccccaagggtgccaccttcacctggaacccctccaaagggaaggaacccatccagaagaatcctgagcgtgactcctgtggctgctacagtgtgtccagtgtcctaccaggctgtgctgatccatggaaccatggggacaccttctcctgcacagccacccaccctgaatccaagagcccgatcactgtcagcatcaccaaaaccaca

SEQ ID NO: 401 - DNA, 387 bp, Canis lupus familiaris
gagcacatcccgccccaggtccacctgctgccgccgccgtcggaagagctggccctcaatgagctggtgacactgacgtgcttggtgaggggcttcaaaccaaaagatgtgctcgtacgatggctgcaagggacccaggagctaccccaagagaagtacttgacctgggagcccctgaaggagcctgaccagaccaacatgtttgccgtgaccagcatgctgagggtgacagccgaagactggaagcagggggagaagttctcctgcatggtgggccacgaggctctgcccatgtccttcacccagaagaccatcgaccgcctggcgggtaaacccacccacgtcaacgtgtctgtggtcatggcagaggtggacggcatctgctac

SEQ ID NO: 402 - DNA, 213 bp, Canis lupus familiaris
gactcacagtgtcttgcaggttaccgggagccacttccctggctggtgctggacctgtcgcaggaggacctggaggaggatgccccaggagccagcctgtggcccactaccgtcacccttctcaccctcttcctactgagtctcttctacagcacagcactgactgtgacaagcgtgcggggcccaactgacagcagagagggcccccagtac

SEQ ID NO: 403 - DNA, 285 bp, Canis lupus familiaris
gaatcgtcacttctgctccccttggtctcaggatgtaaggtcccaaaaaatggtgaggacataaccctggcctgcttggcaaaaggacccttcctagattctgtgcgggtcacgacaggcccagagtcacaggcccagatggaaaagaccacactgaagatgctaaagataccggaccacactcaggtgtctctcctgtccaccccctggaaaccaggcctgcactactgcgaagccatcaggaaagataacaaagagaagctgaagaaagccatccactggcca

SEQ ID NO: 404 - DNA, 90 bp, Canis lupus familiaris
gcatcctgggaaactgctatctccctgttgactcatgcgccatcccgaccccaggaccacacccaagcccccagcatggccagggtctca

SEQ ID NO: 405 - DNA, 69 bp, Canis lupus familiaris
gtgcctcccaccagccacacccagacgcaagcccaggagccaggatgcccagtggacaccatcctcaga

SEQ ID NO: 406 - DNA, 327 bp, Canis lupus familiaris
gagtgttggaaccacacccaccctcccagcctctacatgctgcgccctcccctgcggggaccatggctccagggagaagctgctttcacctgcctggtggtgggagatgaccttcagaaggcccacctgtcctgggaggtagccggggcgccccccagcgaggctgtggaggagaggccactgcaggagcatgagaatggctcccagagctggagcagccgcctggtcttgcccatatccctgtgggcctcaggagccaacatcacctgcacgctgagcctccccagcatgccttcccaggtggtgtccgcagcagccagagagcat

SEQ ID NO: 407 - DNA, 312 bp, Canis lupus familiaris
gctgccagagcacccagcagcctcaatgtccatgccctgaccatgcccagagcagcctcctggttcctgtgcgaggtgtccggcttctcaccccctgacatcctcctcacctggatcaaggaccagattgaggtggacccttcttggttcgccactgcaccccccatggcccagccgggcagtggcacgttccagacctggagtctcctgcgtgtcctcgctccccagggccctcacccgcccacctacacgtgtgtagtcaggcacgaggcctcccggaagctgctcaacaccagctggagcctggacagt

SEQ ID NO: 408 - DNA, 150 bp, Canis lupus familiaris
ggtctgaccatgacccccccagcccctcagagccacgacgagagcagcggggactccatggatctggaagatgccagcggactgtggcccacgttcgctgccctcttcgtcctcactctgctctacagcggcttcgtcaccttcctcaaa

SEQ ID NO: 409 - DNA, 6 bp, Canis lupus familiaris
gtgaag

SEQ ID NO: 410 - DNA, 297 bp, Canis lupus familiaris
  modified_base (1)..(1): a, c, t, g, unknown or other
nccaccagccaggacctgtctgtgttccccttggcctcctgctgtaaagacaacatcgccagtacctctgttacactgggctgtctggtcaccggctatctccccatgtcgacaactgtgacctgggacacggggtctctaaataagaatgtcacgaccttccccaccaccttccacgagacctacggcctccacagcatcgtcagccaggtgaccgcctcgggcgagtgggccaaacagaggttcacctgcagcgtggctcacgctgagtccaccgccatcaacaagaccttcagt

SEQ ID NO: 411 - DNA, 324 bp, Canis lupus familiaris
gcatgtgccttaaacttcattccgcctaccgtgaagctcttccactcctcctgcaaccccgtcggtgatacccacaccaccatccagctcctgtgcctcatctctggctacgtcccaggtgacatggaggtcatctggctggtggatgggcaaaaggctacaaacatattcccatacactgcacccggcacaaaggagggcaacgtgacctctacccacagcgagctcaacatcacccagggcgagtgggtatcccaaaaaacctacacctgccaggtcacctatcaaggctttacctttaaagatgaggctcgcaagtgctca

SEQ ID NO: 412 - DNA, 321 bp, Canis lupus familiaris
gagtccgacccccgaggcgtgagcagctacctgagcccacccagcccccttgacctgtatgtccacaaggcgcccaagatcacctgcctggtagtggacctggccaccatggaaggcatgaacctgacctggtaccgggagagcaaagaacccgtgaacccgggccctttgaacaagaaggatcacttcaatgggacgatcacagtcacgtctaccctgccagtgaacaccaatgactggatcgagggcgagacctactattgcagggtgacccacccgcacctgcccaaggacatcgtgcgctccattgccaaggcccct

SEQ ID NO: 413 - DNA, 339 bp, Canis lupus familiaris
ggcaagcgtgcccccccggatgtgtacttgttcctgccaccggaggaggagcaggggaccaaggacagagtcaccctcacgtgcctgatccagaacttcttccccgcggacatttcagtgcaatggctgcgaaacgacagccccatccagacagaccagtacaccaccacggggccccacaaggtctcgggctccaggcctgccttcttcatcttcagccgcctggaggttagccgggtggactgggagcagaaaaacaaattcacctgccaagtggtgcatgaggcgctgtccggctctaggatcctccagaaatgggtgtccaaaacccccggtaaa

SEQ ID NO: 414 - DNA, 129 bp, Canis lupus familiaris
gagctccaggagctgtgcgcggatgccactgagagtgaggagctggacgagctgtgggccagcctgctcatcttcatcaccctcttcctgctcagcgtgagctacggcgccaccagcaccctcttcaag

SEQ ID NO: 415 - DNA, 81 bp, Canis lupus familiaris
gtgaagtgggtactcgccaccgtcctgcaggagaagccacaggccgcccaagactacgccaacatcgtgcggccggcacag

SEQ ID NO: 416 - DNA, 291 bp, Canis lupus familiaris
gcctccaccacggccccctcggttttcccactggcccccagctgcgggtccacttccggctccacggtggccctggcctgcctggtgtcaggctacttccccgagcctgtaactgtgtcctggaattccggctccttgaccagcggtgtgcacaccttcccgtccgtcctgcagtcctcagggcttcactccctcagcagcatggtgacagtgccctccagcaggtggcccagcgagaccttcacctgcaacgtggtccacccagccagcaacactaaagtagacaagcca

SEQ ID NO: 417 - DNA, 42 bp, Canis lupus familiaris
gtgttcaatgaatgcagatgcactgatacacccccatgccca

SEQ ID NO: 418 - DNA, 330 bp, Canis lupus familiaris
gtccctgaacctctgggagggccttcggtcctcatctttcccccgaaacccaaggacatcctcaggattacccgaacacccgaggtcacctgtgtggtgttagatctgggccgtgaggaccctgaggtgcagatcagctggttcgtggatggtaaggaggtgcacacagccaagacccagtctcgtgagcagcagttcaacggcacctaccgtgtggtcagcgtcctccccattgagcaccaggactggctcacagggaaggagttcaagtgcagagtcaaccacatagacctcccgtctcccatcgagaggaccatctctaaggccaga

SEQ ID NO: 419 - DNA, 330 bp, Canis lupus familiaris
gggagggcccataagcccagtgtgtatgtcctgccgccatccccaaaggagttgtcatccagtgacacagtcagcatcacctgcctgataaaagacttctacccacctgacattgatgtggagtggcagagcaatggacagcaggagcccgagaggaagcaccgcatgaccccgccccagctggacgaggacgggtcctacttcctgtacagcaagctctctgtggacaagagccgctggcagcagggagaccccttcacatgtgcggtgatgcatgaaactctacagaaccactacacagatctatccctctcccattctccgggtaaa

SEQ ID NO: 420 - DNA, 291 bp, Canis lupus familiaris
  modified_base (1)..(1): a, c, t, g, unknown or other
ncctccaccacggccccctcggttttcccactggcccccagctgcgggtccacttccggctccacggtggccctggcctgcctggtgtcaggctacttccccgagcctgtaactgtgtcctggaattccggctccttgaccagcggtgtgcacaccttcccgtccgtcctgcagtcctcagggctctactccctcagcagcatggtgacagtgccctccagcaggtggcccagcgagaccttcacctgcaacgtggcccacccggccagcaaaactaaagtagacaagcca

SEQ ID NO: 421 - DNA, 57 bp, Canis lupus familiaris
gtgcccaaaagagaaaatggaagagttcctcgcccacctgattgtcccaaatgccca

SEQ ID NO: 422 - DNA, 330 bp, Canis lupus familiaris
gcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccaga

SEQ ID NO: 423 - DNA, 327 bp, Canis lupus familiaris
gggcaggcccatcaacccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacagaaatccctctcccattctccgggtaaa

SEQ ID NO: 424 - DNA, 132 bp, Canis lupus familiaris
gagctgatcctggatgacagctgtgctgaggaccaggacggggagctggacgggctgtggaccaccatctccatcttcatcaccctcttcctgctcagcgtgtgctacagcgccactgtcaccctcttcaag

SEQ ID NO: 425 - DNA, 81 bp, Canis lupus familiaris
gtgaagtggatcttctcatcagtggtggagctgaagcgcacgattgtccccgactacaggaatatgatcgggcagggggcc

SEQ ID NO: 426 - DNA, 291 bp, Canis lupus familiaris
gcctccaccacggccccctcggttttcccactggcccccagctgtgggtcccaatccggctccacggtggccctggcctgcctggtgtcaggctacatccccgagcctgtaactgtgtcctggaattccgtctccttgaccagcggtgtgcacaccttcccgtccgtcctgcagtcctcagggctctactccctcagcagcatggtgacagtgccctccagcaggtggcccagcgagaccttcacctgcaatgtggcccacccggccaccaacactaaagtagacaagcca

SEQ ID NO: 427 - DNA, 51 bp, Canis lupus familiaris
gtggccaaagaatgcgagtgcaagtgtaactgtaacaactgcccatgccca

SEQ ID NO: 428 - DNA, 330 bp, Canis lupus familiaris
ggttgtggcctgctgggagggccttcggtcttcatctttcccccaaaacccaaggacatcctcgtgactgcccggacacccacagtcacttgtgtggtggtggatctggacccagaaaaccctgaggtgcagatcagctggttcgtggatagtaagcaggtgcaaacagccaacacgcagcctcgtgaggagcagtccaatggcacctaccgtgtggtcagtgtcctccccattgggcaccaggactggctttcagggaagcagttcaagtgcaaagtcaacaacaaagccctcccatcccccattgaggagatcatctccaagacccca

SEQ ID NO: 429 - DNA, 327 bp, Canis lupus familiaris
gggcaggcccatcagcctaatgtgtatgtcctgccgccatcgcgggatgagatgagcaagaatacggtcaccctgacctgtctggtcaaagacttcttcccacctgagattgatgtggagtggcagagcaatggacagcaggagcctgagagcaagtaccgcatgaccccgccccagctggatgaagatgggtcctacttcctatacagcaagctctccgtggacaagagccgctggcagcggggagacaccttcatatgtgcggtgatgcatgaagctctacacaaccactacacacagatatccctctcccattctccgggtaaa

SEQ ID NO: 430 - DNA, 291 bp, Canis lupus familiaris
gcctccaccacggccccctcggttttcccactggcccccagctgcgggtccacttccggctccacggtggccctggcctgcctggtgtcaggctacttccccgagcctgtaactgtgtcctggaattccggctccttgaccagcggtgtgcacaccttcccgtccgtcctgcagtcctcagggctctactccctcagcagcacggtgacagtgccctccagcaggtggcccagcgagaccttcacctgcaacgtggtccacccggccagcaacactaaagtagacaagcca

SEQ ID NO: 431 - DNA, 42 bp, Canis lupus familiaris
gtgcccaaagagtccacctgcaagtgtatatccccatgccca

SEQ ID NO: 432 - DNA, 330 bp, Canis lupus familiaris
gtccctgaatcactgggagggccttcggtcttcatctttcccccgaaacccaaggacatcctcaggattacccgaacacccgagatcacctgtgtggtgttagatctgggccgtgaggaccctgaggtgcagatcagctggttcgtggatggtaaggaggtgcacacagccaagacgcagcctcgtgagcagcagttcaacagcacctaccgtgtggtcagcgtcctccccattgagcaccaggactggctcaccggaaaggagttcaagtgcagagtcaaccacataggcctcccgtcccccatcgagaggactatctccaaagccaga

SEQ ID NO: 433 - DNA, 330 bp, Canis lupus familiaris
gggcaagcccatcagcccagtgtgtatgtcctgccaccatccccaaaggagttgtcatccagtgacacggtcaccctgacctgcctgatcaaagacttcttcccacctgagattgatgtggagtggcagagcaatggacagccggagcccgagagcaagtaccacacgactgcgccccagctggacgaggacgggtcctacttcctgtacagcaagctctctgtggacaagagccgctggcagcagggagacaccttcacatgtgcggtgatgcatgaagctctacagaaccactacacagatctatccctctcccattctccgggtaaa

SEQ ID NO: 434 - DNA, 318 bp, Canis lupus familiaris
  modified_base (1)..(1): a, c, t, g, unknown or other
nagagtccatcccctccaaacctcttccccctcatcacctgtgagaactccctgtccgatgagaccctcgtggccatgggctgcctggcccgggacttcctgcctggctccatcaccttctcctggaagtacgagaacctcagtgcaatcaacaaccaggacattaagaccttcccttcagttctgagagagggcaagtatgtggcgacctctcaggtgttcctgccctccgtggacatcatccagggttcagacgagtacatcacatgcaacgtcaagcactccaatggtgacaaatctgtgaacgtgcccatcaca

SEQ ID NO: 435 - DNA, 339 bp, Canis lupus familiaris
gggcctgtaccaacgtctcccaacgtgactgtcttcatcccaccccgcgacgccttctctggcaatggccagcgcaagtcccagctcatctgccaggctgcaggtttcagccccaagcagatttccgtgtcttggttccgtgatggaaagcagattgagtctggcttcaacacagggaaggcagaggccgaggagaaagagcatgggcctgtgacctacagcatcctcagcatgctgaccatcaccgagagtgcctggctcagccagagcgtgttcacctgccacgtggagcacaatgggatcatcttccagaagaacgtgtcctccatgtgcacctcc

SEQ ID NO: 436 - DNA, 318 bp, Canis lupus familiaris
aatacacccgttggcatcagcatcttcaccatccccccctcctttgccagcatcttcaacaccaagtcagccaagctgtcctgcctggtcactgacctggccacttatgacagcctgaccatctcctggacccgtcagaatggcgaggctctgaaaacccacaccaacatctctgagagccatcccaacaacaccttcagtgccatgggggaagccactgtctgcgtggaggaatgggagtcaggcgagcagttcacctgcacagtgacccacacagatctgccctcaccgctgaagaagaccatctccaggcccaag

SEQ ID NO: 437 - DNA, 393 bp, Canis lupus familiaris
gatgtcaacaagcacatgccttctgtctacgtcctgcccccgagccgggagcagctgagcctgcgggaatcggcctcactcacctgcctggtgaaaggcttctcacccccagatgtgttcgtgcagtggctgcagaagggccagcccgtgccccctgacagctacgtgaccagcgccccgatgcccgagccccaagcccccggcctctactttgtccacagcatcctgaccgtgagtgaggaggactggaatgccggggagacctacacctgtgttgtaggccatgaggccctgccccatgtggtgaccgagaggagcgtggacaagtccaccggtaaacccaccttgtacaacgtgtccctggtcttatctgacacagccagcacctgctac

SEQ ID NO: 438 - DNA, 117 bp, Canis lupus familiaris
gggggggaggtgagtgccgaggaggaaggcttcgagaacctgaataccatggcatccaccttcatcgtcctcttcctcctcagtgtcttctacagcaccacagtcactctgttcaag

SEQ ID NO: 439 - DNA, 6 bp, Canis lupus familiaris
gtgaaa

SEQ ID NO: 440 - DNA, 330 bp, Canis lupus familiaris
cggaatgatgcccagccagccgtctatttgttccaaccatctccagaccagttacacacaggaagtgcctctgttgtgtgcttgctgaatagcttctaccccaaagacatcaatgtcaagtggaaagtggatggtgtcatccaagacacaggcatccaggaaagtgtcacagagcaggacaaggacagtacctacagcctcagcagcaccctgacgatgtccagtactgagtacctaagtcatgagttgtactcctgtgagatcactcacaagagcctgccctccaccctcatcaagagcttccaaaggagcgagtgtcagagagtggac

SEQ ID NO: 441 - DNA, 318 bp, Canis lupus familiaris
ggtcagcccaagtcctcccccttggtcacactcttcccgccctcctctgaggagctcggcgccaacaaggctaccctggtgtgcctcatcagcgacttctaccccagtggcctgaaagtggcttggaaggcagatggcagcaccatcatccagggcgtggaaaccaccaagccctccaagcagagcaacaacaagtacacggccagcagctacctgagcctgacgcctgacaagtggaaatctcacagcagcttcagctgcctggtcacgcaccaggggagcaccgtggagaagaaggtggcccctgcagagtgctct

SEQ ID NO: 442 - DNA, 318 bp, Canis lupus familiaris
ggtcagcccaaggcctccccctcagtcacactcttcccaccctcctctgaggagctcggcgccaacaaggccaccctggtgtgcctcatcagcgacttctaccccagcggcgtgacggtggcctggaaggcagacggcagccccggcatccagggcgtggagaccaccaagccctccaagcagagcaacaacaagtacgcggccagcagctacctgagcctgacgcctgacaagtggaaatctcacagcagcttcagctgcctggtcacgcatgaggggagcaccgtggagaagaaggtggcccccgcagagtgctct

SEQ ID NO: 443 - DNA, 318 bp, Canis lupus familiaris
ggtcagcccaaggcctccccctcggtcacactcttcccgccctcctctgaggagctcggcgccaacaaggccaccctggtgtgcctcatcagcgacttctaccccagtggcgtgacggtggcctggaaggcagacggcagccccgtcacccagggcgtggagaccaccaagccctccaagcagagcaacaacaagtacgcggccagcagctacctgagcctgacgcctgacaagtggaaatctcacagcagcttcagctgcctggtcacacacgaggggagcaccgtggagaagaaggtggcccccgcagagtgctct

SEQ ID NO: 444 - DNA, 289 bp, Canis lupus familiaris
ggtcagcccaaggcctccccctcggtcacactcttcccgccctcctctgaggagctcggcgccaacaaggccaccctggtgtgcctcatcagcgacttctaccccagcggtgtgacggtggcctggaaggcagacggcagccccgtcacccagggcgtggagaccaccaagccctccaagcagagcaacaacaagtacgcggccagcagctacctgagcctgacgcctgacaagtggaaatctcacagcagcttcagctgcctggtcacacacgaggggagcactgtgg

SEQ ID NO: 445 - DNA, 318 bp, Canis lupus familiaris
ggtcagcccaaggcctccccttcggtcacactcttcccgccctcctctgaggagcttggcgccaacaaggccaccctggtgtgcctcatcagcgacttctaccccagcggcgtgacagtggcctggaaggcagacggcagccccatcacccagggtgtggagaccaccaagccctccaagcagagcaacaacaagtacgcggccagcagctacctgagcctgacgcctgacaagtggaaatctcacagcagcttcagctgcctggtcacgcacgaggggagcaccgtggagaagaaggtggcccccgcagagtgctct

SEQ ID NO: 446 - DNA, 318 bp, Canis lupus familiaris
ggtcagcccaaggcctccccctcggtcacactcttcccgccctcctctgaggagctcggcgccaacaaggccaccctggtgtgcctcatcagcgacttctaccccagcggtgtgacggtggcctggaaggcagacggcagccccgtcacccagggcgtggagaccaccaagccctccaagcagagcaacaacaagtacgcggccagcagctacctgagcctgacgcctgacaagtggaaatctcacagcagcttcagctgcctggtcacgcacgaggggagcaccgtggagaagaaggtggcccccgcagagtgctct

SEQ ID NO: 447 - DNA, 318 bp, Canis lupus familiaris
ggtcagcccaaggcctccccctcggtcacactcttcccgccctcctctgaggagctcggcgccaacaaggccaccctggtgtgcctcatcagcgacttctaccccagcggcgtgacggtggcctggaaggcagacggcagccccgtcacccagggcgtggagaccaccaagccctccaagcagagcaacaacaagtacgcggccagcagctacctgagcctgacgcctgacaagtggaaatctcacagcagcttcagctgcctggtcacgcacgaggggagcaccgtggagaagaaggtggcccccgcagagtgctct

SEQ ID NO: 448 - DNA, 318 bp, Canis lupus familiaris; identical to SEQ ID NO: 447

SEQ ID NO: 449 - DNA, 318 bp, Canis lupus familiaris
ggtcagcccaaggcctccccctcggtcacactcttcccgccctcctctgaggagctcggcgccaacaaggccaccctggtgtgcctcatcagcgacttctaccccagcggcgtgacggtggcctggaaggcagacggcagccccatcacccagggcgtggagaccaccaagccctccaagcagagcaacaacaagtacgcggccagcagctacctgagcctgacgcctgacaagtggaaatctcacagcagcttcagctgcctggtcacgcacgaggggagcactgtggagaagaaggtggcccccgcagagtgctct

SEQ ID NO: 450 - DNA, 24 bp, Artificial Sequence
  Description of Artificial Sequence Synthetic primer
acataatacactgaaatggagccc

SEQ ID NO: 451 - DNA, 20 bp, Artificial Sequence
  Description of Artificial Sequence Synthetic primer
gtccttggtcaacgtgaggg

SEQ ID NO: 452 - DNA, 24 bp, Artificial Sequence
  Description of Artificial Sequence Synthetic primer
cataatacactgaaatggagccct

SEQ ID NO: 453 - DNA, 20 bp, Artificial Sequence
  Description of Artificial Sequence Synthetic primer
gcaacagtggtaggtcgctt

SEQ ID NO: 454 - DNA, 100 bp, Mus musculus
atttctgtacctgatctatgtcaatatctgtaccatggctctagcagagatgaaatatgagacagtctgatgtcatgtggccatgcctggtccagacttg

SEQ ID NO: 455 - DNA, 100 bp, Mus musculus
gtcaatcagcagaaatccatcatacatgagacaaagttataatcaagaaatgttgcccataggaaacagaggatatctctagcactcagagactgagcac

SEQ ID NO: 456 - DNA, 1103 bp, Artificial Sequence
  Description of Artificial Sequence Synthetic polynucleotide
gcattgaataaaccagtataaacaagcaagcaaagatagatagatagatagatagatagatagatagatacatagatagatagatagatagatagatgatagatagatagatagatagatagatttttacgtataatacaataaaaacattcattgtccctctattggtgactactcaaggaaaaaaatgttcatatgcaagaaaaaatgttatcattaccagatgatccagcaatctagcaatatatatattgtttattcacaaaacatgaatgaaccttttaagaagctgttacagtgtaaaaattaagttaaatcactgaagaacatatactgtgtgatttcattcaaatgaaatttgagaagtaaatatatatgtatatatatatatatgtaaaaaatataagtctgaactacaaaaattcaatttgtttgatatgtaagaataagaaaaattgacccccaaaatttgttaataattaggtatgtgtatttttatgaatatataagtataataatgcttatagtatacactattctgaatcacatttattccctaagtgtgttcccttgattataattaaaagtatattttttaaatacagagtcagagtacagtcaataaggcgaaaatatagttgaatgatttgcttcagcttttgtaatgtactagagattgtgagtacaaagtctcagagctcattttatccctgacaataaccagctctgtgcttcaagtacatttccatctttctctgaaatttagtcttatatagatagacaaaatttaagtaaatttcaaactacacagaacaactaagttgttgtttcatattgataatggatttgaactgcattaacagaactttaacatcctgcttattctcccttcagccatcatattttgctttattattttcactttttgagttatttttcacattcagaaagctcacataattgtcacttctttgtatactggtatacagaccagaacatttgcatattgttccctggggaggtctttgccctgttggcctgagataaaacctcaagtgtcctcttgcctccactgatcactctcctatgtttatttcctcaaa

SEQ ID NO: 457 - DNA, 46 bp, Artificial Sequence
  Description of Artificial Sequence Synthetic oligonucleotide
atgaggttcccttctcagctcctggggctgctgatgctctggatcc

SEQ ID NO: 458 - DNA, 377 bp, Artificial Sequence
  Description of Artificial Sequence Synthetic polynucleotide
caggtaaggacagggcggagatgaggaaagacatgggggcgtggatggtgagctcccctggtgctgtttctctccctgtgtattctgtgcatgggacagattgccctccaacagggggaatttaatttttagactgtgagaattaagaagaatataaaatatttgatgaacagtactttagtgagatgctaaagaagaaagaagtcactctgtcttgctatcttgggttttccatgataattgaatagatttaaaatataaatcaaaatcaaaatatgatttagcctaaaatatacaaaacccaaaatgattgaaatgtcttatactgtttctaacacaacttgtacttatctctcattattttaggatccagtggg

SEQ ID NO: 459 - DNA, 13 bp, Artificial Sequence
  Description of Artificial Sequence Synthetic oligonucleotide
aggatccagtggg

SEQ ID NO: 460 - DNA, 297 bp, Artificial Sequence
  Description of Artificial Sequence Synthetic polynucleotide
gatattgtcatgacacagaccccactgtccctgtctgtcagccctggagagactgcctccatctcctgcaaggccagtcagagcctcctgcacagtgatggaaacacgtatttgaactggttccgacagaagccaggccagtctccacagcgtttaatctataaggtctccaacagagaccctggggtcccagacaggttcagtggcagcgggtcagggacagatttcaccctgagaatcagcagagtggaggctgacgatactggagtttattactgcgggcaaggtatacaagat

SEQ ID NO: 461 - DNA, 7 bp, Artificial Sequence
  Description of Artificial Sequence Synthetic oligonucleotide
cacagtg

SEQ ID NO: 462 - DNA, 142 bp, Artificial Sequence
  Description of Artificial Sequence Synthetic polynucleotide
atacagactctatcaaaaacttccttgcctggggcagcccagctgacaatgtgcaatctgaagaggagcagagagcatcttgtgtctgtgtgagaaggaggggctgggatacatgagtaattctttgcagctgtgagctctg

SEQ ID NO: 463 - DNA, 1103 bp, Artificial Sequence; identical to SEQ ID NO: 456
  Description of Artificial Sequence Synthetic polynucleotide

SEQ ID NO: 464 - DNA, 49 bp, Artificial Sequence
  Description of Artificial Sequence Synthetic oligonucleotide
atgatgagtcctgcccagttcctgtttctgttagtgctctggattcagg

SEQ ID NO: 465 - DNA, 386 bp, Artificial Sequence
  Description of Artificial Sequence Synthetic polynucleotide
gtaaggagttttggaatgtgagggatgagaatggggatggagggtgatctctggatgcctatgtgtgctgtttatttgtggtggggcaggtcatatcttccagaatgtgaggttttgttacatcctaatgagatattccacatggaacagtatctgtactaagatcagtattctgacatagattggatggagtggtatagactccatctataatggatgatgtttagaaacttcaacacttgttttatgacaaagcatttgatatataatatttttaaatctgaaaaactgctaggatcttacttgaaaggaatagcataaaagatttcacaaaggttgctcaggatctttgcacatgattttccactattgtattgtaatttcag

SEQ ID NO: 466 - DNA, 11 bp, Artificial Sequence
  Description of Artificial Sequence Synthetic oligonucleotide
aaaccaacggt

SEQ ID NO: 467 - DNA, 297 bp, Artificial Sequence; identical to SEQ ID NO: 460
  Description of Artificial Sequence Synthetic polynucleotide

SEQ ID NO: 468 - DNA, 7 bp, Artificial Sequence
  Description of Artificial Sequence Synthetic oligonucleotide
cacagtg

SEQ ID NO: 469 - DNA, 142 bp, Artificial Sequence; identical to SEQ ID NO: 462
  Description of Artificial Sequence Synthetic polynucleotide

SEQ ID NO: 470 - DNA, 13 bp, Mus musculus
cttccttcctcag

SEQ ID NO: 471 - DNA, 13 bp, Artificial Sequence
  Description of Artificial Sequence Synthetic oligonucleotide
aaattaattaacc

(19) United States
(12) Patent Application Publication
(10) Pub. No.: US 2021/0000087 A1
(43) Pub. Date: Jan. 7, 2021

(54) TRANSGENIC MAMMALS AND METHODS OF USE THEREOF

(71) Applicant: TRIANNI, INC., San Francisco, CA (US)

(72) Inventors: Bao DUONG, Pacifica, CA (US); Peter Daniel BURROWS, Birmingham, AL (US); Werner MUELLER, Köln (DE); Gloria ESPOSITO, Vienna (AT); Matthias WABL, San Francisco, CA (US)

(21) Appl. No.: 16/916,492

(22) Filed: Jun. 30, 2020

Related U.S. Application Data
(60) Provisional application No. 62/869,435, filed on Jul. 1, 2019.

(51) Int. Cl. A01K 67/027; C12N 5/0781; C12N 5/16; C07K 16/46

(52) U.S. Cl. CPC A01K 67/0275; C12N 5/0635; C12N 5/163; C07K 2317/24; A01K 2267/01; A01K 2227/105; A01K 2217/05; C07K 16/462

(57) ABSTRACT

Transgenic mammals that express canine-based immunoglobulins are described herein, including transgenic rodents that express canine-based immunoglobulins for the development of canine therapeutic antibodies.

### CROSS-REFERENCE TO RELATED APPLICATIONS

This application claims priority to U.S. Provisional Patent Application No. 62/869,435, filed Jul. 1, 2019, the disclosure of which is incorporated herein by reference.

### SEQUENCE LISTING

The instant application contains a Sequence Listing which has been submitted electronically in ASCII format and is hereby incorporated by reference in its entirety. Said ASCII copy, created on Jun. 24, 2020, is named 0133-0006US1_SL.txt and is 218,648 bytes in size.

### FIELD OF THE INVENTION

This invention relates to production of immunoglobulin molecules, including methods for generating transgenic mammals capable of producing canine antigen-specific antibody-secreting cells for the generation of monoclonal antibodies.

### BACKGROUND

In the following discussion certain articles and methods are described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Antibodies have emerged as important biological pharmaceuticals because they (i) exhibit exquisite binding properties that can target antigens of diverse molecular forms, (ii) are physiological molecules with desirable pharmacokinetics that make them well tolerated in treated humans and animals, and (iii) are associated with powerful immunological properties that naturally ward off infectious agents. Furthermore, established technologies exist for the rapid isolation of antibodies from laboratory animals, which can readily mount a specific antibody response against virtually any foreign substance not present natively in the body.

In their most elemental form, antibodies are composed of two identical heavy (H) chains that are each paired with an identical light (L) chain. The N-termini of both H and L chains includes a variable domain (V_{H} and V_{L}, respectively) that together provide the paired H-L chains with a unique antigen-binding specificity.

The exons that encode the antibody V_{H} and V_{L} domains do not exist in the germline DNA. Instead, each V_{H} exon is generated by the recombination of randomly selected V_{H}, D, and J_{H} gene segments present in the immunoglobulin H chain locus (IGH); likewise, individual V_{L} exons are produced by the chromosomal rearrangements of randomly selected V_{L} and J_{L} gene segments in a light chain locus.

The canine genome contains two alleles that can express the H chain (one allele from each parent), two alleles that can express the kappa (κ) L chain, and two alleles that can express the lambda (λ) L chain. There are multiple V_{H}, D, and J_{H} gene segments at the H chain locus as well as multiple V_{L} and J_{L} gene segments at both the immunoglobulin (IGK) and immunoglobulin λ (IGL) L chain loci (Collins and Watson (2018) Immunoglobulin Light Chain Gene Rearrangements, Receptor Editing and the Development of a Self-Tolerant Antibody Repertoire. Front. Immunol. 9:2249. (doi: 10.3389/fimmu.2018.02249)).

In a typical immunoglobulin heavy chain variable region gene locus, V_{H} gene segments lie upstream (5′) of J_{H} gene segments, with D gene segments located between the V_{H} and J_{H} gene segments. Downstream (3′) of the J_{H} gene segments of the IGH locus are clusters of exons that encode the constant region (C_{H}) of the antibody. Each cluster of C_{H} exons encodes a different antibody class (isotype). Eight classes of antibody exist in mouse: IgM, IgD, IgG3, IgG1, IgG2a (or IgG2c), IgG2b, IgE, and IgA (at the nucleic acid level, they are respectively referred to as: μ, δ, γ3, γ1, γ2a/c, γ2b, ε, and α). In canine animals (e.g., the domestic dog and wolf), the putative isotypes are IgM, IgD, IgG1, IgG2, IgG3, IgG4, IgE, and IgA (FIG. 12A).

At the IGK locus of most mammalian species, a cluster of V_{κ} gene segments are located upstream of a small number of J_{κ} gene segments, with the J_{κ} gene segment cluster located upstream of a single C_{κ} gene. This organization of the κ locus can be represented as (V_{κ})ₐ . . . (J_{κ})_{b} . . . C_{κ}, wherein a and b, independently, are an integer of 1 or more. The dog κ locus is unusual in that half the V_{κ} genes are located upstream, and half are located downstream of the J_{κ} and C_{κ} gene segments (see schematics of the mouse IGK locus in FIG. 1C and dog IGK locus in FIG. 12C).

The IGL locus of most species includes a set of V_{λ} gene segments that are located 5′ to a variable number of J-C tandem cassettes, each made up of a J_{λ} gene segment and a C_{λ} gene segment (see schematic of the canine IGL locus in FIG. 12B). The organization of the λ locus can be represented as (V_{λ})ₐ . . . (J_{λ}-C_{λ})_{b}, wherein a and b are, independently, an integer of 1 or more. The mouse IGL locus is unusual in that it contains two units of (V_{λ})ₐ . . . (J_{λ}-C_{λ})_{b}.

During B cell development, gene rearrangements occur first on one of the two homologous chromosomes that contain the H chain variable gene segments. The resultant V_{H} exon is then spliced at the RNA level to the C_{μ} exons for IgM H chain expression. Subsequently, the V_{L}-J_{L} rearrangements occur on one L chain allele at a time until a functional L chain is produced, after which the L chain polypeptides can associate with the IgM H chain homodimers to form a fully functional B cell receptor (BCR) for antigen. In mouse and human, as B cells continue to mature, IgD is co-expressed with IgM as alternatively spliced forms, with IgD being expressed at a level 10 times higher than IgM in the main B cell population. This contrasts with B cell development in the dog, in which the C_{δ} exons are likely to be nonfunctional.

It is widely accepted by experts in the field that in mouse and human, V_{L}-J_{L} rearrangements first occur at the IGK locus on both chromosomes before the IGL light chain locus on either chromosome becomes receptive for V_{L}-J_{L} recombination. This is supported by the observation that in mouse B cells that express κ light chains, the λ locus on both chromosomes is usually inactivated by non-productive rearrangements. This may explain the predominant κ L chain usage in mouse, which is >90% κ and <10% λ.

However, immunoglobulins in the dog immune system are dominated by λ light chain usage, which has been estimated to be at least 90% λ to <10% κ. It is not known mechanistically whether V_{κ}-J_{κ} rearrangements preferentially occur first over V_{λ}-J_{λ} rearrangements in canines.

Upon encountering an antigen, the B cell then may undergo another round of DNA recombination at the IGH locus to remove the C_{μ} and C_{δ} exons, effectively switching the C_{H} region to one of the downstream isotypes (this process is called class switching). In the dog, although cDNA clones identified as encoding canine IgG1-IgG4 have been isolated (Tang, et al. (2001) Cloning and characterization of cDNAs encoding four different canine immunoglobulin γ chains. Vet. Immunol. and Immunopath. 80:259 PMID 11457479), only the IgG2 constant region gene has been physically mapped to the canine IGH locus on chromosome 8 (Martin, et al. (2018) Comprehensive annotation and evolutionary insights into the canine (Canis lupus familiaris) antigen receptor loci. Immunogenet. 70:223 doi: 10.1007/s00251-017-1028-0).

The genes encoding various canine and mouse immunoglobulins have been extensively characterized. Priat, et al., describe whole-genome radiation mapping of the dog genome in Genomics, 54:361-78 (1998), and Bao, et al., describe the molecular characterization of the V_{H} repertoire in Canis familiaris in Veterinary Immunology and Immunopathology, 137:64-75 (2010). Martin et al. provide an annotation of the canine (Canis lupus familiaris) immunoglobulin kappa and lambda (IGK, IGL) loci, and an update to the annotation of the IGH locus in Immunogenetics, 70(4):223-236 (2018).

Blankenstein and Krawinkel describe the mouse variable heavy chain region locus in Eur. J. Immunol., 17:1351-1357 (1987). Transgenic animals are routinely used in various research and development applications. For example, the generation of transgenic mice containing immunoglobulin genes is described in International Application WO 90/10077 and WO 90/04036. WO 90/04036 describes a transgenic mouse with an integrated human immunoglobulin “mini” locus. WO 90/10077 describes a vector containing the immunoglobulin dominant control region for use in generating transgenic animals.

Numerous methods have been developed for modifying the mouse endogenous immunoglobulin variable region gene locus with, e.g., human immunoglobulin sequences to create partly or fully human antibodies for drug discovery purposes. Examples of such mice include those described in, e.g., U.S. Pat. Nos. 7,145,056; 7,064,244; 7,041,871; 6,673,986; 6,596,541; 6,570,061; 6,162,963; 6,130,364; 6,091,001; 6,023,010; 5,593,598; 5,877,397; 5,874,299; 5,814,318; 5,789,650; 5,661,016; 5,612,205; and 5,591,669. However, many of the fully humanized immunoglobulin transgenic mice exhibit suboptimal antibody production because B cell development in these mice is severely hampered by inefficient V(D)J recombination, and by inability of the fully human antibodies/BCRs to function optimally with mouse signaling proteins. Other humanized immunoglobulin transgenic mice, in which the mouse coding sequences have been “swapped” with human sequences, are very time consuming and expensive to create due to the approach of replacing individual mouse exons with the syntenic human counterpart.

The use of antibodies that function as drugs is not limited to the prevention or therapy of human disease. Companion animals such as dogs suffer from some of the same afflictions as humans, e.g., cancer, atopic dermatitis and chronic pain. Monoclonal antibodies targeting IL31, CD20, IgE and Nerve Growth Factor, respectively, are already in veterinary use as for treatment of these conditions. However, before clinical use these monoclonal antibodies, which were made in mice, had to be caninized, i.e., their amino acid sequence had to be changed from mouse to dog, in order to prevent an immune response in the recipient dogs. Importantly, due to immunological tolerance, canine antibodies to canine proteins cannot be easily raised in dogs. Based on the foregoing, it is clear that a need exists for efficient and cost-effective methods to produce canine antibodies for the treatment of diseases in dogs. More particularly, there is a need in the art for small, rapidly breeding, non-canine mammals capable of producing antigen-specific canine immunoglobulins. Such non-canine mammals are useful for generating hybridomas capable of large-scale production of canine monoclonal antibodies.

PCT Publication No. 2018/189520 describes rodents and cells with a genome that is engineered to express exogenous animal immunoglobulin variable region genes from companion animals such as dogs, cats, horses, birds, rabbits, goats, reptiles, fish and amphibians.

However, there still remains a need for improved methods for generating transgenic nonhuman animals which are capable of producing an antibody with canine V regions.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

Described herein is a non-canine mammalian cell and a non-canine mammal having a genome comprising an exogenously introduced partly canine immunoglobulin locus, where the introduced locus comprises coding sequences of the canine immunoglobulin variable region gene segments and non-coding sequences based on the endogenous immunoglobulin variable region locus of the non-canine mammalian host. Thus, the non-canine mammalian cell or mammal is capable of expressing a chimeric B cell receptor (BCR) or antibody comprising H and L chain variable regions that are fully canine in conjunction with the respective constant regions that are native to the non-canine mammalian host cell or mammal. Preferably, the transgenic cells and animals have genomes in which part or all of the endogenous immunoglobulin variable region gene locus is removed.

At a minimum, the production of chimeric canine monoclonal antibodies in a non-canine mammalian host requires the host to have at least one locus that expresses chimeric canine immunoglobulin H or L chain. In most aspects, there are one heavy chain locus and two light chain loci that, respectively, express chimeric canine immunoglobulin H and L chains.

In some aspects, the partly canine immunoglobulin locus comprises canine V_{H} coding sequences and non-coding regulatory or scaffold sequences present in the endogenous V_{H} gene locus of the non-canine mammalian host. In these aspects, the partly canine immunoglobulin locus further comprises canine D and J_{H} gene segment coding sequences in conjunction with the non-coding regulatory or scaffold sequences present in the vicinity of the endogenous D and J_{H} gene segments of the non-canine mammalian host cell genome. In one aspect, the partly canine immunoglobulin locus comprises canine V_{H}, D and J_{H} gene segment coding sequences embedded in non-coding regulatory or scaffold sequences present in an endogenous immunoglobulin heavy chain locus of the non-canine mammalian host. In one aspect, the partly canine immunoglobulin locus comprises canine V_{H}, D and J_{H} gene segment coding sequences embedded in non-coding regulatory or scaffold sequences present in an endogenous immunoglobulin heavy chain locus of a rodent, such as a mouse. In other aspects, the partly canine immunoglobulin locus comprises canine V_{L} coding sequences and non-coding regulatory or scaffold sequences present in the endogenous V_{L} gene locus of the non-canine mammalian host. In one aspect, the exogenously introduced, partly canine immunoglobulin locus comprising canine V_{L} coding sequences further comprises canine L-chain J gene segment coding sequences and non-coding regulatory or scaffold sequences present in the vicinity of the endogenous L-chain J gene segments of the non-canine mammalian host cell genome. In one aspect, the partly canine immunoglobulin locus comprises canine Vλ and J_{λ} gene segment coding sequences embedded in non-coding regulatory or scaffold sequences of an immunoglobulin light chain locus in the non-canine mammalian host cell. In one aspect, the partly canine immunoglobulin locus comprises canine V_{κ} and J_{κ} gene segment coding sequences embedded in non-coding regulatory or scaffold sequences of an immunoglobulin locus of the non-canine mammalian host. In one aspect, the endogenous κ locus of the non-canine mammalian host is inactivated or replaced by sequences encoding canine λ chain, to increase production of canine λ immunoglobulin light chain over canine κ chain. In one aspect, the endogenous κ locus of the non-canine mammalian host is inactivated but not replaced by sequences encoding canine λ chain.

In certain aspects, the non-canine mammal is a rodent, for example, a mouse or rat.

In one aspect, the engineered immunoglobulin locus includes a partly canine immunoglobulin light chain locus that includes one or more canine λ variable region gene segment coding sequences. In one aspect, the engineered immunoglobulin locus is a partly canine immunoglobulin light chain locus that includes one or more canine κ variable region gene segment coding sequences.

In one aspect, a transgenic rodent or rodent cell is provided that has a genome comprising an engineered partly canine immunoglobulin locus. In one aspect, a transgenic rodent or rodent cell is provided that has a genome comprising an engineered partly canine immunoglobulin light chain locus. In one aspect, the partly canine immunoglobulin light chain locus of the rodent or rodent cell includes one or more canine immunoglobulin variable region gene segment coding sequences. In one aspect, the partly canine immunoglobulin light chain locus of the rodent or rodent cell includes one or more canine immunoglobulin κ variable region gene segment coding sequences. In one aspect, the engineered immunoglobulin locus is capable of expressing immunoglobulin comprising canine variable domains.

In one aspect, a transgenic rodent that produces more immunoglobulin comprising λ light chain than immunoglobulin comprising κ light chain is provided. In one aspect, the transgenic rodent produces at least about 25%, 30%, 35%, 40%, 45%, 50%, 55%, 60%, 65%, 70%, 75%, 80%, 85%, 90% or 95% and up to about 100% λ light chain immunoglobulin. In one aspect, the transgenic rodent produces at least about 25%, 30%, 35%, 40%, 45%, 50%, 55%, 60%, 65%, 70%, 75%, 80%, 85%, 90% or 95% and up to about 100% λ light chain immunoglobulin comprising a canine variable domain. In one aspect, more λ light chain-producing cells than κ light chain-producing cells are likely to be isolated from the transgenic rodent. In one aspect, more cells producing λ light chain with a canine variable domain are likely to be isolated from the transgenic rodent than cells producing κ light chain with a canine variable domain.

In one aspect, a transgenic rodent cell is provided that is more likely to produce immunoglobulin comprising λ light chain than immunoglobulin comprising κ light chain. In one aspect, the rodent cell is isolated from a transgenic rodent described herein. In one aspect, the rodent cell is recombinantly produced as described herein. In one aspect, the transgenic rodent cell or its progeny, has at least about a 25%, 30%, 35%, 40%, 45%, 50%, 55%, 60%, 65%, 70%, 75%, 80%, 85%, 90%, or 95% and up to about 100%, probability of producing λ light chain immunoglobulin. In one aspect, the transgenic rodent cell or its progeny, has at least about a 25%, 30%, 35%, 40%, 45%, 50%, 55%, 60%, 65%, 70%, 75%, 80%, 85%, 90%, or 95%, and up to about 100%, probability of producing λ light chain immunoglobulin with a canine variable domain

In one aspect, the engineered partly canine immunoglobulin locus comprises canine V_{λ} gene segment coding sequences and J_{λ} gene segment coding sequences and non-coding sequences such as regulatory or scaffold sequences of a rodent immunoglobulin light chain variable region gene locus.

In one aspect, the engineered immunoglobulin locus comprises canine V_{λ} and J_{λ} gene segment coding sequences embedded in rodent non-coding regulatory or scaffold sequences of a rodent immunoglobulin λ light chain variable region gene locus. In one aspect, the engineered immunoglobulin locus comprises canine V_{λ} and J_{λ} gene segment coding sequences embedded in non-coding regulatory or scaffold sequences of the rodent immunoglobulin κ light chain variable region gene locus. In one aspect, the partly canine immunoglobulin locus comprises one or more canine V_{λ} gene segment coding sequences and J_{λ} gene segment coding sequences and one or more rodent immunoglobulin λ constant region coding sequences.

In one aspect, the engineered immunoglobulin variable region locus comprises one or more canine V_{λ} gene segment coding sequences and one or more J-C units wherein each J-C unit comprises a canine J_{λ} gene segment coding sequence and rodent region C_{λ} coding sequence. In one aspect, the engineered immunoglobulin variable region locus comprises one or more canine V_{λ} gene segment coding sequences and one or more J-C units wherein each J-C unit comprises a canine J_{λ} gene segment coding sequence and rodent C_{λ} region coding and non-coding sequences. In one aspect, the rodent C_{λ} region coding sequence is selected from a rodent C_{λ1}, C_{λ2} or C_{λ3} coding sequence. In one aspect, one or more canine V_{λ} gene segment coding sequences are located upstream of one or more J-C units, wherein each J-C unit comprises a canine J_{λ} gene segment coding sequence and a rodent C_{λ} gene segment coding sequence. In one aspect, one or more canine V_{λ} gene segment coding sequences are located upstream of one or more J-C units, wherein each J-C unit comprises a canine J_{λ} gene segment coding sequence and a rodent C_{λ} gene segment coding sequence and rodent C_{λ} non-coding sequences. In one aspect, the J-C units comprise canine J_{λ} gene segment coding sequences and rodent C_{λ} region coding sequences embedded in non-coding regulatory or scaffold sequences of a rodent immunoglobulin κ light chain locus.

In one aspect, a transgenic rodent or rodent cell is provided with an engineered immunoglobulin locus that includes a rodent immunoglobulin κ locus in which one or more rodent V_{κ} gene segment coding sequences and one or more rodent J_{κ} gene segment coding sequences have been deleted and replaced with one or more canine V_{λ} gene segment coding sequences and one or more J_{λ} gene segment coding sequences, respectively, and in which rodent C_{κ} coding sequence in the locus has been replaced by rodent C_{λ1}, C_{λ2}, or C_{λ3} coding sequence(s).

In one aspect, the engineered immunoglobulin locus includes one or more canine V_{λ} gene segment coding sequences upstream and in the same transcriptional orientation as one or more canine J_{λ} gene segment coding sequences which are upstream of one or more rodent C_{λ} coding sequences.

In one aspect, the engineered immunoglobulin locus includes one or more canine V_{λ} gene segment coding sequences upstream and in the opposite transcriptional orientation as one or more canine J_{λ} gene segment coding sequences which are upstream of one or more rodent C_{λ} coding sequences.

In one aspect, a transgenic rodent or rodent cell is provided in which an endogenous rodent immunoglobulin κ light chain locus is deleted, inactivated, or made nonfunctional by one or more of:
a. deleting or mutating all endogenous rodent V_{κ} gene segment coding sequences;
b. deleting or mutating all endogenous rodent J_{κ} gene segment coding sequences;
c. deleting or mutating endogenous rodent C_{κ} coding sequence;
d. deleting or mutating a splice donor site, pyrimidine tract, or splice acceptor site within the intron between a J_{κ} gene segment and C_{κ} exon; and
e. deleting, mutating, or disrupting an endogenous intronic κ enhancer (iE_{κ}), an 3′ enhancer sequence (3′E_{κ}), or a combination thereof.

In one aspect, a transgenic rodent or rodent cell is provided in which expression of an endogenous rodent immunoglobulin λ light chain variable domain is suppressed or inactivated by one or more of:
a. deleting or mutating all endogenous rodent V_{λ} gene segments;
b. deleting or mutating all endogenous rodent J_{λ} gene segments;
c. deleting or mutating all endogenous rodent C_{λ} coding sequences; and
d. deleting or mutating a splice donor site, pyrimidine tract, splice acceptor site within the intron between a J_{λ} gene segment and C_{λ} exon, or a combination thereof.

In one aspect, a transgenic rodent or rodent cell is provided in which the engineered immunoglobulin locus expresses immunoglobulin light chains comprising a canine variable domain and a rodent constant domain. In one aspect, a transgenic rodent or rodent cell is provided in which the engineered immunoglobulin locus expresses immunoglobulin light chains comprising a canine λ variable domain and rodent λ constant domain. In one aspect, a transgenic rodent or rodent cell is provided in which the engineered immunoglobulin locus expresses immunoglobulin light chains comprising a canine κ variable domain and rodent κ constant domain.

In one aspect, a transgenic rodent or rodent cell is provided in which the genome of the transgenic rodent or rodent cell comprises an engineered immunoglobulin locus comprising canine V_{κ} and J_{κ} gene segment coding sequences. In one aspect, the canine V_{κ} and J_{κ} gene segment coding sequences are inserted into a rodent immunoglobulin κ light chain locus. In one aspect, the canine V_{κ} and J_{κ} gene segment coding sequences are embedded in rodent non-coding regulatory or scaffold sequences of the rodent immunoglobulin κ light chain variable region gene locus. In one aspect, the canine V_{κ} and J_{κ} coding sequences are inserted upstream of a rodent immunoglobulin κ light chain constant region coding sequence.

In one aspect, a transgenic rodent or rodent cell is provided in which the genome of the transgenic rodent or rodent cell comprises an engineered immunoglobulin locus comprising canine V_{κ} and J_{κ} gene segment coding sequences inserted into a rodent immunoglobulin λ light chain locus. In one aspect, the canine V_{κ} and J_{κ} gene segment coding sequences are embedded in rodent non-coding regulatory or scaffold sequences of the rodent immunoglobulin λ light chain variable region gene locus. In one aspect, the genome of the transgenic rodent or rodent cell includes a rodent immunoglobulin κ light chain constant region coding sequence inserted downstream of the canine V_{κ} and J_{κ} gene segment coding sequences. In one aspect, the rodent immunoglobulin κ light chain constant region is inserted upstream of an endogenous rodent C_{λ} coding sequence. In one aspect, the rodent immunoglobulin κ light chain constant region is inserted upstream of an endogenous rodent C_{λ2} coding sequence. In one aspect, expression of an endogenous rodent immunoglobulin λ light chain variable domain is suppressed or inactivated by one or more of:
a. deleting or mutating all endogenous rodent V_{λ} gene segment coding sequences.
b. deleting or mutating all endogenous rodent J_{λ} gene segment coding sequences;
c. deleting or mutating all endogenous C_{λ} coding sequences; and
d. deleting or mutating a splice donor site, pyrimidine tract, or splice acceptor site within the intron between a J_{λ} gene segment and C_{λ} exon.

In one aspect, the engineered partly canine immunoglobulin light chain locus comprises a rodent intronic κ enhancer (iEκ) and 3′ κ enhancer (3′Eκ) regulatory sequences.

In one aspect, the transgenic rodent or rodent cell further comprises an engineered partly canine immunoglobulin heavy chain locus comprising canine immunoglobulin heavy chain variable region gene segment coding sequences and non-coding regulatory and scaffold sequences of the rodent immunoglobulin heavy chain locus. In one aspect, the engineered canine immunoglobulin heavy chain locus comprises canine V_{H}, D and J_{H} gene segment coding sequences. In one aspect, each canine/rodent chimeric V_{H}, D or J_{H} gene segment comprises V_{H}, D or J_{H} coding sequence embedded in non-coding regulatory and scaffold sequences of the rodent immunoglobulin heavy chain locus. In one aspect, the heavy chain scaffold sequences are interspersed by one or both functional ADAM6 genes.

In one aspect, the rodent regulatory and scaffold sequences comprise one or more enhancers, promoters, splice sites, introns, recombination signal sequences, or a combination thereof.

In one aspect, an endogenous rodent immunoglobulin locus of the transgenic rodent or rodent cell has been inactivated. In one aspect, an endogenous rodent immunoglobulin locus of the transgenic rodent or rodent cell has been deleted and replaced with the engineered partly canine immunoglobulin locus.

In one aspect, the rodent is a mouse or a rat. In one aspect, the rodent cell is an embryonic stem (ES) cell or a cell of an early stage embryo. In one aspect, the rodent cell is a mouse or rat embryonic stem (ES) cell, or mouse or rat cell of an early stage embryo.

In one aspect, a cell of B lymphocyte lineage is provided that is obtained from a transgenic rodent described herein, wherein the B cell expresses or is capable of expressing a chimeric immunoglobulin heavy chain or light chain comprising a canine variable region and a rodent immunoglobulin constant region. In one aspect, a hybridoma cell or immortalized cell line is provided that is derived from a cell of B lymphocyte lineage obtained from a transgenic rodent or rodent cell described herein.

In one aspect, antibodies or antigen binding portions thereof are provided that are produced by a cell from a transgenic rodent or rodent cell described herein.

In one aspect, a nucleic acid sequence of a V_{H}, D, or J_{H}, or a V_{L} or J_{L} gene segment coding sequence is provided that is derived from an immunoglobulin produced by a transgenic rodent or rodent cell described herein. In one aspect, a method for generating a non-canine mammalian cell comprising a partly canine immunoglobulin locus is provided, said method comprising: a) introducing two or more recombinase targeting sites into the genome of a non-canine mammalian host cell and integrating at least one site upstream and at least one site downstream of a genomic region comprising endogenous immunoglobulin variable region genes wherein the endogenous immunoglobulin variable genes comprise V_{H}, D and J_{H} gene segments, or V_{κ} and J_{κ} gene segments, or V_{λ} and J_{λ} gene segments, or V_{λ}, J_{λ} and C_{λ} gene segments; and b) introducing into the non-canine mammalian host cell via recombinase-mediated cassette exchange (RMCE) an engineered partly canine immunoglobulin variable gene locus comprising canine immunoglobulin variable region gene coding sequences and non-coding regulatory or scaffold sequences corresponding to the non-coding regulatory or scaffold sequences present in the endogenous immunoglobulin variable region gene locus of the non-canine mammalian host.

In another aspect, the method further comprises deleting the genomic region flanked by the two exogenously introduced recombinase targeting sites prior to step b.

In a specific aspect of this method, the exogenously introduced, engineered partly canine immunoglobulin heavy chain locus is provided that comprises canine V_{H} gene segment coding sequences, and further comprises i) canine D and J_{H} gene segment coding sequences and ii) non-coding regulatory or scaffold sequences upstream of the canine D gene segments (pre-D sequences, FIG. 1A) that correspond to the sequences present upstream of the endogenous D gene segments in the genome of the non-canine mammalian host. In one aspect, these upstream scaffold sequences are interspersed by non-immunoglobulin genes, such as ADAM6A or ADAM6B (FIG. 1A) needed for male fertility (Nishimura et al. Developmental Biol. 233(1): 204-213 (2011)). The partly canine immunoglobulin heavy chain locus is introduced into the host cell using recombinase targeting sites that have been previously introduced upstream of the endogenous immunoglobulin V_{H} gene locus and downstream of the endogenous J_{H} gene locus on the same chromosome. In other aspects, the non-coding regulatory or scaffold sequences derive (at least partially) from other sources, e.g., they could be rationally designed artificial sequences or otherwise conserved sequences of unknown functions, sequences that are a combination of canine and artificial or other designed sequences, or sequences from other species. As used herein, “artificial sequence” refers to a sequence of a nucleic acid not derived from a sequence naturally occurring at a genetic locus. In one aspect, the non-coding regulatory or scaffold sequences are derived from non-coding regulatory or scaffold sequences of a rodent immunoglobulin heavy chain variable region locus. In one aspect, the non-coding regulatory or scaffold sequences have at least about 75%, 80%, 85%, 90%, 95% or 100% sequence identity to non-coding regulatory or scaffold sequences of a rodent immunoglobulin heavy chain variable region locus. In another aspect, the non-coding regulatory or scaffold sequences are rodent immunoglobulin heavy chain variable region non-coding or scaffold sequences.

In yet another specific aspect of the method, the introduced engineered partly canine immunoglobulin locus comprises canine immunoglobulin V_{L} gene segment coding sequences, and further comprises i) canine L-chain J gene segment coding sequences and ii) non-coding regulatory or scaffold sequences corresponding to the non-coding regulatory or scaffold sequences present in the endogenous L chain locus of the non-canine mammalian host cell genome. In one aspect, the engineered partly canine immunoglobulin locus is introduced into the host cell using recombinase targeting sites that have been previously introduced upstream of the endogenous immunoglobulin V_{L} gene locus and downstream of the endogenous J gene locus on the same chromosome.

In a more particular aspect of this method, an exogenously introduced, engineered partly canine immunoglobulin light chain locus is provided that comprises canine V_{λ} gene segment coding sequences and canine J_{λ} gene segment coding sequences. In one aspect, the partly canine immunoglobulin light chain locus is introduced into the host cell using recombinase targeting sites that have been previously introduced upstream of the endogenous immunoglobulin V_{λ} gene locus and downstream of the endogenous J_{λ} gene locus on the same chromosome.

In one aspect, the exogenously introduced, engineered partly canine immunoglobulin light chain locus comprises canine V_{κ} gene segment coding sequences and canine J_{κ} gene segment coding sequences. In one aspect, the partly canine immunoglobulin light chain locus is introduced into the host cell using recombinase targeting sites that have been previously introduced upstream of the endogenous immunoglobulin V_{κ} gene locus and downstream of the endogenous J_{κ} gene locus on the same chromosome.

In one aspect, the non-coding regulatory or scaffold sequences are derived from non-coding regulatory or scaffold sequences of a rodent λ immunoglobulin light chain variable region locus. In one aspect, the non-coding regulatory or scaffold sequences have at least about 75%, 80%, 85%, 90%, 95% or 100% sequence identity to non-coding regulatory or scaffold sequences of a rodent immunoglobulin λ light chain variable region locus. In another aspect, the non-coding regulatory or scaffold sequences are rodent immunoglobulin λ light chain variable region non-coding or scaffold sequences.

In one aspect, the non-coding regulatory or scaffold sequences are derived from non-coding regulatory or scaffold sequences of a rodent immunoglobulin κ light chain variable region locus. In one aspect, the non-coding regulatory or scaffold sequences have at least about 75%, 80%, 85%, 90%, 95% or 100% sequence identity to non-coding regulatory or scaffold sequences of a rodent immunoglobulin κ light chain variable region locus. In another aspect, the non-coding regulatory or scaffold sequences are rodent immunoglobulin κ light chain variable region non-coding or scaffold sequences.

In one aspect, the engineered partly canine immunoglobulin locus is synthesized as a single nucleic acid, and introduced into the non-canine mammalian host cell as a single nucleic acid region. In one aspect, the engineered partly canine immunoglobulin locus is synthesized in two or more contiguous segments, and introduced to the mammalian host cell as discrete segments. In another aspect, the engineered partly canine immunoglobulin locus is produced using recombinant methods and isolated prior to being introduced into the non-canine mammalian host cell.

In another aspect, methods for generating a non-canine mammalian cell comprising an engineered partly canine immunoglobulin locus are provided, said method comprising: a) introducing into the genome of a non-canine mammalian host cell two or more sequence-specific recombination sites that are not capable of recombining with one another, wherein at least one recombination site is introduced upstream of an endogenous immunoglobulin variable region gene locus while at least one recombination site is introduced downstream of the endogenous immunoglobulin variable region gene locus on the same chromosome; b) providing a vector comprising an engineered partly canine immunoglobulin locus having i) canine immunoglobulin variable region gene coding sequences and ii) non-coding regulatory or scaffold sequences based on an endogenous immunoglobulin variable region gene locus of the host cell genome, wherein the partly canine immunoglobulin locus is flanked by the same two sequence-specific recombination sites that flank the endogenous immunoglobulin variable region gene locus of the host cell of a); c) introducing into the host cell the vector of step b) and a site specific recombinase capable of recognizing the two recombinase sites; d) allowing a recombination event to occur between the genome of the cell of a) and the engineered partly canine immunoglobulin locus, resulting in a replacement of the endogenous immunoglobulin variable region gene locus with the engineered partly canine immunoglobulin variable region gene locus.

In one aspect, the partly canine immunoglobulin locus comprises V_{H} immunoglobulin gene segment coding sequences, and further comprises i) canine D and J_{H} gene segment coding sequences, ii) non-coding regulatory or scaffold sequences surrounding the codons of individual V_{H}, D, and J_{H} gene segments present endogenously in the genome of the non-canine mammalian host, and iii) pre-D sequences based on the endogenous genome of the non-canine mammalian host cell. The recombinase targeting sites are introduced upstream of the endogenous immunoglobulin V_{H} gene locus and downstream of the endogenous D and J_{H} gene locus.

In one aspect, there is provided a transgenic rodent with a genome deleted of a rodent endogenous immunoglobulin variable gene locus and in which the deleted rodent endogenous immunoglobulin variable gene locus has been replaced with an engineered partly canine immunoglobulin locus comprising canine immunoglobulin variable gene coding sequences and non-coding regulatory or scaffold sequences based on the rodent endogenous immunoglobulin variable gene locus, wherein the engineered partly canine immunoglobulin locus of the transgenic rodent is functional and expresses immunoglobulin chains with canine variable domains and rodent constant domains. In some aspects, the engineered partly canine immunoglobulin locus comprises canine V_{H}, D, and J_{H} coding sequences, and in some aspects, the engineered partly canine immunoglobulin locus comprises canine V_{L} and J_{L} coding sequences. In one aspect, the partly canine immunoglobulin locus comprises canine V_{λ} and J_{λ} coding sequences. In another aspect, the partly canine immunoglobulin locus comprises canine V_{κ} and J_{κ} coding sequences.

Some aspects provide a cell of B lymphocyte lineage from the transgenic rodent, a part or whole immunoglobulin molecule comprising canine variable domains and rodent constant domains obtained from the cell of B lymphocyte lineage, a hybridoma cell derived from the cell of B lymphocyte lineage, a part or whole immunoglobulin molecule comprising canine variable domains and rodent constant domains obtained from the hybridoma cell, a part or whole immunoglobulin molecule comprising canine variable domains derived from an immunoglobulin molecule obtained from the hybridoma cell, an immortalized cell derived from the cell of B lymphocyte lineage, a part or whole immunoglobulin molecule comprising canine variable domains and rodent constant domains obtained from the immortalized cell, a part or whole immunoglobulin molecule comprising canine variable domains derived from an immunoglobulin molecule obtained from the immortalized cell.

In one aspect, a transgenic rodent is provided, wherein the engineered partly canine immunoglobulin locus comprises canine V_{L} and J_{L} coding sequences, and a transgenic rodent, wherein the engineered partly canine immunoglobulin loci comprise canine V_{H}, D, and J_{H} or V_{L} and J_{L} coding sequences. In some aspects, the rodent is a mouse. In some aspects, the non-coding regulatory sequences comprise the following sequences of endogenous host origin: promoters preceding each V gene segment coding sequence, introns, splice sites, and recombination signal sequences for V(D)J recombination; in other aspects, the engineered partly canine immunoglobulin locus further comprises one or more of the following sequences of endogenous host origin: ADAM6A or ADAM6B gene, a Pax-5-Activated Intergenic Repeat (PAIR) elements, or CTCF binding sites from a heavy chain intergenic control region 1.

In one aspect, the non-canine mammalian cell for use in each of the above methods is a mammalian cell, for example, a mammalian embryonic stem (ES) cell. In one aspect, the mammalian cell is a cell of an early stage embryo. In one aspect, the non-canine mammalian cell is a rodent cell. In one aspect, the non-canine mammalian cell is a mouse cell.

Once the cells have been subjected to the replacement of the endogenous immunoglobulin variable region gene locus by the introduced partly canine immunoglobulin variable region gene locus, the cells can be selected and isolated. In one aspect, the cells are non-canine mammalian ES cells, for example, rodent ES cells, and at least one isolated ES cell clone is then utilized to create a transgenic non-canine mammal expressing the engineered partly canine immunoglobulin variable region gene locus.

In one aspect, a method for generating the transgenic rodent is provided, said method comprising: a) integrating at least one target site for a site-specific recombinase in a rodent cell's genome upstream of an endogenous immunoglobulin variable gene locus and at least one target site for a site-specific recombinase downstream of the endogenous immunoglobulin variable gene locus, wherein the endogenous immunoglobulin variable locus comprises V_{H}, D and J_{H} gene segments, or V_{κ} and J_{κ} gene segments, or V_{λ} and J_{λ} gene segments, or V_{λ}, J_{λ} and C_{λ} gene segments; b) providing a vector comprising an engineered partly canine immunoglobulin locus, said engineered partly canine immunoglobulin locus comprising chimeric canine immunoglobulin gene segments, wherein each of the partly canine immunoglobulin gene segment comprises canine immunoglobulin variable gene coding sequences and rodent non-coding regulatory or scaffold sequences, with the partly canine immunoglobulin variable gene locus being flanked by target sites for a site-specific recombinase wherein the target sites are capable of recombining with the target sites introduced into the rodent cell; c) introducing into the cell the vector and a site-specific recombinase capable of recognizing the target sites; d) allowing a recombination event to occur between the genome of the cell and the engineered partly canine immunoglobulin locus resulting in a replacement of the endogenous immunoglobulin variable gene locus with the engineered partly canine immunoglobulin locus; e) selecting a cell that comprises the engineered partly canine immunoglobulin variable locus generated in step d); and utilizing the cell to create a transgenic rodent comprising partly canine the engineered partly canine immunoglobulin variable locus. In some aspects, the cell is a rodent embryonic stem (ES) cell, and in some aspects the cell is a mouse embryonic stem (ES) cell. Some aspects of this method further comprise after, after step a) and before step b), a step of deleting the endogenous immunoglobulin variable gene locus by introduction of a recombinase that recognizes a first set of target sites, wherein the deleting step leaves in place at least one set of target sites that are not capable of recombining with one another in the rodent cell's genome. In some aspects, the vector comprises canine V_{H}, D, and J_{H}, coding sequences, and in some aspects the vector comprises canine V_{L} and J_{L} coding sequences. In some aspects, the vector further comprises rodent promoters, introns, splice sites, and recombination signal sequences of variable region gene segments.

In another aspect, a method for generating a transgenic non-canine mammal comprising an exogenously introduced, engineered partly canine immunoglobulin variable region gene locus is provided, said method comprising: a) introducing into the genome of a non-canine mammalian host cell one or more sequence-specific recombination sites that flank an endogenous immunoglobulin variable region gene locus and are not capable of recombining with one another; b) providing a vector comprising a partly canine immunoglobulin locus having i) canine variable region gene coding sequences and ii) non-coding regulatory or scaffold sequences based on the endogenous host immunoglobulin variable region gene locus, wherein the coding and non-coding regulatory or scaffold sequences are flanked by the same sequence-specific recombination sites as those introduced to the genome of the host cell of a); c) introducing into the cell the vector of step b) and a site-specific recombinase capable of recognizing one set of recombinase sites; d) allowing a recombination event to occur between the genome of the cell of a) and the engineered partly canine immunoglobulin variable region gene locus, resulting in a replacement of the endogenous immunoglobulin variable region gene locus with the partly canine immunoglobulin locus; e) selecting a cell which comprises the partly canine immunoglobulin locus; and f) utilizing the cell to create a transgenic animal comprising the partly canine immunoglobulin locus.

In a specific aspect, the engineered partly canine immunoglobulin locus comprises canine V_{H}, D, and J_{H} gene segment coding sequences, and non-coding regulatory and scaffold pre-D sequences (including a fertility-enabling gene) present in the endogenous genome of the non-canine mammalian host. In one aspect, the sequence-specific recombination sites are then introduced upstream of the endogenous immunoglobulin V_{H} gene segments and downstream of the endogenous J_{H} gene segments.

In one aspect, a method for generating a transgenic non-canine animal comprising an engineered partly canine immunoglobulin locus is provided, said method comprising: a) providing a non-canine mammalian cell having a genome that comprises two sets of sequence-specific recombination sites that are not capable of recombining with one another, and which flank a portion of an endogenous immunoglobulin variable region gene locus of the host genome; b) deleting the portion of the endogenous immunoglobulin locus of the host genome by introduction of a recombinase that recognizes a first set of sequence-specific recombination sites, wherein such deletion in the genome retains a second set of sequence-specific recombination sites; c) providing a vector comprising an engineered partly canine immunoglobulin variable region gene locus having canine coding sequences and non-coding regulatory or scaffold sequences based on the endogenous immunoglobulin variable region gene locus, where the coding and non-coding regulatory or scaffold sequences are flanked by the second set of sequence-specific recombination sites; d) introducing the vector of step c) and a site-specific recombinase capable of recognizing the second set of sequence-specific recombination sites into the cell; e) allowing a recombination event to occur between the genome of the cell and the partly canine immunoglobulin locus, resulting in a replacement of the endogenous immunoglobulin locus with the engineered partly canine immunoglobulin variable locus; f) selecting a cell that comprises the partly canine immunoglobulin variable region gene locus; and g) utilizing the cell to create a transgenic animal comprising the engineered partly canine immunoglobulin variable region gene locus.

In one aspect, a method for generating a transgenic non-canine mammal comprising an engineered partly canine immunoglobulin locus is provided, said method comprising: a) providing a non-canine mammalian embryonic stem ES cell having a genome that contains two sequence-specific recombination sites that are not capable of recombining with each other, and which flank the endogenous immunoglobulin variable region gene locus; b) providing a vector comprising an engineered partly canine immunoglobulin locus comprising canine immunoglobulin variable gene coding sequences and non-coding regulatory or scaffold sequences based on the endogenous immunoglobulin variable region gene locus, where the partly canine immunoglobulin locus is flanked by the same two sequence-specific recombination sites that flank the endogenous immunoglobulin variable region gene locus in the ES cell; c) bringing the ES cell and the vector into contact with a site-specific recombinase capable of recognizing the two recombinase sites under appropriate conditions to promote a recombination event resulting in the replacement of the endogenous immunoglobulin variable region gene locus with the engineered partly canine immunoglobulin variable region gene locus in the ES cell; d) selecting an ES cell that comprises the engineered partly canine immunoglobulin locus; and e) utilizing the cell to create a transgenic animal comprising the engineered partly canine immunoglobulin locus.

In one aspect, the transgenic non-canine mammal is a rodent, e.g., a mouse or a rat.

In one aspect, a non-canine mammalian cell and a non-canine transgenic mammal are provide that express an introduced immunoglobulin variable region gene locus having canine variable region gene coding sequences and non-coding regulatory or scaffold sequences based on the endogenous non-canine immunoglobulin locus of the host genome, where the non-canine mammalian cell and transgenic animal express chimeric antibodies with fully canine H or L chain variable domains in conjunction with their respective constant regions that are native to the non-canine mammalian cell or animal.

Further, B cells from transgenic animals are provided that are capable of expressing partly canine antibodies having fully canine variable sequences, wherein such B cells are immortalized to provide a source of a monoclonal antibody specific for a particular antigen. In one aspect, a cell of B lymphocyte lineage from a transgenic animal is provided that is capable of expressing partly canine heavy or light chain antibodies comprising a canine variable region and a rodent constant region.

In one aspect, canine immunoglobulin variable region gene sequences cloned from B cells are provided for use in the production or optimization of antibodies for diagnostic, preventative and therapeutic uses.

In one aspect, hybridoma cells are provided that are capable of producing partly canine monoclonal antibodies having fully canine immunoglobulin variable region sequences. In one aspect, a hybridoma or immortalized cell line of B lymphocyte lineage is provided.

In another aspect, antibodies or antigen binding portions thereof produced by a transgenic animal or cell described herein are provided. In another aspect, antibodies or antigen binding portions thereof comprising variable heavy chain or variable light chain sequences derived from antibodies produced by a transgenic animal or cell described herein are provided.

In one aspect, methods for determining the sequences of the H and L chain immunoglobulin variable domains from the monoclonal antibody-producing hybridomas or primary plasma cells or B cells and combining the V_{H} and V_{L} sequences with canine constant regions are provided for creating a fully canine antibody that is not immunogenic when injected into dogs.

These and other aspects, objects and features are described in more detail below.

### BRIEF DESCRIPTION OF THE FIGURES

FIG. 1A is a schematic diagram of the endogenous mouse IGH locus located at the telomeric end of chromosome 12.

FIG. 1B is a schematic diagram of the endogenous mouse IGL locus located on chromosome 16.

FIG. 1C is a schematic diagram of the endogenous mouse IGK locus located on chromosome 6.

FIG. 2 is a schematic diagram illustrating the strategy of targeting by homologous recombination to introduce a first set of sequence-specific recombination sites into a region upstream of the H chain variable region gene locus in the genome of a non-canine mammalian host cell.

FIG. 3 is another schematic diagram illustrating the strategy of targeting by homologous recombination to introduce a first set of sequence-specific recombination sites into a region upstream of the H chain variable region gene locus in the genome of a non-canine mammalian host cell.

FIG. 4 is a schematic diagram illustrating the introduction of a second set of sequence-specific recombination sites into a region downstream of the H chain variable region gene locus in the genome of a non-canine mammalian cell via a homology targeting vector.

FIG. 5 is a schematic diagram illustrating deletion of the endogenous immunoglobulin H chain variable region gene locus from the genome of the non-canine mammalian host cell.

FIG. 6 is a schematic diagram illustrating the RMCE strategy to introduce an engineered partly canine immunoglobulin H chain locus into the non-canine mammalian host cell genome that has been previously modified to delete the endogenous immunoglobulin H chain variable region gene locus.

FIG. 7 is a schematic diagram illustrating the RMCE strategy to introduce an engineered partly canine immunoglobulin H chain locus comprising additional regulatory sequences into the non-canine mammalian host cell genome that has been previously modified to delete the endogenous immunoglobulin H chain variable region genes.

FIG. 8 is a schematic diagram illustrating the introduction of an engineered partly canine immunoglobulin H chain variable region gene locus into the endogenous immunoglobulin H chain locus of the mouse genome.

FIG. 9 is a schematic diagram illustrating the introduction of an engineered partly canine immunoglobulin κ L chain variable region gene locus into the endogenous immunoglobulin κ L chain locus of the mouse genome.

FIG. 10 is a schematic diagram illustrating the introduction of an engineered partly canine immunoglobulin λ L chain variable region gene locus into the endogenous immunoglobulin λ L chain locus of the mouse genome.

FIG. 11 is a schematic diagram illustrating the introduction of an engineered partly canine immunoglobulin locus comprising a canine V_{H} minilocus via RMCE.

FIG. 12A is a schematic diagram of the endogenous canine IGH locus located on chromosome 8 showing the entire IGH locus (1201) and an expanded view of the IGHC region (1202).

FIG. 12B is a schematic diagram of the endogenous canine IGL locus located on chromosome 26.

FIG. 12C is a schematic diagram of the endogenous canine IGK locus located on chromosome 17. Arrows indicate the transcriptional orientation of the V_{κ} gene segments. In the native canine IGK locus (1220) some V_{κ} gene segments are downstream of the C_{κ} exon. In the partly canine Ig_{κ} locus described herein (1221), all of the V_{κ} gene segment coding sequences are upstream of the C_{κ} exon and in the same transcriptional orientation as the Cκ exon (See Example 4).

FIG. 13 is a schematic diagram illustrating an engineered partly canine immunoglobulin light chain variable region locus in which one or more canine V_{λ} gene segment coding sequences are inserted into a rodent immunoglobulin κ light chain locus upstream of one or more canine J_{λ} gene segment coding sequences, which are upstream of one or more rodent C_{λ} region coding sequences.

FIG. 14 is a schematic diagram illustrating the introduction of an engineered partly canine light chain variable region locus in which one or more canine V_{λ} gene segment coding sequences are inserted into a rodent immunoglobulin κ light chain locus upstream of an array of J_{λ}-C_{λ} tandem cassettes in which the J_{λ} is of canine origin and the C_{λ} is of mouse origin, C_{λ1}, C_{λ2} or C_{λ3}.

FIG. 15 shows flow cytometry profiles of 293T/17 cells transfected with expression vectors encoding human CD4 (hCD4), canine IGHV3-5-mouse C_{μ} membrane form IgM^{b} allotype, and canine IGLV3-28/J_{λ}6 attached to various combinations of mouse C_{κ} and C_{λ} (1501), or canine IGKV2-5/J_{κ}1 attached to various combinations of mouse C_{κ} and C_{λ} (1502). The cells have been stained for cell surface hCD4 (1509) or for mouse IgM^{b} (1510).

FIG. 16 shows flow cytometry profiles of 293T/17 cells transfected with expression vectors encoding human CD4 (hCD4), canine IGHV3-5-mouse C_{μ} membrane form IgM^{b} allotype, and canine IGLV3-28/J_{λ}6 attached to various combinations of mouse C_{κ} and C_{λ} (1601), or canine IGKV2-5/J_{κ}1 attached to various combinations of mouse C_{κ} and C_{λ} (1602). The cells have been stained for cell surface mouse λLC (1601) or mouse κLC (1602).

FIG. 17 shows flow cytometry profiles of 293T/17 cells transfected with expression vectors encoding human CD4 (hCD4), canine IGHV4-1-mouse C_{μ} membrane form IgM^{b} allotype, and canine IGLV3-28/J_{κ}6 attached to various combinations of mouse C_{κ} and C_{λ} (1701), or canine IGKV2-5/J_{κ}1 attached to various combinations of mouse C_{κ} and C_{λ} (1702). The cells have been stained for cell surface hCD4 (1709) or for mouse IgM^{b} (1710).

FIG. 18 shows flow cytometry profiles of 293T/17 cells transfected with expression vectors encoding human CD4 (hCD4), canine IGHV3-19-mouse C_{μ} membrane form IgM^{b} allotype, and canine IGLV3-28/J_{λ}6 attached to various combinations of mouse C_{κ} and C_{λ} (1801), or canine IGKV2-5/J_{κ}1 attached to various combinations of mouse C_{κ} and C_{λ} (1802). The cells have been stained for cell surface hCD4 (1809) or for mouse IgM^{b} (1810).

FIG. 19A shows western blots of culture supernatants and FIG. 19B shows western blots of cell lysates of 393T/17 cells transfected with expression vectors encoding canine IGHV3-5 attached to mouse C_{γ2α} (1901), IGHV3-19 attached to mouse C_{γ2α} (1902) or IGHV4-1 attached to mouse C_{γ2α} (1903) and canine IGLV3-28/J_{κ}6 attached to various combinations of mouse C_{κ} (1907) and C_{λ} (1908-1910). The samples were electrophoresed under reducing conditions and the blot was probed with an anti-mouse IgG2a antibody.

FIG. 20A shows western blot loading control Myc for the cell lysates from FIG. 18 and FIG. 20B shows western blot loading control GAPDH for the cell lysates from FIG. 18.

FIG. 21A shows western blots of culture supernatants (non-reducing conditions) and FIG. 21B shows western blots of cell lysates (reducing conditions) of 393T/17 cells transfected with expression vectors encoding canine IGHV3-5-mouse C_{γ2α} and canine IGLV3-28/J_{κ}6 attached to various combinations of mouse C_{κ} (2102) and C_{λ} (2103, 2104) or transfected with expression vectors encoding canine IGHV3-5-mouse C_{γ2α} and canine IGKV2-5/J_{κ}1 attached to various combinations of mouse C_{κ} (2105) and C_{λ} (2106, 2107). The blots in FIG. 21A were probed with antibodies to mouse IgG2a and the blots in FIG. 21B were probed with antibodies to mouse κ LC.

FIG. 22 shows flow cytometry profiles of 293T/17 cells transfected with expression vectors encoding human CD4 (hCD4), canine IGHV3-5 attached to mouse C_{δ} membrane form, and canine IGKV2-5/J_{κ}1 attached to mouse C_{κ} (2201) or canine IGLV3-28/J_{κ}6 attached to mouse C_{λ1}, C_{λ2} or C_{λ3} (2202-2204). The cells have been stained for cell surface hCD4 (2205), mouse CD79b (2206), mouse IgD (2207), mouse κ LC (2208), or mouse λ LC (2209).

FIG. 23 shows flow cytometry profiles of 293T/17 cells transfected with expression vectors encoding human CD4 (hCD4), canine IGHV3-19 attached to mouse C_{δ} membrane form, and canine IGKV2-5/J_{κ}1 attached to mouse C_{κ} (2301) or canine IGLV3-28/J_{κ}6 attached to mouse C_{λ1}, C_{λ2} or C_{λ3} (2302-2304). The cells have been stained for cell surface hCD4 (2205), mouse CD79b (2206), mouse IgD (2207), mouse κ LC (2208), or mouse λ LC (2209).

FIG. 24 shows flow cytometry profiles of 293T/17 cells transfected with expression vectors encoding human CD4 (hCD4), canine IGHV4-1 attached to mouse C_{δ} membrane form, and canine IGKV2-5/J_{κ}1 attached to mouse C_{κ} (2401) or canine IGLV3-28/J_{κ}6 attached to mouse C_{λ1}, C_{λ2} or C_{λ3} (2402-2404). The cells have been stained for cell surface hCD4 (2405), mouse CD79b (2406), mouse IgD (2407), mouse κ LC (2408), or mouse λ LC (2409).

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “locus” as used herein refers to a chromosomal segment or nucleic acid sequence that, respectively, is present endogenously in the genome or is (or about to be) exogenously introduced into the genome. For example, an immunoglobulin locus may include part or all of the genes (i.e., V, D, J gene segments as well as constant region genes) and intervening sequences (i.e., introns, enhancers, etc.) supporting the expression of immunoglobulin H or L chain polypeptides. Thus, a locus (e.g., immunoglobulin heavy chain variable region gene locus) may refer to a specific portion of a larger locus (e.g., a portion of the immunoglobulin H chain locus that includes the V_{H}, D_{H} and J_{H} gene segments). Similarly, an immunoglobulin light chain variable region gene locus may refer to a specific portion of a larger locus (e.g., a portion of the immunoglobulin L chain locus that includes the V_{L} and J_{L} gene segments). The term “immunoglobulin variable region gene” as used herein refers to a V, D, or J gene segment that encodes a portion of an immunoglobulin H or L chain variable domain. The term “immunoglobulin variable region gene locus” as used herein refers to part of, or the entire, chromosomal segment or nucleic acid strand containing clusters of the V, D, or J gene segments and may include the non-coding regulatory or scaffold sequences.

The term “gene segment” as used herein, refers to a nucleic acid sequence that encodes a part of the heavy chain or light chain variable domain of an immunoglobulin molecule. A gene segment can include coding and non-coding sequences. The coding sequence of a gene segment is a nucleic acid sequence that can be translated into a polypeptide, such the leader peptide and the N-terminal portion of a heavy chain or light chain variable domain. The non-coding sequences of a gene segment are sequences flanking the coding sequence, which may include the promoter, 5′ untranslated sequence, intron intervening the coding sequences of the leader peptide, recombination signal sequence(s) (RSS), and splice sites. The gene segments in the immunoglobulin heavy chain (IGH) locus comprise the V_{H}, D and J_{H} gene segments (also referred to as IGHV, IGHD and IGHJ, respectively). The light chain variable region gene segments in the immunoglobulin κ and λ light loci can be referred to as V_{L} and J_{L} gene segments. In the κ light chain, the V_{L} and J_{L} gene segments can be referred to as V_{κ} and J_{κ} gene segments or IGKV and IGKJ. Similarly, in the λ light chain, the V_{L} and J_{L} gene segments can be referred to as V_{λ} and J_{λ} gene segments or IGLV and IGLJ.

The heavy chain constant region can be referred to as C_{H} or IGHC. The C_{H} region exons that encode IgM, IgD, IgG1-4, IgE, or IgA can be referred to as, respectively, C_{μ}, C_{δ}, C_{γ1-4}, C_{ε} or C_{α}. Similarly, the immunoglobulin κ or λ constant region can be referred to as C_{κ} or C_{λ}, as well as IGKC or IGLC, respectively.

“Partly canine” as used herein refers to a strand of nucleic acids, or their expressed protein and RNA products, comprising sequences corresponding to the sequences found in a given locus of both a canine and a non-canine mammalian host. “Partly canine” as used herein also refers to an animal comprising nucleic acid sequences from both a canine and a non-canine mammal, for example, a rodent. In one aspect, the partly canine nucleic acids have coding sequences of canine immunoglobulin H or L chain variable region gene segments and sequences based on the non-coding regulatory or scaffold sequences of the endogenous immunoglobulin locus of the non-canine mammal.

The term “based on” when used with reference to endogenous non-coding regulatory or scaffold sequences from a non-canine mammalian host cell genome refers to the non-coding regulatory or scaffold sequences that are present in the corresponding endogenous locus of the mammalian host cell genome. In one aspect, the term “based on” means that the non-coding regulatory or scaffold sequences that are present in the partly canine immunoglobulin locus share a relatively high degree of homology with the non-coding regulatory or scaffold sequences of the endogenous locus of the host mammal. In one aspect, the non-coding sequences in the partly canine immunoglobulin locus share at least about 80%, 90%, 95%, 96%, 97%, 98%, 99% or 100% homology with the corresponding non-coding sequences found in the endogenous locus of the host mammal. In one aspect, the non-coding sequences in the partly canine immunoglobulin locus are retained from an immunoglobulin locus of the host mammal. In one aspect, the canine coding sequences are embedded in the non-regulatory or scaffold sequences of the immunoglobulin locus of the host mammal. In one aspect, the host mammal is a rodent, such as a rat or mouse.

“Non-coding regulatory sequences” refer to sequences that are known to be essential for (i) V(D)J recombination, (ii) isotype switching, (iii) proper expression of the full-length immunoglobulin H or L chains following V(D)J recombination, and (iv) alternate splicing to generate, e.g., membrane and secreted forms of the immunoglobulin H chain. “Non-coding regulatory sequences” may further include the following sequences of endogenous origin: enhancer and locus control elements such as the CTCF and PAIR sequences (Proudhon, et al., Adv. Immunol. 128:123-182 (2015)); promoters preceding each endogenous V gene segment; splice sites; introns; recombination signal sequences flanking each V, D, or J gene segment. In one aspect, the “non-coding regulatory sequences” of the partly canine immunoglobulin locus share at least about 70%, 75%, 80%, 85%, 90%, 95%, 96%, 97%, 98%, 99% and up to 100% homology with the corresponding non-coding sequences found in the targeted endogenous immunoglobulin locus of the non-canine mammalian host cell.

“Scaffold sequences” refer to sequences intervening the gene segments present in the endogenous immunoglobulin locus of the host cell genome. In certain aspects, the scaffold sequences are interspersed by sequences essential for the expression of a functional non-immunoglobulin gene, for example, ADAM6A or ADAM6B. In certain aspects, the scaffold sequences are derived (at least partially) from other sources—e.g., they could be rationally designed or artificial sequences, sequences present in the immunoglobulin locus of the canine genome, sequences present in the immunoglobulin locus of another species, or combinations thereof. It is to be understood that the phrase “non-coding regulatory or scaffold sequence” is inclusive in meaning (i.e., referring to both the non-coding regulatory sequence and the scaffold sequence existing in a given locus).

The term “homology targeting vector” refers to a nucleic acid sequence used to modify the endogenous genome of a mammalian host cell by homologous recombination; such nucleic acid sequence may comprise (i) targeting sequences with significant homologies to the corresponding endogenous sequences flanking a locus to be modified that is present in the genome of the non-canine mammalian host, (ii) at least one sequence-specific recombination site, (iii) non-coding regulatory or scaffold sequences, and (iv) optionally one or more selectable marker genes. As such, a homology targeting vector can be used to introduce a sequence-specific recombination site into particular region of a host cell genome.

“Site-specific recombination” or “sequence-specific recombination” refers to a process of DNA rearrangement between two compatible recombination sequences (also referred to as “sequence-specific recombination sites” or “site-specific recombination sequences”) including any of the following three events: a) deletion of a preselected nucleic acid flanked by the recombination sites; b) inversion of the nucleotide sequence of a preselected nucleic acid flanked by the recombination sites, and c) reciprocal exchange of nucleic acid sequences proximate to recombination sites located on different nucleic acid strands. It is to be understood that this reciprocal exchange of nucleic acid segments can be exploited as a targeting strategy to introduce an exogenous nucleic acid sequence into the genome of a host cell.

The term “targeting sequence” refers to a sequence homologous to DNA sequences in the genome of a cell that flank or are adjacent to the region of an immunoglobulin locus to be modified. The flanking or adjacent sequence may be within the locus itself or upstream or downstream of coding sequences in the genome of the host cell. Targeting sequences are inserted into recombinant DNA vectors which are used to transfect, e.g., ES cells, such that sequences to be inserted into the host cell genome, such as the sequence of a recombination site, are flanked by the targeting sequences of the vector.

The term “site-specific targeting vector” as used herein refers to a vector comprising a nucleic acid encoding a sequence-specific recombination site, an engineered partly canine locus, and optionally a selectable marker gene, which is used to modify an endogenous immunoglobulin locus in a host using recombinase-mediated site-specific recombination. The recombination site of the targeting vector is suitable for site-specific recombination with another corresponding recombination site that has been inserted into a genomic sequence of the host cell (e.g., via a homology targeting vector), adjacent to an immunoglobulin locus that is to be modified. Integration of an engineered partly canine sequence into a recombination site in an immunoglobulin locus results in replacement of the endogenous locus by the exogenously introduced partly canine region.

The term “transgene” is used herein to describe genetic material that has been or is about to be artificially inserted into the genome of a cell, and particularly a cell of a mammalian host animal. The term “transgene” as used herein refers to a partly canine nucleic acid, e.g., a partly canine nucleic acid in the form of an engineered expression construct or a targeting vector.

“Transgenic animal” refers to a non-canine animal, usually a mammal, having an exogenous nucleic acid sequence present as an extrachromosomal element in a portion of its cells or stably integrated into its germ line DNA (i.e., in the genomic sequence of most or all of its cells). In one aspect, a partly canine nucleic acid is introduced into the germ line of such transgenic animals by genetic manipulation of, for example, embryos or embryonic stem cells of the host animal according to methods well known in the art.

A “vector” includes plasmids and viruses and any DNA or RNA molecule, whether self-replicating or not, which can be used to transform or transfect a cell.

Note that as used herein and in the appended claims, the singular forms “a,” “an,” and “the” include plural referents unless the context clearly dictates otherwise. Thus, for example, reference to “a locus” refers to one or more loci, and reference to “the method” includes reference to equivalent steps and methods known to those skilled in the art, and so forth.

As used herein, the term “or” can mean “and/or”, unless explicitly indicated to refer only to alternatives or the alternatives are mutually exclusive. The terms “including,” “includes” and “included”, are not limiting.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, polymerase chain reaction, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach and Veksler, Eds. (2007), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3.sup.rd Ed., W. H. Freeman Pub., New York, N.Y.; and Berg et al. (2002) Biochemistry, 5.sup.th Ed., W.H. Freeman Pub., New York, N.Y., all of which are herein incorporated in their entirety by reference for all purposes.

### DETAILED DESCRIPTION

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Described herein is a transgenic rodent or rodent cell having a genome comprising an engineered partly canine immunoglobulin heavy chain or light chain locus. In one aspect, the partly canine immunoglobulin heavy chain locus comprises one or more canine immunoglobulin heavy chain variable region gene segments. In one aspect, the partly canine immunoglobulin light chain locus comprises one or more canine immunoglobulin λ light chain variable region gene segments. In one aspect, the partly canine immunoglobulin light chain locus comprises one or more canine immunoglobulin κ light chain variable region gene segments.

In one aspect, non-canine mammalian cells are provided that comprise an exogenously introduced, engineered partly canine nucleic acid sequence comprising coding sequences for canine variable regions and non-coding regulatory or scaffold sequences present in the immunoglobulin locus of the mammalian host genome, e.g., mouse genomic non-coding sequences when the host mammal is a mouse. In one aspect, one or more coding sequences for canine variable region gene segments are embedded in non-coding regulatory or scaffold sequences corresponding to those of an immunoglobulin locus in a mammalian host genome. In one aspect, the coding sequences for canine variable region gene segments are embedded in non-coding regulatory or scaffold sequences of a rodent or mouse immunoglobulin locus.

In one aspect, the partly canine immunoglobulin locus is synthetic and comprises canine V_{H}, D, or J_{H} or V_{L} or J_{L} gene segment coding sequences that are under the control of regulatory elements of the endogenous host. In one aspect, the partly canine immunoglobulin locus comprises canine V_{H}, D, or J_{H} or V_{L} or J_{L} gene segment coding sequences embedded in non-coding regulatory or scaffold sequences corresponding to those of an immunoglobulin locus in a mammalian host genome.

Methods are also provided for generating a transgenic rodent or rodent ES cell comprising exogenously introduced, engineered partly canine immunoglobulin loci, wherein the resultant transgenic rodent is capable of producing more immunoglobulin comprising λ light chain than immunoglobulin comprising κ light chain.

There are many challenges presented when generating a non-canine mammal such as a transgenic mouse or rat, that is capable of producing antigen-specific canine antibodies that are addressed by the constructs and methods described herein, including, but not limited to:
1. How to obtain λ:κ light chain usage ratio of 90:10 in an organism such as a mouse or rat that preferentially uses 90% κ light chains;
2. Whether mouse B cells can express a large number of dog V_{λ} gene segments (the dog λ locus contains at least 70 functional, unique V_{λ} gene segments) when the mouse λ locus contains only 3 functional V_{λ} gene segments;
3. How to improve expression and usage of canine V_{λ} in a non-canine mammal, such as a mouse, in view of the differences in structure between the mouse and dog λ light chain loci locus.
   a. The mouse λ light chain loci locus contains 2 clusters of V_{λ} gene segment(s), J_{λ} gene segment(s), and C_{λ} exon(s):
      i. V_{λ2}-V_{λ3}-J_{λ2}-C_{λ2}
      ii. V_{λ1}-J_{λ3}-C_{λ3}-J_{λ1}-C_{λ1}; and
   b. the dog λ locus contains tandem V_{λ} gene segments upstream of J_{λ}-C_{λ} clusters.
4. Whether mouse B cells can develop normally if mouse IgD is expressed with dog V_{H}, in view of the fact that canine IgD is not functional and IgM and IgD are co-expressed as alternatively spliced forms in mouse and rat B cells.

### Immunoglobulin Loci in Mice and Dog

In the humoral immune system, a diverse antibody repertoire is produced by combinatorial and junctional diversity of IGH and IGL chain gene loci by a process termed V(D)J recombination. In the developing B cell, the first recombination event to occur is between one D and one J_{H} gene segment of the heavy chain locus, and the DNA between these two gene segments is deleted. This D-J_{H} recombination is followed by the joining of one V_{H} gene segment from a region upstream of the newly formed DJ_{H} complex, forming a rearranged V_{H}DJ_{H} exon. All other sequences between the recombined V_{H} and D gene segments of the newly generated V_{H}DJ_{H} exon are deleted from the genome of the individual B cell. This rearranged exon is ultimately expressed on the B cell surface as the variable region of the H-chain polypeptide, which is associated with an L-chain polypeptide to form the B cell receptor (BCR).

The light chain repertoire in the mouse is believed to be shaped by the order of gene rearrangements. The IGK light chain locus on both chromosomes is believed to undergo V_{κ}-J_{κ} rearrangements first before the IGL light chain locus on either chromosome becomes receptive for V_{λ}-J_{λ} recombination. If an initial κ rearrangement is unproductive, additional rounds of secondary rearrangement can proceed, in a process known as receptor editing (Collins and Watson. (2018) Immunoglobulin light chain gene rearrangements, receptor editing and the development of a self-tolerant antibody repertoire. Front. Immunol. 9:2249.) A process of serial rearrangement of the κ chain locus may continue on one chromosome until all possibilities of recombination are exhausted. Recombination will then proceed on the second κ chromosome. A failure to produce a productive rearrangement on the second chromosome after multiple rounds of rearrangement will be followed by rearrangement on the λ loci (Collins and Watson (2018) Immunoglobulin light chain gene rearrangements, receptor editing and the development of a self-tolerant antibody repertoire. Front. Immunol. 9:2249.)

This preferential order of light chain rearrangements is believed to give rise to a light chain repertoire in mouse that is >90% κ and <10% λ. However, immunoglobulins in the dog immune system are dominated by λ light chain usage, which has been estimated to be at least 90% λ to <10% κ (Arun et al. (1996) Immunohistochemical examination of light-chain expression (λ/κ ratio) in canine, feline, equine, bovine and porcine plasma cells. Zentralbl Veterinarmed A. 43(9):573-6).

The murine and canine Ig loci are highly complex in the numbers of features they contain and in how their coding regions are diversified by V(D)J rearrangement; however, this complexity does not extend to the basic details of the structure of each variable region gene segment. The V, D and J gene segments are highly uniform in their compositions and organizations. For example, V gene segments have the following features that are arranged in essentially invariant sequential fashion in immunoglobulin loci: a short transcriptional promoter region (<600 bp in length), an exon encoding the 5′ UTR and the majority of the signal peptide for the antibody chain; an intron; an exon encoding a small part of the signal peptide of the antibody chain and the majority of the antibody variable domain, and a 3′ recombination signal sequence necessary for V(D)J rearrangement. Similarly, D gene segments have the following necessary and invariant features: a 5′ recombination signal sequence, a coding region and a 3′ recombination signal sequence. The J gene segments have the following necessary and invariant features: a 5′ recombination signal sequence, a coding region and a 3′ splice donor sequence.

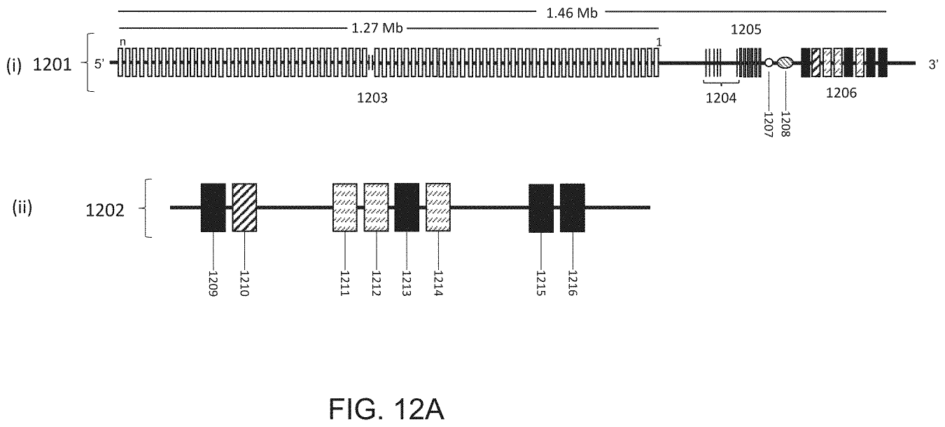

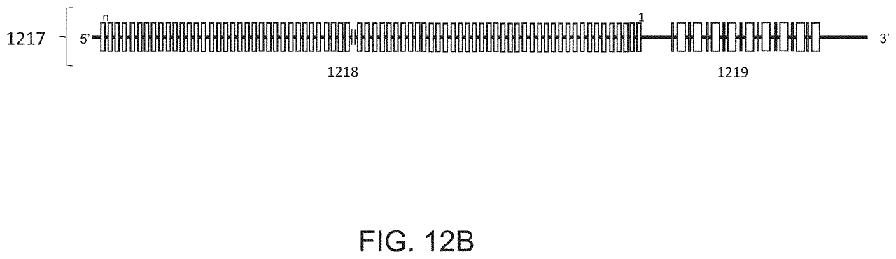

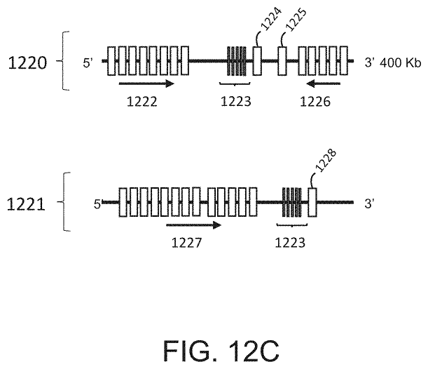

The canine genome V_{H} region comprises approximately 39 functional V_{H}, 6 functional D and 5 functional J_{H} gene segments mapping to a 1.46 Mb region of canine chromosome 8. There are also numerous V_{H} pseudogenes and one J_{H} gene segment (IGHJ1) and one D gene segment (IGHD5) that are thought to be non-functional because of non-canonical heptamers in their RSS. (Such gene segments are referred to as Open Reading Frames (ORFs).) FIG. 12A provides a schematic diagram of the endogenous canine IGH locus (1201) as well as an expanded view of the IGHC region (1202). The canine immunoglobulin heavy chain variable region locus, which includes V_{H} (1203), D (1204) and J_{H} (1205) gene segments, has all functional genes in the same transcriptional orientation as the constant region genes (1206), with two pseudogenes (IGHV3-4 and IGHV1-4-1) in the reverse transcriptional orientation (not shown). A transcriptional enhancer (1207) and the (1208) μ switch region are located within the J_{H}-Cμ intron. See, Martin et al. (2018) Comprehensive annotation and evolutionary insights into the canine (Canis lupus familiaris) antigen receptor loci. Immunogenetics. 70:223-236. Among the IGHC genes, C_{δ} (1210) is thought to be non-functional. Moreover, although cDNA clones identified as encoding canine IgG1 (1212), IgG2 (1213), IgG3 (1211) and IgG4 (1214) have been isolated (Tang, et al. (2001) Cloning and characterization of cDNAs encoding four different canine immunoglobulin γ chains. Vet. Immunol. and Immunopath. 80:259 PMID 11457479), only the IgG2 constant region gene has been physically mapped to the canine IGHC locus on chromosome 8. Functional versions of C_{μ} (1209), C_{ε} (1215) and C_{α} (1216) have also been physically mapped there.

The sequences of the canine IGHC are in Table 4.

The canine IGL locus maps to canine chromosome 26, while the canine IGK coding region maps to canine chromosome 17. FIGS. 12B and 12C provide schematic diagrams of the endogenous canine IGL and IGK loci, respectively.

The sequences of the canine IGKC and IGLC are in Table 4.

The canine λ locus (1217) is large (2.6 Mbp) with 162 V_{λ} genes (1218), of which at least 76 are functional. The canine λ locus also includes 9 tandem cassettes or J-C units, each containing a J_{λ} gene segment and a C_{λ} exon (1219). See, Martin et al. (2018) Comprehensive annotation and evolutionary insights into the canine (Canis lupus familiaris) antigen receptor loci. Immunogenetics. 70:223-236.

The canine κ locus (1220) is small (400 Kbp) and has an unusual structure in that eight of the functional V_{κ} gene segments are located upstream (1222) and five are located downstream (1226) of the J_{κ} (1223) gene segments and C_{κ} (1224) exon. The canine upstream V_{κ} region has all functional gene segments in the same transcriptional orientation as the J_{κ} gene segment and C_{κ} exon, with two pseudogenes (IGKV3-3 and IGKV7-2) and one ORF (IGKV4-1) in the reverse transcriptional orientation (not shown). The canine downstream V_{κ} region has all functional gene segments in the opposite transcriptional orientation as the J_{κ} gene segment and C_{κ} exon and includes six pseudogenes. The Ribose 5-Phosphate Isomerase A (RPIA) gene (1225) is also found in the downstream V_{κ} region, between C_{κ} and IGKV2S19. See, Martin et al. (2018) Comprehensive annotation and evolutionary insights into the canine (Canis lupus familiaris) antigen receptor loci. Immunogenetics. 70:223-236.

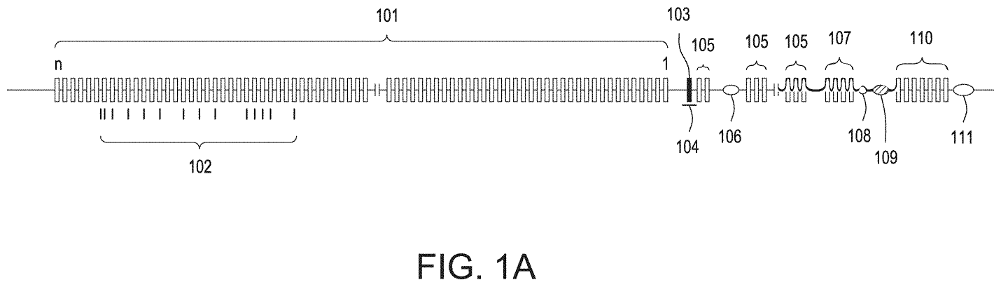

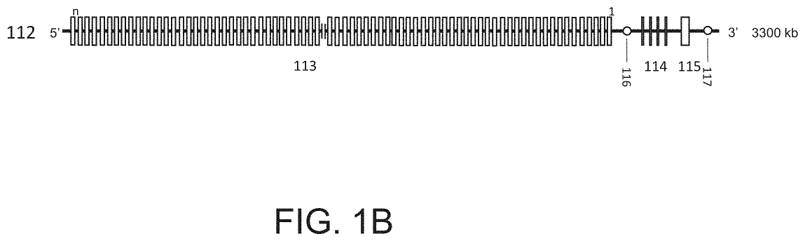

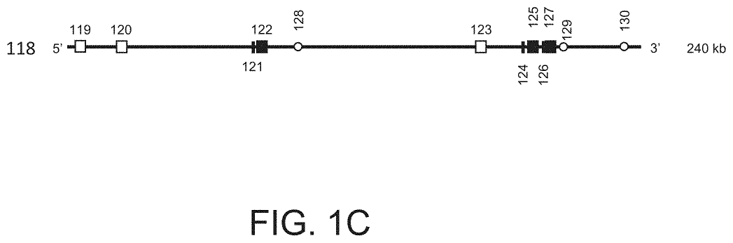

The mouse immunoglobulin κ locus is located on chromosome 6. FIG. 1B provides a schematic diagram of the endogenous mouse IGK locus. The IGK locus (112) spans 3300 Kbp and includes more than 100 variable V_{κ} gene segments (113) located upstream of 5 joining (J_{κ}) gene segments (114) and one constant (C_{κ}) gene (115). The mouse κ locus includes an intronic enhancer (iE_{κ}, 116) located between J_{κ} and C_{κ} that activates κ rearrangement and helps maintain the earlier or more efficient rearrangement of κ versus λ (Inlay et al. (2004) Important Roles for E Protein Binding Sites within the Immunoglobulin κ chain intronic enhancer in activating V_{κ}J_{κ} rearrangement. J. Exp. Med. 200(9):1205-1211). Another enhancer, the 3′ enhancer (3′E_{κ}, 117) is located 9.1 Kb downstream of the C_{κ} exon and is also involved in κ rearrangement and transcription; mutant mice lacking both iE_{κ} and 3′Eκ have no V_{κ}J_{κ} rearrangements in the κ locus (Inlay et al. (2002) Essential roles of the kappa light chain intronic enhancer and 3′ enhancer in kappa rearrangement and demethylation. Nature Immunol. 3(5):463-468). However, disrupting the iE_{κ}, for example, by insertion of a neomycin-resistance gene is also sufficient to abolish most V_{κ}J_{κ} rearrangements (Xu et al. (1996) Deletion of the Igκ Light Chain Intronic Enhancer/Matrix Attachment Region Impairs but Does Not Abolish V_{κ}J_{κ} Rearrangement).

The mouse immunoglobulin λ locus is located on chromosome 16. FIG. 1C provides a schematic diagram of the endogenous mouse IGL locus (118). The organization of the mouse immunoglobulin λ locus is different from the mouse immunoglobulin κ locus. The locus spans 240 kb, with two clusters comprising 3 functional variable (V_{λ}) gene segments (IGLV2, 119; IGLV3, 120 and IGLV1, 123) and 3 tandem cassettes of λ joining (J_{λ}) gene segments and constant (C_{λ}) gene segments (IGLJ2, 121; IGLC2, 122; IGLJ3, 124: IGLC3, 125; IGLJ1, 126; IGLC1, 127) in which the V_{λ} gene segments are located upstream (5′) from a variable number of J-C tandem cassettes. The locus also contains three transcriptional enhancers (E_{κ2-4}, 128; E_{λ}, 129; _{Eλ3-1}, 130).

The partly canine nucleic acid sequence described herein allows the transgenic animal to produce a heavy chain or light chain repertoire comprising canine V_{H} or V_{L} regions, while retaining the regulatory sequences and other elements that can be found within the intervening sequences of the host genome (e.g., rodent) that help to promote efficient antibody production and antigen recognition in the host.

In one aspect, synthetic, or recombinantly produced, partly canine nucleic acids are engineered to comprise both canine coding sequences and non-canine non-coding regulatory or scaffold sequences of an immunoglobulin V_{H}, V_{λ} or V_{κ} locus, or, in some aspects, a combination thereof.

In one aspect, a transgenic rodent or rodent cell that expresses immunoglobulin with a canine variable region can be generated by inserting one or more canine V_{H} gene segment coding sequences into a V_{H} locus of a rodent heavy chain immunoglobulin locus. In another aspect, a transgenic rodent or rodent cell that expresses immunoglobulin with canine a variable region can be generated by inserting one or more canine V_{L} gene segment coding sequences into a V_{L} locus of a rodent light chain immunoglobulin locus.

The existence of two light chain loci—κ and λ—means that a variety of light chain insertion combinations are possible for generating a transgenic rodent or rodent cell that expresses immunoglobulin with canine a variable region, including but not limited to: inserting one or more canine V_{λ} or J_{λ} gene segment coding sequences into a rodent V_{λ} locus, inserting one or more canine V_{κ} or J_{κ} gene segment coding sequences into a rodent V_{κ} locus, inserting one or more canine V_{λ} or J_{λ} gene segment coding sequences into a rodent V_{κ} locus and inserting one or more canine V_{κ} or J_{κ} gene segment coding sequences into a rodent V_{λ} locus.

The selection and development of a transgenic rodent or rodent cell that expresses partly canine immunoglobulin is complicated by the fact that more than 90% of light chains produced by mice are κ and less than 10% are λ whereas more than 90% of light chains produced by dogs are λ and less than 10% κ and the fact that the canine immunoglobulin locus is large and includes over 100 V_{λ} gene segments, whereas the mouse immunoglobulin λ includes only 3 functional V_{λ} gene segments.

Since mice produce mainly κ LC-containing antibodies, one reasonable method to increase production of λ LC-containing partly canine immunoglobulin by the transgenic rodent would be to insert one or more canine V_{λ} or J_{λ} gene segment coding sequences into a rodent κ locus. However, as shown in the Example 9 below, coupling canine V_{λ} region exon with rodent C_{κ} region exon results in sub-optimal expression of the partly canine immunoglobulin in vitro.

Provided herein is a transgenic rodent or rodent cell that is capable of expressing immunoglobulin comprising canine variable domains, wherein the transgenic rodent produces more or is more likely to produce immunoglobulin comprising λ light chain than immunoglobulin comprising κ light chain. While not wishing to be bound by theory, it is believed that a transgenic rodent or rodent cell that produces more, or is more likely to produce, immunoglobulin comprising λ light chain will result in a fuller antibody repertoire for the development of therapeutics.

A transgenic rodent or rodent cell having a genome comprising an engineered partly canine immunoglobulin light chain locus is provided herein. In one aspect, the partly canine immunoglobulin light chain locus comprises canine immunoglobulin λ light chain variable region gene segments. In one aspect, the engineered immunoglobulin locus is capable of expressing immunoglobulin comprising a canine variable domain. In one aspect, the engineered immunoglobulin locus is capable of expressing immunoglobulin comprising a canine λ variable domain. In one aspect, the engineered immunoglobulin locus is capable of expressing immunoglobulin comprising a canine κ variable domain. In one aspect, the engineered immunoglobulin locus expresses immunoglobulin light chains comprising a canine variable domain and a rodent constant domain. In one aspect, the engineered immunoglobulin locus expresses immunoglobulin light chains comprising a canine λ variable domain and a rodent λ constant domain. In one aspect, the engineered immunoglobulin locus expresses immunoglobulin light chains comprising a canine κ variable domain and a rodent κ constant domain.

In one aspect, the transgenic rodent or rodent cell produces more, or is more likely to produce, immunoglobulin comprising λ light chain than immunoglobulin comprising κ light chain. In one aspect, a transgenic rodent is provided in which more λ light chain producing cells than κ light chain producing cells are likely to be isolated from the rodent. In one aspect, a transgenic rodent is provided that produces at least about 25%, 30%, 35%, 40%, 45%, 50%, 55%, 60%, 65%, 70%, 75%, 80%, 85%, 90% or 95% and up to about 100% immunoglobulin comprising λ light chain. In one aspect, a transgenic rodent cell, or its progeny, is provided that is more likely to produce immunoglobulin with λ light chain than immunoglobulin with κ light chain. In one aspect, the transgenic rodent cell, or its progeny, has at least about a 25%, 30%, 35%, 40%, 45%, 50%, 55%, 60%, 65%, 70%, 75%, 80%, 85%, 90%, or 95% and up to about 100%, probability of producing immunoglobulin comprising λ light chain. In one aspect, a transgenic rodent or rodent cell is provided in which an endogenous rodent light chain immunoglobulin locus has been deleted and replaced with an engineered partly canine light chain immunoglobulin locus. In one aspect, the transgenic rodent is a mouse.

### Immunoglobulin Light Chain Locus

In one aspect, a transgenic rodent or rodent cell is provided that has a genome comprising a recombinantly produced partly canine immunoglobulin variable region locus. In one aspect, the partly canine immunoglobulin variable region locus is a light chain variable region (V_{L}) locus. In one aspect, the partly canine immunoglobulin variable region locus comprises one or more canine V_{λ} gene segment coding sequences or one or more canine J_{λ} gene segment coding sequences. In one aspect, the partly canine immunoglobulin variable region locus comprises one or more canine V_{κ} gene segment coding sequences or one or more canine J_{κ} gene segment coding sequences. In one aspect, the partly canine immunoglobulin variable region locus comprises one or more rodent constant domain genes or coding sequences. In one aspect, the partly canine immunoglobulin variable region locus comprises one or more rodent C_{λ} genes or coding sequences. In one aspect, the partly canine immunoglobulin variable region locus comprises one or more rodent C_{κ} genes or coding sequences. In one aspect, an endogenous rodent light chain immunoglobulin locus has been inactivated. In one aspect, an endogenous rodent light chain immunoglobulin locus has been deleted and replaced with an engineered partly canine light chain immunoglobulin locus.

In one aspect, the engineered immunoglobulin locus expresses immunoglobulin light chains comprising a canine λ variable domain and rodent λ constant domain. In one aspect, the engineered immunoglobulin locus expresses immunoglobulin light chains comprising a canine κ variable domain and rodent κ constant domain.

In one aspect, the engineered partly canine immunoglobulin variable region locus comprises a V_{L} locus comprising most or all of the V_{λ} gene segments coding sequences from a canine genome. In one aspect, the engineered partly canine immunoglobulin locus variable region comprises a V_{L} locus comprising at least 20, 30, 40, 50, 60, 70 and up to 76 canine V_{λ} gene segment coding sequences. In one aspect the engineered partly canine immunoglobulin variable region locus comprises a V_{L} locus comprising at least about 50%, 60%, 70%, 80%, 90% and up to 100% of the V_{λ} gene segment coding sequences from a canine genome.

In one aspect, the engineered partly canine immunoglobulin locus variable region comprises a V_{L} locus comprising most or all of the J_{λ} gene segment coding sequences found in the canine genome. In one aspect, the engineered partly canine immunoglobulin locus variable region comprises a V_{L} locus comprising at least 1, 2, 3, 4, 5, 6, 7, 8, or 9 canine J_{λ} gene segment coding sequences. In one aspect the engineered partly canine immunoglobulin variable region locus comprises a V_{L} locus comprising at least about 50%, 75%, and up to 100% of the J_{λ} gene segment coding sequences found in the canine genome.

In one aspect, the engineered partly canine immunoglobulin locus variable region comprises a V_{L} locus comprising most or all of the V_{λ} and J_{λ} gene segment coding sequences from the canine genome. In one aspect the engineered partly canine immunoglobulin variable region locus comprises a V_{L} locus comprising at least about 50%, 60%, 70%, 80%, 90% and up to 100% of the V_{λ} and J_{λ} gene segment coding sequences from the canine genome.

In one aspect, the engineered partly canine immunoglobulin locus variable region comprises a V_{L} locus comprising most or all of the V_{κ} gene segment coding sequences from the canine genome. In one aspect, the engineered partly canine immunoglobulin locus variable region comprises a V_{L} locus comprising at least 4, 5, 6, 7, 8, 9, 10, 11, 12, 13, and up to 14 canine V_{κ} gene segment coding sequences. In one aspect the engineered partly canine immunoglobulin variable region locus comprises a V_{L} locus comprising at least about 50%, 60%, 70%, 80%, 90% and up to 100% of the V_{κ} gene segment coding sequences from the canine genome.

In one aspect, the engineered partly canine immunoglobulin locus variable region comprises a V_{L} locus comprising most or all of the J_{κ} gene segment coding sequences found in the canine genome. In one aspect, the engineered partly canine immunoglobulin locus variable region comprises a V_{L} locus comprising at least 1, 2, 3, 4 or 5 canine J_{κ} gene segment coding sequences. In one aspect the engineered partly canine immunoglobulin variable region locus comprises a V_{L} locus comprising at least about 50%, 75%, and up to 100% of the J_{κ} gene segment coding sequences found in the canine genome.

In one aspect, the engineered partly canine immunoglobulin locus variable region comprises a V_{L} locus comprising most or all of the V_{κ} and J_{κ} gene segment coding sequences from the canine genome. In one aspect the engineered partly canine immunoglobulin variable region locus comprises a V_{L} locus comprising at least about 50%, 60%, 70%, 80%, 90% and up to 100% of the V_{κ} and J_{κ} gene segment coding sequences from the canine genome.

In one aspect, the engineered immunoglobulin locus comprises canine V_{L} gene segment coding sequences and rodent non-coding regulatory or scaffold sequences from a rodent immunoglobulin light chain variable region gene locus. In one aspect, the engineered immunoglobulin locus comprises canine V_{λ} or J_{λ} gene segment coding sequences and rodent non-coding regulatory or scaffold sequences from a rodent immunoglobulin light chain variable region gene locus. In one aspect, the rodent non-coding regulatory or scaffold sequences are from a rodent immunoglobulin λ light chain variable region gene locus. In one aspect, the rodent non-coding regulatory or scaffold sequences are from a rodent immunoglobulin κ light chain variable region locus. In one aspect, the engineered immunoglobulin locus comprises canine V_{λ} and J_{λ} gene segment coding sequences and rodent non-coding regulatory or scaffold sequences from a rodent immunoglobulin λ light chain variable region gene locus. In one aspect, the partly canine immunoglobulin locus comprises one or more rodent immunoglobulin λ constant region (C_{λ}) coding sequences. In one aspect, the partly canine immunoglobulin locus comprises one or more canine V_{λ} and J_{λ} gene segment coding sequences and one or more rodent immunoglobulin C_{λ} coding sequences. In one aspect, the engineered immunoglobulin locus comprises canine V_{λ} and J_{λ} gene segment coding sequences and one or more rodent C_{λ} coding sequences embedded in rodent non-coding regulatory or scaffold sequences of a rodent immunoglobulin λ light chain variable region gene locus.

In one aspect, the engineered immunoglobulin locus comprises canine V_{λ} or J_{λ} gene segment coding sequences and rodent non-coding regulatory or scaffold sequences from a rodent immunoglobulin κ light chain variable region gene locus. In one aspect, the engineered immunoglobulin locus comprises canine V_{λ} or J_{λ} gene segment coding sequences embedded in rodent non-coding regulatory or scaffold sequences of a rodent immunoglobulin κ light chain variable region gene locus. In one aspect, the engineered immunoglobulin locus comprises canine V_{λ} and J_{λ} gene segment coding sequences and one or more rodent immunoglobulin C_{λ} coding sequences and rodent non-coding regulatory or scaffold sequences from a rodent immunoglobulin κ light chain variable region gene locus. In one aspect, the engineered immunoglobulin locus comprises canine V_{λ} and J_{λ} gene segment coding sequences and one or more rodent immunoglobulin C_{λ} coding sequences embedded in rodent non-coding regulatory or scaffold sequences of a rodent immunoglobulin κ light chain variable region gene locus.

In one aspect, one or more canine V_{λ} gene segment coding sequences are located upstream of one or more J_{λ} gene segment coding sequences, which are located upstream of one or more rodent C_{λ} genes. In one aspect, one or more canine V_{λ} gene segment coding sequences are located upstream and in the same transcriptional orientation as one or more J_{λ} gene segment coding sequences, which are located upstream of one or more rodent lambda C_{λ} genes.

In one aspect, the engineered immunoglobulin variable region locus comprises one or more canine V_{λ} gene segment coding sequences, one or more canine J_{λ} gene segment coding sequences and one or more rodent C_{λ} genes. In one aspect, the engineered immunoglobulin variable region locus comprises one or more canine V_{λ} gene segment coding sequences, one or more canine J_{λ} gene segment coding sequence and one or more rodent C_{λ} region genes, wherein the V_{λ} and J_{λ} gene segment coding sequences and the rodent C_{λ} region genes are inserted into a rodent immunoglobulin κ light chain locus. In one aspect, the engineered immunoglobulin variable region locus comprises one or more canine V_{λ} gene segment coding sequences, one or more canine J_{λ} gene segment coding sequence and one or more rodent C_{λ} genes, wherein the V_{λ} and J_{λ} gene segment coding sequences and the rodent (C_{λ}) region genes are embedded in non-coding regulatory or scaffold sequences of a rodent immunoglobulin κ light chain locus.

In one aspect, one or more canine V_{λ} gene segment coding sequences are located upstream of one or more J_{λ} gene segment coding sequences, which are located upstream of one or more rodent C_{λ} genes, wherein the V_{λ} and J_{λ} gene segment coding sequences and rodent C_{λ} genes are inserted into a rodent immunoglobulin κ light chain locus. In one aspect, one or more canine V_{λ} gene segment coding sequences are located upstream of one or more J_{λ} gene segment coding sequences, which are located upstream of one or more rodent C_{λ} genes, wherein the V_{λ} and J_{λ} gene segment coding sequences and rodent C_{λ} genes are embedded in non-coding regulatory or scaffold sequences of a rodent immunoglobulin κ light chain locus.

In one aspect, the rodent C_{λ} coding sequence is selected from a rodent C_{λ1}, C_{λ2}, or C_{λ3} coding sequence.

In one aspect, a transgenic rodent or rodent cell is provided, wherein the engineered immunoglobulin locus comprises a rodent immunoglobulin κ locus in which one or more rodent V_{κ} gene segment coding sequences and one or more rodent J_{κ} gene segment coding sequences have been deleted and replaced by one or more canine V_{λ} gene segment coding sequences and one or more J_{λ} gene segment coding sequences, respectively, and in which rodent C_{κ} coding sequences in the locus have been replaced by rodent C_{λ1}, C_{λ2}, or C_{λ3} coding sequence.

In one aspect, the engineered immunoglobulin variable region locus comprises one or more canine V_{λ} gene segment coding sequences and one or more J-C units wherein each J-C unit comprises a canine J_{λ} gene segment coding sequence and a rodent C_{λ} gene. In one aspect, the engineered immunoglobulin variable region locus comprises one or more canine V_{λ} gene segment coding sequences and one or more J-C units wherein each J-C unit comprises a canine J_{λ} gene segment coding sequence and rodent C_{λ} region coding sequence, wherein the V_{λ} gene segment coding sequences and the J-C units are inserted into a rodent immunoglobulin κ light chain locus. In one aspect, the engineered immunoglobulin variable region locus comprises one or more canine V_{λ} gene segment coding sequences and one or more J-C units wherein each J-C unit comprises a canine J_{λ} gene segment coding sequence and rodent C_{λ} coding sequence, wherein the V_{λ} gene segment coding sequences and the J-C units are embedded in non-coding regulatory or scaffold sequences of a rodent immunoglobulin κ light chain locus.

In one aspect, one or more canine V_{λ} gene segment coding sequences are located upstream and in the same transcriptional orientation as one or more J-C units, wherein each J-C unit comprises a canine J_{λ} gene segment coding sequence and a rodent C_{λ} gene. In one aspect, one or more canine V_{λ} gene segment coding sequences are located upstream and in the same transcriptional orientation as one or more J-C units, wherein each J-C unit comprises a canine J_{λ} gene segment coding sequence and a rodent C_{λ} coding sequence. In one aspect, the engineered immunoglobulin variable region locus comprises one or more canine V_{λ} gene segment coding sequences located upstream of one or more J-C units wherein each J-C unit comprises a canine J_{λ} gene segment coding sequence and rodent Cλ coding sequence, wherein the V_{λ} gene segment coding sequences and the J-C units are inserted into a rodent immunoglobulin κ light chain locus. In one aspect, the engineered immunoglobulin variable region locus comprises one or more canine V_{λ} gene segment coding sequences upstream and in the same transcriptional orientation as one or more J-C units wherein each J-C unit comprises a canine J_{λ} gene segment coding sequence and rodent Cλ coding sequence, wherein the V_{λ} gene segment coding sequences and the J-C units are embedded in non-coding regulatory or scaffold sequences of a rodent immunoglobulin κ light chain locus. In one aspect, the rodent C_{λ} coding sequence is selected from a rodent C_{λ1}, C_{λ2}, or C_{λ3} coding sequence.

In one aspect, the engineered immunoglobulin locus comprises canine V_{κ} coding sequences and rodent non-coding regulatory or scaffold sequences from a rodent immunoglobulin light chain variable region gene locus. In one aspect, the engineered immunoglobulin locus comprises canine V_{κ} or J_{κ} gene segment coding sequences and rodent non-coding regulatory or scaffold sequences from a rodent immunoglobulin light chain variable region gene locus. In one aspect, the rodent non-coding regulatory or scaffold sequences are from a rodent immunoglobulin λ light chain variable region gene locus. In one aspect, the rodent non-coding regulatory or scaffold sequences are from a rodent immunoglobulin κ light chain variable region locus. In one aspect, the engineered immunoglobulin locus comprises canine V_{κ} and J_{κ} gene segment coding sequences and rodent non-coding regulatory or scaffold sequences from a rodent immunoglobulin κ light chain variable region gene locus. In one aspect, the engineered immunoglobulin locus comprises canine V_{κ} and J_{κ} gene segment coding sequences and rodent non-coding regulatory or scaffold sequences from a rodent immunoglobulin λ light chain variable region gene locus. In one aspect, the partly canine immunoglobulin locus comprises one rodent immunoglobulin C_{κ} coding sequences. In one aspect, the partly canine immunoglobulin locus comprises one or more rodent immunoglobulin C_{λ} coding sequences. In one aspect, the partly canine immunoglobulin locus comprises one or more canine V_{κ} and J_{κ} gene segment coding sequences and one rodent immunoglobulin C_{κ} coding sequences. In one aspect, the engineered immunoglobulin locus comprises canine V_{κ} and J_{κ} gene segment coding sequences and one rodent immunoglobulin C_{κ} coding sequences embedded in rodent non-coding regulatory or scaffold sequences of a rodent κ light chain variable region gene locus. In one aspect, the engineered immunoglobulin locus comprises canine V_{κ} and J_{κ} gene segment coding sequences and one rodent immunoglobulin C_{κ} coding sequences embedded in rodent non-coding regulatory or scaffold sequences of a rodent immunoglobulin λ light chain variable region gene locus.

While not wishing to be bound by theory, it is believed that inactivating or rendering nonfunctional an endogenous rodent κ light chain locus may increase expression of λ light chain immunoglobulin from the partly canine immunoglobulin locus. This has been shown to be the case in otherwise conventional mice in which the κ light chain locus has been inactivated in the germline (Zon, et al. (1995) Subtle differences in antibody responses and hypermutation of λ light chains in mice with a disrupted κ constant region. Eur. J. Immunol. 25:2154-2162). In one aspect, inactivating or rendering nonfunctional an endogenous rodent κ light chain locus may increase the relative amount of immunoglobulin comprising λ light chain relative to the amount of immunoglobulin comprising κ light chain produced by the transgenic rodent or rodent cell.

In one aspect, a transgenic rodent or rodent cell is provided in which an endogenous rodent immunoglobulin κ light chain locus is deleted, inactivated, or made nonfunctional. In one aspect, the endogenous rodent immunoglobulin κ light chain locus is inactivated or made nonfunctional by one or more of the following deleting or mutating all endogenous rodent V_{κ} gene segment coding sequences; deleting or mutating all endogenous rodent J_{κ} gene segment coding sequences; deleting or mutating the endogenous rodent C_{κ} coding sequence; deleting, mutating, or disrupting the endogenous intronic κ enhancer (iE_{κ}) and 3′ enhancer sequence (3′E_{κ}); or a combination thereof.

In one aspect, a transgenic rodent or rodent cell is provided in which an endogenous rodent immunoglobulin λ light chain variable domain is deleted, inactivated, or made nonfunctional. In one aspect, the endogenous rodent immunoglobulin λ light chain variable domain is inactivated or made nonfunctional by one or more of the following: deleting or mutating all endogenous rodent V_{κ} gene segments; deleting or mutating all endogenous rodent J_{λ} gene segments; deleting or mutating all endogenous rodent C_{λ} coding sequences; or a combination thereof.

In one aspect, the partly canine immunoglobulin locus comprises rodent regulatory or scaffold sequences, including, but not limited to enhancers, promoters, splice sites, introns, recombination signal sequences, and combinations thereof. In one aspect, the partly canine immunoglobulin locus comprises rodent λ regulatory or scaffold sequences. In one aspect, the partly canine immunoglobulin locus comprises rodent κ regulatory or scaffold sequences.

In one aspect, the partly canine immunoglobulin locus includes a promoter to drive gene expression. In one aspect, the partly canine immunoglobulin locus includes a κ V-region promoter. In one aspect, the partly canine immunoglobulin locus includes a λ V-region promoter. In one aspect, the partly canine immunoglobulin locus includes a λ V-region promoter to drive expression of one or more λ LC gene coding sequences created after V_{λ} to J_{λ} gene segment rearrangement. In one aspect, the partly canine immunoglobulin locus includes a λ V-region promoter to drive expression of one or more κ LC gene coding sequences created after V_{κ} to J_{κ} gene segment rearrangement. In one aspect, the partly canine immunoglobulin locus includes a κ V-region promoter to drive expression of one or more λ LC gene coding sequences created after V_{λ} to J_{λ} gene segment rearrangement. In one aspect, the partly canine immunoglobulin locus includes a κ V-region promoter to drive expression of one or more κ LC gene coding sequences created after V_{κ} to J_{κ} gene segment rearrangement.

In one aspect, the partly canine immunoglobulin locus includes one or more enhancers. In one aspect, the partly canine immunoglobulin locus includes a mouse κ iE_{κ} or 3′Eκ enhancer. In one aspect, the partly canine immunoglobulin locus includes one or more V_{λ} or J_{λ} gene segment coding sequences and a moue κ iE_{κ} or 3′E_{κ} enhancer. In one aspect, the partly canine immunoglobulin locus includes one or more V_{κ} or J_{κ} gene segment coding sequences and a κ iEκ or 3′Eκ enhancer.

### Immunoglobulin Heavy Chain Locus

In one aspect, a transgenic rodent or rodent cell has a genome comprising a recombinantly produced partly canine immunoglobulin heavy chain variable region (V_{H}) locus. In one aspect, the partly canine immunoglobulin variable region locus comprises one or more canine V_{H}, D or J_{H} gene segment coding sequences. In one aspect, the partly canine immunoglobulin heavy chain variable region locus comprises one or more rodent constant domain (C_{H}) genes or coding sequences. In one aspect, an endogenous rodent heavy chain immunoglobulin locus has been inactivated. In one aspect, an endogenous rodent heavy chain immunoglobulin locus has been deleted and replaced with an engineered partly canine heavy chain immunoglobulin locus.

In one aspect, the synthetic H chain DNA segment contains the ADAM6A or ADAM6B gene needed for male fertility, Pax-5-Activated Intergenic Repeats (PAIR) elements involved in IGH locus contraction and CTCF binding sites from the heavy chain intergenic control region 1, involved in regulating normal VDJ rearrangement ((Proudhon, et al., Adv. Immunol., 128:123-182 (2015)), or various combinations thereof. The locations of these endogenous non-coding regulatory and scaffold sequences in the mouse IGH locus are depicted in FIG. 1, which illustrates from left to right: the ˜100 functional heavy chain variable region gene segments (101); PAIR, Pax-5 Activated Intergenic Repeats involved in IGH locus contraction for VDJ recombination (102); ADAM6A or ADAM6B, a disintegrin and metallopeptidase domain 6A gene required for male fertility (103); Pre-D region, a 21609 bp fragment upstream of the most distal D_{H} gene segment, IGHD-5 D (104); Intergenic Control Region 1 (IGCR1) that contains CTCF insulator sites to regulate V_{H} gene segment usage (106); D, diversity gene segments (10-15 depending on the mouse strain) (105); four joining J_{H} gene segments (107); E_{μ}, the intronic enhancer involved in VDJ recombination (108); S_{μ}, the μ switch region for isotype switching (109); eight heavy chain constant region genes: C_{μ}, C_{δ}, C_{γ3}, C_{γ1}, C_{γ2b}, C_{γa/c}, C_{ε}, and C_{α} (110); 3′ Regulatory Region (3′RR) that controls isotype switching and somatic hypermutation (111). FIG. 1A is modified from a figure taken from Proudhon, et al., Adv. Immunol., 128:123-182 (2015).

In one aspect, the engineered partly canine region to be integrated into a mammalian host cell comprises all or a substantial number of the known canine V_{H} gene segments. In some instances, however, it may be desirable to use a subset of such V_{H} gene segments, and in specific instances even as few as one canine V_{H} coding sequence may be introduced into the cell or the animal.

In one aspect, the engineered partly canine immunoglobulin locus variable region comprises a V_{H} locus comprising most or all of the V_{H} gene segment coding sequences from the canine genome. In one aspect, the engineered partly canine immunoglobulin locus variable region comprises a V_{H} locus comprising at least 20, 30 and up to 39 functional canine V_{H} gene segment coding sequences. In one aspect the engineered partly canine immunoglobulin variable region locus comprises a V_{H} locus comprising at least about 50%, 60%, 70%, 80%, 90% and up to 100% of the V_{H} gene segment coding sequences from the canine genome.

In one aspect, the engineered partly canine immunoglobulin locus variable region comprises a V_{H} locus comprising most or all of the V_{H} gene segment coding sequences from the canine genome. In one aspect, the engineered partly canine immunoglobulin locus variable region comprises a V_{H} locus comprising at least 20, 30, 40, 50, 60, 70 and up to 80 canine V_{H} gene segment coding sequences. In this aspect the V_{H} gene segment pseudogenes are reverted to restore their functionality, e.g., by mutating an in-frame stop codon into a functional codon, using methods well known in the art. In one aspect the engineered partly canine immunoglobulin variable region locus comprises a V_{H} locus comprising at least about 50%, 60%, 70%, 80%, 90% and up to 100% of the V_{H} gene segment coding sequences from the canine genome.

In one aspect, the engineered partly canine immunoglobulin locus variable region comprises a V_{H} locus comprising most or all of the D gene segment coding sequences found in the canine genome. In one aspect, the engineered partly canine immunoglobulin locus variable region comprises a V_{H} locus comprising at least 1, 2, 3, 4, 5 and up to 6 canine D gene segment coding sequences. In one aspect the engineered partly canine immunoglobulin variable region locus comprises a V_{H} locus comprising at least about 50%, 60%, 70%, 80%, 90% and up to 100% of the D gene segment coding sequences found in the canine genome.

In one aspect, the engineered partly canine immunoglobulin locus variable region comprises a V_{H} locus comprising most or all of the J_{H} gene segment coding sequences found in the canine genome. In one aspect, the engineered partly canine immunoglobulin locus variable region comprises a V_{H} locus comprising at least 1, 2, 3, 4, 5 and up to 6 canine J_{H} gene segment coding sequences. In one aspect the engineered partly canine immunoglobulin variable region locus comprises a V_{H} locus comprising at least about 50%, 75%, and up to 100% of J_{H} gene segment coding sequences found in the canine genome.

In one aspect, the engineered partly canine immunoglobulin locus variable region comprises a V_{H} locus comprising most or all of the V_{H}, D and J_{H} gene segment coding sequences from the canine genome. In one aspect the engineered partly canine immunoglobulin variable region locus comprises a V_{H} locus comprising at least about 50%, 60%, 70%, 80%, 90% and up to 100% of the V_{H}, D and J_{H} gene segment coding sequences from the canine genome.

In one aspect, a transgenic rodent or rodent cell is provided that includes an engineered partly canine immunoglobulin heavy chain locus comprising canine immunoglobulin heavy chain variable region gene coding sequences and non-coding regulatory or scaffold sequences of the rodent immunoglobulin heavy chain locus. In one aspect, the engineered canine immunoglobulin heavy chain locus comprises canine V_{H}, D or J_{H} gene segment coding sequences. In one aspect, the engineered canine immunoglobulin heavy chain locus comprises canine V_{H}, D or J_{H} gene segment coding sequences embedded in non-coding regulatory or scaffold sequences of a rodent immunoglobulin heavy chain locus.

In one aspect, non-canine mammals and mammalian cells comprising an engineered partly canine immunoglobulin locus that comprises coding sequences of canine V_{H}, canine D, and canine J_{H} genes are provided that further comprises non-coding regulatory and scaffold sequences, including pre-D sequences, based on the endogenous IGH locus of the non-canine mammalian host. In certain aspects, the exogenously introduced, engineered partly canine region can comprise a fully recombined V(D)J exon.

In one aspect, the transgenic non-canine mammal is a rodent, for example, a mouse, comprising an exogenously introduced, engineered partly canine immunoglobulin locus comprising codons for multiple canine V_{H}, canine D, and canine J_{H} genes with intervening sequences, including a pre-D region, based on the intervening (non-coding regulatory or scaffold) sequences in the rodent. In one aspect, the transgenic rodent further comprises partly canine IGL loci comprising coding sequences of canine V_{κ} or V_{λ} genes and J_{κ} or J_{λ} genes, respectively, in conjunction with their intervening (non-coding regulatory or scaffold) sequences corresponding to the immunoglobulin intervening sequences present in the IGL loci of the rodent.

In an exemplary embodiment, as set forth in more detail in the Examples section, the entire endogenous V_{H} immunoglobulin locus of the mouse genome is deleted and subsequently replaced with a partly canine immunoglobulin locus comprising 39 canine V_{H} gene segments containing interspersed non-coding sequences corresponding to the non-coding sequences of the J558 V_{H} locus of the mouse genome. The complete, exogenously introduced, engineered immunoglobulin locus further comprises canine D and J_{H} gene segments, as well as the mouse pre-D region. Thus, the canine V_{H}, D and J_{H} codon sequences are embedded in the rodent intergenic and intronic sequences.

### Preparation of a Partly Canine Immunoglobulin Locus

In one aspect, an endogenous immunoglobulin locus variable region of a non-canine mammal, such as a rodent, for example a rat or mouse, which contains V_{H}, D and J_{H} or V_{L} and J_{L} gene segments, is deleted using site-specific recombinases and replaced with an engineered partly canine immunoglobulin locus. In one aspect, the partly canine immunoglobulin locus is inserted into the genome of the host animal as a single nucleic acid or cassette. Because a cassette that includes the partly canine immunoglobulin locus is used to replace the endogenous immunoglobulin locus variable region, the canine coding sequences can be inserted into the host genome in a single insertion step, thus providing a rapid and straightforward process for obtaining a transgenic animal.

In one aspect, the engineered partly canine immunoglobulin locus variable region is prepared by deleting murine V_{H}, D and J_{H} or V_{L} and J_{L} coding sequences from a mouse immunoglobulin locus variable region and replacing the murine coding sequences with canine coding sequences. In one aspect, the non-coding flanking sequences of the murine immunoglobulin locus, which include regulatory sequences and other elements, are left intact.

In one aspect, the nucleotide sequence for the engineered partly canine immunoglobulin locus is prepared in silico and the locus is synthesized using known techniques for gene synthesis. In one aspect, coding sequences from a canine immunoglobulin variable region locus and sequences of the host animal immunoglobulin locus are identified using a search tool such as BLAST (Basic Local Alignment Search Tool). After obtaining the genomic sequences of the host immunoglobulin locus and the coding sequences of the canine immunoglobulin variable region locus, the host coding sequences can be replaced in silico with the canine coding sequences using known computational approaches to locate and delete the endogenous host animal immunoglobulin coding segments and replace the coding sequences with canine coding sequences, leaving the endogenous regulatory and flanking sequences intact.

### Homologous Recombination

In one aspect, a combination of homologous recombination and site-specific recombination is used to create the cells and animals described herein. In some embodiments, a homology targeting vector is first used to introduce the sequence-specific recombination sites into the mammalian host cell genome at a desired location in the endogenous immunoglobulin loci. In one aspect, in the absence of a recombinase protein, the sequence-specific recombination site inserted into the genome of a mammalian host cell by homologous recombination does not affect expression and amino acid codons of any genes in the mammalian host cell. This approach maintains the proper transcription and translation of the immunoglobulin genes which produce the desired antibody after insertion of recombination sites and, optionally, any additional sequence such as a selectable marker gene. However, in some cases it is possible to insert a recombinase site and other sequences into an immunoglobulin locus sequence such that an amino acid sequence of the antibody molecule is altered by the insertion, but the antibody still retains sufficient functionality for the desired purpose. Examples of such codon-altering homologous recombination may include the introduction of polymorphisms into the endogenous locus and changing the constant region exons so that a different isotype is expressed from the endogenous locus. In one aspect, the immunoglobulin locus includes one or more of such insertions.

In one aspect, the homology targeting vector can be utilized to replace certain sequences within the endogenous genome as well as to insert certain sequence-specific recombination sites and one or more selectable marker genes into the host cell genome. It is understood by those of ordinary skill in the art that a selectable marker gene as used herein can be exploited to weed out individual cells that have not undergone homologous recombination and cells that harbor random integration of the targeting vector.

Exemplary methodologies for homologous recombination are described in U.S. Pat. Nos. 6,689,610; 6,204,061; 5,631,153; 5,627,059; 5,487,992; and 5,464,764, each of which is incorporated by reference in its entirety.

### Site/Sequence-Specific Recombination

Site/sequence-specific recombination differs from general homologous recombination in that short specific DNA sequences, which are required for recognition by a recombinase, are the only sites at which recombination occurs. Depending on the orientations of these sites on a particular DNA strand or chromosome, the specialized recombinases that recognize these specific sequences can catalyze i) DNA excision or ii) DNA inversion or rotation. Site-specific recombination can also occur between two DNA strands if these sites are not present on the same chromosome. A number of bacteriophage- and yeast-derived site-specific recombination systems, each comprising a recombinase and specific cognate sites, have been shown to work in eukaryotic cells and are therefore applicable for use in connection with the methods described herein, and these include the bacteriophage P1 Cre/lox, yeast FLP-FRT system, and the Dre system of the tyrosine family of site-specific recombinases. Such systems and methods of use are described, e.g., in U.S. Pat. Nos. 7,422,889; 7,112,715; 6,956,146; 6,774,279; 5,677,177; 5,885,836; 5,654,182; and 4,959,317, each of which is incorporated herein by reference to teach methods of using such recombinases.

Other systems of the tyrosine family of site-specific recombinases such as bacteriophage lambda integrase, HK2022 integrase, and in addition systems belonging to the separate serine family of recombinases such as bacteriophage phiC31, R4Tp901 integrases are known to work in mammalian cells using their respective recombination sites, and are also applicable for use in the methods described herein.

Since site-specific recombination can occur between two different DNA strands, site-specific recombination occurrence can be utilized as a mechanism to introduce an exogenous locus into a host cell genome by a process called recombinase-mediated cassette exchange (RMCE). The RMCE process can be exploited by the combined usage of wild-type and mutant sequence-specific recombination sites for the same recombinase protein together with negative selection. For example, a chromosomal locus to be targeted may be flanked by a wild-type LoxP site on one end and by a mutant LoxP site on the other. Likewise, an exogenous vector containing a sequence to be inserted into the host cell genome may be similarly flanked by a wild-type LoxP site on one end and by a mutant LoxP site on the other. When this exogenous vector is transfected into the host cell in the presence of Cre recombinase, Cre recombinase will catalyze RMCE between the two DNA strands, rather than the excision reaction on the same DNA strands, because the wild-type LoxP and mutant LoxP sites on each DNA strand are incompatible for recombination with each other. Thus, the LoxP site on one DNA strand will recombine with a LoxP site on the other DNA strand; similarly, the mutated LoxP site on one DNA strand will only recombine with a likewise mutated LoxP site on the other DNA strand.

In one aspect, combined variants of the sequence-specific recombination sites are used that are recognized by the same recombinase for RMCE. Examples of such sequence-specific recombination site variants include those that contain a combination of inverted repeats or those which comprise recombination sites having mutant spacer sequences. For example, two classes of variant recombinase sites are available to engineer stable Cre-loxP integrative recombination. Both exploit sequence mutations in the Cre recognition sequence, either within the 8 bp spacer region or the 13-bp inverted repeats. Spacer mutants such as lox511 (Hoess, et al., Nucleic Acids Res, 14:2287-2300 (1986)), lox5171 and lox2272 (Lee and Saito, Gene, 216:55-65 (1998)), m2, m3, m7, and mu11 (Langer, et al., Nucleic Acids Res, 30:3067-3077 (2002)) recombine readily with themselves but have a markedly reduced rate of recombination with the wild-type site. This class of mutants has been exploited for DNA insertion by RMCE using non-interacting Cre-Lox recombination sites and non-interacting FLP recombination sites (Baer and Bode, Curr Opin Biotechnol, 12:473-480 (2001); Albert, et al., Plant J, 7:649-659 (1995); Seibler and Bode, Biochemistry, 36:1740-1747 (1997); Schlake and Bode, Biochemistry, 33:12746-12751 (1994)).

Inverted repeat mutants represent the second class of variant recombinase sites. For example, LoxP sites can contain altered bases in the left inverted repeat (LE mutant) or the right inverted repeat (RE mutant). An LE mutant, lox71, has 5 bp on the 5′ end of the left inverted repeat that is changed from the wild type sequence to TACCG (Araki, et al, Nucleic Acids Res, 25:868-872 (1997)). Similarly, the RE mutant, lox66, has the five 3′-most bases changed to CGGTA. Inverted repeat mutants are used for integrating plasmid inserts into chromosomal DNA with the LE mutant designated as the “target” chromosomal loxP site into which the “donor” RE mutant recombines. Post-recombination, loxP sites are located in cis, flanking the inserted segment. The mechanism of recombination is such that post-recombination one loxP site is a double mutant (containing both the LE and RE inverted repeat mutations) and the other is wild type (Lee and Sadowski, Prog Nucleic Acid Res Mol Biol, 80:1-42 (2005); Lee and Sadowski, J Mol Biol, 326:397-412 (2003)). The double mutant is sufficiently different from the wild-type site that it is unrecognized by Cre recombinase and the inserted segment is not excised.

In certain aspects, sequence-specific recombination sites can be introduced into introns, as opposed to coding nucleic acid regions or regulatory sequences. This avoids inadvertently disrupting any regulatory sequences or coding regions necessary for proper antibody expression upon insertion of sequence-specific recombination sites into the genome of the animal cell.

Introduction of the sequence-specific recombination sites may be achieved by conventional homologous recombination techniques. Such techniques are described in references such as e.g., Sambrook and Russell (2001) (Molecular cloning: a laboratory manual 3rd ed. (Cold Spring Harbor, N.Y.: Cold Spring Harbor Laboratory Press) and Nagy, A. (2003). (Manipulating the mouse embryo: a laboratory manual, 3rd ed. (Cold Spring Harbor, N.Y.: Cold Spring Harbor Laboratory Press). Renault and Duchateau, Eds. (2013) (Site-directed insertion of transgenes. Topics in Current Genetics 23. Springer). Tsubouchi, H. Ed. (2011) (DNA recombination, Methods and Protocols. Humana Press).

Specific recombination into the genome can be facilitated using vectors designed for positive or negative selection as known in the art. In order to facilitate identification of cells that have undergone the replacement reaction, an appropriate genetic marker system may be employed and cells selected by, for example, use of a selection tissue culture medium. However, in order to ensure that the genome sequence is substantially free of extraneous nucleic acid sequences at or adjacent to the two end points of the replacement interval, desirably the marker system/gene can be removed following selection of the cells containing the replaced nucleic acid.

In one aspect, cells in which the replacement of all or part of the endogenous immunoglobulin locus has taken place are negatively selected against upon exposure to a toxin or drug. For example, cells that retain expression of HSV-TK can be selected against by using nucleoside analogues such as ganciclovir. In another aspect, cells comprising the deletion of the endogenous immunoglobulin locus may be positively selected for by use of a marker gene, which can optionally be removed from the cells following or as a result of the recombination event. A positive selection system that may be used is based on the use of two non-functional portions of a marker gene, such as HPRT, that are brought together through the recombination event. These two portions are brought into functional association upon a successful replacement reaction being carried out and wherein the functionally reconstituted marker gene is flanked on either side by further sequence-specific recombination sites (which are different from the sequence-specific recombination sites used for the replacement reaction), such that the marker gene can be excised from the genome, using an appropriate site-specific recombinase.

The recombinase may be provided as a purified protein, or as a protein expressed from a vector construct transiently transfected into the host cell or stably integrated into the host cell genome. Alternatively, the cell may be used first to generate a transgenic animal, which then may be crossed with an animal that expresses said recombinase.

Because the methods described herein can take advantage of two or more sets of sequence-specific recombination sites within the engineered genome, multiple rounds of RMCE can be exploited to insert the partly canine immunoglobulin variable region genes into a non-canine mammalian host cell genome.

Although not yet routine for the insertion of large DNA segments, CRISPR-Cas technology is another method to introduce the chimeric canine Ig locus.

### Generation of Transgenic Animals

In one aspect, methods for the creation of transgenic animals, for example rodents, such as mice, are provided that comprise the introduced partly canine immunoglobulin locus.

In one aspect, the host cell utilized for replacement of the endogenous immunoglobulin genes is an embryonic stem (ES) cell, which can then be utilized to create a transgenic mammal. In one aspect, the host cell is a cell of an early stage embryo. In one aspect, the host cell is a pronuclear stage embryo or zygote. Thus, in accordance with one aspect, the methods described herein further comprise: isolating an embryonic stem cell or a cell of an early stage embryo such as a pronuclear stage embryo or zygote, which comprises the introduced partly canine immunoglobulin locus and using said ES cell to generate a transgenic animal that contains the replaced partly canine immunoglobulin locus.

### Methods of Use

In one aspect, a method of producing antibodies comprising canine variable regions is provided. In one aspect, the method includes providing a transgenic rodent or rodent cell described herein and isolating antibodies comprising canine variable regions expressed by the transgenic rodent. In one aspect, a method of producing monoclonal antibodies comprising canine variable regions is provided. In one aspect, the method includes providing B-cells from a transgenic rodent or cell described herein, immortalizing the B-cells; and isolating antibodies comprising canine variable domains expressed by the immortalized B-cells.

In one aspect, the antibodies expressed by the transgenic rodent or rodent cell comprise canine HC variable domains. In one aspect, the antibodies expressed by the transgenic rodent or rodent cell comprise mouse HC constant domains. These can be of any isotype, IgM, IgD, IgG1, IgG2a/c, IgG2b, IgG3, IgE or IgA.

In one aspect, the antibodies expressed by the transgenic rodent or rodent cell comprise canine HC variable domains and mouse HC constant domains. In one aspect, the antibodies expressed by the transgenic rodent or rodent cell comprise canine LC variable domains and mouse LC constant domains. In one aspect, the antibodies expressed by the transgenic rodent or rodent cell comprise canine HC variable domains and canine LC variable domains and mouse HC constant domains and mouse LC constant domains.

In one aspect, the antibodies expressed by the transgenic rodent or rodent cell comprise canine λ LC variable domains. In one aspect, the antibodies expressed by the transgenic rodent or rodent cell comprise mouse λ constant domains. In one aspect, the antibodies expressed by the transgenic rodent or rodent cell comprise canine λ LC variable domains and mouse λ constant domains. In one aspect, the antibodies expressed by the transgenic rodent or rodent cell comprise canine κ LC variable domains. In one aspect, the antibodies expressed by the transgenic rodent or rodent cell comprise mouse κ constant domains. In one aspect, the antibodies expressed by the transgenic rodent or rodent cell comprise canine κ LC variable domains and mouse κ constant domains.

In one aspect, a method of producing antibodies or antigen binding fragments comprising canine variable regions is provided. In one aspect, the method includes providing a transgenic rodent or cell described herein and isolating antibodies comprising canine variable regions expressed by the transgenic rodent or rodent cell. In one aspect, the variable regions of the antibody expressed by the transgenic rodent or rodent cell are sequenced. Antibodies comprising canine variable regions obtained from the antibodies expressed by the transgenic rodent or rodent cell can be recombinantly produced using known methods.

In one aspect, a method of producing an immunoglobulin specific to an antigen of interest is provided. In one aspect, the method includes immunizing a transgenic rodent as described herein with the antigen and isolating immunoglobulin specific to the antigen expressed by the transgenic rodent or rodent cell. In one aspect, the variable domains of the antibody expressed by the rodent or rodent cell are sequenced and antibodies comprising canine variable regions that specifically bind the antigen of interest are recombinantly produced using known methods. In one aspect, the recombinantly produced antibody or antigen binding fragment comprises canine HC and LC, κ or λ, constant domains.

### INCORPORATION BY REFERENCE

All references cited herein, including patents, patent applications, papers, text books and the like, and the references cited therein, to the extent that they are not already, are hereby incorporated herein by reference in their entirety for all purposes.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations or modifications may be made to the invention as shown in the specific embodiments without departing from the spirit or scope of the invention as broadly described. The present embodiments are, therefore, to be considered in all respects as illustrative and not restrictive.

Efforts have been made to ensure accuracy with respect to terms and numbers used (e.g., vectors, amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

The examples illustrate targeting by both a 5′ vector and a 3′ vector that flank a site of recombination and introduction of synthetic DNA. It will be apparent to one skilled in the art upon reading the specification that the 5′ vector targeting can take place first followed by the 3′, or the 3′ vector targeting can take place first followed by the 5′ vector. In some circumstances, targeting can be carried out simultaneously with dual detection mechanisms.

### Example 1: Introduction of an Engineered Partly Canine Immunoglobulin Variable Region Gene Locus into the Immunoglobulin H Chain Variable Region Gene Locus of a Non-Canine Mammalian Host Cell Genome

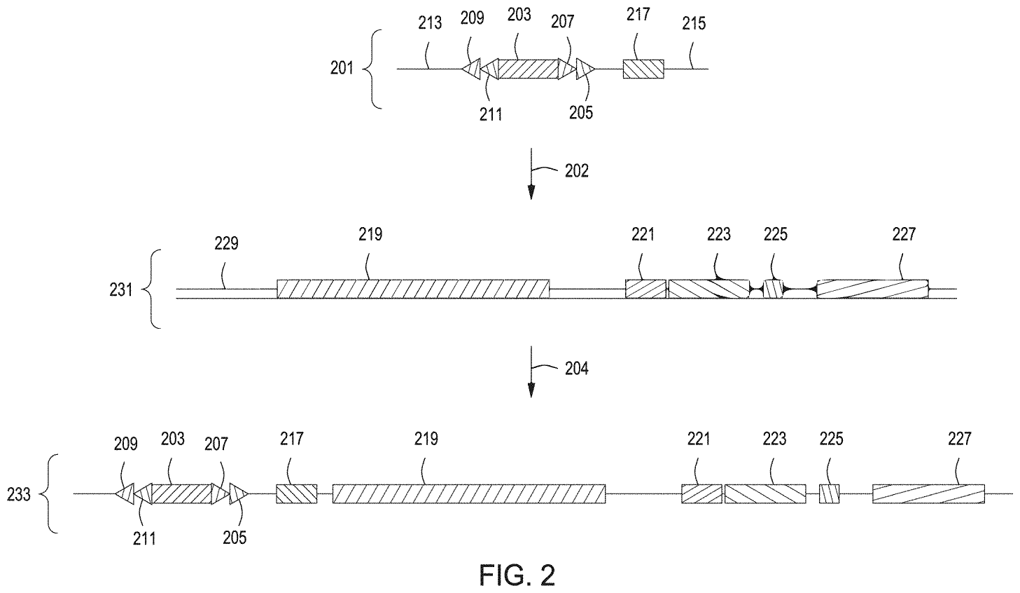

An exemplary method illustrating the introduction of an engineered partly canine immunoglobulin locus into the genomic locus of a non-mammalian ES cell is illustrated in more detail in FIGS. 2-6. In FIG. 2, a homology targeting vector (201) is provided comprising a puromycin phosphotransferase-thymidine kinase fusion protein (puro-TK) (203) flanked by two different recombinase recognition sites (e.g., FRT (207) and loxP (205) for Flp and Cre, respectively) and two different mutant sites (e.g., modified mutant FRT (209) and mutant loxP (211)) that lack the ability to recombine with their respective wild-type counterparts/sites (i.e., wild-type FRT (207) and wild-type loxP (205)). The targeting vector comprises a diphtheria toxin receptor (DTR) cDNA (217) for use in negative selection of cells containing the introduced construct in future steps. The targeting vector also optionally comprises a visual marker such as a green fluorescent protein (GFP) (not shown). The regions 213 and 215 are homologous to the 5′ and 3′ portions, respectively, of a contiguous region (229) in the endogenous non-canine locus that is 5′ of the genomic region comprising the endogenous non-canine V_{H} gene segments (219). The homology targeting vector (201) is introduced (202) into the ES cell, which has an immunoglobulin locus (231) comprising endogenous V_{H} gene segments (219), the pre-D region (221), the D gene segments (223), J_{H} gene segments (225), and the immunoglobulin constant gene region genes (227). The site-specific recombination sequences and the DTR cDNA from the homology targeting vector (201) are integrated (204) into the non-canine genome at a site 5′ of the endogenous mouse V_{H} gene locus, resulting in the genomic structure illustrated at 233. The ES cells that do not have the exogenous vector (201) integrated into their genome can be selected against (killed) by including puromycin in the culture medium; only the ES cells that have stably integrated the exogenous vector (201) into their genome and constitutively express the puro-TK gene are resistant to puromycin.

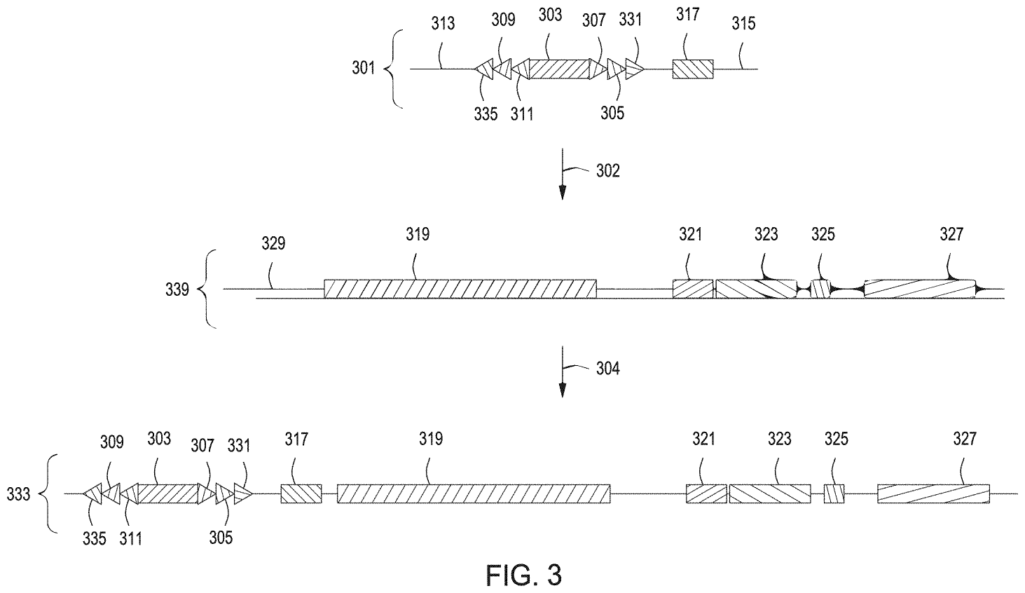

FIG. 3 illustrates effectively the same approach as FIG. 2, except that an additional set of sequence-specific recombination sites is added, e.g., a Rox site (331) and a modified Rox site (335) for use with the Dre recombinase. In FIG. 3, a homology targeting vector (301) is provided comprising a puro-TK fusion protein (303) flanked by wild type recombinase recognition sites for FRT (307), loxP (305), and Rox (331) and mutant sites for FRT (309) loxP (311) and Rox (335) recombinases that lack the ability to recombine with the wild-type sites 307, 305 and 331, respectively. The targeting vector also comprises a diphtheria toxin receptor (DTR) cDNA (317). The regions 313 and 315 are homologous to the 5′ and 3′ portions, respectively, of a contiguous region (329) in the endogenous non-canine locus that is 5′ of the genomic region comprising the endogenous mouse V_{H} gene segments (319). The homology targeting is introduced (302) into the mouse immunoglobulin locus (339), which comprises the endogenous V_{H} gene segments (319), the pre-D region (321), the D gene segments (323), J_{H} (325) gene segments, and the constant region genes (327) of the IGH locus. The site-specific recombination sequences and the DTR cDNA (317) in the homology targeting vector (301) are integrated (304) into the mouse genome at a site 5′ of the endogenous mouse V_{H} gene locus, resulting in the genomic structure illustrated at 333.

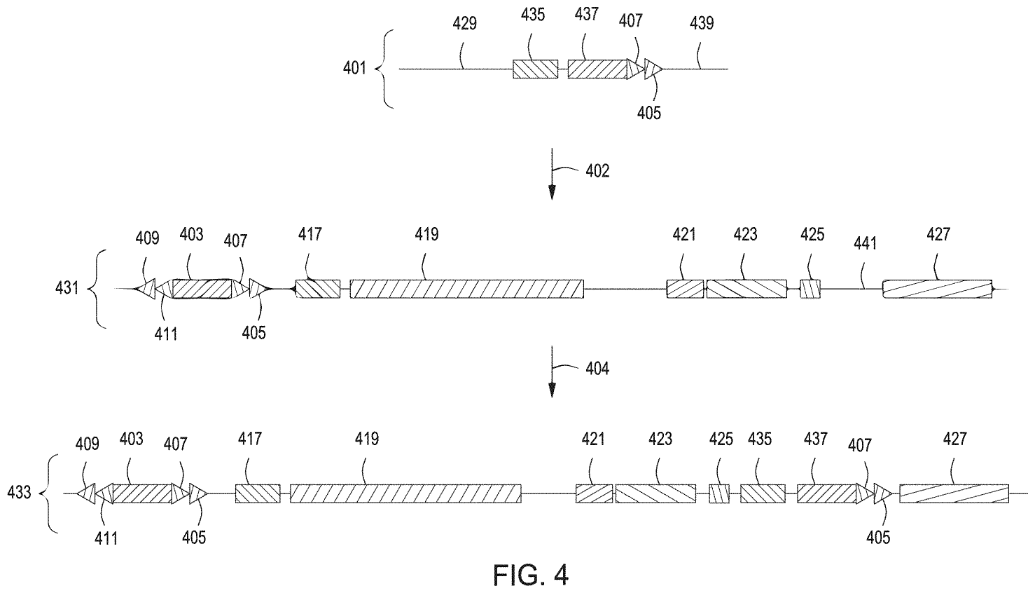

As illustrated in FIG. 4, a second homology targeting vector (401) is provided comprising an optional hypoxanthine-guanine phosphoribosyltransferase (HPRT) gene (435) that can be used for positive selection in HPRT-deficient ES cells; a neomycin resistance gene (437); recombinase recognition sites FRT (407) and loxP (405), for Flp and Cre, respectively, which have the ability to recombine with FRT (407) and loxP (405) sites previously integrated into the mouse genome from the first homology targeting vector. The previous homology targeting vector also includes mutant FRT site (409), mutant loxP site (411), a puro-TK fusion protein (403), and a DTR cDNA at a site 5′ of the endogenous mouse V_{H} gene locus (419). The regions 429 and 439 are homologous to the 5′ and 3′ portions, respectively, of a contiguous region (441) in the endogenous mouse non-canine locus that is downstream of the endogenous J_{H} gene segments (425) and upstream of the constant region genes (427). The homology targeting vector is introduced (402) into the modified mouse immunoglobulin locus (431), which comprises the endogenous V_{H} gene segments (419), the pre-D region (421), the D gene segments (423) the J_{H} gene segments (425), and the constant region genes (427). The site-specific recombination sequences (407, 405), the HPRT gene (435) and a neomycin resistance gene (437) of the homology targeting vector are integrated (404) into the mouse genome upstream of the endogenous mouse constant region genes (427), resulting in the genomic structure illustrated at 433.

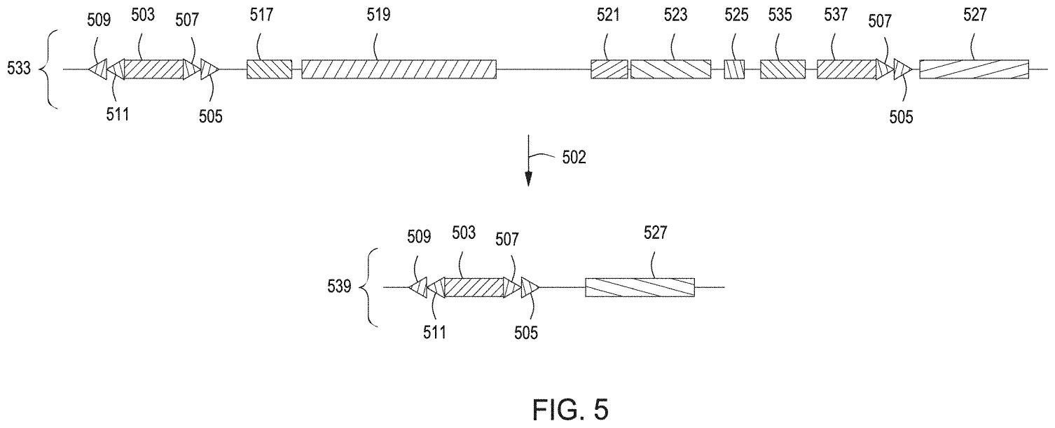

Once the recombination sites are integrated into the mammalian host cell genome, the endogenous region of the immunoglobulin domain is then subjected to recombination by introducing one of the recombinases corresponding to the sequence-specific recombination sites integrated into the genome, e.g., either Flp or Cre. Illustrated in FIG. 5 is a modified IGH locus of the mammalian host cell genome comprising two integrated DNA fragments. One fragment comprising mutant FRT site (509), mutant LoxP site (511), puro-TK gene (503), wild-type FRT site (507), and wild-type LoxP site (505), and DTR cDNA (517) is integrated upstream of the V_{H} gene locus (519). The other DNA fragment comprising HPRT gene (535), neomycin resistance gene (537), wild-type FRT site (507), and wild-type LoxP site (505) is integrated downstream of the pre-D (521), D (523) and J_{H} (525) gene loci, but upstream of the constant region genes (527). In the presence of Flp or Cre (502), all the intervening sequences between the wild-type FRT or wild-type LoxP sites including the DTR gene (517), the endogenous IGH variable region gene loci (519, 521, 525), and the HPRT (535) and neomycin resistance (537) genes are deleted, resulting in a genomic structure illustrated at 539. The procedure depends on the second targeting having occurred on the same chromosome rather than on its homolog (i.e., in cis rather than in trans). If the targeting occurs in cis as intended, the cells are not sensitive to negative selection after Cre- or Flp-mediated recombination by diphtheria toxin introduced into the media, because the DTR gene which causes sensitivity to diphtheria toxin in rodents should be absent (deleted) from the host cell genome. Likewise, ES cells that harbor random integration of the first or second targeting vector(s) are rendered sensitive to diphtheria toxin by presence of the undeleted DTR gene.

ES cells that are insensitive to diphtheria toxin are then screened for the deletion of the endogenous variable region gene loci. The primary screening method for the deleted endogenous immunoglobulin locus can be carried out by Southern blotting, or by polymerase chain reaction (PCR) followed by confirmation with a secondary screening technique such as Southern blotting.

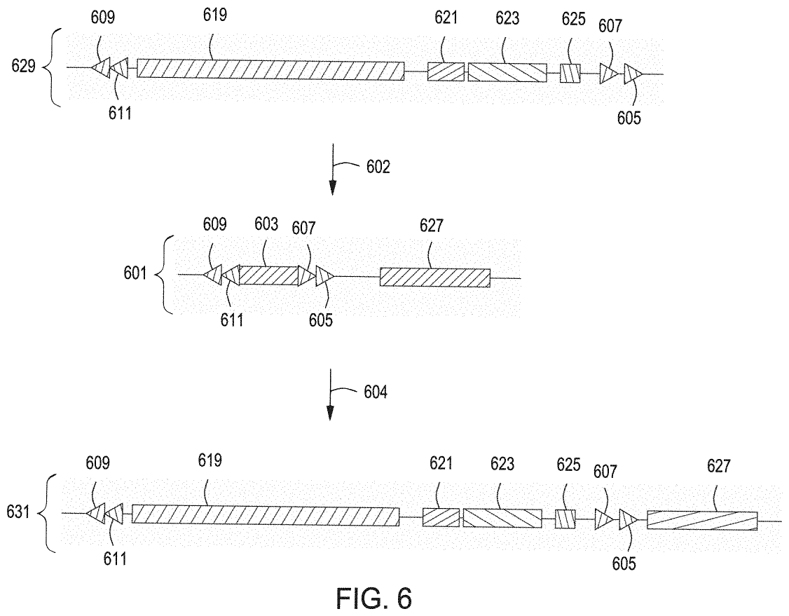

FIG. 6 illustrates introduction of the engineered partly canine sequence into a non-canine genome previously modified to delete part of the endogenous IGH locus (V_{H}, D and JO that encodes the heavy chain variable region domains as well as all the intervening sequences between the V_{H} and J_{H} gene locus. A site-specific targeting vector (629) comprising partly canine V_{H} gene locus (619), endogenous non-canine pre-D gene region (621), partly canine D gene locus (623), partly canine J_{H} gene locus (625), as well as flanking mutant FRT (609), mutant LoxP (611), wild-type FRT (607), and wild-type LoxP (605) sites is introduced (602) into the host cell. Specifically, the partly canine V_{H} locus (619) comprises 39 functional canine V_{H} coding sequences in conjunction with the intervening sequences based on the endogenous non-canine genome sequences; the pre-D region (621) comprises a 21.6 kb mouse sequence with significant homology to the corresponding region of the endogenous canine IGH locus; the D gene locus (623) comprises codons of 6 D gene segments embedded in the intervening sequences surrounding the endogenous non-canine D gene segments; and the J_{H} gene locus (625) comprises codons of 6 canine J_{H} gene segments embedded in the intervening sequences based on the endogenous non-canine genome. The IGH locus (601) of the host cell genome has been previously modified to delete all the V_{H}, D, and J_{H} gene segments including the intervening sequences as described in FIG. 5. As a consequence of this modification, the endogenous non-canine host cell IGH locus (601) is left with a puro-TK fusion gene (603), which is flanked by a mutant FRT site (609) and a mutant LoxP site (611) upstream as well as a wild-type FRT (607) and a wild-type LoxP (605) downstream. Upon introduction of the appropriate recombinase (604), the partly canine immunoglobulin locus is integrated into the genome upstream of the endogenous non-canine constant region genes (627), resulting in the genomic structure illustrated at 631.

The sequences of the canine V_{H}, D and J_{H} gene segment coding regions are in Table 1.

Primary screening procedure for the introduction of the partly canine immunoglobulin locus can be carried out by Southern blotting, or by PCR followed by confirmation with a secondary screening method such as Southern blotting. The screening methods are designed to detect the presence of the inserted V_{H}, D and J_{H} gene loci, as well as all the intervening sequences.

### Example 2: Introduction of an Engineered Partly Canine Immunoglobulin Variable Region Gene Locus Comprising Additional Non-Coding Regulatory or Scaffold Sequences into the Immunoglobulin H Chain Variable Region Gene Locus of a Non-Canine Mammalian Host Cell Genome

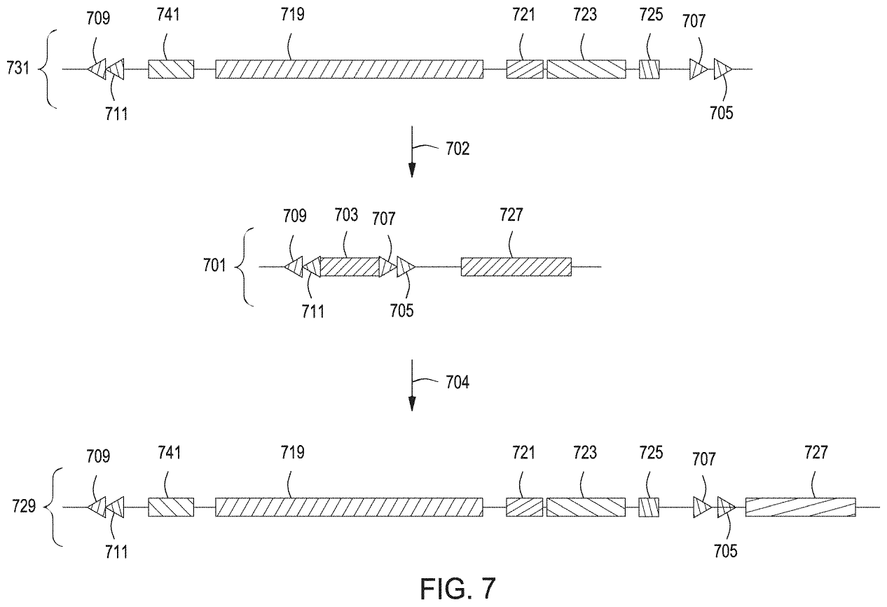

In certain aspects, the partly canine immunoglobulin locus comprises the elements as described in Example 1, but with additional non-coding regulatory or scaffold sequences e.g., sequences strategically added to introduce additional regulatory sequences, to ensure the desired spacing within the introduced immunoglobulin locus, to ensure that certain coding sequences are in adequate juxtaposition with other sequences adjacent to the replaced immunoglobulin locus, and the like. FIG. 7 illustrates the introduction of a second exemplary engineered partly canine sequence into the modified non-canine genome as produced in FIGS. 2-5 and described in Example 1 above.

FIG. 7 illustrates introduction of the engineered partly canine sequence into the mouse genome previously modified to delete part of the endogenous non-canine IGH locus (V_{H}, D and J_{H}) that encodes the heavy chain variable region domains as well as all the intervening sequences between the endogenous V_{H} and J_{H} gene loci. A site-specific targeting vector (731) comprising an engineered partly canine immunoglobulin locus to be inserted into the non-canine host genome is introduced (702) into the genomic region (701). The site-specific targeting vector (731) comprising a partly canine V_{H} gene locus (719), mouse pre-D region (721), partly canine D gene locus (723), partly canine J_{H} gene locus (725), PAIR elements (741), as well as flanking mutant FRT (709), mutant LoxP (711) wild-type FRT (707) and wild-type LoxP (705) sites is introduced (702) into the host cell. Specifically, the engineered partly canine V_{H} gene locus (719) comprises 80 canine V_{H} gene segment coding regions in conjunction with intervening sequences based on the endogenous non-canine genome sequences; the pre-D region (721) comprises a 21.6 kb non-canine sequence present upstream of the endogenous non-canine genome; the D region (723) comprises codons of 6 canine D gene segments embedded in the intervening sequences surrounding the endogenous non-canine D gene segments; and the J_{H} gene locus (725) comprises codons of 6 canine J_{H} gene segments embedded in the intervening sequences based on the endogenous non-canine genome sequences. The IGH locus (701) of the host cell genome has been previously modified to delete all the V_{H}, D and J_{H} gene segments including the intervening sequences as described in relation to FIG. 5. As a consequence of this modification, the endogenous non-canine IGH locus (701) is left with a puro-TK fusion gene (703), which is flanked by a mutant FRT site (709) and a mutant LoxP site (711) upstream as well as a wild-type FRT (707) and a wild-type LoxP (705) downstream. Upon introduction of the appropriate recombinase (704), the engineered partly canine immunoglobulin locus is integrated into the genome upstream of the endogenous mouse constant region genes (727), resulting in the genomic structure illustrated at 729.

The primary screening procedure for the introduction of the engineered partly canine immunoglobulin region can be carried out by Southern blotting, or by PCR with confirmation by a secondary screening method such as Southern blotting. The screening methods are designed to detect the presence of the inserted PAIR elements, the V_{H}, D and J_{H} gene loci, as well as all the intervening sequences.

### Example 3: Introduction of an Engineered Partly Canine Immunoglobulin Locus into the Immunoglobulin Heavy Chain Gene Locus of a Mouse Genome

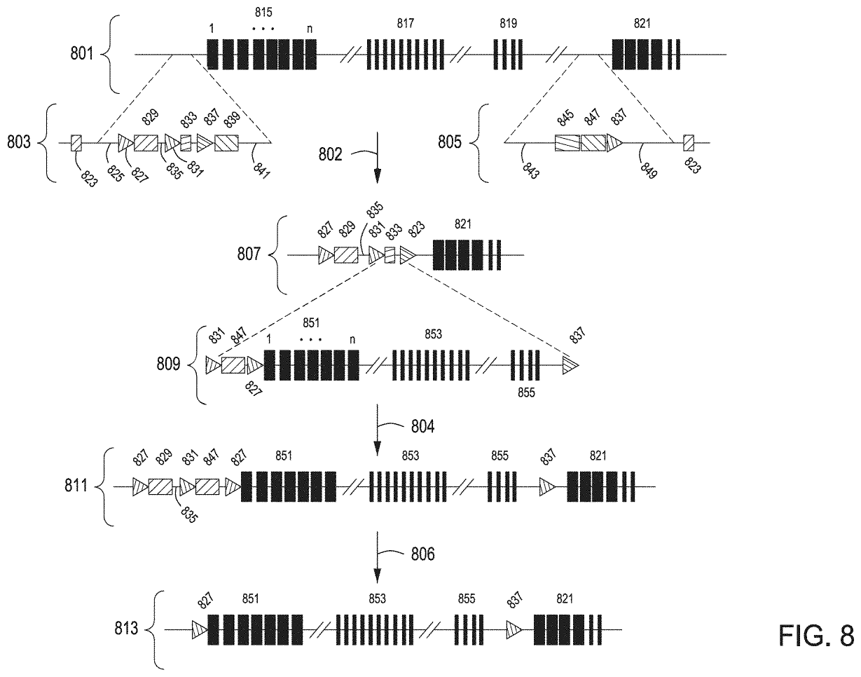

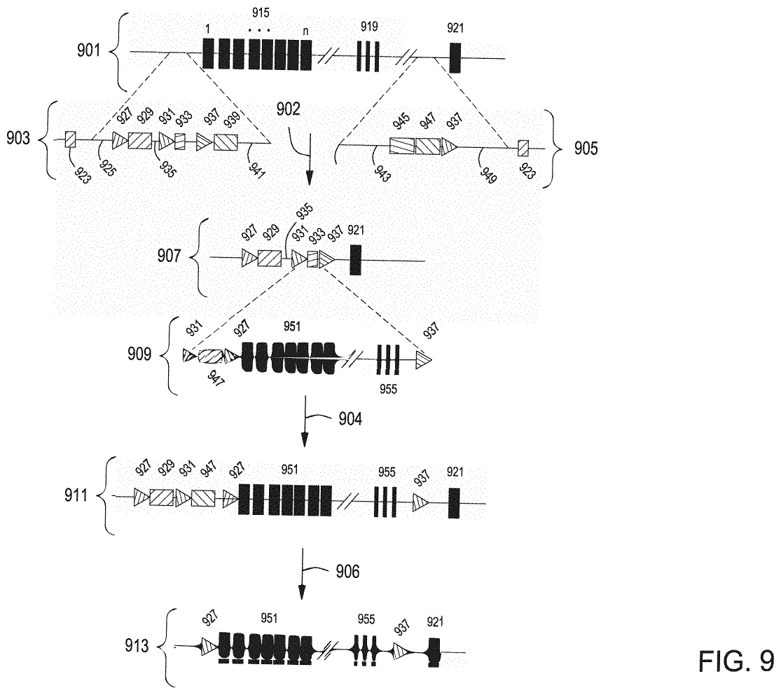

A method for replacing a portion of a mouse genome with an engineered partly canine immunoglobulin locus is illustrated in FIG. 8. This method uses introduction of a first site-specific recombinase recognition sequence into the mouse genome followed by the introduction of a second site-specific recombinase recognition sequence into the mouse genome. The two sites flank the entire clusters of endogenous mouse V_{H}, D and J_{H} region gene segments. The flanked region is deleted using the relevant site-specific recombinase, as described herein.

The targeting vectors (803, 805) employed for introducing the site-specific recombinase sequences on either side of the V_{H} (815), D (817) and J_{H} (819) gene segment clusters and upstream of the constant region genes (821) in the wild-type mouse immunoglobulin locus (801) include an additional site-specific recombination sequence that has been modified so that it is still recognized efficiently by the recombinase, but does not recombine with unmodified sites. This mutant modified site (e.g., lox5171) is positioned in the targeting vector such that after deletion of the endogenous V_{H}, D_{H} and J_{H} gene segments (802) it can be used for a second site-specific recombination event in which a non-native piece of DNA is moved into the modified IGH locus by RMCE. In this example, the non-native DNA is a synthetic nucleic acid comprising both canine and non-canine sequences (809).

Two gene targeting vectors are constructed to accomplish the process just outlined. One of the vectors (803) comprises mouse genomic DNA taken from the 5′ end of the IGH locus, upstream of the most distal V_{H} gene segment. The other vector (805) comprises mouse genomic DNA taken from within the locus downstream of the J_{H} gene segments.

The key features of the 5′ vector (803) in order from 5′ to 3′ are as follows: a gene encoding the diphtheria toxin A (DTA) subunit under transcriptional control of a modified herpes simplex virus type I thymidine kinase gene promoter coupled to two mutant transcriptional enhancers from the polyoma virus (823); 4.5 Kb of mouse genomic DNA mapping upstream of the most distal V_{H} gene segment in the IGH locus (825); a FRT recognition sequence for the Flp recombinase (827); a piece of genomic DNA containing the mouse Polr2a gene promoter (829); a translation initiation sequence (methionine codon embedded in a “Kozak” consensus sequence, 835)); a mutated loxP recognition sequence (lox5171) for the Cre recombinase (831); a transcription termination/polyadenylation sequence (pA. 833); a loxP recognition sequence for the Cre recombinase (837); a gene encoding a fusion protein with a protein conferring resistance to puromycin fused to a truncated form of the thymidine kinase (pu-TK) under transcriptional control of the promoter from the mouse phosphoglycerate kinase 1 gene (839); and 3 Kb of mouse genomic DNA (841) mapping close to the 4.5 Kb mouse genomic DNA sequence present near the 5′ end of the vector and arranged in the native relative orientation.

The key features of the 3′ vector (805) in order from 5′ to 3′ are as follows; 3.7 Kb of mouse genomic DNA mapping within the intron between the J_{H} and C_{H} gene loci (843); an HPRT gene under transcriptional control of the mouse Polr2a gene promoter (845); a neomycin resistance gene under the control of the mouse phosphoglycerate kinase 1 gene promoter (847); a loxP recognition sequence for the Cre recombinase (837); 2.1 Kb of mouse genomic DNA (849) that maps immediately downstream of the 3.7 Kb mouse genomic DNA fragment present near the 5′ end of the vector and arranged in the native relative orientation; and a gene encoding the DTA subunit under transcriptional control of a modified herpes simplex virus type I thymidine kinase gene promoter coupled to two mutant transcriptional enhancers from the polyoma virus (823).

Mouse embryonic stem (ES) cells (derived from C57B1/6NTac mice) are transfected by electroporation with the 3′ vector (805) according to widely used procedures. Prior to electroporation, the vector DNA is linearized with a rare-cutting restriction enzyme that cuts only in the prokaryotic plasmid sequence or the polylinker associated with it. The transfected cells are plated and after ˜24 hours they are placed under positive selection for cells that have integrated the 3′ vector into their DNA by using the neomycin analogue drug G418. There is also negative selection for cells that have integrated the vector into their DNA but not by homologous recombination. Non-homologous recombination results in retention of the DTA gene (823), which kills the cells when the gene is expressed, whereas the DTA gene is deleted by homologous recombination since it lies outside of the region of vector homology with the mouse IGH locus. Colonies of drug-resistant ES cells are physically extracted from their plates after they became visible to the naked eye about a week later. These picked colonies are disaggregated, re-plated in micro-well plates, and cultured for several days. Thereafter, each of the clones of cells is divided such that some of the cells can be frozen as an archive, and the rest used for isolation of DNA for analytical purposes.

DNA from the ES cell clones is screened by PCR using a widely practiced gene-targeting assay design. For this assay, one of the PCR oligonucleotide primer sequences maps outside the region of identity shared between the 3′ vector (805) and the genomic DNA, while the other maps within the novel DNA between the two arms of genomic identity in the vector, i.e., in the HPRT (845) or neomycin resistance (847) genes. According to the standard design, these assays detect pieces of DNA that would only be present in clones of ES cells derived from transfected cells that undergo fully legitimate homologous recombination between the 3′ targeting vector and the endogenous mouse IGH locus. Two separate transfections are performed with the 3′ vector (805). PCR-positive clones from the two transfections are selected for expansion followed by further analysis using Southern blot assays.

The Southern blot assays are performed according to widely used procedures using three probes and genomic DNA digested with multiple restriction enzymes chosen so that the combination of probes and digests allow the structure of the targeted locus in the clones to be identified as properly modified by homologous recombination. One of the probes maps to DNA sequence flanking the 5′ side of the region of identity shared between the 3′ targeting vector and the genomic DNA; a second probe maps outside the region of identity but on the 3′ side; and the third probe maps within the novel DNA between the two arms of genomic identity in the vector, i.e., in the HPRT (845) or neomycin resistance (847) genes. The Southern blot identifies the presence of the expected restriction enzyme-generated fragment of DNA corresponding to the correctly mutated, i.e., by homologous recombination with the 3′ IGH targeting vector, part of the IGH locus as detected by one of the external probes and by the neomycin or HPRT probe. The external probe detects the mutant fragment and also a wild-type fragment from the non-mutant copy of the immunoglobulin IGH locus on the homologous chromosome.

Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. ES cell clones that are judged to have the expected correct genomic structure based on the Southern blot data—and that also do not have detectable chromosomal aberrations based on the karyotype analysis—are selected for further use.

Acceptable clones are then modified with the 5′ vector (803) using procedures and screening assays that are similar in design to those used with the 3′ vector (805) except that puromycin selection is used instead of G418/neomycin for selection. The PCR assays, probes and digests are also tailored to match the genomic region being modified by the 5′ vector (805).

Clones of ES cells that have been mutated in the expected fashion by both the 3′ and the 5′ vectors, i.e., doubly targeted cells carrying both engineered mutations, are isolated following vector targeting and analysis. The clones must have undergone gene targeting on the same chromosome, as opposed to homologous chromosomes (i.e., the engineered mutations created by the targeting vectors must be in cis on the same DNA strand rather than in trans on separate homologous DNA strands). Clones with the cis arrangement are distinguished from those with the trans arrangement by analytical procedures such as fluorescence in situ hybridization of metaphase spreads using probes that hybridize to the novel DNA present in the two gene targeting vectors (803 and 805) between their arms of genomic identity. The two types of clones can also be distinguished from one another by transfecting them with a vector expressing the Cre recombinase, which deletes the pu-TK (839), HPRT (845) and neomycin resistance (847) genes if the targeting vectors have been integrated in cis, and then comparing the number of colonies that survive ganciclovir selection against the thymidine kinase gene introduced by the 5′ vector (803) and by analyzing the drug resistance phenotype of the surviving clones by a “sibling selection” screening procedure in which some of the cells from the clone are tested for resistance to puromycin or G418/neomycin. Cells with the cis arrangement of mutations are expected to yield approximately 10³ more ganciclovir-resistant clones than cells with the trans arrangement. The majority of the resulting cis-derived ganciclovir-resistant clones are also sensitive to both puromycin and G418/neomycin, in contrast to the trans-derived ganciclovir-resistant clones, which should retain resistance to both drugs. Doubly targeted clones of cells with the cis-arrangement of engineered mutations in the heavy chain locus are selected for further use.

The doubly targeted clones of cells are transiently transfected with a vector expressing the Cre recombinase and the transfected cells subsequently are placed under ganciclovir selection, as in the analytical experiment summarized above. Ganciclovir-resistant clones of cells are isolated and analyzed by PCR and Southern blot for the presence of the expected deletion between the two engineered mutations created by the 5′ (803) and the 3′ (805) targeting vectors. In these clones, the Cre recombinase causes a recombination (802) to occur between the loxP sites (837) introduced into the heavy chain locus by the two vectors to create the genomic DNA configuration shown at 807. Because the loxP sites are arranged in the same relative orientations in the two vectors, recombination results in excision of a circle of DNA comprising the entire genomic interval between the two loxP sites. The circle does not contain an origin of replication and thus is not replicated during mitosis and therefore is lost from the cells as they undergo proliferation. The resulting clones carry a deletion of the DNA that was originally between the two loxP sites. Clones that have the expected deletion are selected for further use.

ES cell clones carrying the deletion of sequence in one of the two homologous copies of their immunoglobulin heavy chain locus are retransfected (804) with a Cre recombinase expression vector together with a piece of DNA (809) comprising a partly canine immunoglobulin heavy chain locus containing canine V_{H}, D and J_{H} region gene coding region sequences flanked by mouse regulatory and flanking sequences. The key features of this piece of synthetic DNA (809) are the following: a lox5171 site (831); a neomycin resistance gene open reading frame (847) lacking the initiator methionine codon, but in-frame and contiguous with an uninterrupted open reading frame in the lox5171 site a FRT site (827); an array of 39 functional canine V_{H} heavy chain variable region genes (851), each with canine coding sequences embedded in mouse noncoding sequences; optionally a 21.6 kb pre-D region from the mouse heavy chain locus (not shown); a 58 Kb piece of DNA containing the 6 canine D_{H} gene segments (853) and 6 canine J_{H} gene segments (855) where the canine V_{H}, D and J_{H} coding sequences are embedded in mouse noncoding sequences; a loxP site (837) in opposite relative orientation to the lox5171 site (831).

The transfected clones are placed under G418 selection, which enriches for clones of cells that have undergone RMCE in which the engineered partly canine donor immunoglobulin locus (809) is integrated in its entirety into the deleted endogenous immunoglobulin heavy chain locus between the lox5171 (831) and loxP (837) sites to create the DNA region illustrated at 811. Only cells that have properly undergone RMCE have the capability to express the neomycin resistance gene (847) because the promoter (829) as well as the initiator methionine codon (835) required for its expression are not present in the vector (809) but are already pre-existing in the host cell IGH locus (807). The remaining elements from the 5′ vector (803) are removed via Flp-mediated recombination (806) in vitro or in vivo, resulting in the final canine-based locus as shown at 813.

G418-resistant ES cell clones are analyzed by PCR and Southern blot to determine if they have undergone the expected RMCE process without unwanted rearrangements or deletions. Clones that have the expected genomic structure are selected for further use.

ES cell clones carrying the partly canine immunoglobulin heavy chain DNA (813) in the mouse heavy chain locus are microinjected into mouse blastocysts from strain DBA/2 to create partially ES cell-derived chimeric mice according to standard procedures. Male chimeric mice with the highest levels of ES cell-derived contribution to their coats are selected for mating to female mice. The female mice of choice here are of C57B1/6NTac strain, and also carry a transgene encoding the Flp recombinase that is expressed in their germline. Offspring from these matings are analyzed for the presence of the partly canine immunoglobulin heavy chain locus, and for loss of the FRT-flanked neomycin resistance gene that was created in the RMCE step. Mice that carry the partly canine locus are used to establish a colony of mice.

### Example 4: Introduction of an Engineered Partly Canine Immunoglobulin Locus into the Immunoglobulin κ Chain Gene Locus of a Mouse Genome

Another method for replacing a portion of a mouse genome with partly canine immunoglobulin locus is illustrated in FIG. 9. This method includes introducing a first site-specific recombinase recognition sequence into the mouse genome, which may be introduced either 5′ or 3′ of the cluster of endogenous V_{κ} (915) and J_{κ} (919) region gene segments of the mouse genome, followed by the introduction of a second site-specific recombinase recognition sequence into the mouse genome, which in combination with the first sequence-specific recombination site flanks the entire locus comprising clusters of V_{κ} and J_{κ} gene segments upstream of the constant region gene (921). The flanked region is deleted and then replaced with a partly canine immunoglobulin locus using the relevant site-specific recombinase, as described herein.

The targeting vectors employed for introducing the site-specific recombination sequences on either side of the V_{κ} (915) and J_{κ} (919) gene segments also include an additional site-specific recombination sequence that has been modified so that it is still recognized efficiently by the recombinase, but does not recombine with unmodified sites. This site is positioned in the targeting vector such that after deletion of the V_{κ} and J_{κ} gene segment clusters it can be used for a second site specific recombination event in which a non-native piece of DNA is moved into the modified V_{κ} locus via RMCE. In this example, the non-native DNA is a synthetic nucleic acid comprising canine V_{κ} and J_{κ} gene segment coding sequences embedded in mouse regulatory and flanking sequences.

Two gene targeting vectors are constructed to accomplish the process just outlined. One of the vectors (903) comprises mouse genomic DNA taken from the 5′ end of the locus, upstream of the most distal V_{κ} gene segment. The other vector (905) comprises mouse genomic DNA taken from within the locus downstream (3′) of the J_{κ} gene segments (919) and upstream of the constant region genes (921).

The key features of the 5′ vector (903) are as follows: a gene encoding the diphtheria toxin A (DTA) subunit under transcriptional control of a modified herpes simplex virus type I thymidine kinase gene promoter coupled to two mutant transcriptional enhancers from the polyoma virus (923); 6 Kb of mouse genomic DNA (925) mapping upstream of the most distal variable region gene in the κ chain locus; a FRT recognition sequence for the Flp recombinase (927); a piece of genomic DNA containing the mouse Polr2a gene promoter (929); a translation initiation sequence (935, methionine codon embedded in a “Kozak” consensus sequence); a mutated loxP recognition sequence (lox5171) for the Cre recombinase (931); a transcription termination/polyadenylation sequence (933); a loxP recognition sequence for the Cre recombinase (937); a gene encoding a fusion protein with a protein conferring resistance to puromycin fused to a truncated form of the thymidine kinase (pu-TK) under transcriptional control of the promoter from the mouse phosphoglycerate kinase 1 gene (939); 2.5 Kb of mouse genomic DNA (941) mapping close to the 6 Kb sequence at the 5′ end in the vector and arranged in the native relative orientation.

The key features of the 3′ vector (905) are as follows: 6 Kb of mouse genomic DNA (943) mapping within the intron between the J_{κ} (919) and C_{κ} (921) gene loci; a gene encoding the human hypoxanthine-guanine phosphoribosyl transferase (HPRT) under transcriptional control of the mouse Polr2a gene promoter (945); a neomycin resistance gene under the control of the mouse phosphoglycerate kinase 1 gene promoter (947); a loxP recognition sequence for the Cre recombinase (937); 3.6 Kb of mouse genomic DNA (949) that maps immediately downstream in the genome of the 6 Kb DNA fragment included at the 5′ end in the vector, with the two fragments oriented in the same transcriptional orientation as in the mouse genome; a gene encoding the diphtheria toxin A (DTA) subunit under transcriptional control of a modified herpes simplex virus type I thymidine kinase gene promoter coupled to two mutant transcriptional enhancers from the polyoma virus (923).

Mouse embryonic stem (ES) cells derived from C57B1/6NTac mice are transfected by electroporation with the 3′ vector (905) according to widely used procedures. Prior to electroporation, the vector DNA is linearized with a rare-cutting restriction enzyme that cuts only in the prokaryotic plasmid sequence or the polylinker associated with it. The transfected cells are plated and after ˜24 hours they are placed under positive selection for cells that have integrated the 3′ vector into their DNA by using the neomycin analogue drug G418. There is also negative selection for cells that have integrated the vector into their DNA but not by homologous recombination. Non-homologous recombination results in retention of the DTA gene, which kills the cells when the gene is expressed, whereas the DTA gene is deleted by homologous recombination since it lies outside of the region of vector homology with the mouse IGK locus. Colonies of drug-resistant ES cells are physically extracted from their plates after they became visible to the naked eye about a week later. These picked colonies are disaggregated, re-plated in micro-well plates, and cultured for several days. Thereafter, each of the clones of cells is divided such that some of the cells could be frozen as an archive, and the rest used for isolation of DNA for analytical purposes.

DNA from the ES cell clones is screened by PCR using a widely used gene-targeting assay design. For this assay, one of the PCR oligonucleotide primer sequences maps outside the region of identity shared between the 3′ vector (905) and the genomic DNA (901), while the other maps within the novel DNA between the two arms of genomic identity in the vector, i.e., in the HPRT (945) or neomycin resistance (947) genes. According to the standard design, these assays detect pieces of DNA that are only present in clones of ES cells derived from transfected cells that had undergone fully legitimate homologous recombination between the 3′ vector (905) and the endogenous mouse IGK locus. Two separate transfections are performed with the 3′ vector (905). PCR-positive clones from the two transfections are selected for expansion followed by further analysis using Southern blot assays.

The Southern blot assays are performed according to widely used procedures; they involve three probes and genomic DNA digested with multiple restriction enzymes chosen so that the combination of probes and digests allowed for conclusions to be drawn about the structure of the targeted locus in the clones and whether it is properly modified by homologous recombination. One of the probes maps to DNA sequence flanking the 5′ side of the region of identity shared between the 3′ κ targeting vector (905) and the genomic DNA; a second probe also maps outside the region of identity but on the 3′ side; the third probe maps within the novel DNA between the two arms of genomic identity in the vector, i.e., in the HPRT (945) or neomycin resistance (947) genes. The Southern blot identifies the presence of the expected restriction enzyme-generated fragment of DNA corresponding to the correctly mutated, i.e., by homologous recombination with the 3′ κ targeting vector (905) part of the κ locus, as detected by one of the external probes and by the neomycin resistance or HPRT gene probe. The external probe detects the mutant fragment and also a wild-type fragment from the non-mutant copy of the immunoglobulin κ locus on the homologous chromosome.

Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. Karyotypically normal clones that are judged to have the expected correct genomic structure based on the Southern blot data are selected for further use.

Acceptable clones are then modified with the 5′ vector (903) using procedures and screening assays that are similar in design to those used with the 3′ vector (905), except that puromycin selection is used instead of G418/neomycin selection, and the protocols are tailored to match the genomic region modified by the 5′ vector (903). The goal of the 5′ vector (903) transfection experiments is to isolate clones of ES cells that have been mutated in the expected fashion by both the 3′ vector (905) and the 5′ vector (903), i.e., doubly targeted cells carrying both engineered mutations. In these clones, the Cre recombinase causes a recombination (902) to occur between the loxP sites introduced into the κ locus by the two vectors, resulting in the genomic DNA configuration shown at 907.

Further, the clones must have undergone gene targeting on the same chromosome, as opposed to homologous chromosomes; i.e., the engineered mutations created by the targeting vectors must be in cis on the same DNA strand rather than in trans on separate homologous DNA strands. Clones with the cis arrangement are distinguished from those with the trans arrangement by analytical procedures such as fluorescence in situ hybridization of metaphase spreads using probes that hybridize to the novel DNA present in the two gene targeting vectors (903 and 905) between their arms of genomic identity. The two types of clones can also be distinguished from one another by transfecting them with a vector expressing the Cre recombinase, which deletes the pu-Tk (939), HPRT (945) and neomycin resistance (947) genes if the targeting vectors have been integrated in cis, and comparing the number of colonies that survive ganciclovir selection against the thymidine kinase gene introduced by the 5′ vector (903) and by analyzing the drug resistance phenotype of the surviving clones by a “sibling selection” screening procedure in which some of the cells from the clone are tested for resistance to puromycin or G418/neomycin. Cells with the cis arrangement of mutations are expected to yield approximately 10³ more ganciclovir-resistant clones than cells with the trans arrangement. The majority of the resulting cis-derived ganciclovir-resistant clones should also be sensitive to both puromycin and G418/neomycin, in contrast to the trans-derived ganciclovir-resistant clones, which should retain resistance to both drugs. Clones of cells with the cis-arrangement of engineered mutations in the κ chain locus are selected for further use.

The doubly targeted clones of cells are transiently transfected with a vector expressing the Cre recombinase (902) and the transfected cells are subsequently placed under ganciclovir selection, as in the analytical experiment summarized above. Ganciclovir-resistant clones of cells are isolated and analyzed by PCR and Southern blot for the presence of the expected deletion (907) between the two engineered mutations created by the 5′ vector (903) and the 3′ vector (905). In these clones, the Cre recombinase has caused a recombination to occur between the loxP sites (937) introduced into the κ chain locus by the two vectors. Because the loxP sites are arranged in the same relative orientations in the two vectors, recombination results in excision of a circle of DNA comprising the entire genomic interval between the two loxP sites. The circle does not contain an origin of replication and thus is not replicated during mitosis and is therefore lost from the clones of cells as they undergo clonal expansion. The resulting clones carry a deletion of the DNA that was originally between the two loxP sites. Clones that have the expected deletion are selected for further use.

The ES cell clones carrying the deletion of sequence in one of the two homologous copies of their immunoglobulin κ chain locus are retransfected (904) with a Cre recombinase expression vector together with a piece of DNA (909) comprising a partly canine immunoglobulin κ chain locus containing V_{κ} (951) and J_{κ} (955) gene segment coding sequences. The key features of this piece of DNA (referred to as “K-K”) are the following: a lox5171 site (931); a neomycin resistance gene open reading frame (947, lacking the initiator methionine codon, but in-frame and contiguous with an uninterrupted open reading frame in the lox5171 site (931)); a FRT site (927); an array of 14 canine V_{κ} gene segments (951), each with canine coding sequences embedded in mouse noncoding sequences; optionally a 13.5 Kb piece of genomic DNA from immediately upstream of the cluster of J_{κ} region gene segments in the mouse κ chain locus (not shown); a 2 Kb piece of DNA containing the 5 canine J_{κ} region gene segments (955) embedded in mouse noncoding DNA; a loxP site (937) in opposite relative orientation to the lox5171 site (931).

The sequences of the canine V_{κ} and J_{κ} gene coding regions are in Table 2.

In a second independent experiment, an alternative piece of partly canine DNA (909) is used in place of the K-K DNA. The key features of this DNA (referred to as “L-K”) are the following: a lox5171 site (931); a neomycin resistance gene open reading frame (947) lacking the initiator methionine codon, but in-frame and contiguous with an uninterrupted open reading frame in the lox5171 site (931); a FRT site (927); an array of 76 functional canine V), variable region gene segments (951), each with canine coding sequences embedded in mouse noncoding regulatory or scaffold sequences; optionally, a 13.5 Kb piece of genomic DNA from immediately upstream of the cluster of the J_{κ} region gene segments in the mouse κ chain locus (not shown); a 2 Kb piece of DNA containing 7 canine J_{λ} region gene segments embedded in mouse noncoding DNA (955); a loxP site (937) in opposite relative orientation to the lox5171 site (931). (The dog has 9 functional J_{λ} region gene segments, however, the encoded protein sequence of J_{λ4} and J_{λ9} and of J_{λ7} and J_{λ8} are identical, and so only 7 J_{λ} gene segments are included.)

The transfected clones from the K-K and L-K transfection experiments are placed under G418 selection, which enriches for clones of cells that have undergone RMCE, in which the partly canine donor DNA (909) is integrated in its entirety into the deleted immunoglobulin κ chain locus between the lox5171 (931) and loxP (937) sites that were placed there by 5′ (903) and 3′ (905) vectors, respectively. Only cells that have properly undergone RMCE have the capability to express the neomycin resistance gene (947) because the promoter (929) as well as the initiator methionine codon (935) required for its expression are not present in the vector (909) and are already pre-existing in the host cell IGH locus (907). The DNA region created using the K-K sequence is illustrated at 911. The remaining elements from the 5′ vector (903) are removed via Flp-mediated recombination (906) in vitro or in vivo, resulting in the final canine-based light chain locus as shown at 913.

G418-resistant ES cell clones are analyzed by PCR and Southern blotting to determine if they have undergone the expected RMCE process without unwanted rearrangements or deletions. Both K-K and L-K clones that have the expected genomic structure are selected for further use.

The K-K ES cell clones and the L-K ES cell clones carrying the partly canine immunoglobulin DNA in the mouse κ chain locus (913) are microinjected into mouse blastocysts from strain DBA/2 to create partly ES cell-derived chimeric mice according to standard procedures. Male chimeric mice with the highest levels of ES cell-derived contribution to their coats are selected for mating to female mice. The female mice of choice for use in the mating are of the C57B1/6NTac strain, and also carry a transgene encoding the Flp recombinase that is expressed in their germline. Offspring from these matings are analyzed for the presence of the partly canine immunoglobulin κ or λ light chain locus, and for loss of the FRT-flanked neomycin resistance gene that was created in the RMCE step. Mice that carry the partly canine locus are used to establish colonies of K-K and L-K mice.

Mice carrying the partly canine heavy chain locus, produced as described in Example 3, can be bred with mice carrying a canine-based κchain locus. Their offspring are in turn bred together in a scheme that ultimately produces mice that are homozygous for both canine-based loci, i.e., canine-based for heavy chain and κ. Such mice produce partly canine heavy chains with canine variable domains and mouse constant domains. They also produce partly canine κ proteins with canine κ variable domains and the mouse κ constant domain from their κ loci. Monoclonal antibodies recovered from these mice have canine heavy chain variable domains paired with canine κ variable domains.

A variation on the breeding scheme involves generating mice that are homozygous for the canine-based heavy chain locus, but heterozygous at the κ locus such that on one chromosome they have the K-K canine-based locus and on the other chromosome they have the L-K canine-based locus. Such mice produce partly canine heavy chains with canine variable domains and mouse constant domains. They also produce partly canine κ proteins with canine κ variable domains and the mouse κ constant domain from one of their κ loci. From the other κ locus, they produce partly canine λ proteins with canine λ variable domains the mouse κ constant domain. Monoclonal antibodies recovered from these mice have canine variable domains paired in some cases with canine κ variable domains and in other cases with canine λ variable domains.

### Example 5: Introduction of an Engineered Partly Canine Immunoglobulin Locus into the Immunoglobulin λ Chain Gene Locus of a Mouse Genome

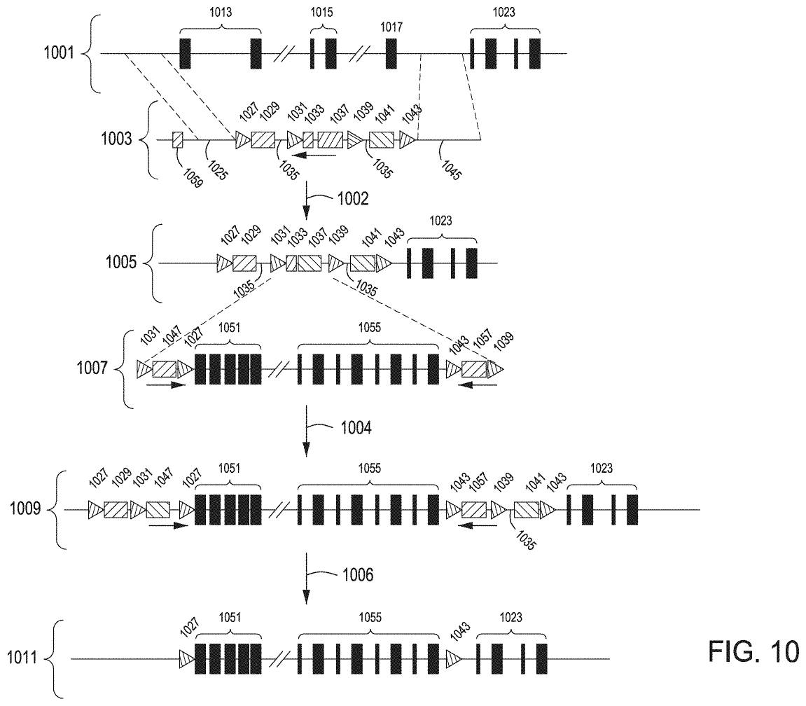

Another method for replacing a portion of a mouse genome with an engineered partly canine immunoglobulin locus is illustrated in FIG. 10. This method comprises deleting approximately 194 Kb of DNA from the wild-type mouse immunoglobulin λ locus (1001)—comprising V_{λx}/V_{λ2} gene segments (1013), J_{λ2}/C_{λ2} gene cluster (1015), and V_{λ1} gene segment (1017)—by a homologous recombination process involving a targeting vector (1003) that shares identity with the locus both upstream of the V_{λx}/V_{λ2} gene segments (1013) and downstream of the V_{λ1} gene segment (1017) in the immediate vicinity of the J_{λ3}, C_{λ3}, J_{λ1} λ and C21 X gene cluster (1023). The vector replaces the 194 Kb of DNA with elements designed to permit a subsequent site-specific recombination in which a non-native piece of DNA is moved into the modified V_{λ} locus via RMCE (1004). In this example, the non-native DNA is a synthetic nucleic acid comprising both canine and mouse sequences.

The key features of the gene targeting vector (1003) for accomplishing the 194 Kb deletion are as follows: a negative selection gene such as a gene encoding the A subunit of the diphtheria toxin (DTA, 1059) or a herpes simplex virus thymidine kinase gene (not shown); 4 Kb of genomic DNA from 5′ of the mouse V_{λx}/V_{λ2} variable region gene segments in the λ locus (1025); a FRT site (1027); a piece of genomic DNA containing the mouse Polr2a gene promoter (1029); a translation initiation sequence (methionine codon embedded in a “Kozak” consensus sequence) (1035); a mutated loxP recognition sequence (lox5171) for the Cre recombinase (1031); a transcription termination/polyadenylation sequence (1033); an open reading frame encoding a protein that confers resistance to puromycin (1037), whereas this open reading frame is on the antisense strand relative to the Polr2a promoter and the translation initiation sequence next to it and is followed by its own transcription termination/polyadenylation sequence (1033); a loxP recognition sequence for the Cre recombinase (1039); a translation initiation sequence (a methionine codon embedded in a “Kozak” consensus sequence) (1035) on the same, antisense strand as the puromycin resistance gene open reading frame; a chicken beta actin promoter and cytomegalovirus early enhancer element (1041) oriented such that it directs transcription of the puromycin resistance open reading frame, with translation initiating at the initiation codon downstream of the loxP site and continuing back through the loxP site into the puromycin open reading frame all on the antisense strand relative to the Polr2a promoter and the translation initiation sequence next to it; a mutated recognition site for the Flp recombinase known as an “F3” site (1043); a piece of genomic DNA upstream of the R3, C_{λ3}, J_{λ1} and C_{λ1} gene segments (1045).

Mouse embryonic stem (ES) cells derived from C57B1/6 NTac mice are transfected (1002) by electroporation with the targeting vector (1003) according to widely used procedures. Homologous recombination replaces the native DNA with the sequences from the targeting vector (1003) in the 196 Kb region resulting in the genomic DNA configuration depicted at 1005.

Prior to electroporation, the vector DNA is linearized with a rare-cutting restriction enzyme that cuts only in the prokaryotic plasmid sequence or the polylinker associated with it. The transfected cells are plated and after ˜24 hours placed under positive drug selection using puromycin. There is also negative selection for cells that have integrated the vector into their DNA but not by homologous recombination. Non-homologous recombination results in retention of the DTA gene, which kills the cells when the gene is expressed, whereas the DTA gene is deleted by homologous recombination since it lies outside of the region of vector homology with the mouse IGL locus. Colonies of drug-resistant ES cells are physically extracted from their plates after they became visible to the naked eye approximately a week later. These picked colonies are disaggregated, re-plated in micro-well plates, and cultured for several days. Thereafter, each of the clones of cells are divided such that some of the cells are frozen as an archive, and the rest used for isolation of DNA for analytical purposes.

DNA from the ES cell clones is screened by PCR using a widely used gene-targeting assay design. For these assays, one of the PCR oligonucleotide primer sequences maps outside the regions of identity shared between the targeting vector and the genomic DNA, while the other maps within the novel DNA between the two arms of genomic identity in the vector, e.g., in the puro gene (1037). According to the standard design, these assays detect pieces of DNA that would only be present in clones of cells derived from transfected cells that had undergone fully legitimate homologous recombination between the targeting vector (1003) and the native DNA (1001).

Six PCR-positive clones from the transfection (1002) are selected for expansion followed by further analysis using Southern blot assays. The Southern blots involve three probes and genomic DNA from the clones that has been digested with multiple restriction enzymes chosen so that the combination of probes and digests allow identification of whether the ES cell DNA has been properly modified by homologous recombination.

Karyotypes of the six PCR- and Southern blot-positive clones of ES cells are analyzed using an in situ fluorescence hybridization procedure designed to distinguish the most common chromosomal aberrations that arise in mouse ES cells. Clones that show evidence of aberrations are excluded from further use. Karyotypically normal clones that are judged to have the expected correct genomic structure based on the Southern blot data are selected for further use.

The ES cell clones carrying the deletion in one of the two homologous copies of their immunoglobulin λ chain locus are retransfected (1004) with a Cre recombinase expression vector together with a piece of DNA (1007) comprising a partly canine immunoglobulin λ chain locus containing V_{λ}, J_{λ} and C_{λ} region gene segments. The key features of this piece of DNA (1007) are as follows: a lox5171 site (1031); a neomycin resistance gene open reading frame lacking the initiator methionine codon, but in-frame and contiguous with an uninterrupted open reading frame in the lox5171 site (1047); a FRT site 1027); an array of 76 functional canine λ region gene segments, each with canine λ coding sequences embedded in mouse λ noncoding sequences (1051); an array of J-C units where each unit has a canine J_{λ} gene segment and a mouse λ constant domain gene segment embedded within noncoding sequences from the mouse λ locus (1055) (the canine J_{λ} gene segments are those encoding J_{λ1}, J_{λ2}, J_{λ3}, J_{λ4}, J_{λ5}, J_{λ6}, and J_{λ7}, while the mouse λ constant domain gene segments are C_{λ1} or C_{λ2} or C_{λ3}); a mutated recognition site for the Flp recombinase known as an “F3” site (1043); an open reading frame conferring hygromycin resistance (1057), which is located on the antisense strand relative to the immunoglobulin gene segment coding information in the construct; a loxP site (1039) in opposite relative orientation to the lox5171 site.

The sequences of the canine V_{λ} and J_{λ} gene coding regions are in Table 3.

The transfected clones are placed under G418 or hygromycin selection, which enriches for clones of cells that have undergone a RMCE process, in which the partly canine donor DNA is integrated in its entirety into the deleted immunoglobulin λ chain locus between the lox5171 and loxP sites that were placed there by the gene targeting vector. The remaining elements from the targeting vector (1003) are removed via FLP-mediated recombination (1006) in vitro or in vivo resulting in the final caninized locus as shown at 1011.

G418/hygromycin-resistant ES cell clones are analyzed by PCR and Southern blotting to determine if they have undergone the expected recombinase-mediated cassette exchange process without unwanted rearrangements or deletions. Clones that have the expected genomic structure are selected for further use.

The ES cell clones carrying the partly canine immunoglobulin DNA (1011) in the mouse λ chain locus are microinjected into mouse blastocysts from strain DBA/2 to create partially ES cell-derived chimeric mice according to standard procedures. Male chimeric mice with the highest levels of ES cell-derived contribution to their coats are selected for mating to female mice. The female mice of choice here are of the C57B1/6NTac strain, which carry a transgene encoding the Flp recombinase expressed in their germline. Offspring from these matings are analyzed for the presence of the partly canine immunoglobulin λ chain locus, and for loss of the FRT-flanked neomycin resistance gene and the F3-flanked hygromycin resistance gene that were created in the RMCE step. Mice that carry the partly canine locus are used to establish a colony of mice.

In some aspects, the mice comprising the canine-based heavy chain and κ locus (as described in Examples 3 and 4) are bred to mice that carry the canine-based λ locus. Mice generated from this type of breeding scheme are homozygous for the canine-based heavy chain locus, and can be homozygous for the K-K canine-based locus or the L-K canine-based locus. Alternatively, they can be heterozygous at the κ locus carrying the K-K locus on one chromosome and the L-K locus on the other chromosome. Each of these mouse strains is homozygous for the canine-based λ locus. Monoclonal antibodies recovered from these mice has canine heavy chain variable domains paired in some cases with canine κ variable domains and in other cases with canine λ variable domains. The λ variable domains are derived from either the canine-based L-K locus or the canine-based λ locus.

### Example 6: Introduction of an Engineered Partly Canine Immunoglobulin Minilocus into a Mouse Genome

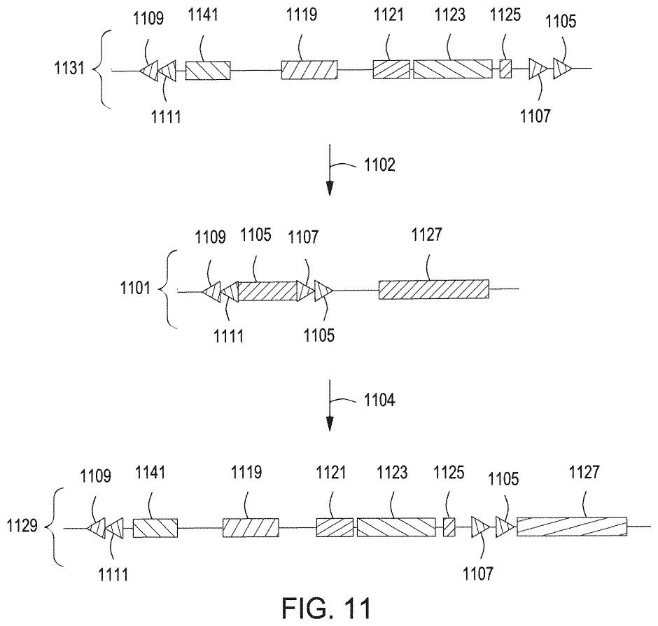

In certain other aspects, the partly canine immunoglobulin locus comprises a canine variable domain minilocus such as the one illustrated in FIG. 11. Here instead of a partly canine immunoglobulin locus comprising all or substantially all of the canine V_{H} gene segment coding sequences, the mouse immunoglobulin locus is replaced with a minilocus (1119) comprising fewer chimeric canine V_{H} gene segments, e.g. 1-39 canine V_{H} gene segments determined to be functional; that is, not pseudogenes.

A site-specific targeting vector (1131) comprising the partly canine immunoglobulin locus to be integrated into the mammalian host genome is introduced (1102) into the genomic region (1101) with the deleted endogenous immunoglobulin locus comprising the puro-TK gene (1105) and the following flanking sequence-specific recombination sites: mutant FRT site (1109), mutant LoxP site (1111), wild-type FRT site (1107), and wild-type LoxP site (1105). The site-specific targeting vector comprises i) an array of optional PAIR elements (1141); ii) a V_{H} locus (1119) comprising, e.g., 1-39 functional canine V_{H} coding regions and intervening sequences based on the mouse genome endogenous sequences; iii) a 21.6 kb pre-D region (1121) comprising mouse sequence; iv) a D locus (1123) and a J_{H} locus (1125) comprising 6 D and 6 J_{H} canine coding sequences and intervening sequences based on the mouse genome endogenous sequences. The partly canine immunoglobulin locus is flanked by recombination sites—mutant FRT (1109), mutant LoxP (1111), wild-type FRT (1107), and wild-type LoxP (1105)—that allow recombination with the modified endogenous locus. Upon introduction of the appropriate recombinase, e.g., Cre) (1104), the partly canine immunoglobulin locus is integrated into the genome upstream of the constant gene region (1127) as shown at 1129.

As described in Example 1, the primary screening for introduction of the partly canine immunoglobulin variable region locus is carried out by primary PCR screens supported by secondary Southern blotting assays. The deletion of the puro-TK gene (1105) as part of the recombination event allows identification of the cells that did not undergo the recombination event using ganciclovir negative selection.

### Example 7: Introduction of an Engineered Partly Canine Immunoglobulin Locus with Canine λ Variable Region Coding Sequences with Mouse λ Constant Region Sequences Embedded in κ Immunoglobulin Non-Coding Sequences

Dog antibodies mostly contain λ light chains, whereas mouse antibodies mostly contain κ light chains. To increase production of antibodies containing a λ LC, the endogenous mouse V_{κ} and J_{κ} are replaced with a partly canine locus containing V_{λ} and J_{λ} gene segment coding sequences embedded in mouse V_{κ} region flanking and regulatory sequences, the L-K mouse of Example 4. In such a mouse, the endogenous regulatory sequences promoting high level κ locus rearrangement and expression are predicted to have an equivalent effect on the ectopic λ locus. However, in vitro studies demonstrated that canine V_{λ} domains do not function well with mouse C_{κ} (see Example 9). Thus, the expected increase in λ LC-containing antibodies in the L-K mouse might not occur. As an alternate strategy, the endogenous mouse V_{κ} and J_{κ} are replaced with a partly canine locus containing V_{λ} and J_{λ} gene segment coding sequences embedded in mouse V_{κ} region flanking and regulatory sequences and mouse C_{κ} is replaced with mouse C_{λ}.

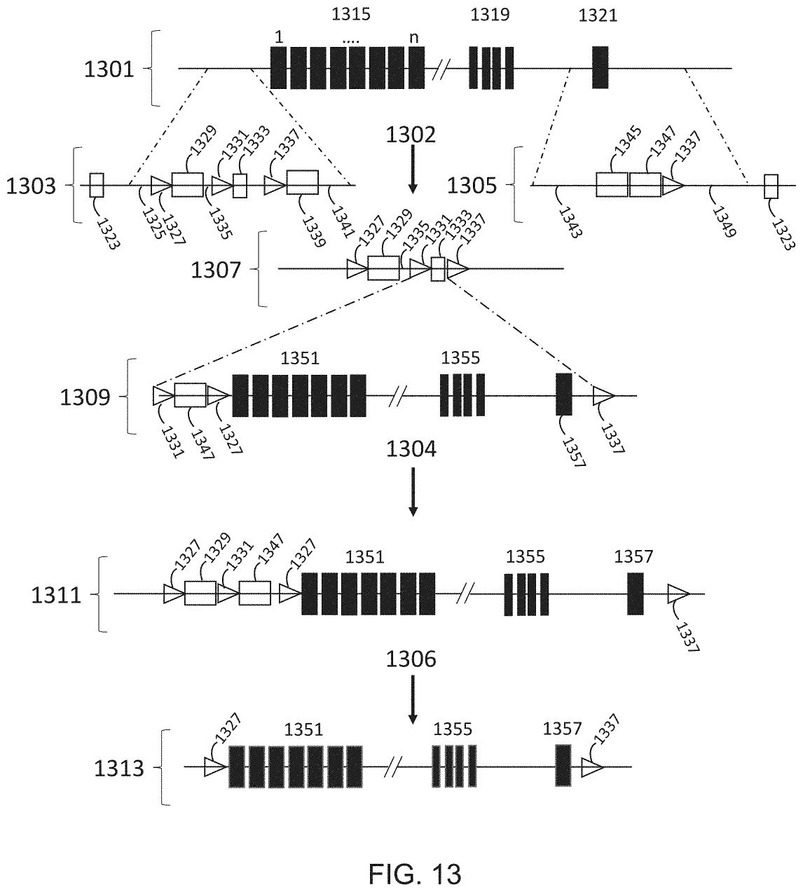

FIG. 13 is a schematic diagram illustrating the introduction of an engineered partly canine light chain variable region locus in which one or more canine V_{λ} gene segment coding sequences are inserted into a rodent immunoglobulin κ light chain locus upstream of one or more canine J_{λ} gene segment coding sequences, which are upstream of one or more rodent C_{λ} region coding sequences.

The method for replacing a portion of a mouse genome with a partly canine immunoglobulin locus is illustrated in FIG. 13. This method includes introducing a first site-specific recombinase recognition sequence into the mouse genome, which may be introduced either 5′ or 3′ of the cluster of endogenous V_{κ} (1315) and J_{κ} (1319) region gene segments and the C_{κ} (1321) exon of the mouse genome, followed by the introduction of a second site-specific recombinase recognition sequence into the mouse genome, which in combination with the first sequence-specific recombination site flanks the entire locus comprising clusters of V_{κ} and J_{κ} gene segments and the C_{κ} exon. The flanked region is deleted and then replaced with a partly canine immunoglobulin locus using the relevant site-specific recombinase, as described herein.

The targeting vectors employed for introducing the site-specific recombination sequences on either side of the V_{κ} (1315) gene segments and the C_{κ} exon (1321) also include an additional site-specific recombination sequence that has been modified so that it is still recognized efficiently by the recombinase, but does not recombine with unmodified sites. This site is positioned in the targeting vector such that after deletion of the V_{κ} and J_{κ} gene segment clusters and the C_{κ} exon it can be used for a second site specific recombination event in which a non-native piece of DNA is moved into the modified V_{κ} locus via RMCE. In this example, the non-native DNA is a synthetic nucleic acid comprises canine V_{λ} and J_{λ} gene segment coding sequences and mouse C_{λ} exon(s) embedded in mouse IGK regulatory and flanking sequences.

Two gene targeting vectors are constructed to accomplish the process just outlined. One of the vectors (1303) comprises mouse genomic DNA taken from the 5′ end of the locus, upstream of the most distal V_{κ} gene segment. The other vector (1305) comprises mouse genomic DNA taken from within the locus in a region spanning upstream (5′) and downstream (3′) of the C_{κ} exon (1321).

The key features of the 5′ vector (1303) are as follows: a gene encoding the diphtheria toxin A (DTA) subunit under transcriptional control of a modified herpes simplex virus type I thymidine kinase gene promoter coupled to two mutant transcriptional enhancers from the polyoma virus (1323); 6 Kb of mouse genomic DNA (1325) mapping upstream of the most distal variable region gene in the κ chain locus; a FRT recognition sequence for the Flp recombinase (1327); a piece of genomic DNA containing the mouse Polr2a gene promoter (1329); a translation initiation sequence (1335, methionine codon embedded in a “Kozak” consensus sequence); a mutated loxP recognition sequence (lox5171) for the Cre recombinase (1331); a transcription termination/polyadenylation sequence (1333); a loxP recognition sequence for the Cre recombinase (1337); a gene encoding a fusion protein with a protein conferring resistance to puromycin fused to a truncated form of the thymidine kinase (pu-TK) under transcriptional control of the promoter from the mouse phosphoglycerate kinase 1 gene (1339); 2.5 Kb of mouse genomic DNA (1341) mapping close to the 6 Kb sequence at the 5′ end in the vector and arranged in the native relative orientation.

The key features of the 3′ vector (1305) are as follows: 6 Kb of mouse genomic DNA (1343) mapping within the locus in a region spanning upstream (5′) and downstream (3′) of the C_{κ} exon (1321); a gene encoding the human hypoxanthine-guanine phosphoribosyl transferase (HPRT) under transcriptional control of the mouse Polr2a gene promoter (1345); a neomycin resistance gene under the control of the mouse phosphoglycerate kinase 1 gene promoter (1347); a loxP recognition sequence for the Cre recombinase (1337); 3.6 Kb of mouse genomic DNA (1349) that maps immediately downstream in the genome of the 6 Kb DNA fragment included at the 5′ end in the vector, with the two fragments oriented in the same transcriptional orientation as in the mouse genome; a gene encoding the diphtheria toxin A (DTA) subunit under transcriptional control of a modified herpes simplex virus type I thymidine kinase gene promoter coupled to two mutant transcriptional enhancers from the polyoma virus (1323).

One strategy to delete the endogenous mouse IGK locus is to insert the 3′ vector (1305) in the flanking region downstream of the mouse C_{κ} exon (1321). However, the 3′κ enhancer, which needs to be retained in the modified locus, is located 9.1 Kb downstream of the C_{κ} exon, which is too short to accommodate the upstream and downstream homology arms of the 3′ vector, which total 9.6 Kb. Therefore, the upstream region of homology was extended.

Mouse embryonic stem (ES) cells derived from C57B1/6NTac mice are transfected by electroporation with the 3′ vector (1305) according to widely used procedures. Prior to electroporation, the vector DNA is linearized with a rare-cutting restriction enzyme that cuts only in the prokaryotic plasmid sequence or the polylinker associated with it. The transfected cells are plated and after ˜24 hours they are placed under positive selection for cells that have integrated the 3′ vector into their DNA using the neomycin analogue drug G418. There is also negative selection for cells that have integrated the vector into their DNA but not by homologous recombination. Non-homologous recombination retains the DTA gene, which kills the cells when the gene is expressed, but the DTA gene is deleted by homologous recombination since it lies outside of the region of vector homology with the mouse IGK locus. Colonies of drug-resistant ES cells are physically extracted from their plates after they are visible to the naked eye about a week later. These colonies are disaggregated, re-plated in micro-well plates, and cultured for several days. Thereafter, each of the clones of cells is divided—some of the cells are frozen as an archive, and the rest are used to isolate DNA for analytical purposes.

DNA from the ES cell clones is screened by PCR using a widely used gene-targeting assay design. For this assay, one of the PCR oligonucleotide primer sequences maps outside the region of identity shared between the 3′ vector (1305) and the genomic DNA (1301), while the other maps within the novel DNA between the two arms of genomic identity in the vector, i.e., in the HPRT (1345) or neomycin resistance (1347) genes. According to the standard design, these assays detect pieces of DNA that are only present in clones of ES cells derived from transfected cells that had undergone fully legitimate homologous recombination between the 3′ vector (1305) and the endogenous mouse IGK locus. Two separate transfections are performed with the 3′ vector (1305). PCR-positive clones from the two transfections are selected for expansion followed by further analysis using Southern blot assays.

Southern blot assays are performed according to widely used procedures using three probes and genomic DNA digested with multiple restriction enzymes chosen so that the combination of probes and digests allowed for conclusions to be drawn about the structure of the targeted locus in the clones and whether it is properly modified by homologous recombination. A first probe maps to DNA sequence flanking the 5′ side of the region of identity shared between the 3′ κ targeting vector (1305) and the genomic DNA; a second probe also maps outside the region of identity but on the 3′ side; a third probe maps within the novel DNA between the two arms of genomic identity in the vector, i.e., in the HPRT (1345) or neomycin resistance (1347) genes. The Southern blot identifies the presence of the expected restriction enzyme-generated fragment of DNA corresponding to the correctly mutated, i.e., by homologous recombination with the 3′ κ targeting vector (1305) part of the κ locus, as detected by one of the external probes and by the neomycin resistance or HPRT gene probe. The external probe detects the mutant fragment and also a wild-type fragment from the non-mutant copy of the immunoglobulin κ locus on the homologous chromosome.

Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. Karyotypically normal clones that are judged to have the expected correct genomic structure based on the Southern blot data are selected for further use.

Acceptable clones are then modified with the 5′ vector (1303) using procedures and screening assays that are similar in design to those used with the 3′ vector (1305), except that puromycin selection is used instead of G418/neomycin selection, and the protocols are tailored to match the genomic region modified by the 5′ vector (1303). The goal of the 5′ vector (1303) transfection experiments is to isolate clones of ES cells that have been mutated in the expected fashion by both the 3′ vector (1305) and the 5′ vector (1303), i.e., doubly targeted cells carrying both engineered mutations. In these clones, the Cre recombinase causes a recombination (1302) to occur between the loxP sites introduced into the κ locus by the two vectors, resulting in the genomic DNA configuration shown at 1307.

Further, the clones must have undergone gene targeting on the same chromosome, as opposed to homologous chromosomes; i.e., the engineered mutations created by the targeting vectors must be in cis on the same DNA strand rather than in trans on separate homologous DNA strands. Clones with the cis arrangement are distinguished from those with the trans arrangement by analytical procedures such as fluorescence in situ hybridization of metaphase spreads using probes that hybridize to the novel DNA present in the two gene targeting vectors (1303 and 1305) between their arms of genomic identity. The two types of clones can also be distinguished from one another by transfecting them with a vector expressing the Cre recombinase, which deletes the pu-Tk (1339), HPRT (1345) and neomycin resistance (1347) genes if the targeting vectors have been integrated in cis, and comparing the number of colonies that survive ganciclovir selection against the thymidine kinase gene introduced by the 5′ vector (1303) and by analyzing the drug resistance phenotype of the surviving clones by a “sibling selection” screening procedure in which some of the cells from the clone are tested for resistance to puromycin or G418/neomycin. Cells with the cis arrangement of mutations are expected to yield approximately 10³ more ganciclovir-resistant clones than cells with the trans arrangement. The majority of the resulting cis-derived ganciclovir-resistant clones should also be sensitive to both puromycin and G418/neomycin, in contrast to the trans-derived ganciclovir-resistant clones, which should retain resistance to both drugs. Clones of cells with the cis-arrangement of engineered mutations in the κ chain locus are selected for further use.

The doubly targeted clones of cells are transiently transfected with a vector expressing the Cre recombinase (1302) and the transfected cells are subsequently placed under ganciclovir selection, as in the analytical experiment summarized above. Ganciclovir-resistant clones of cells are isolated and analyzed by PCR and Southern blot for the presence of the expected deletion (1307) between the two engineered mutations created by the 5′ vector (1303) and the 3′ vector (1305). In these clones, the Cre recombinase causes a recombination to occur between the loxP sites (1337) introduced into the κ chain locus by the two vectors. Because the loxP sites are arranged in the same relative orientations in the two vectors, recombination results in excision of a circle of DNA comprising the entire genomic interval between the two loxP sites. The circle does not contain an origin of replication and thus is not replicated during mitosis and is therefore lost from the clones of cells as they undergo clonal expansion. The resulting clones carry a deletion of the DNA that was originally between the two loxP sites and have the genomic structure show at 1307. Clones that have the expected deletion are selected for further use.

The ES cell clones carrying the sequence deletion in one of the two homologous copies of their immunoglobulin κ chain locus are retransfected (1304) with a Cre recombinase expression vector together with a piece of DNA (1309) comprising a partly canine immunoglobulin λ chain locus containing V_{λ} (1351) and J_{λ} (1355) gene segment coding sequences and mouse C_{λ} exon(s) (1357). The key features of this piece of DNA are the following: a lox5171 site (1331); a neomycin resistance gene open reading frame (1347, lacking the initiator methionine codon, but in-frame and contiguous with an uninterrupted open reading frame in the lox5171 site (1331); a FRT site (1327); an array of 1-76 functional canine V_{λ} variable region gene segments (1351), each with canine coding sequences embedded in mouse noncoding regulatory or scaffold sequences; optionally, a 13.5 Kb piece of genomic DNA from immediately upstream of the cluster of the J_{κ} region gene segments in the mouse κ chain locus (not shown); a 2 Kb piece of DNA containing 1-7 canine J_{λ} region gene segments embedded in mouse noncoding DNA (1355) and mouse C_{λ} exon(s) (1357); a loxP site (1337) in opposite relative orientation to the lox5171 site (1331). The piece of DNA also contains the deleted iEκ (not shown).

The sequences of the canine V_{λ} and J_{λ} gene coding regions are in Table 3.

The transfected cells are placed under G418 selection, which enriches for clones of cells that have undergone RMCE, in which the partly canine donor DNA (1309) is integrated in its entirety into the deleted immunoglobulin κ chain locus between the lox5171 (1331) and loxP (1337) sites that were placed there by 5′ (1303) and 3′ (1305) vectors, respectively. Only cells that have properly undergone RMCE have the capability to express the neomycin resistance gene (1347) because the promoter (1329) as well as the initiator methionine codon (1335) required for its expression are not present in the vector (1309) and are already pre-existing in the host cell IGK locus (1307). The DNA region created by RMCE is illustrated at 1311. The remaining elements from the 5′ vector (1303) are removed via Flp-mediated recombination (1306) in vitro or in vivo, resulting in the final canine-based light chain locus as shown at 1313.

G418-resistant ES cell clones are analyzed by PCR and Southern blotting to determine if they have undergone the expected RMCE process without unwanted rearrangements or deletions. Clones that have the expected genomic structure are selected for further use.

Clones carrying the partly canine immunoglobulin DNA in the mouse κ chain locus (1313) are microinjected into mouse blastocysts from strain DBA/2 to create partly ES cell-derived chimeric mice according to standard procedures. Male chimeric mice with the highest levels of ES cell-derived contribution to their coats are selected for mating to female mice. The female mice of choice for use in the mating are of the C57B1/6NTac strain, and also carry a transgene encoding the Flp recombinase that is expressed in their germline. Offspring from these matings are analyzed for the presence of the partly canine immunoglobulin λ light chain locus, and for loss of the FRT-flanked neomycin resistance gene that was created in the RMCE step. Mice that carry the partly canine locus are used to establish colonies of mice.

Mice carrying the partly canine heavy chain locus, produced as described in Example 3, can be bred with mice carrying a canine λ-based κ chain locus. Their offspring are in turn bred together in a scheme that ultimately produces mice that are homozygous for both canine-based loci, i.e., canine-based for heavy chain and λ-based λ. Such mice produce partly canine heavy chains with canine variable domains and mouse constant domains. They also produce partly canine λ proteins with canine λ variable domains and the mouse λ constant domain from their κ loci. Monoclonal antibodies recovered from these mice have canine heavy chain variable domains paired with canine λ variable domains.

A variation on the breeding scheme involves generating mice that are homozygous for the canine-based heavy chain locus, but heterozygous at the κ locus such that on one chromosome they have the K-K canine-based locus described in Example 4 and on the other chromosome they have the partly canine λ-based κ locus described in this example. Such mice produce partly canine heavy chains with canine variable domains and mouse constant domains. They also produce partly canine κ proteins with canine κ variable domains and the mouse κ constant domain from one of their κ loci. From the other κ locus, partly canine λ proteins comprising canine λ variable domains and the mouse λ constant domain are produced. Monoclonal antibodies recovered from these mice include canine variable domains paired in some cases with canine κ variable domains and in other cases with canine λ variable domains.

### Example 8. Introduction of an Engineered Partly Canine Immunoglobulin Locus with Canine λ Variable Region Coding Sequences with Mouse λ Constant Region Sequences Embedded in Mouse κ Immunoglobulin Non-Coding Sequences

This example describes an alternate strategy to Example 7 in which the endogenous mouse V_{κ} and J_{κ} are replaced with a partly canine locus containing canine V_{λ} and J_{λ} gene segment coding sequences embedded in mouse V_{κ} region flanking and regulatory sequences and mouse C_{κ} is replaced with mouse C_{λ}. However, in this example the structure of the targeting vector containing the partly canine locus is different. The canine V gene locus coding sequences include an array of anywhere from 1 to 76 functional V_{λ} gene segment coding sequences, followed by an array of J_{λ}-C_{λ} tandem cassettes in which the J_{λ} is of canine origin and the C_{λ} is of mouse origin, for example, C_{λ1}, C_{λ2} or C_{λ3}. The number of cassettes ranges from one to seven, the number of unique functional canine J_{λ} gene segments. The overall structure of the partly canine λ locus in this example is similar to the endogenous mouse λ locus, whereas the structure of the locus in Example 7 is similar to the endogenous mouse κ locus, which is being replaced by the partly canine λ locus in that example.

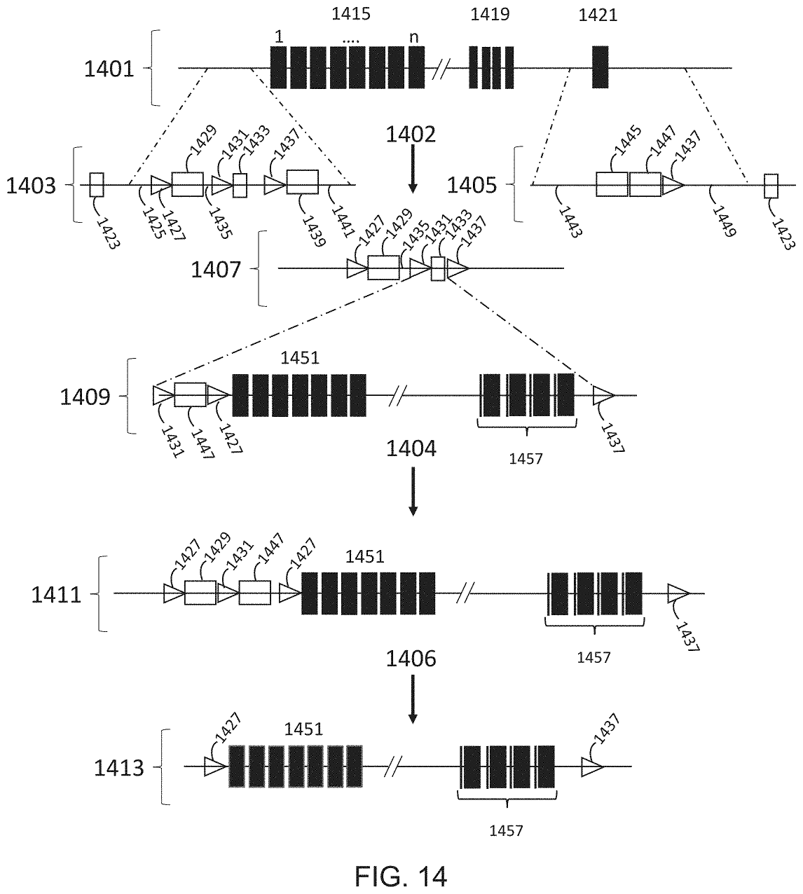

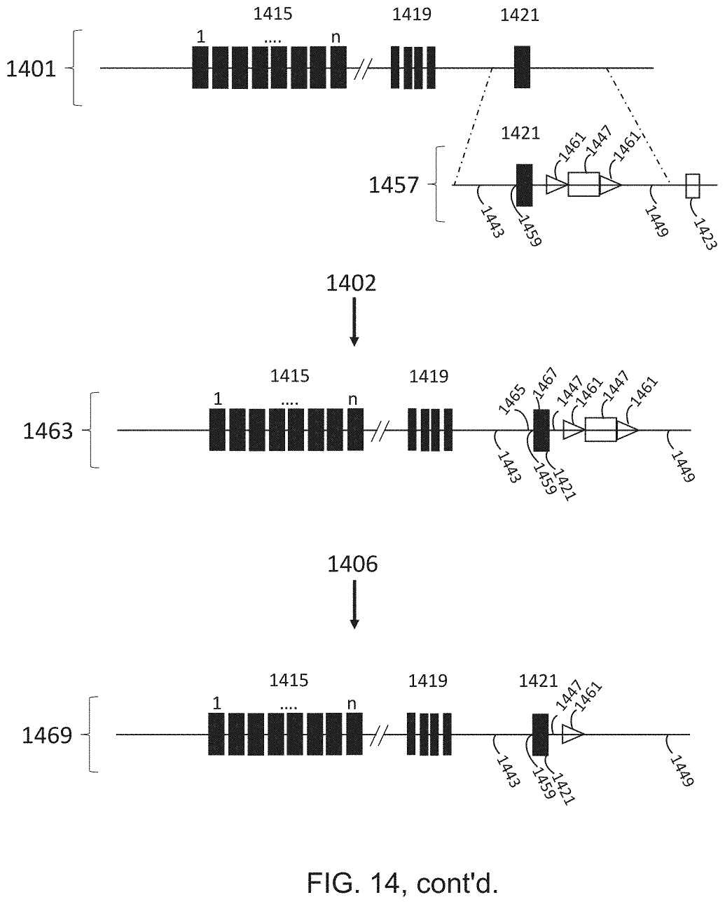

FIG. 14 is a schematic diagram illustrating the introduction of an engineered partly canine light chain variable region locus in which one or more canine V_{λ} gene segment coding sequences are inserted into a rodent immunoglobulin κ light chain locus upstream of an array of J_{λ}-C_{λ} tandem cassettes in which the J_{λ} is of canine origin and the C_{λ} is of mouse origin, for example, C_{λ1}, C_{λ2} or C_{λ3}.

The method for replacing a portion of a mouse genome with a partly canine immunoglobulin locus is illustrated in FIG. 14. This method provides introducing a first site-specific recombinase recognition sequence into the mouse genome, which may be introduced either 5′ or 3′ of the cluster of endogenous V_{κ} (1415) and J_{κ} (1419) region gene segments and the C_{κ} (1421) exon of the mouse genome, followed by the introduction of a second site-specific recombinase recognition sequence into the mouse genome, which in combination with the first sequence-specific recombination site flanks the entire locus comprising clusters of V_{κ} and J_{κ} gene segments and the C_{κ} exon. The flanked region is deleted and then replaced with a partly canine immunoglobulin locus using the relevant site-specific recombinase, as described herein.

The targeting vectors employed for introducing the site-specific recombination sequences on either side of the V_{κ} (1415) gene segments and the C_{κ} exon (1421) also include an additional site-specific recombination sequence that has been modified so that it is still recognized efficiently by the recombinase, but does not recombine with unmodified sites. This site is positioned in the targeting vector such that after deletion of the V_{κ} and J_{κ} gene segment clusters and the C_{κ} exon it can be used for a second site specific recombination event in which a non-native piece of DNA is moved into the modified V_{κ} locus via RMCE. In this example, the non-native DNA is a synthetic nucleic acid comprising an array of canine V_{λ} gene segment coding sequences and an array of J_{λ}-C_{λ} tandem cassettes in which the J_{λ} is of canine origin and the C_{λ} is of mouse origin, for example, C_{λ1}, C_{λ2} or C_{λ3} embedded in mouse IGK regulatory and flanking sequences.

Two gene targeting vectors are constructed to accomplish the process just outlined. One of the vectors (1403) comprises mouse genomic DNA taken from the 5′ end of the locus, upstream of the most distal V_{κ} gene segment. The other vector (1405) comprises mouse genomic DNA taken from within the locus in a region spanning upstream (5′) and downstream (3′) of the C_{κ} exon (1321).

The key features of the 5′ vector (1403) and the 3′ vector (1405) are described in Example 7.

Mouse embryonic stem (ES) cells derived from C57B1/6NTac mice are transfected by electroporation with the 3′ vector (1405) according to widely used procedures as described in Example 7. DNA from the ES cell clones is screened by PCR using a widely used gene-targeting assay as described in Example 7. The Southern blot assays are performed according to widely used procedures as described in Example 7.

Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. Karyotypically normal clones that are judged to have the expected correct genomic structure based on the Southern blot data are selected for further use.

Acceptable clones are modified with the 5′ vector (1403) using procedures and screening assays as described in Example 7. The resulting correctly targeted ES clones have the genomic DNA configuration of the endogenous κ locus in which the 5′ vector (1403) is inserted upstream of endogenous V_{κ} gene segments and the 3′ vector (1405) is inserted downstream of the endogenous C_{κ}. In these clones, the Cre recombinase causes recombination (1402) to occur between the loxP sites introduced into the κ locus by the two vectors, resulting in the genomic DNA configuration shown at 1407.

Acceptable clones undergo gene targeting on the same chromosome, as opposed to homologous chromosomes; such that the engineered mutations created by the targeting vectors are in cis on the same DNA strand rather than in trans on separate homologous DNA strands. Clones with the cis arrangement are distinguished from those with the trans arrangement by analytical procedures as described in Example 7.

The doubly targeted clones of cells are transiently transfected with a vector expressing the Cre recombinase (1402) and the transfected cells are subsequently placed under ganciclovir selection and analyses using procedures described in Example 7. In selected clones, the Cre recombinase has caused a recombination to occur between the loxP sites (1437) introduced into the κ chain locus by the two vectors. Because the loxP sites are arranged in the same relative orientations in the two vectors, recombination results in excision of a circle of DNA comprising the entire genomic interval between the two loxP sites. The circle does not contain an origin of replication and thus is not replicated during mitosis and is therefore lost from the clones of cells as they undergo clonal expansion. The resulting clones carry a deletion of the DNA that was originally between the two loxP sites and have the genomic structure show at 1407. Clones that have the expected deletion are selected for further use.

The ES cell clones carrying the deletion of sequence in one of the two homologous copies of their immunoglobulin κ chain locus are retransfected (1404) with a Cre recombinase expression vector together with a piece of DNA (1409) comprising a partly canine immunoglobulin λ chain locus containing V_{λ} (1451) segment coding sequences and a tandem array of cassettes containing canine J_{λ} gene segment coding sequences and mouse C_{λ} exon(s) embedded in mouse IGK flanking and regulatory DNA sequences (1457). The key features of this piece of DNA are the following: a lox5171 site (1431); a neomycin resistance gene open reading frame (1447, lacking the initiator methionine codon, but in-frame and contiguous with an uninterrupted open reading frame in the lox5171 site (1431); a FRT site (1427); an array of 1-76 functional canine V_{λ} variable region gene segments (1451), each containing canine coding sequences embedded in mouse noncoding regulatory or scaffold sequences; optionally, a 13.5 Kb piece of genomic DNA from immediately upstream of the cluster of the J_{κ} region gene segments in the mouse κ chain locus (not shown); DNA containing a tandem array of cassettes containing canine J_{λ} gene segment coding sequences and mouse C_{λ} exon(s) embedded in mouse IGK flanking and regulatory DNA sequences (1457); a loxP site (1437) in opposite relative orientation to the lox5171 site (1431).

The sequences of the canine V_{λ} and J_{λ} gene coding regions are in Table 3.

The transfected cells are placed under G418 selection, which enriches for clones of cells that have undergone RMCE, in which the partly canine donor DNA (1409) is integrated in its entirety into the deleted immunoglobulin κ chain locus between the lox5171 (1431) and loxP (1437) sites placed there by the 5′ (1403) and 3′ (1405) vectors, respectively. Only cells that properly undergo RMCE have the capability to express the neomycin resistance gene (1447) because the promoter (1429) as well as the initiator methionine codon (1435) required for its expression are not present in the vector (1409) and are already pre-existing in the host cell IGK locus (1407). The DNA region created by RMCE is illustrated at 1411. The remaining elements from the 5′ vector (1403) are removed via Flp-mediated recombination (1406) in vitro or in vivo, resulting in the final canine-based light chain locus as shown at 1413.

G418-resistant ES cell clones are analyzed by PCR and Southern blotting to determine if they have undergone the expected RMCE process without unwanted rearrangements or deletions. Clones that have the expected genomic structure are selected for further use.

Clones carrying the partly canine immunoglobulin DNA in the mouse κ chain locus (1413) are microinjected into mouse blastocysts from strain DBA/2 to create partly ES cell-derived chimeric mice according to standard procedures. Male chimeric mice with the highest levels of ES cell-derived contribution to their coats are selected for mating to female mice. The female mice of choice for use in the mating are of the C57B1/6NTac strain, and also carry a transgene encoding the Flp recombinase that is expressed in their germline. Offspring from these matings are analyzed for the presence of the partly canine immunoglobulin λ light chain locus, and for loss of the FRT-flanked neomycin resistance gene that was created in the RMCE step. Mice that carry the partly canine locus are used to establish colonies of mice.

Mice carrying the partly canine heavy chain locus, produced as described in Example 3, can be bred with mice carrying a canine λ-based κ chain locus. Their offspring are in turn bred together in a scheme that ultimately produces mice that are homozygous for both canine-based loci, i.e., canine-based for heavy chain and λ-based κ. Such mice produce partly canine heavy chains with canine variable domains and mouse constant domains. They also produce partly canine λ proteins with canine λ variable domains and the mouse λ constant domain from their κ loci. Monoclonal antibodies recovered from these mice have canine heavy chain variable domains paired with canine λ variable domains.

A variation on the breeding scheme involves generating mice that are homozygous for the canine-based heavy chain locus, but heterozygous at the κ locus such that on one chromosome they have the K-K canine-based locus described in Example 4 and on the other chromosome they have the partly canine λ-based κ locus described in this example. Such mice produce partly canine heavy chains with canine variable domains and mouse constant domains. They also produce partly canine κ proteins with canine κ variable domains and the mouse κ constant domain from one of their κ loci. From the other κ locus, they produce partly canine λ proteins with canine λ variable domains and the mouse λ constant domain. Monoclonal antibodies recovered from these mice have canine variable domains paired in some cases with canine κ variable domains and in other cases with canine λ variable domains.

The method described above for introducing an engineered partly canine immunoglobulin locus with canine λ variable region coding sequences and mouse λ constant region sequences embedded in mouse κ immunoglobulin non-coding sequences involve deletion of the mouse C_{κ} exon. An alternate method involves inactivating the C_{κ} exon by mutating its splice acceptor site. Introns must be removed from primary mRNA transcripts by a process known as RNA splicing in which the spliceosome, a large molecular machine located in the nucleus, recognizes sequences at the 5′ (splice donor) and 3′ (splice acceptor) ends of the intron, as well as other features of the intron including a polypyrimidine tract located just upstream of the splice acceptor. The splice donor sequence in the DNA is NGT, where “N” is any deoxynucleotide and the splice acceptor is AGN (Cech T R, Steitz J A and Atkins J F Eds. (2019) (RNA Worlds: New Tools for Deep Exploration, CSHL Press) ISBN 978-1-621822-24-0).

The mouse C_{κ} exon is inactivated by mutating its splice acceptor sequence and the polypyrimidine tract. The wild type sequence upstream of the C_{κ} exon is CTTCCTTCCTCAG (SEQ ID NO: 470) (the splice acceptor site is underlined). It is mutated to AAATTAATTAACC (SEQ ID NO: 471), resulting in a non-functional splice acceptor site and thus a non-functional C_{κ} exon. The mutant sequence also introduces a PacI restriction enzyme site (underlined). As an eight base pair recognition sequence, this restriction site is expected to be present only rarely in the mouse genome (˜every 65,000 bp), making it simple to detect whether the mutant sequence has been inserted into the IGK locus by Southern blot analysis of the ES cell DNA that has been digested with PacI and another, more frequently cutting restriction enzyme. The wild type sequence is replaced with the mutant sequence by homologous recombination, a technique widely known in the art, as to insert the 3′ RMCE vector. The key features of the homologous recombination vector (MSA, 1457) to mutate the C_{κ} exon splice acceptor sequence and the polypyrimidine tract are as follows: 6 Kb of mouse genomic DNA (1443) mapping within the κ locus in a region spanning upstream (5′) and downstream (3′) of the C_{κ} exon (1421) and containing the mutant AAATTAATTAACC (SEQ ID NO: 471) (1459) sequence instead of the wild type CTTCCTTCCTCAG (SEQ ID NO: 470) sequence in its natural position just upstream of the C_{κ} exon; a neomycin resistance gene under the control of the mouse phosphoglycerate kinase 1 gene promoter (1447) and flanked by mutant FRT sites (1461); 3.6 Kb of mouse genomic DNA (1449) that maps immediately downstream in the genome of the 6 Kb DNA fragment included at the 5′ end in the vector, with the two fragments oriented in the same transcriptional orientation as in the mouse genome; a gene encoding the diphtheria toxin A (DTA) subunit under transcriptional control of a modified herpes simplex virus type I thymidine kinase gene promoter coupled to two mutant transcriptional enhancers from the polyoma virus (1423). Mutant FRT sites (1461), e.g., FRT F3 or FRT F5 (Schlake and Bode (1994) Use of mutated FLP recognition target (FRT) sites for the exchange of expression cassettes at defined chromosomal loci. Biochemistry 33:12746-12751 PMID: 7947678 DOI: 10.1021/bi00209a003), are being used here because, once the spicing mutation is introduced and the Neo gene is deleted by transient transfection of a FLP recombinase expression vector (1406), the ES cells are subjected to further genetic manipulation. This process requires wild type FRT sites to delete another Neo selection gene (1447 at 1403). If the FRT site (1461) remaining in the IGK locus (1469) after introduction of the splicing mutation is wild type, attempted FRT-mediated deletion of this second Neo gene (1406 at 1413) may inadvertently result in deletion of the entire newly-introduced partly canine locus and the inactivated mouse C_{κ} exon.

Mouse embryonic stem (ES) cells derived from C57B1/6NTac mice are transfected by electroporation with the MSA vector (1457) according to widely used procedures. Prior to electroporation, the vector DNA is linearized with a rare-cutting restriction enzyme that cuts only in the prokaryotic plasmid sequence or the polylinker associated with it. The transfected cells are plated and after ˜24 hours they are placed under positive selection for cells that have integrated the MSA vector into their DNA by using the neomycin analogue drug G418. There is also negative selection for cells that have integrated the vector into their DNA but not by homologous recombination. Non-homologous recombination results in retention of the DTA gene, which kills the cells when the gene is expressed, whereas the DTA gene is deleted by homologous recombination since it lies outside of the region of vector homology with the mouse IGK locus. Colonies of drug-resistant ES cells are physically extracted from their plates after they became visible to the naked eye about a week later. These picked colonies are disaggregated, re-plated in micro-well plates, and cultured for several days. Thereafter, each of the clones of cells is divided such that some of the cells are frozen as an archive, and the rest used to isolate DNA for analytical purposes.

The IGK locus in ES cells that are correctly targeted by homologous recombination has the configuration depicted at 1463.

DNA from the ES cell clones is screened by PCR using a widely used gene-targeting assay design. For this assay, one of the PCR oligonucleotide primer sequences maps outside the region of identity shared between the MSA vector (1457) and the genomic DNA (1401), while the other maps within the novel DNA between the two arms of genomic identity in the vector, i.e., the neomycin resistance (1447) gene. According to the standard design, these assays detect pieces of DNA that are only present in clones of ES cells derived from transfected cells that had undergone fully legitimate homologous recombination between the MSA vector (1457) and the endogenous mouse IGK locus. Two separate transfections are performed with the MSA vector (1457). PCR-positive clones from the two transfections are selected for expansion followed by further analysis using Southern blot assays.

The Southern blot assays are performed according to widely used procedure using three probes and genomic DNA digested with multiple restriction enzymes chosen so that the combination of probes and digests allowed for conclusions to be drawn about the structure of the targeted locus in the clones and whether it is properly modified by homologous recombination. In in this particular example, the DNA is double digested with Pac1 and another restriction enzyme such as EcoRI or HindIII, as only cells with the integrated MSA vector contains the PacI site. A first probe maps to DNA sequence flanking the 5′ side of the region of identity shared between the MSA vector (1457) and the genomic DNA; a second probe also maps outside the region of identity but on the 3′ side; a third probe maps within the novel DNA between the two arms of genomic identity in the vector, i.e., in the neomycin resistance (1447) gene. The Southern blot identifies the presence of the expected restriction enzyme-generated fragment of DNA corresponding to the correctly mutated, i.e., by homologous recombination with the MSA κ targeting vector (1457) part of the κ locus, as detected by one of the external probes and by the neomycin resistance gene probe. The external probe detects the mutant fragment and also a wild-type fragment from the non-mutant copy of the immunoglobulin κ locus on the homologous chromosome. The Southern blot assays are performed according to widely used procedures described in Example 7.

Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. Karyotypically normal clones that are judged to have the expected correct genomic structure based on the Southern blot data are selected for further use.

Although the ability of the ES cell DNA to be digested by PacI in the mutated IGK allele confirms the presence of the TTAATTAA sequence, DNA sequencing focusing on the region upstream of the C_{κ} exon is performed to confirm the presence of the complete expected splicing mutation. The region is amplified by genomic PCR using primers that flank the mutation [1465 and 1467 (Table 6: SEQ ID NO: 450 and SEQ ID NO:451)]. An alternate primer pair is shown in SEQ ID NO: 452 and SEQ ID NO: 453. These primers are designed using NCBI Primer-Blast and verified in silico to lack any predicted off-target binding sites in the mouse genome.

Sequence-verified ES cell clones are transiently transfected (1406) with a FLP recombinase expression vector to delete the neomycin resistance gene (1427). The cells are then subcloned and the deletion is confirmed by PCR. The IGK locus in the ES cells have the genomic configuration depicted at 1469.

The ES cells are electroporated with the 5′ and 3′ RMCE vectors, as described above. The only differences are that the 3′ vector (1405) is inserted upstream of the mutant C_{κ} exon at the position shown in FIG. 9 at 901 and upstream and downstream homology arms of the 3′ vector (1405) is replaced by the sequences 943 and 949, respectively of the 3′ vector (905) shown in FIG. 9. As a result, PCR primers and Southern blot probes used to test for correct integration of the 3′ vector (1405) are derived from sequences 943 and 949 instead of 1443 and 1449. The iEκ enhancer is not included in the targeting vector (1409), since this sequence was not deleted.

### Example 9: Canine Vλ Domains do not Function Well with Mouse Cκ Domains and Canine Vκ Domains do not Function Well with Mouse Cλ Domains

For the proposed L-K mouse (Example 4), canine V_{λ} and J_{λ} gene segment coding sequences flanked by mouse non-coding and regulatory sequences are embedded in the mouse IGK locus from which endogenous V_{κ} and J_{κ} gene segments have been deleted. After productive V_{λ}→J_{λ} gene rearrangement, the resulting Ig gene encodes a LC with a canine λ variable domain and a mouse κ constant domain. To test whether such a hybrid LC was properly expressed and forms an intact Ig molecule, a series of transient transfection assays were performed with different combinations of Vs, both V_{κ} and V_{κ}, and C light chain exons, both C_{κ} and C_{λ}, together with an Ig HC and tested for cell surface and intracellular expression and secretion of the encoded Ig.

For these experiments canine IGHV3-5 (Accession No. MF785020.1), IGHV3-19 (Accession No. FJ197781.1) or IGHV4-1 (Accession No. DN362337.1) linked to a mouse IgM^{b} allotype HC was individually cloned into a pCMV vector. Each V_{H}-encoding DNA contained the endogenous canine L1-intron-L2 and germline, i.e., unmutated VDJ sequence. Unmutated canine IGLV3-28 (Accession No. EU305423) or IGKV2-5 (Accession No. EU295719.1) were cloned into a pFUSE vector. Each canine V_{L} exon was linked to the constant region of mouse C_{κ}, C_{λ1} or C_{λ2} (C_{λ3} was presumed to have the same properties as C_{λ2} since they have nearly identical protein sequence.) L1-intron-L2 sequences in each VL were of canine origin. 293T/17 cells were co-transfected with a human CD4 expression vector as a transfection control plus one of the HC and LC constructs and a CD79a/b expression vector. The CD79a/b heterodimer was required for cell surface expression of the IgM. Approximately 24 h later, the transfected cells were subjected to cell surface or intracellular staining by flow cytometry. For analysis of Ig secretion, the same V_{H} genes as above were cloned into a pFUSE vector containing mouse IgG2a Fc. 293T/17 cells were co-transfected with a human CD4 (hCD4) expression vector as a transfection control plus one of the HC and LC constructs described above. (In these experiments C_{λ3} was also tested.) Approximately 48 hr later, the transfected cells and their corresponding supernatants were harvested and analyzed for HC/LC expression/secretion by western blotting.

To summarize the data obtained from these experiments, when canine IGLV3-28 was linked to mouse C_{κ}, IgM expression on the cell surface was at least two times less than when the same dog V_{λ} was linked to C_{λ1} or C_{λ2}. Likewise, when IGKV2-5 was linked to mouse C_{λ} the level of surface IgM was drastically decreased. The extent of the expression defect was dependent of the particular V_{H} gene being used; some V_{H} genes allowed for some cell surface expression of the hybrid light chains, but others were more stringent. The same trends were seen with Ig secretion.

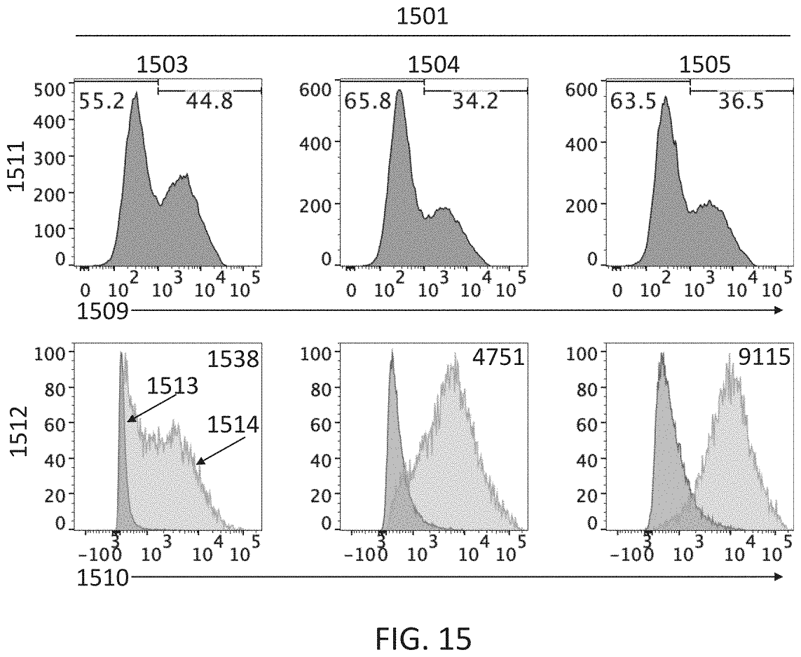

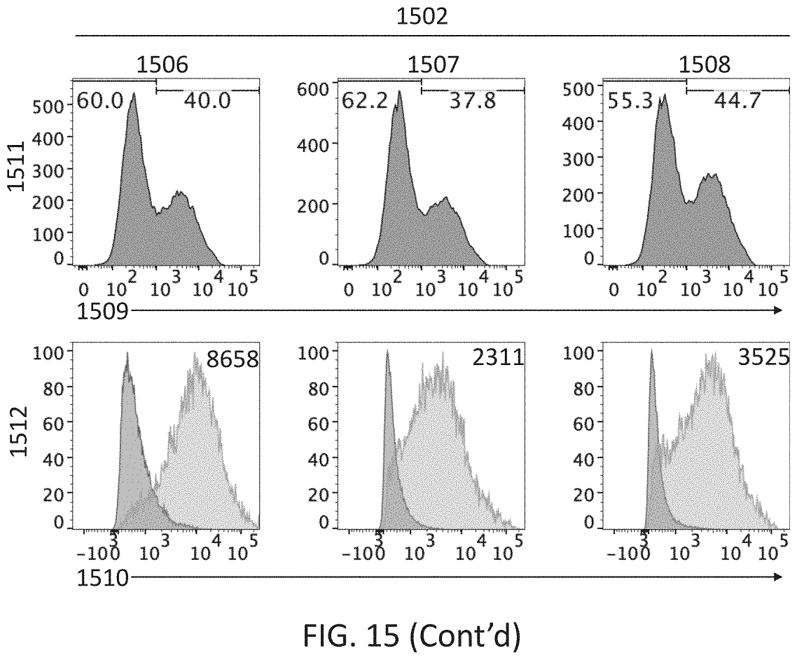

FIG. 15 shows the results of flow cytometry analysis of cells expressing IGHV3-5, which was one of the less stringent V_{H} genes, with canine IGVL3-28/IGLJ6 (1501) or with canine IGVK2-5/IGJK1 (1502). Row 1509 panels are transfection controls stained with hCD4 mAb antibody and row 11510 panels were stained with mouse IgM^{b} allotype mAb. The frequency of non-transfected, hCD4− cells is indicated by the number in the upper left of each panel in row 1509 and the frequency of transfected, hCD4+ cells is indicated by the number in the upper right of each panel in the row. Transfection efficiency was similar in all cases. The different shaded histograms in all panels in row 1510 indicate negative (1513) and positive (1514) staining by the mouse IgM^{b} allotype mAb, gated on the transfected hCD4+ cells. (Shown as an example in column 1503, row 1510). When canine V_{λ} was linked to mouse C_{κ} (1503, bottom row) IgM expression on the cell surface was less than when the same canine V_{λ} was linked to mouse C_{λ1} or C_{λ2} (1504, 1505, bottom row) Similarly, the canine IgM with V_{κ} was expressed better when linked to C_{κ} (1506, bottom row) than to C_{λ1} or C_{λ2} (1507, 1508, bottom row). The numbers in the upper right of each panel in the bottom row indicate the mean fluorescence intensity (MFI) of the cell surface IgM^{b} staining, which is a quantitative indication of the level of expression.

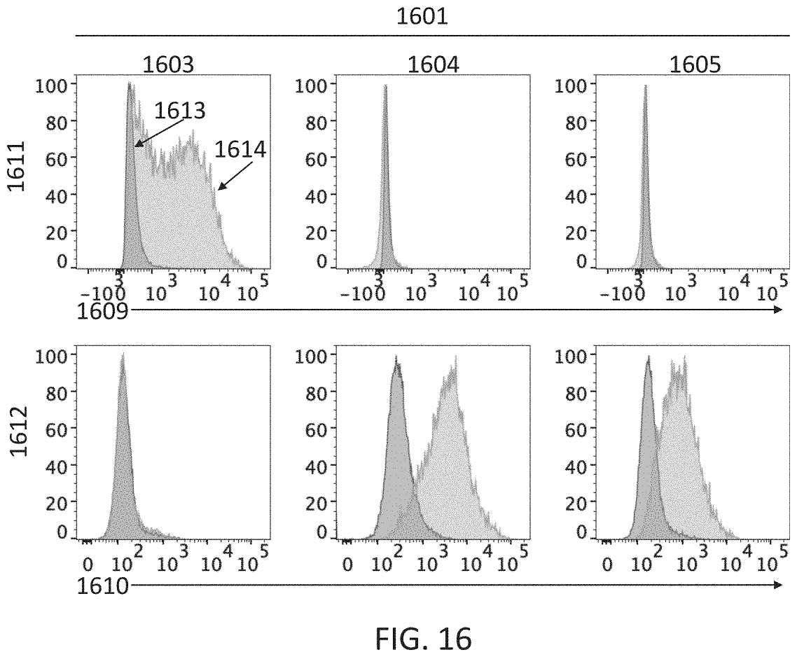

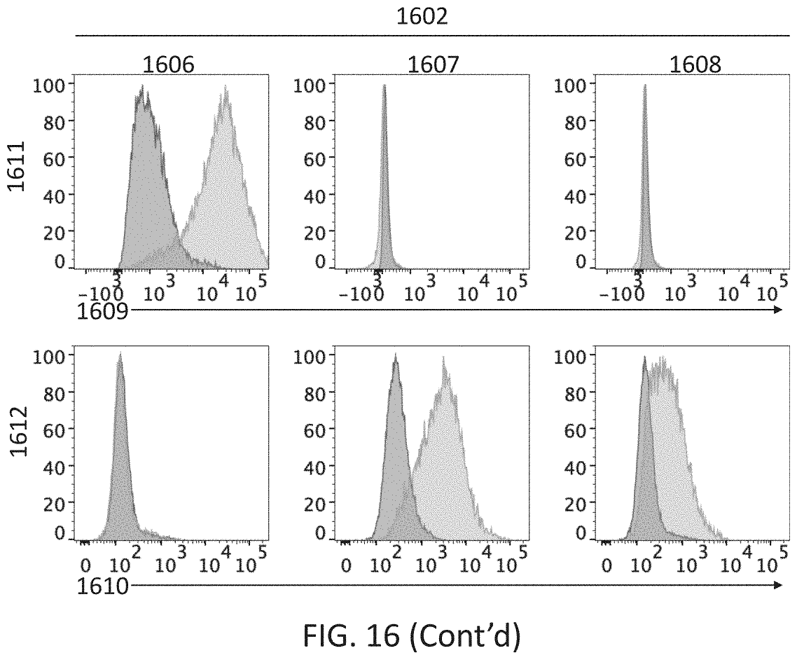

FIG. 16 shows the results of flow cytometry analysis of cells expressing IGHV3-5, which was one of the less stringent V_{H} genes, with canine IGVL3-28/IGLJ6 (1601) or with canine IGVK2-5/IGJK1 (1602). These were the same cells as in FIG. 15 but were stained for cell surface mouse κ LC (1609) or mouse λ LC (1610), confirming the results shown in FIG. 15. The different shaded histograms in all panels in rows 1609 and 1610 indicate negative (1613) and positive (1614) staining by the particular antibody being used in each row, gated on the transfected hCD4+ cells. (Shown as an example in column 1603, row 1609).

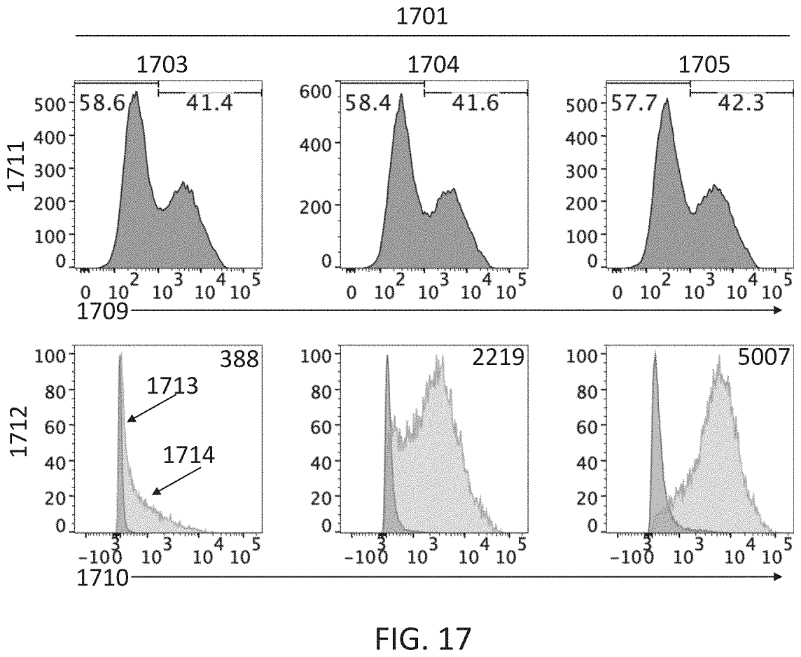

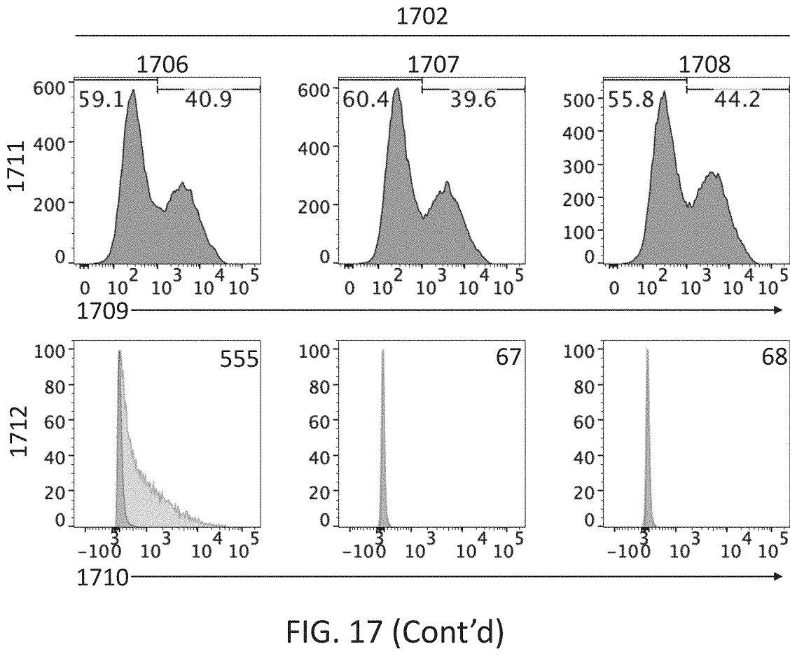

FIG. 17 shows the results of flow cytometry analysis of cells expressing IGHV4-1, which was more stringent than IGHV3-5, with canine IGVL3-28/IGLJ6 (1701) or with canine IGVK2-5/IGJK1 (1702). The top row panels are transfection controls stained with hCD4 mAb antibody (1709) and the bottom panels are stained with mouse IgM^{b} allotype mAb (1710). The frequency of non-transfected, hCD4− cells is indicated by the number in the upper left of each panel in the top row and the frequency of transfected, hCD4+ cells is indicated by the number in the upper right of each panel in the top row. Transfection efficiency was similar in all cases. The different shaded histograms in all panels in row 1710 indicate negative (1713) and positive (1714) staining by the mouse IgM^{b} allotype mAb, gated on the transfected hCD4+ cells. (Shown as an example in column 1703, row 1710). When canine V_{λ} was linked to mouse C_{κ} (1703, bottom row) IgM expression on the cell surface was much less than when the same canine V_{λ} was linked to mouse C_{λ1} or C_{λ2} (1704, 1705, bottom row), although the best expression in this case was with C_{λ2} (1705, bottom row). Similarly, the canine IgM with V_{κ} was expressed much better when linked to C_{κ} (1706, bottom row) than to C_{λ1} or C_{λ2} (1707, 1708, bottom row). In fact, in this case, expression of IgM with C_{λ1} or C_{λ2} was essentially undetectable. The numbers in the upper right of each panel in the bottom row indicate the mean fluorescence intensity (MFI) of the cell surface IgM^{b} staining, which is a quantitative indication of the level of expression. Staining with antibodies specific for mouse λ LC or κ LC was performed in all experiments and confirmed the results of staining with the IgM^{b} allotype mAb (not shown).

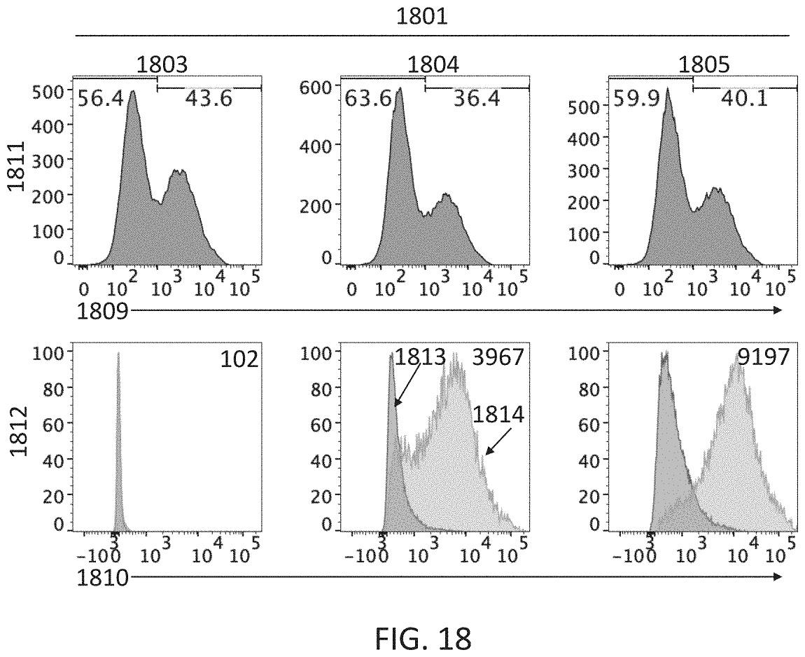

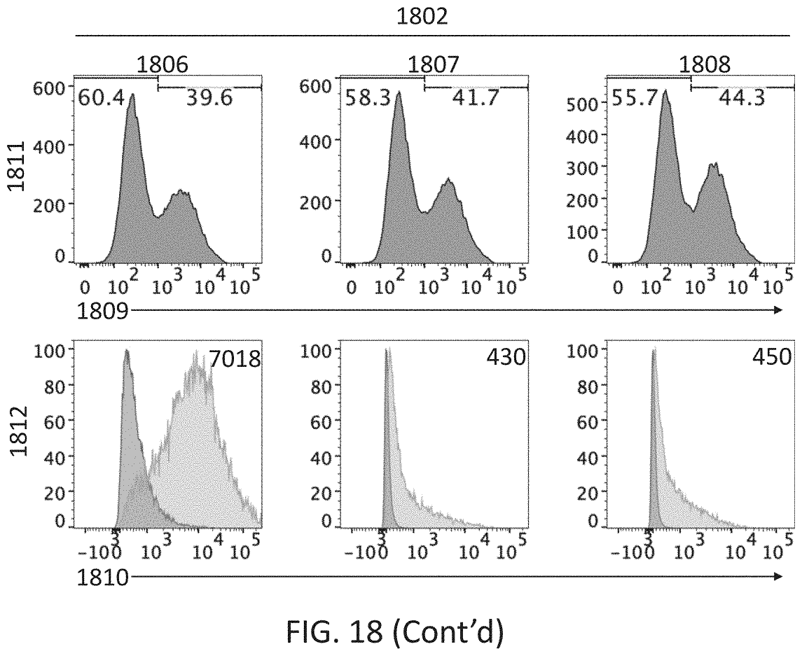

FIG. 18 shows the results of flow cytometry analysis of cells expressing IGHV3-19, which was the most stringent of the IGHV genes tested in terms of the ability of canine V_{λ} to function with mouse C_{κ}, with canine IGVL3-28/IGLJ6 (1801) or with canine IGVK2-5/IGJK1 (1802). Row 1809 panels are transfection controls stained with hCD4 mAb antibody and row 1810 panels are stained with mouse IgM^{b} allotype mAb. The frequency of non-transfected, hCD4− cells is indicated by the number in the upper left of each panel in row 1809 and the frequency of transfected, hCD4+ cells is indicated by the number in the upper right of each panel in the row. Transfection efficiency was similar in all cases. The different shaded histograms in all panels in row 1810 indicate negative (1813) and positive (1814) staining by the mouse IgM^{b} allotype mAb, gated on the transfected hCD4+ cells. (Shown as an example in column 1804, row 1810). There was essentially no surface IgM expression when the canine V), was linked to mouse C_{κ} (1803, bottom row) and only low-level expression when the canine V_{κ} was linked to mouse C_{λ1} or C_{λ2} (1807, 1808, bottom row). The numbers in the upper right of each panel in the bottom row indicate the mean fluorescence intensity (MFI) of the cell surface IgM^{b} staining, which is a quantitative indication of the level of expression. Staining with antibodies specific for mouse λ LC or κ LC was performed in all experiments and confirmed the results of staining with the IgM^{b} allotype mAb (not shown).

The results of this analysis indicate that hybrid light chains that include canine V), and mouse C_{κ} or canine V_{κ} and mouse C_{λ1} or C_{λ2} were often poorly expressed on the cell surface with μHC. The level of cell surface IgM was dependent on the particular V_{H} used by the μHC, but there was no discernable pattern that would allow prediction of whether a particular V_{H} would allow modest or no cell surface IgM expression. Since B cell survival depends on IgM BCR expression, pairing of canine V_{λ} and mouse C_{κ} would result in a major reduction in the development of λLC-expressing B cells. Similarly, pairing of canine V_{κ} with mouse C_{λ1} or C_{λ2} would reduce the development of κ-LC expressing B cells.

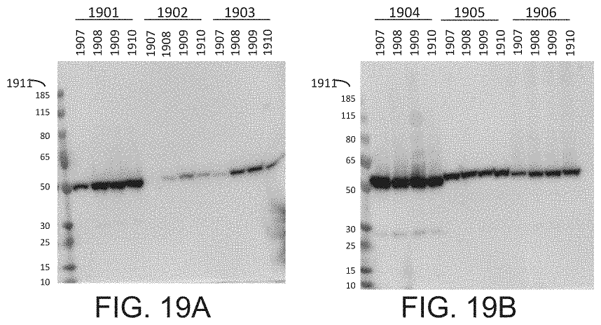

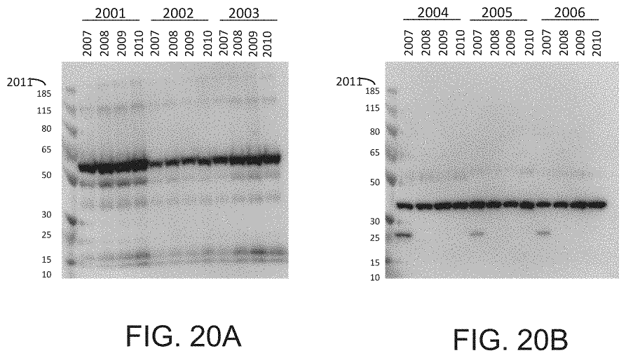

Expression and secretion of the Ig with hybrid or homologous LC was also tested. Supernatants and cell lysates of the transiently transfected cells were analyzed by western blotting. FIG. 19A shows the results of supernatants of cells using canine IGVL3-28 paired with mouse C_{κ}, C_{λ1}, C_{λ2} or C_{λ3} and a mouse IgG2a HC containing canine IGHVH3-5 (1901), IGHVH3-19 (1902) or IGHVH4-1 (1903). FIG. 19B shows the results of lysates of cells using canine IGVL3-28 paired with mouse C_{κ}, C_{λ1}, C_{λ2} or C_{λ3} and a mouse IgG2a HC containing canine IGHVH3-5 (1904), IGHVH3-19 (1905) or IGHVH4-1 (1906). The samples were electrophoresed under non-reducing (not shown) or reducing conditions and the blot was probed with an IgG2a antibody. The amount of IgG2a secreted when canine IGVL3-28 was paired with mouse C_{κ} (1907) was consistently much less than when it was paired with C_{λ1} (1908) C_{λ2} (1909) or C_{λ3} (1910) (FIG. 18A). This difference was not due to lower expression or enhanced degradation of the γ2a HC in the canine IGVL3-28-mouse C_{κ} cells, since the levels were similar in each group of the transfectants (FIG. 19B), or to less protein being analyzed. Loading controls, Myc (FIG. 20A) and GAPDH (FIG. 20B) showed that protein amounts in each group were nearly identical. (The blot used in FIG. 19B was stripped and sequentially reprobed with antibodies to Myc and GAPDH and so the lanes in FIGS. 20A and 20B are identical to FIG. 19B.

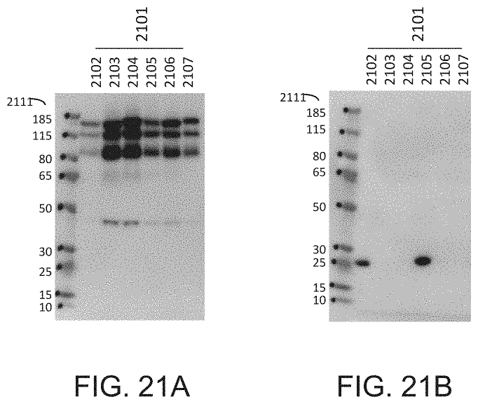

In another set of experiments, the stability of the canine IGVL3-28-mouse C_{κ} LC in transfected cells (FIG. 21B, reducing conditions) was examined in parallel with the secretion analysis (FIG. 21A, non-reducing conditions). Again, much less IgG2a was secreted when the LC was canine IGVL3-28-mouse C_{κ} (FIG. 2A, 2102) than when it was canine IGVL3-28-mouse C_{λ1} (FIG. 2A, 2103) or IGVL3-28-mouse C_{λ2} (FIG. 2A, 2104) However there was a significant amount of intracellular κLC in IGVL3-28-mouse C_{κ} cell lysates detectable with an anti-κ antibody (FIG. 2B, 2102), similar to the levels seen when the LC was canine IGVK2-5-mouse C_{κ} (FIG. 20B, 2105). Thus, the hybrid IGVL3-28-mouse C_{κ} was expressed well and not rapidly degraded intracellularly. In this particular canine VH-VK combination, the secretion of canine IgG2a using VK2-5 was similar when it was attached to V_{κ} (2105), C_{λ1} (2106) or C_{λ2} (2107).

The results in FIGS. 21A and 21B, indicate that the reduced secretion of Ig molecules bearing a hybrid canine V_{λ}-mouse C_{κ} was due to an inability to fold or to pair correctly with the γ2a HC. While not wishing to be bound by theory, it is believed that this results in retention of the incompletely assembled IgG2a molecule in the endoplasmic reticulum (ER) by ER quality control mechanisms such as the Ig HC retention molecule BiP (Haas and Wabl (1983) Immunoglobulin Heavy Chain Binding Protein. Nature 306:387-389 PMID 6417546; Bole, et al. (1986) Posttranslational association of immunoglobulin heavy chain binding protein with nascent heavy chains in nonsecreting and secreting hybridomas. J. Cell Biology 102:1558-1566 PMID 3084497).

### Example 10: Expression of Partly Canine Immunoglobulin with Mouse IgD

IgD is co-expressed with IgM on mature B cells in most mammals. However, the issue of whether dogs have a functional constant region gene to encode the δHC is quite controversial. Early serological studies using a mAb identified an “IgD-like” molecule that was expressed on canine lymphocytes (Yang, et al. (1995) Identification of a dog IgD-like molecule by a monoclonal antibody. Vet. Immunol. and Immunopath. 47:215-224. PMID: 8571542). However, serum levels of this IgD increased upon immunization of dogs with ragweed extract. This is not typical of bona fide IgD, which is present in vanishingly small amounts in serum and is not boosted by immunization; IgD is primarily a BCR isotype, especially in mice. Later, Rogers, et al. ((2006) Molecular characterization of immunoglobulin D in mammals: immunoglobulin heavy constant delta genes in dogs, chimpanzees and four old world monkey species. Immunol. 118:88-100 (doi:10.1111/j.1365-2567.2006.02345.x)) cloned a cDNA by RT-PCR of RNA isolated from dog blood that, by sequence homology, encoded an authentic δHC. However, the most recent annotation of the canine IGH locus by the international ImMunoGeneTics information System®/www.imgt.org, (IMGT) lists Co as a non-functional open reading frame because of a non-canonical splice donor site, NGC instead of NGT, for the hinge 2 exon. It is possible that some low level of correct “leaky” splicing and IgD expression may occur in the dog, thus accounting for the ability of Rogers, et al. to isolate a Cδ cDNA clone. However, the concern was that the canine V_{H} domains might not fold properly when linked to mouse Cδ, since the dog V_{H} gene region has apparently been evolving with a partial or completely non-functional C_{δ} gene. A problem with partial or absent assembly of the partly canine IgD could disturb normal B cell development.

To test whether canine V_{H} domains with a Cδ backbone can assemble into an IgD molecule expressible on the cell membrane, transient transfection and flow cytometry analyses were conducting using methods similar to those described in Example 8.

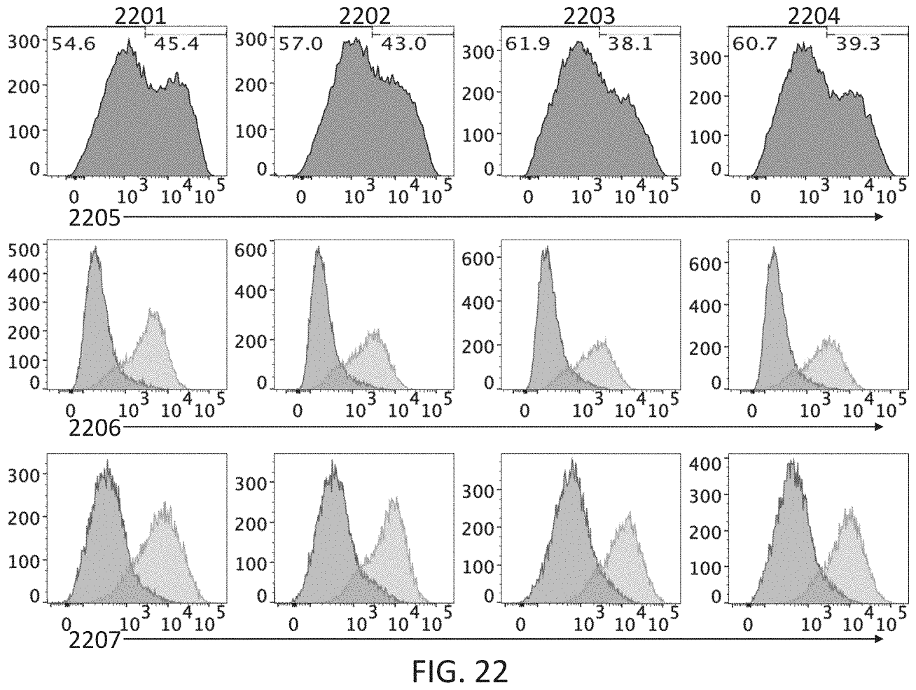

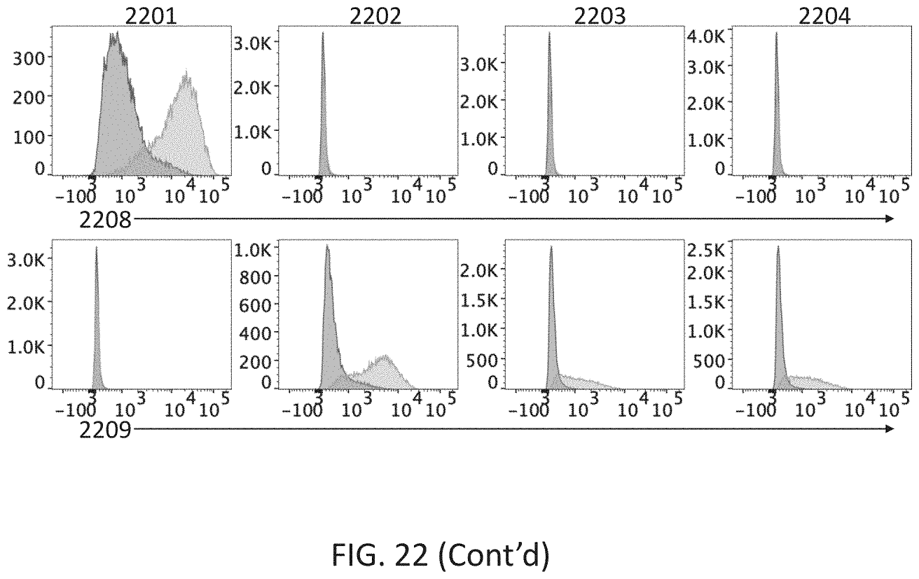

293T/17 cells were co-transfected with a human CD4 (hCD4) expression vector as a transfection control plus one of the HC constructs from Example 8, except that Cμ was replaced with Cδ, and one of the κ or λ LC constructs, along with a CD79a/b expression vector. As can be seen in FIGS. 22-24, the HC with canine VH domains with a mouse IgD backbone were expressed on the cell surface when paired with a canine V_{κ}-mouse C_{κ} or a canine C_{λ}-mouse C_{λ} LC.

FIG. 22 shows expression of cell surface canine IGHV3-5 with a mouse IgD backbone and canine IGKV2-5/IGKJ1-C_{κ} (column 2201) and canine IGLV3-28/IGLJ6 attached to mouse C_{λ1} (2202), C_{λ2} (2203) or C_{λ3} (2204). In these studies, the top row (2205) shows staining for cell surface hCD4, the control for transfection efficiency. Row 2206 shows staining for CD79b, an obligate component of the BCR, which confirms cell surface IgD expression. Row 2207 shows IgD staining, 2208 shows κ LC, and 2209 shows λ LC. These particular canine V_{H}/V_{κ} or V_{H}/V_{λ} LC combinations were expressed well on the cell surface.

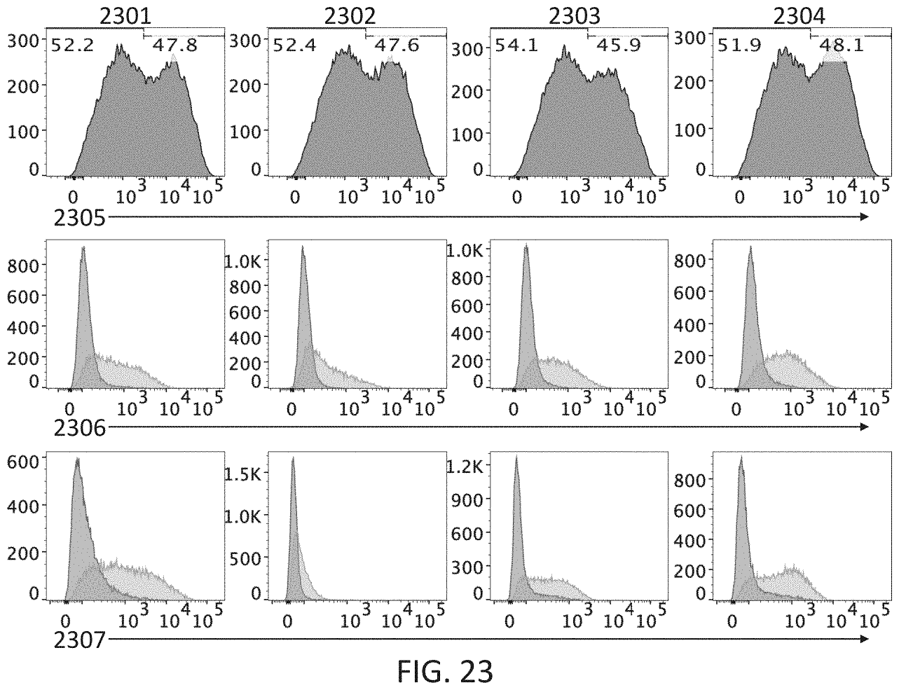

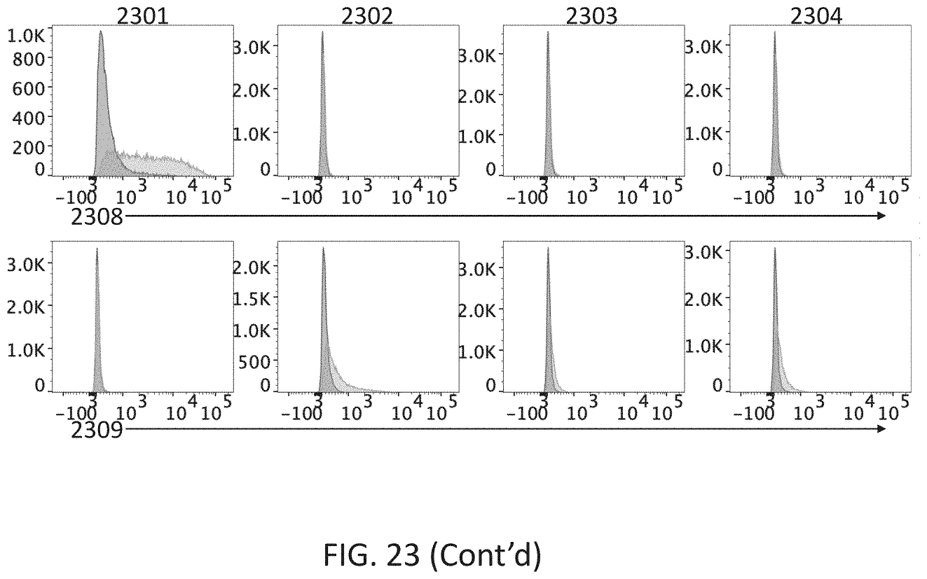

FIG. 23 shows expression of cell surface canine IGHV3-19 with a mouse IgD backbone and canine IGKV2-5/IGKJ1-C_{κ} (column 2301) and canine IGLV3-28/IGLJ6 attached to mouse C_{λ1} (2302), C_{λ2} (2303) or C_{λ3} (2304). (The cell surface staining data is arranged the same as in FIG. 22.) The cell surface expression of IgD with these particular canine V_{H}/V_{κ} or V_{H}/V_{λ} LC combinations was not as high as in FIG. 22. Recall that canine IGHV3-19 was also the most stringent V_{H} in terms of its ability to associate with a canine V_{κ}-mouse C_{λ} LC. (FIG. 19).

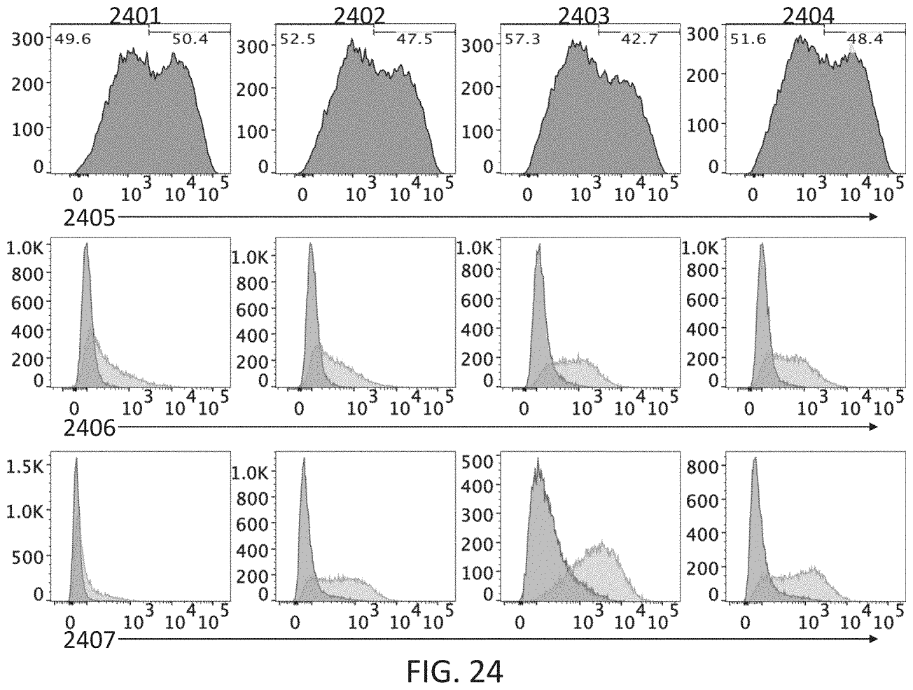

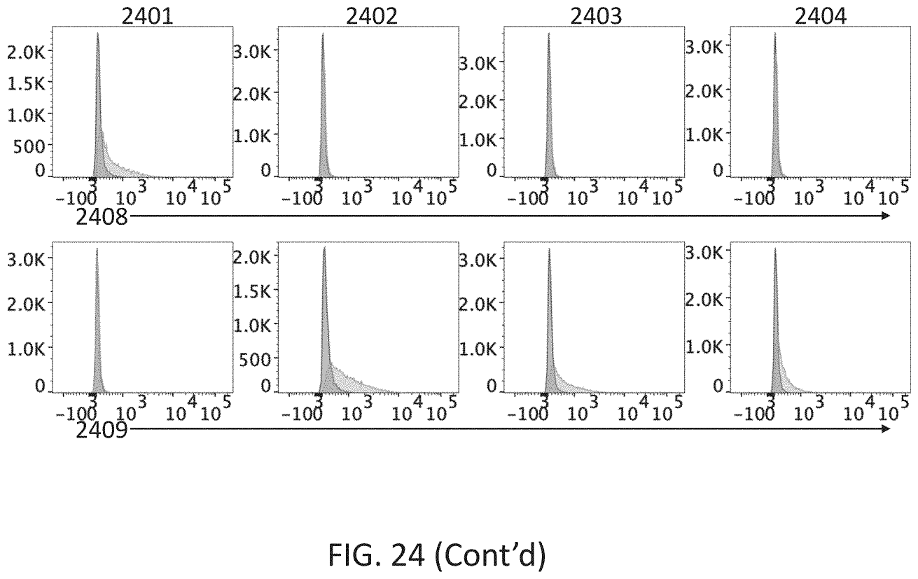

FIG. 24 shows expression of cell surface canine IGHV4-1 with a mouse IgD backbone and canine IGKV2-5/IGKJ1-C_{κ} (column 2401) and canine IGLV3-28/IGLJ6 attached to mouse C_{λ1} (2402), C_{λ2} (2403) or C_{λ3} (2404). (The cell surface staining data is arranged the same as in FIG. 22.) The cell surface expression of IgD with these particular canine V_{H}/V_{κ} or V_{H}/V_{λ} LC combinations was intermediate between that observed in FIG. 22 and FIG. 23.

This data demonstrates that canine V_{H} genes were expressed with a mouse IgD backbone, although the level of cell surface expression varied depending on the particular HC/LC combination. It is believed that HC/LC combinations that can be expressed as IgD on the cell surface are selected into the follicular B cell compartment during B cell development, generating a diverse BCR repertoire.

The preceding merely illustrates the principles of the methods described herein. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims. In the claims that follow, unless the term “means” is used, none of the features or elements recited therein should be construed as means-plus-function limitations pursuant to 35 U.S.C. § 112 ¶6. All references cited herein are incorporated by reference in their entirety for all purposes.

### Sequence Tables

### Canine Ig

(NB, the sequence and annotation of the dog genome is still incomplete. These tables do not necessarily describe the complete canine V_{H}, D and J_{H}, V_{κ}AND J_{κ}, or V_{λ} and J_{λ} gene segment repertoire.)
(F=Functional, ORF=open reading frame, P=pseudogene, *0X indicates the IMGT allele number)

TABLE 1
Canine IGH locus
Germline V_{H} sequences
SEQ ID NO. 1 IGHV1-4-1 (P)
>IGHV1-4-1*01|Canis lupus familiaris_boxer|P|V-REGION|
gaggtccagctggtgcagtctggggctgaggtgaggaaaccagtttcatctgtgaaggtc
tcctggaaggcatctggatacacctacatggatgcttatatgcactggttatgacaagct
tcaggaataaggtttgggtgtatgggatggattggtcccaaagatggtgccacaagatat
tcacagaagttccacagcagagtctccctgatggcagacatgtccaaagcacagcctaca
tgctgctgagcagtcagaggcctgaggacacacctgcatattactgtgtgggacact
SEQ ID NO. 2 IGHV1-15 (P)
>IGHV1-15*01|Canis lupus familiaris_boxer|P|V-REGION|
gaggtccagctggtgcagtctggggctgaggtgaagaagccaggtacatccgtgaaggtc
tcatgcaagacatctggatacaccttcactgactactatatgtactgggtacgacaggct
tcaggagcagggcttgattggatgggacagattggtccctaagatggtgccacaaggtat
gcacagaagtttcagggcagagtcaccctgtcaacagacacatccacaagcacagcctac
atggagctgagcagtctgagagctgaggacacagccatgtactactctgtgaga
SEQ ID NO. 3 IGHV1-17 (P)
>IGHV1-17*01|Canis lupus familiaris_boxer|P|V-REGION|
gaggtccagctggtgcagtctggggctgaggtgaagaagctaagggcatcagtgatagtc
ccctgcaagacatctggatacagcttcactgactacattttggaatgggtatgacaggct
ccaggaccagggcttgagtggatgggatggattggtcctgaagatggtgagacaaagtat
gtgcagaagttccaggcagagtcaccctgatggcagacacaaccacaagcacagccaaca
tggagctgaccagtctgagagctgaggacacagccatgtactactgtgtga
SEQ ID NO. 4 IGHV1-30 (F)
>IGHV1-30*01|Canis lupus familiaris_boxer|F|V-REGION|
gaggtccagctggtgcagtctggggctgaggtgaagaagccaggggcatctgtgaaggtc
tcctgcaagacatctggatacaccttcattaactactatatgatctgggtacgacaggct
ccaggagcagggcttgattggatgggacagattgatcctgaagatggtgccacaagttat
gcacagaagttccagggcagagtcaccctgacagcagacacatccacaagcacagcctac
atggagctgagcagtctgagagctggggacatagctgtgtactactgtgcgaga
SEQ ID NO. 5 IGHV3-2 (F)
>IGHV3-2*01|Canis lupus familiaris_boxer|F|V-REGION|
gaggtgcagctggtggagtctgggggagacctggtgaagcctggggggtccctgagactc
tcctgtgtggcctctggattcaccttcagtagcaactacatgagctggatccgccaggct
ccagggaaggggctgcagtgggtctcacaaattagcagtgatggaagtagcacaagctac
gcagacgctgtgaagggccgattcaccatctccagagacaatgccaagaacacgctgtat
ctgcagatgaacagcctgagagatgaggacacggcagtgtattactgtgcaaggga
SEQ ID NO. 6 IGHV3-3 (F)
>IGHV3-3*01|Canis lupus familiaris_boxer|F|V-REGION|
gaggtgcagctggtggagtctgggggagacatggtgaagcctggggggtccctgagactc
tcctgtgtggcctctggatttaccttcagtagttactacatgtattgggcccgccaggct
ccagggaaggggcttcagtgggtctcacacattaacaaagatggaagtagcacaagctat
gcagacgctgtgaagggccgattcaccatctccagagacaacgcaaagaatacgctgtat
ctgcagatgaacagcctgagagctgaggacacagcggtgtattactgtgcaaagga
SEQ ID NO. 7 IGHV3-4 (P)
>IGHV3-4*01|Canis lupus familiaris_boxer|P|V-REGION|
gaggtgcagctggtggagtctgggggagacctgatgaagcctgggggggtccctgagact
ctcctgtgtggcctctgaattcatcttcagtggctactggaagtactggatccaccaagc
tccagggaaggggctgcagtgggtcacatggattagcaatgatggaagtagcaaaagcta
tgcagacgctgtgaagggccaattcaccatctccaaagacaatgccaaatacacgctgta
tctgcagatgaacagcctgagagccgaggacatggccgtgtattactgtatgatgca
SEQ ID NO. 8 IGHV3-5 (F)
>IMGT000001|IGHV3-5*01|Canis lupus familiaris_boxer|F|V-REGION|
gaggtgcagctggtggagtctgggggagacctggtgaagcctggggggtccctgagactt
tcctgtgtggcctctggattcaccttcagtagctaccacatgagctgggtccgccaggct
ccagggaaggggcttcagtgggtcgcatacattaacagtggtggaagtagcacaagctat
gcagacgctgtgaagggccgattcaccatctccagagacaacgccaagaacacgctgtat
cttcagatgaacagcctgagagccgaggacacggccgtgtattactgtgcgagtga
SEQ ID NO. 9 IGHV3-5-1 (P)
>IMGT000001|IGHV3-5-1*01|Canis lupus familiaris_boxer|P|V-REGION|
gaggtgcagctggtggagtctgggggagccctggtgaagcctgggggggtccctgagact
ctcctatgtggcctctggattcaccttcagtagctaccacatgagctgggtccgccaggc
tccagggaaggggctgcagtgggtcgcatacattaacagtggtggaagtagggatccctg
ggtggcgcagtggtttggcgcctgcctttggcccagggcacgatcctggagacccgggat
cgaatcccacgtcgggctccctgcatggagcctgcttctccctctgcctgtgtctct
SEQ ID NO. 10 IGHV3-6 (F)
>IGHV3-6*01|Canis lupus familiaris_boxer|F|V-REGION|
gaggtgcagctggtggagtctgggggagacctggtgaagcctggggggtccctgagactc
tcctgtgtagcctctggattcaccttcagtagctccgacatgagctggatccgccaggct
ccaggaaaggggcttcagtgggtcgcatacattagcaatgatggaagtagcacaagctac
gcagacgctgtgaagggccgattcaccatctccagagacaacgccaagaacacgctctat
ctgcagatgaacagcctcagagccgaggacacggccgtgtattactgtgcaga
SEQ ID NO. 11 IGHV3-7 (F)
>IGHV3-7*01|Canis lupus familiaris_boxer|F|V-REGION|
gaggagcaactggtggagtttggaggacacatggtgaatcctgggggttccctgggtctc
tcctgtcaggcctctggattcaccttcagtagctatggcatgagctgggtccgccaggct
caaaagaaggggctgcagtgggtcggacatattagctatgatggaagtagtacatactac
gcagacactttgagggacagattcaccatctccagagacaacaccaagaacatgctgtat
ctgcagatgaacagcctgagagccgaggacacagccgtgtattactgcatgaggaa
SEQ ID NO. 12 IGHV3-8 (F)
>IGHV3-8*01|Canis lupus familiaris_boxer|F|V-REGION|
gaggtgcagctggtggagtctgggggagacctggtgaagcctggggggtccctgagactc
tcctgtgtggcctctggattcaccttcagtaactacgaaatgtactgggtccgccaggct
ccagggaaagggctggagtgggtcgcaaggatttatgagagtggaagtaccacatactat
gcagaagctgtaaagggccgattcaccatctccagagacaacgccaagaacatggcgtat
ctgcagatgaacagcctgagagccgaggacacggccgtgtattactgtgcgagtga
SEQ ID NO. 13 IGHV3-9 (F)
>IGHV3-9*01|Canis lupus familiaris_boxer|F|V-REGION|
gaggtgcagctggtggagtctggaggagacctggtgaagcctggggggtccctgagactt
tcctgtgtggcctctggattcaccttcagtagctatgacatggactgggtccgccaggct
ccagggaaggggctgcagtggctctcagaaattagcagtagtggaagtagcacatactac
gcagacgctgtgaagggccgattcaccatctccagagacaacgccaagaacacgctgtat
ctgcagatgaacagcctgagagccgaggacacggccgtgtattactgtgcaaggga
SEQ ID NO. 14 IGHV3-10 (F)
>IGHV3-10*01|Canis lupus familiaris_boxer|F|V-REGION|
gaggtgcagctggtggagactgagggagacctggtgaagcctgggggatccctgagactt
tcctgtgtggcctctggattcaccttcagtagctacgacatggactgggtctaccaggct
ccagggaaagggttacagtgggtcacatacattagcaatggtggaagtagcacaaggtat
gcagacgctgtgaagggccaattcaccatctccagagacaacgccaggaacacgctctat
ctgcagatgaacagcctgagagacaaggacatggccgtgtattactgtgtgagtga
SEQ ID NO. 15 IGHV3-11 (P)
>IMGT000001|IGHV3-11*01|Canis lupus familiaris_boxer|P|V-REGION|
gaggtgcagctggtggagtctaggggagacgtggtgaagcctggggaggtccctctcctg
tgtggcctctagattcaccttcagtagctactacatgggctgggtccactaggctccagg
gaaggggctgcagtgggtcgcaggtattaccaatgatagaagtagcacaagctatgcaga
cgctgtgaagggccgattcaccatctccagagacaatgccaagaacacgctgtatctgca
gatgaacagcctgggagccgaggacacggctgtgtattattgtgtgaaacaga
SEQ ID NO. 16 IGHV3-12 (P)
>IGHV3-12*01|Canis lupus familiaris_boxer|P|V-REGION|
gaggtgcagctggtggagtctggggagacctggtgaagcctggggggtctctgagactct
cctgtgtggcctctggattcaccttcagtagctactacatgagctgggtccgccaggctc
cagggaaggggctgcagtgggtcggatacattaacagtggtggaagtagcacatactatg
cagacgctgtgaagggccgattcaccatctccagagacaatgccaagaacacgctgtatc
tgcagatgaacagcctgagagccgaggacacagctgtgtattactgtgggaaggga
SEQ ID NO. 17 IGHV3-13 (F)
>IGHV3-13*01|Canis lupus familiaris_boxer|F|V-REGION|
gaggagcaactggtggagtttggaggacacatggtgaatcctgggggttccctgggtctc
tcctgtcaggcctctggattcaccttcagtagctatggcatgagctgggtccgccaggct
caaaagaaggggctgcagtgggtcggacatattagctatgatggaagtagcacatactac
acagacactgtgagggacagattcaccatctccagagacaacaccaagaacatgctgtat
ctgcagatgaacagcctgagagccgaggacacagccgtgtattactgcatgaggaa
SEQ ID NO. 18 IGHV3-14 (P)
>IGHV3-14*01|Canis lupus familiaris_boxer|P|V-REGION|
gaggtgcagatggtggagtctgggggagacctggtgaagcctgggggatccctgagactc
tcctgtgtggcctctggattcaccttcagtaactacaaaatgtactgggtccaccaggct
ccagggaaagggctggagtgggtcgcaaggatttatgagagtggaagtaccacatactac
gcagaagctgtaaagggccgattcaccatctccagagacaacgccaagaacatggtgtat
ctgcagatgaacagcctgagagcctaggacacggccgtgtattactgtgtgagtga
SEQ ID NO. 19 IGHV3-16 (F)
>IGHV3-16*01|Canis lupus familiaris_boxer|F|V-REGION|
gaggtacagctggtggagtctggaggagacctggtgaagcctggggggtccctgagactc
tcctgtgtggcctctggattcacctttagtagttactacatgttttggatccgccaggca
ccagggaagggcaatcagtgggtcggatatattaacaaagatggaagtagcacatactac
ccagacgctgtgaagggccgattcaccatctccagagacaacgccaagaacacactgtat
ctgcagatgaacagcctgacagtggaggacacagccctttattactgtgcgagaga
SEQ ID NO. 20 IGHV3-18 (F)
>IGHV3-18*01|Canis lupus familiaris_boxer|F|V-REGION|
gaggtgcagctggtggagtctgggggagaccttgtgaaacctgaggggtccctgagactc
tcctgtgtggtctctggcttcaccttcagtagctacgacatgagctgggtccgccaggct
ccagggaaggggctgcagtgggtcgcatacattagcagtgatggaaggagcacaagttac
acagacgctgtgaagggccgattcaccatctccagagacaatgccaagaacacgctgtat
ctgcagatgaacagcctgagaactgaggacacagccgtgtattactgtgcgaagga
SEQ ID NO. 21 IGHV3-19 (F)
>IGHV3-19*01|Canis lupus familiaris_boxer|F|V-REGION|
gaggtgcagctggtggagtctgggggagacctggtgaagcctgcggggtccctgagactg
tcctgtgtggcctctggattcaccttcagtagctacagcatgagctgggtccgccaggct
cctgagaaggggctgcagttggtcgcaggtattaacagcggtggaagtagcacatactac
acagacgctgtgaagggccgattcaccatctccagagacaacgccaagaacacagtgtat
ctgcagatgaacagcctgagagccgaggacacggccatgtattactgtgcaaagga
SEQ ID NO. 22 IGHV3-20 (P)
>IGHV3-20*01|Canis lupus familiaris_boxer|P|V-REGION|
gaggtgcagctggtggagtctgggggatacctggtgaagcctggagggtcctgagactct
cctctgtgtcctctggattcaccttcagtatctactgcatgtgatgggtctgccaggctc
caggaaaggggctgcagtgagtcgcatacagtaacagtggtggaagtagcactaggtaca
cagacgctgtgaagggctgattcaccacctccagagacaatgccaagaacacactgtatc
tgcagatgaacagcctgagagtgaggacacagcggtgtattactgtgcaggtga
SEQ ID NO. 23 IGHV3-21 (P)
>IGHV3-21*01|Canis lupus familiaris_boxer|P|V-REGION|
gaggtgcagctgttggagtctgggggagacctggtgaagcctggggggtccctgagactg
tcctgtgtggtctctggattcaccttcagtaagtatggcatgagctgggtctgccaggct
ttggggaaggggctacagttggtcgcagctattagctaagatggaaggagcacatactac
acagacactgtgaagggccgattcaccatctccagagacaatgccaagaacacgctgtac
ctgcagatgaacagcttgagagctgaggacacggccgtgtattactgtgagagtga
SEQ ID NO. 24 IGHV3-21-1 (P)
>IGHV3-21-1*01|Canis lupus familiaris_boxer|P|V-REGION|
gaggtgaagctagtggagtctgggggagacctggtgaagcctgggggatcaattagactc
tcctatgtgacctctggattcaccttcaggagctactggatgagctgggtcagccaggct
ccagggaaggggctgcagtgggtcatatgggttaatactggtggaagcagaaaaagctat
gcagatgctgtgaaggggtgattcaccatctccagagacaatgccaagaacacgctgtat
ctgcatatgaacagcctgagagccctgtattattatgtgagtga
SEQ ID NO. 25 IGHV3-22 (P)
>IGHV3-22*01|Canis lupus familiaris_boxer|P|V-REGION|
gaggtgcagatgatggagtctgggggagaactgatgaagcctgcaggatccctgagacct
cctgtgtggcctctggattcaccttcagtagctactggatgtactggatccaccaaactc
cggggaaggggctgcagtgggtcgcaggtattagcacagatggaagtagcacaagctacg
tagacgctctgaagggctgattcaccatctccagagacaacgccaagaacacgctctatc
tgcagatgaacagcctgagagccgaggacatggccatgtattactgtgcaga
SEQ ID NO. 26 IGHV3-23 (F)
>IGHV3-23*01|Canis lupus familiaris_boxer|F|V-REGION|
gaggtgcagctggtggagtctgggggagacctggagaagcctgggggatccctgagactg
tcctgtgtggcctctggattcaccttcagtagctacggcatgagctgggtccgccaggct
ccagggaaggggctgcagggggtctcattgattaggtatgatggaagtagcacaaggtat
gcagacgctgtgaagggccgattcaccatctccagagacaatgccaagaacacgctgtat
ctgcagatgaacagcctgagagccgaggacacagccgtgtattcctgtgcgaagga
SEQ ID NO. 27 IGHV3-24 (F)
>IGHV3-24*01|Canis lupus familiaris_boxer|F|V-REGION|
gaggtgcagctggtggagtctgggggagaccttgtgaagcctgaggggtccctgagactc
tcctgtgtggcctctggattcaccttcagtagcttctacatgagctggttctgccaggct
ccaaggaaggggctacagtgggttgcagaaattagcagtagtggaagtagcacaagctac
gcagacattgtgaagggccgattcaccatctccagagacaatgccaagaacatgctgtat
ctgcagatgaacagcctgagagccgaggacatggccgtatattattgtgcaaggta
SEQ ID NO. 28 IGHV3-25 (P)
>IGHV3-25*01|Canis lupus familiaris_boxer|P|V-REGION|
gaggtgcagctggtggagcctgggggagaactggtgaagcctggggcgtccctgagactc
tcctgtgtggtccctggattcaccttcagtagctacaacatgggctgggctcaccagcct
ccagggaaggggatgcagtgggtcgcaggttttaacagcggtggaagtagcacaagctac
acagatgctgtgaagggtgaattcaccatctccagagacaatgtcaagaacacgctgtat
ctgcagatgaacagcctgagatccgaggacacggccgtgtattactgtgtgaagga
SEQ ID NO. 29 IGHV3-26 (P)
>IGHV3-26*01|Canis lupus familiaris_boxer|P|V-REGION|
gaggtgtagctggtggagtctgggggagacctggtgaagcctggggggtccctgagactc
tcctgtgtgggctctggattcaccttcagtagctactggatgagctgggtccgccaggct
ccagggaaggggctacagtgggttgcagaaattagcggtagtggaagtagcacaaactat
gcagacgctgtgaagggccgattcatcatctccagagacaatgccaagaacacgctgtat
ctgcagatgaacagcctgagagccgaggacacggccatgtattactgtgcaaggga
SEQ ID NO. 30 IGHV3-27 (P)
>IGHV3-27*01|Canis lupus familiaris_boxer|P|V-REGION|
aaggtgcatctggtggagtctgcgggagacgtggtgaagcctaggaggtccctgagactc
tcctgtgtgggctctggattcaccttcagtagctacagcatgtggtgggcccgtgaggct
cccgggatggggctacagggggtcgcaggtattagatatgatggaagtagcacaagctac
gcagacgctctgaagggccgattcaccatctccagagacaatgccaaaaacacactgtat
ctgtagaagaacagcctgagagccgagggaggacacggccgtgtattactgtgcgaggga
SEQ ID NO. 31 IGHV3-28 (P)
>IGHV3-28*01|Canis lupus familiaris_boxer|P|V-REGION|
gaggtgcagctagtggagtctgggggagacctggtgaagtctgggggggtccctgagagt
ctcctgtgtgggctctggattcaccttcagtagctactggatgtactgggtccaccaggc
tccagggaaggggctccatgggtcgcatggattaggtatgatggaagtagcacaagctac
gcagaagctgtgaaaggccgattcactgtttctagagacaacgccaagaacacgctgtat
ctgcagatgaacagcctgagagccgaggacacggccgtgtattactgtgtgaggga
SEQ ID NO. 32 IGHV3-29 (P)
>IGHV3-29*01|Canis lupus familiaris_boxer|P|V-REGION|
gaggtgcagctggtggagtcctggggagacctggtgaagactggaggtttcctgagactc
tcctgtctcctgtgtggcttccggattcaccttcagtaactacagcatgatctgggtccg
ccaggctccaaggaaggggctgcagtggatcacaactattagcaatagtggaagtagcac
aaatcacgcagacacagtaaagggccgatttaccatctccagagacaacaccaagaacac
gctgtatctacagatgagcagcctgggagccgatgacacggccctgtattactgtgtgag
gga
SEQ ID NO. 33 IGHV3-31 (P)
>IGHV3-31*01|Canis lupus familiaris_boxer|P|V-REGION|
gaggtgcagctggtggagtctgggggagaactggtgaagcctggggggtccctgagactc
tcctgtgtggcctctggattcaccttcagtagctactacatgagctggatccgccaggct
cctgggaaggggctgcagtgggtcgcagatattagtgacagtggaggtagcacatactac
actgacgctgtgaagggccgattcaccatctccagagacaacgtcaagaactcgctgtat
ttgcagatgaacagcctgagagccgaggacacggccgtgtattactgtgcgaagga
SEQ ID NO. 34 IGHV3-32 (ORF)
>IGHV3-32*01|Canis lupus familiaris_boxer|ORF|V-REGION|
ggggtgcagctggtggagtctgggggagacctggtgaagcctggggggtccctgacactc
tcctgtgtggcctatggattcaccttcagtagctacagcatgcaatgggtctgtcaggct
ccagggaagggggtgcagtgggtcgcatacattaacagtggtggaagtagcacaagctcc
gcagatgctgtgaagggtcgattcatcatctccagagacaacgtcaagaacacgctatat
ctgcagatgaacagcctgagagccgaggacaccgccgtgtattactgtgcgggtga
SEQ ID NO. 35 IGHV3-33 (P)
>IGHV3-33*01|Canis lupus familiaris_boxer|P|V-REGION|
gagatgcagctggtggaggctgggggagacctggtgaagcttggggggtccctgagactc
ttctgtgtggcctctggatttaccttcagtagctattggatgagctgggtcggccaggct
ccagggaaagggttgcagtgggttgcatacattaacagtggtggaagtagcacatactat
gcagacgctgtgaagggccgattcaccatctccagagacaatgccaagaacacgctgtat
ctgcagatgaactgcctgagagccgaggacacggccgtatattactgtgtggga
SEQ ID NO. 36 IGHV3-34 (F)
>IGHV3-34*01|Canis lupus familiaris_boxer|F|V-REGION|
cagacactgtgaagggccgattcaccatctccagagacaacgccaagaacacgctctatc
tgcagatgaacagcctgagagctgaggacacggccgtgtattactgtgcgaagga
(Incomplete sequence in database)
SEQ ID NO. 37 IGHV3-35 (F)
>IGHV3-35*01|Canis lupus familiaris_boxer|F|V-REGION|| |
gaggtgcagctggtggagtctgggggagacctggtgaagcctgtgggatccctgagactc
tcctgtgtggcctctggattcaccttcagtagctatgacatgaactgggtccgccaggct
ccagggaaggggctgcagtgggtcgcatacattagcagtggtggaagtagcacatactat
gcagatgctgtgaagggccggttcaccatctccagagacaacgccaagaacacgctgtat
cttcagatgaacagcctgagagccgaggacacggccatgtattactgtgcgggtga
SEQ ID NO. 38 IGHV3-36 (P)
>IGHV3-36*01|Canis lupus familiaris_boxer|P|V-REGION|
gaggggcagctggcggagtctgggggagacctggtgaagcctgagaggtccctgagactc
gcccgtgtggcctctggattcaccttcatttcctataccatgagctgggtccacaaggct
cctgggaaggggctgccgtgagtcgcatgaatttattctagtggaagtaacatgagctat
gcagacgctgtgaagggccgattcaccatctccagagacaatgccaagaacatgctgtat
ctgcagatgaacagcctgagagctgaggacatggccatgtattactgtgtgaatga
SEQ ID NO. 39 IGHV3-37 (F)
>IGHV3-37*01|Canis lupus familiaris_boxer|F|V-REGION|
gaggtacagctggtggagtctggggaagatttggtgaagcctggagggtccctgagactc
tcctgtgtggcctctggattcaccttcagtagcagtgaaatgagctgggtccaccaggct
ccagggcaggggctgcagtgggtctcatggattaggtatgatggaagtatctcaaggtat
gcagacactgtgaagggccgattcaccatctccagagacaatgtcaagaacacgctgtat
ctgcagatgaacagcctgagagccgaggacacggccatatattactgtgcaga
SEQ ID NO. 40 IGHV3-38 (F)
>IGHV3-38*01|Canis lupus familiaris_boxer|F|V-REGION|
gaggtgcagctggtggagtctgggggagacctggtgaagcctggggggaccttgagactg
tcctgtgtggcctctggattcacctttagtagctatgacatgagctgggtccgtcagtct
ccagggaaggggctgcagtgggtcgcagttatttggaatgatggaagtagcacatactac
gcagacgctgtgaagggccgattcaccatctccagagacaacgccaagaacacgctgtat
ctgcagatgaacagcctgagagccgaggacacggccgtgtattactgtgcgaagga
SEQ ID NO. 41 IGHV3-39 (F)
>IGHV3-39*01|Canis lupus familiaris_boxer|F|V-REGION|
gaggtacagctggtggaatctgggggagacctcgtgaagcctgggggttccctgagactc
tcctgtgtggcctcgggattcaccttcagtagctactacatgagctggatccgccaggct
cctgggaaggggctgcagtgggtcgcagatattagtgatagtggaggtagcacaggctac
gcagacgctgtgaagggccggttcaccatctccagagagaacgccaagaacaagctgtat
cttcagatgaacagcctgagagccgaggacacagccgtgtattactgtgcgaagga
SEQ ID NO. 42 IGHV3-40 (P)
>IGHV3-40*01|Canis lupus familiaris_boxer|P|V-REGION|
atgcaatgggtccgtcaggctcctgggaagggggtgcagtgggtcgcatacattaacagt
ggtggaagtagcacaagcttcgcagatgctgtgaagggcatgagctggtttcgccaggct
ccagggaaggggctgcaatgggttacatggattgggtatgatggaagtagcacatactac
acagacactgtaaagggccgattcactatctccatagacaacgccaagaacatgctgtat
ctgcagatgaacagcctgagagccgaggacatagccctgtattactgtgcgaggga
SEQ ID NO. 43 IGHV3-41 (F)
>IGHV3-41*01|Canis lupus familiaris_boxer|F|V-REGION|
gaggtgcagctggtggagtctgggggagacctggtgaagcctggggggtccctgagactc
tcctgtgtagcctctggattcaccttcagtaactacgacatgagctgggtccgccaggct
cctgggaaggggctgcagtgggtcgcagctattagctatgatggaagtagcacatactac
actgacgctgtgaagggccgattcaccatctccagagacaacgccaggaacacagtgtat
ctgcagatgaacagcctgagagccgaggacacggctgtgtattactgtgcgaagga
SEQ ID NO. 44 IGHV3-42 (P)
IGHV3-42*01|Canis lupus familiaris_boxer|P|V-REGION|
gaagtgcagctggtggagtctgggggaagacctggtgaagccaggggggtccctgagact
ctcctgtgtgacctctggattcaccttcagtaggtatgccatgagctgggtcggccaggc
tccagggaagggcctgcagtgggttgcagctattagcagtagtggaagtagcacatacta
cgtagatgctgtgaagggccgattcaccatctccatagacaacgccaagaacatggtgta
tctgcagatgaacagcctgagagctgaggatattgctgtgtattactgtgggaagga
SEQ ID NO. 45 IGHV3-43 (P)
>IGHV3-43*01|Canis lupus familiaris_boxer|P|V-REGION|
aaggtgtagctggtggagtctgggggagacctgatgaagcctgggggttccctgagactg
tcctgtgtggcctctggattcaccttcaggagctatggcatgagctgggtctgccaggct
tcagggaaggggctgcagtgggtcgcagctattagctatgatggaaggagcacatactac
acagacactgtgaagggccgattcaccatctccagagacaatggcaagaacacgctgtac
ctgcagatgaacagcttgagagctgaggacacggccgtgtattactgtgcgagtga
SEQ ID NO. 46 IGHV3-44 (ORF)
>IGHV3-44*01|Canis lupus familiaris_boxer|ORF|V-REGION|
gaggtgcagctggtggagtctgggggagacctggtgaagcctgggggttccctgagactc
tcatgtgtgacttctggattcaccttcagtagctattggatgagctgtgtccgccaggct
ccagggaaggagctgcagtgggtcgcgtacattaacagtggtggaagtagcacatggtac
acagacgctgtgaagggtcgattcaccatctccagagacaacgccaagaacacgctgtat
ctgcagatgaacaacctgagagccgaagacacggccgtgtattactgtgcgaggga
SEQ ID NO. 47 IGHV3-45 (P)
>IGHV3-45*01|Canis lupus familiaris_boxer|P|V-REGION|
gaagtacagctgctggagtctgggggagaccgagtgaaacctggggggtcccagagactc
tcctgtgtggcctcaaggttcaccttcagtagctacagcatgcattgtctccgtcagtct
cctgggatggggctacagtgggtcacatacattagcagtaatggaagcagcacatactat
gcagacgctgtgaagggtcgattcaccatctccagagacaaagccaagaacatgctttat
ctacagatgaacagcctgagagctcaggacatagccctgtattactgtgcagatg
SEQ ID NO. 48 IGHV3-46 (F)
>IGHV3-46*01|Canis lupus familiaris_boxer|F|V-REGION|
gaggtacagctggtggagtctggggaagatttggtgaagcctggagggtccctgagactc
tcctgtgtggcctctggattcaccttcagtagcagtgaaatgagctgggtccaccaggct
ccagggcaggggctgcagtgggtctcatggattaggtatgatggaagtagctcaaggtat
gcagacactgtgaagggccgattcaccatctccagagacaatgccaagaacacgctgtat
ctgcagatgaacagcctgagagccgaggacacggccatatattactgtgcaga
SEQ ID NO. 49 IGHV3-47 (F)
>IGHV3-47*01|Canis lupus familiaris_boxer|F|V-REGION|
gaggtgcagctggtggagtctgggggagacctggcgaagcctggggggtccctgagactc
tcctgtgtggcctctggattaaccttcagtagctacagcatgagctgggtccgccaggct
cctgggaaggggctgcagtgggtcacagctattagctatgatggaagtagcacatactac
actgacgctgtgaagggccgattcaccatctccagagacaacgccaggaacacagtgtat
ctgcagatgaacagcctgagagccgaggacacagctgtgtattactgtgtgga
SEQ ID NO. 50 IGHV3-47-1 (P)
>IGHV3-47-1*01|Canis lupus familiaris_boxer|P|V-REGION|
gaggtgccactggtggaatctgggggagagctggtgaagcctggggggtccctgagactc
tcctttgtagcctctgcattcactttcagtagttactggataagctgggtccgccaagct
ccagggaaagggctgcactgagtctcagtaattaacaaagatggaagtaccacataccac
gcagatgctgtgaagggccgattcaccatctccagagacaatgccaagaacacgctgtat
ctgcagatgaacagcctgagagctgaggacacggctgtgtattactgtgcaca
SEQ ID NO. 51 IGHV3-48 (P)
>IGHV3-48*01|Canis lupus familiaris_boxer|P|V-REGION|
gaggagcagttggtgaaatctaggggagacctggtgaagcctggcgggtccctgagactc
ttctgtgagtcctctacattcacctttcatagcaacagcatacattggctccaccagtct
cccggtagtggctacagtgggtcatatccaatagcagtaatggaagtagcatgtactatg
cagacgctgtaaagggctgattcaccatctccagagacagcaccaggaacatgctgtatc
tgcagatgaacagcctgagagctgaggacacagccgtgcattgctgtgcgaggga
SEQ ID NO. 52 IGHV3-49 (P)
>IGHV3-49*01|Canis lupus familiaris_boxer|P|V-REGION|
gaggtgcagctggtggagtctgggggagacctcatgaagcctggggggtccctgagactc
tcctgtgtggccgctggattcaccttcagtagctacagcatgagctgggtccgccaggct
cccgggaaggggattcagtgggtcgcatggatttaagctagtggaaatagcacaagctac
acagatgctgtgaagggccgattcaccatctccagagaacgccaagaacacagtgtttct
gcagatgaacagcctgagagctgaggacaaggccatgtattactgtgcgaggga
SEQ ID NO. 53 IGHV3-50 (F)
>IGHV3-50*01|Canis lupus familiaris_boxer|F|V-REGION|
gaggtgcagctggtggagtctgggggagacctggtgaagcctggggggtccttgagactc
tcctgtgtggcctctggtttcaccttcagtagcaacgacatggactgggtccgccaggct
ccagggaaggggctgcagtggctcacacggattagcaatgatggaaggagcacaggctac
gcagatgctgtgaagggccgattcaccatctccagagacaacgccaagaacacgctgtat
ctgcagatgaacagcctgagagctgaggacacagccgtgtattactgtgcgaagga
SEQ ID NO. 54 IGHV3-51 (P)
>IGHV3-51*01|Canis lupus familiaris_boxer|P|V-REGION|
gaggtgcagctggaggagtctgggggagacctggtgaagcctggggttccctaagactgt
cctgtgtgacctccggattcactttcagtagctatgccatgcactgggtccgccaggctc
cagggaaggggctgcagtgggtcgcagttattagcagggatggaagtagcacaaactacg
cagacgctgtgaagggccgattcaccatctccagagacaacgccaagaacatgctgtatc
tacagatgaacagcctgagagctgaggacacggccatgtattactgtgcgaagga
SEQ ID NO. 55 IGHV3-52 (P)
>IGHV3-52*01|Canis lupus familiaris_boxer|P|V-REGION||
gaagtgcagctggtggagtatgggggagagctggtgaagcctggggggtccctgagactg
tcctgtgtggcctccggattcaccttcagtatctactacatgcactgggtccaccaggct
ccagggaaggggctgcagtggttcgcatgaattaggagtgatggaagtagcacatactac
actgatgctgtgaagggccgattcaccatctccagagacaattccaagaacactctgtat
ctgcagatgaccagcctgagagccgaggacacggccctatattactgtgcgatgga
SEQ ID NO. 56 IGHV3-53 (P)
>IGHV3-53*01|Canis lupus familiaris_boxer|P|V-REGION|
gagatgcagctggtggagtctagggaggcctggtgaagcctggggggtccctgagactct
cctgtgtggaccctggattcaccttcagtagctactggatgtactgggtccaccaggctc
cagggatggggctgcagtggcttgcagaaattagcagtactggaagtagcacaaactatg
cagacgctgtgaggggcccattcaccatctccagagacaatgccaagaacacgctgtacc
tgcaggtgaacagcctgagagccgaagacacggccgtgtattactgtgtgagtga
SEQ ID NO. 57 IGHV3-54 (F)
>IGHV3-54*01|Canis lupus familiaris_boxer|F|V-REGION|
gaggtgcagctggtggagtctgggggagacctgatgaagcctggggggtccctgagactc
tcctgtgtggcctccggattcactatcagtagcaactacatgaactgggtccgccaggct
ccagggaaggggctgcagtgggtcggatacattagcagtgatggaagtagcacaagctat
gcagacgctgtgaagggccgattcaccatctccagagacaatgccaagaacacgctgtat
ctgcagatgaacagcctgagagccgaggacacggccgtgtattactgtgtgaaggga
SEQ ID NO. 58 IGHV3-55 (P)
>IGHV3-55*01|Canis lupus familiaris_boxer|P|V-REGION|
gaggtgcagctggtggagtctggggaaacctggtgaagcctggggagtctctgagactct
cttgtgtggcctctggattcaccttcagtagctactggatgcattgggtctgccaggctc
cagggaaagggttggggtgggttgcaattattaacagtggtggaggtagcacatactatg
cagacacagtgaagggccaattcaccatcttcagagacaatgccaagaacatgctgtatc
tgcagatgaacagcctgagagcccaggacatgaccgcgtattactgtgtgagtga
SEQ ID NO. 59 IGHV3-56 (P)
>IGHV3-56*01|Canis lupus familiaris_boxer|P|V-REGION|
gaggtgcagctggtggaatctgggggagacctggtgaagcctgggggatccctgagactc
tcctgtgtggcctctggattcaccttcagtagctactatatggaatgggtctgccaggct
ccagggaggggctgaagtgggtcgcacggattagcagtgacggaagtagcacatactaca
cagacgctgtgaagggccgattcaccatctccagagacaatgccaagacggccgtgtatt
actgtgcgaagga
SEQ ID NO. 60 IGHV3-57 (P)
>IGHV3-57*01|Canis lupus familiaris_boxer|P|V-REGION|
gaagtgcagcttgtggagtctgggggagagctggtgaagcctgggggttccctgagactg
tcctgtgtggcctctggattcaccttcagtagctactacatgcactgggtctgcaggctc
cagggaaggggctgcagtgggttgcaagaattaggagtgatggaagtagcacaagctacc
cagacgctgtgaagggcagattcaccatctccagagacaattccaagaacactctgtatc
tgcagatgaacagcctgagagctgatgatacggccctatattactgtgcaaggga
SEQ ID NO. 61 IGHV3-58 (F)
>IGHV3-58*01|Canis lupus familiaris_boxer|F|V-REGION|
gaggtgcagctggtggagtctgggggagacctggtgaagcctgggggatccctgagactc
tcttgtgtggcctccggattcaccttcagtagccatgccaagagctgggtccgccaggct
ccagggaaggggctgaagtgggtagcagttattagcagtagtggaagtagcacaggctcc
gcagacactgtgaagggccgattcaccatctccagagacaatgccaagaacacgctgtat
ctgcagatgaacagcctgagagctgaggacacagccgtgtattactgtgcgaagga
SEQ ID NO. 62 IGHV3-59 (P)
>IGHV3-59*01|Canis lupus familiaris_boxer|P|V-REGION|
gaggtacagctggtggagtctggaggagaccttgtgaagactgagcggtccctgagactc
tcctgtgtggcctctggattcaccttcagtagcttctacatgaggtgtctgccagactcc
agggaagggactacagtgggttgcagaaattagcagtagtggaagtagcacaagctacac
agatgctctgaagggctgattctccatctccaaaaacaatgccaagaacacgctgtatct
gcagatgaacagcctgagagccgaggtcacagccgtatattactgtgcaaggta
SEQ ID NO. 63 IGHV3-60 (P)
>IGHV3-60*01|Canis lupus familiaris_boxer|P|V-REGION|
gaggtgaagctggtggagtctgggggagacctgttgaagcctgggggatcaattaaactc
tcctatgtgacctctggattcaccttcaggagctactggatgagctgggtcagccaggct
ccagggaaggggctgcagtgggtcacatgggttaatactggtggaagcagcaaaagctat
gcagatgctgtgaaggggcaattcaccatctccagagacaatgccaagaacacgctgtat
ctgcatatgaacagcctgatagccctgtattattgtgtgagtga
SEQ ID NO. 64 IGHV3-61 (F)
>IGHV3-61*01|Canis lupus familiaris_boxer|F|V-REGION|
gaggtgcagctggtggagtctggtggaaacctggtgaagcctgggggttccctgagactg
tcctgtgtggcctctggattaaccttctatagctatgccatttactgggtccacgaggct
cctgggaaggggctgcagtgggtcgcagctattaccactgatggaagtagcacatactac
actgacgctgtgaagggccgattcaccatctccagagacaatgccaagaacacgctgtat
ctgcagatgaacagcctgagagctgaggacatgcccgtgtattactgtgcgaggga
SEQ ID NO. 65 IGHV3-62 (P)
>IGHV3-62*01|Canis lupus familiaris_boxer|P|V-REGION|
gaggagcagctggtggagtctcggggagatctggtgaagtctggggggtccctgagactc
tcctgtgtggccccttgattcaccttcagtaactgtgacatgagctgggtccattaggct
ccaggaaagggctgcagtgtgttgcatacattagctatgatggaagtagcacaggttaca
aagacgctgtgaagggccgattcaccatctccagagacaacgccaagaacatgctgtatc
ttcagatgaacagcctgagagctgaggacacggctctgtattactgtgcaga
SEQ ID NO. 66 IGHV3-63 (P)
IGHV3-63*01|Canis lupus familiaris_boxer|P|V-REGION|
gaggagcagttggtgaaatctaggggagacctggtgaagcctggcgggtccctgagactc
ttctgtgagtcctctacgttcacctttcatagctacagcatgcattggctccaccagtct
cccggtagtggctacagtgggtcatatccaatagcagtaatggaagtagcatgtactatg
cagacgctgtaaagggctgatacaccatctccagagacaacaccaggaacatgctgtatc
tgcagatgaataacctgagagctgaggacacagccgtgcattgctgtgcgaggga
SEQ ID NO. 67 IGHV3-64 (P)
>IGHV3-64*01|Canis lupus familiaris_boxer|P|V-REGION|
gaggtgcagctggtggagtctgcgggagaccccgtgaagcctggggggtccctgagactc
tcctgtgtggccgctggattcaccttcagtagctacagcatgagctgggtccgccaggct
cccgggaaggggatgcagtgggtcgcatggatatatgctagcggaagtagcacaagctac
gcagacgctgtgaagggccgattcaccatctccagagacaacgccaagaacacactgttt
ctgcagatgcctgagagctgaggacacggccatgtattcctgtgcagggga
SEQ ID NO. 68 IGHV3-65 (P)
>IGHV3-65*01|Canis lupus familiaris_boxer|P|V-REGION|
gatgtacagctggtggagtctgggggagacctggtgaagcctggggggtccctgagactg
tcctgtgtggcctctggattcacctgcagtagctactacatgtactagacccaccaaatt
ccagggaaggggatgcagggggttgcacggattagctatgatggaagtagcacaagctac
accgacgcaatgaaaggccgattcaccatctccagagacaacgccaagaacatgctgtat
ctgcaatgaacagcctgagagccgaggacacagccgtgtattactgtgtgaagga
SEQ ID NO. 69 IGHV3-66 (P)
>IGHV3-66*01|Canis lupus familiaris_boxer|P|V-REGION|
gaggtgcagctggtggagtctggcggagacctggtgaagcctgggcggtccctgagactg
tcctgtatggcctctggattcacttcagtagctacagcatgagctgtgtccgccaggctc
ctgggaagggctgcagtgggtcgcaaaaattagcaatagtggaagtagcacatactacac
agatgctgtgaagggccgattcaccatctccagagacaatgccaagaacacgctctatct
gcagatgaacagcctgagagccgaggacacggccttgtattactgtgcaga
SEQ ID NO. 70 IGHV3-67 (F)
>IGHV3-67*01|Canis lupus familiaris_boxer|F|V-REGION|
gaggtgcagctggtggagtctgggggagacctggtgaagcctggggggtccctgagactg
tcctgtgtggcctctggattcaccttcagtagctactacatgtactgggtccgccaggct
ccagggaaggggcttcagtgggtcgcacggattagcagtgatggaagtagcacatactac
gcagacgctgtgaagggccgattcaccatctccagagacaatgccaagaacacgctgtat
ctgcagatgaacagcctgagagccgaggacacggctatgtattactgtgcaaagga
SEQ ID NO. 71 IGHV3-68 (P)
>IGHV3-68*01|Canis lupus familiaris_boxer|P|V-REGION|
gaagtgcagctggtggagtctgggggagagctggtgaagcctggggggtccctgagactc
tcctgtgtggcctctggattcaccttcagtagctactacatgtactgggtccgccaggct
ccagggaaatggctgctgtgggtcacatgaattaggagtgatggaagtagcacatataca
ctgatgctgtgaaggaccgatacaccatctccaaagacaattccaagaacattctgtatc
tgcagatgaacagcctgagagccaaggacacggccctatatccctgtgcaatgga
SEQ ID NO. 72 IGHV3-69 (F)
>IGHV3-69*01|Canis lupus familiaris_boxer|F|V-REGION|
gaggtacagctggtggagtctgggggagacctggtgaagcctgggggatccctgagactg
tcctgtgtggcctctggattcaccttcagtagctatgccatgagctgggtccgccaggct
ccagggaaggggctgcagtgggtcgcatacattaacagtggtggaagtagcacatactac
gcagatgctgtgaagggccggttcaccatctccagagacaatgccaggaacacactgtat
ctgcagatgaacagcctgagatccgaggacacagccgtgtattactgtccgaagga
SEQ ID NO. 73 IGHV3-70 (F)
>IGHV3-70*01|Canis lupus familiaris_boxer|F|V-REGION|
gaggtgcagctggtggagtctggaggagaccttgtgaagcctgagcggtccctgagactc
tcctgtgtggcctctggattcaccttcagtagcttctacatgagctggttctgccaggct
ccagggaaggggctacagtgtgttgcagaaattagcagtagtggaaatagcacaagctac
gcagacgctgtgaagggccgattcaccatctccagagacaacgccaagaacacgctgtat
ctacggatgcacagcctgagagccgaggacacggctgtatattactgtgcaaggta
SEQ ID NO. 74 IGHV3-71 (P)
>IGHV3-71*01|Canis lupus familiaris_boxer|P|V-REGION|
gaggtgaagctggtggagtgtgggggagacctggtgaagcccgggggatcgattagactc
tcctttgtgacctctggattcaccttcaggagctattggatgggctgtgtcagccaggct
ccagggaaggggctgcagtgggtcacatgggttaatactggtggaagcagcaaaagctat
gcagatgctatgaaggggcgatttaccatctccaggcacaaagccaagaacacactatct
gcatatgaacagcctgagagccgtgtattattgtgtgagtga
SEQ ID NO. 75 IGHV3-72 (P)
>IGHV3-72*01|Canis lupus familiaris_boxer|P|V-REGION|
gaggtgcagctggtggagtctggcggagacctggtgaagcctggggattccctgagactg
tcctgtgtggcctctggattcaccttcagtagctatgccatgagctgggtccgccaggct
cctaggaaggggctgcagtgggtcggatacattagcagtgatggaagtagcacataatac
gcagacgctgtgaagggccgattcaccatttccagagacaatgccaagaacacgctgtat
ctgcagatgaacagcctgagagctgaggatacggccctgtataactgtgcaaggga
SEQ ID NO. 76 IGHV3-73 (P)
>IGHV3-73*01|Canis lupus familiaris_boxer|P|V-REGION|
gaggtgcagctgatggagtctgggggagacctggtgaagcctggggggtccctgagactc
tcctgtgtggcccctggattcaccttcagtaactatgacatgagctcggtccattagact
ccaggaaagggctgcagtgtattgcatatattagctatgatggaagtagcacaggttaca
aagacgctgtgcagggccgattcaccatctccagagacaacgccaagaacacgctgtatc
ttcagatgaacagcctgagagctgagcacacggccctgtattactgtgcaga
SEQ ID NO. 77 IGHV3-74 (P)
>IGHV3-74*01|Canis lupus familiaris_boxer|P|V-REGION|
gaggtgcagctggtggagtctgggggagacttggtgaagccttgtgggctcctgagactc
tcctgtgtggcttctggattcaccttcagtagctacatcatgagctgggtccgccaggct
ccagggaagtggctgcagtgggtcgcatacattaacagtggtggaagtagcacaaggtac
acagatgctgtgaagggccgattcacctctccagagacaacgccaagaacatgctgtatc
tgcagttgaacagcctgagagccgaggacaccgctgtgtattactgtgcgaggga
SEQ ID NO. 78 IGHV3-75 (F)
>IGHV3-75*01|Canis lupus familiaris_boxer|F|V-REGION|
gaattgcagctggtggagcttgggggagatctggtgaagccaggggggtccctgagactc
tcctgtgtggcctctggattcaccttcagtagctatgccatgagttgggtctgccaggct
ccagggaaggggctgcagtgggttgcagctattagcagtagtggaagtagcacataccat
gtagacgctgtgaagggccgattcaccatctccagagacaacgccaagaacacagtgtat
ctgcagatgaacagcctgagagccgaggacacggccgtgtattactgtgcaga
SEQ ID NO. 79 IGHV3-76 (F)
>IGHV3-76*01|Canis lupus familiaris_boxer|F|V-REGION|
gaggtgccactggtggaatctgggggagagctggtgaagcctgaggggtccctgagattc
tcctgtgtagcctctggattcactttcagtagttactggataagctgggtccgccaagct
ccagggaaagggctgcactgggtctcagtaattaacaaagatggaagtaccacataccac
gcagatgctgtgaagggccgattcaccatctccagagacaatgccaagaacacgctgtat
ctgcagatgaacagcctgagagctgagggcacgactgtgtattactgtgcaca
SEQ ID NO. 80 IGHV3-77 (P)
>IGHV3-77*01|Canis lupus familiaris_boxer|P|V-REGION|
gaggagcagttggtgaagtctgggggagacctggtgaagcttggcaggtccctgagtcct
ctacattcacctttcatagctacagcatgcattggctccaccagtctcccggtagtggct
acagtgggtcatatccaatagcagtaatggaagtagcatgtactatgcagacgctgtaaa
gggttgattcaccatctccagagacaacaccaggaacacgctgtatctgcagatgaacag
cctgagagccgacgacacggccgtgtgttgctgtgcgaggga
SEQ ID NO. 81 IGHV3-78 (P)
>|IGHV3-78*01|Canis lupus familiaris_boxer|P|V-REGION|
gaggtgcagctggtggagtctgggggagaccttgtgaagccggaggggtccctgagactc
tcctgtgtggccgctggattcacctttagtagctacagcatgagctgggtccgccaggct
cccgggaagggggtgcagtgggtcacatagatttatgctagtggaagtagcacaagctac
acagatgctgtgaagggccgattcaccatctccagagacaacgccaagaacacagtgttt
ctgcagatgaacagcctgagagctgagaacacggccatgtattcctgtgcaaggga
SEQ ID NO. 82 IGHV3-79 (P)
>IGHV3-79*01|Canis lupus familiaris_boxer|P|V-REGION|
tggggaattccctctggtgtggcctctggattcacctgcagtagctccctcacctccctc
tcctgtgtggcctctagattcaccttcagtagctactacatatactgtatccaccaagct
ccagggaaggggctgcaggtggtcgcatggattagctatgatggaagtagaacaagctac
gccgacgctatgtagggccaattcatcatctccagagaaaacaccaagaacacgctgtat
ctgtagatgaacagcctgagtgccaaggacacggcactatatccctgtgcgaggaa
SEQ ID NO. 83 IGHV3-80 (F)
>IGHV3-80*01|Canis lupus familiaris_boxer|F|V-REGION|
gaggtgcagctggtggagtctgggggagatctggtgaagcctgggggatccctgagactc
tcttgtgtggcctctggattcaccttcagtagctactacatggaatgggtccgccaggct
ccagggaaggggctgcagtgggtcgcacagattagcagtgatggaagtagcacatactac
ccagacgctgtgaagggtcaattcaccatctccagagacaatgccaagaacacgctgtat
ctgcagatgaacagcctgggagccgaggacacggccgtgtattactgtgcaaagga
SEQ ID NO. 84 IGHV3-81 (F)
>IGHV3-81*01|Canis lupus familiaris_boxer|F|V-REGION|
gaggtgcagctggtggagtctggaggaaacctggtgaagcctggggggtccctgagactc
tcttgtgtggcctctggattcaccttcagtagctactacatggactgggtccgccaggct
ccagggaagaggctgcagtgggtcgcagggattagcagtgatggaagtagcacatactac
ccacaggctgtgaagggccgattcaccatctccagagacaacgccaagaacacgctctat
ctgcagatgaacagcctgagagccgaggactctgctgtgtattactgtgcgatgga
SEQ ID NO. 85 IGHV3-82 (F)
>IGHV3-82*01|Canis lupus familiaris_boxer|F|V-REGION|
gaggtgcagctggtggagtctggaggagacctggtgaagtctggggggtccctgagactc
tcttgtgtggcctctggattcaccttcagtagctactacatgcactgggtccgccaggct
acagggaaggggctgcagtgggtcacaaggattagcaatgatggaagtagcacaaggtac
gcagacgccatgaagggccaatttaccatctccagagacaattccaagaatacgctgtat
ctgcagatgaacagccagagagccgaggacatggccctatattactgtgcaaggga
SEQ ID NO. 86 IGHV3-83 (P)
>IGHV3-83*01|Canis lupus familiaris_boxer|P|V-REGION|
gagttgcagctggtagagtctgggggagacctggtgaagcctggggggtctctgagactt
tcttgtgtgtcctctggattcaccttcagtagctactggatgcactgggtcctccaggct
ccagggaaagggctggagtgggtcgcaattattaacagtggtggaggtagcatatactac
gcagacacagtgaagggccgattcaccatctccagagaaaacgccaagaacacgctctat
ctgcagatgaacagcctgagagctgaggacagggccatgcattactgtgcgaaggga
SEQ ID NO. 87 IGHV4-1 (F)
>IGHV4-1*01|Canis lupus familiaris_boxer|F|V-REGION|
gaactcacactgcaggagtcagggccaggactggtgaagccctcacagaccctctctctc
acctgtgttgtgtccggaggctccgtcaccagcagttactactggaactggatccgccag
cgccctgggaggggactggaatggatggggtactggacaggtagcacaaactacaacccg
gcattccagggacgcatctccatcactgctgacacggccaagaaccagttctccctgcag
ctgagctccatgaccaccgaggacacggccgtgtattactgtgcaagaga
SEQ ID NO. 88 IGHV(II) -1 (P)
>IGHV(II)-1*01|Canis lupus familiaris_boxer|P|V-REGION|
ctggcacccctgcaggagtctgtttctgggctggggaaacccaggcagatccttacactc
acctgctccttctctgggttcttattgagcatgtcagtatgggtgtcacatgggtccttt
acccaccaggggaaggcactggagtcaatgccacatctggtgggagaacgctaagtacca
cagcctgtctctgaacagcagcaagatgtatagaaagtccaacacttggaaagataaagg
attatgtttcacaccagaagcacatctattcaacctgatgaacagccagcctgat
SEQ ID NO. 89 IGHV(II) -2 (P)
>IGHV(II)-2*01|Canis lupus familiaris_boxer|P|V-REGION|
ctggcacccctgcaggagtctgtttctgggctggggaaacccaggcagacccttacactc
acctgctccttctctgggttcttattgagcatgtcagtgtgggtgtcacatgggtccttt
acccaccaggggaaggcactggagtcaatgccacgtctggtgggagaacactaagtacca
cagcctgtctctgaacagcagcaagatgtatagaaagtccaacacttggaaagataaagg
attatgtttcacaccagaagcacatctattcaacctgatgaacaatcagcctgatgaga
Germline D sequences
SEQ ID NO. 90 IGHD1 (F)
>IGHD1*01|Canis lupus familiaris_boxer|F|D-REGION|
gtactactgtactgatgattactgtttcaac
SEQ ID NO. 91 IGHD2 (F)
>IGHD2*01|Canis lupus familiaris_boxer|F|D-REGION|
ctactacggtagctactac
SEQ ID NO. 92 IGHD3 (F)
>IGHD3*01|Canis lupus familiaris_boxer|F|D-REGION|
tatatatatatggatac
SEQ ID NO. 93 IGHD4 (F)
>IGHD4*01|Canis lupus familiaris_boxer|F|D-REGION|
gtatagtagcagctggtac
SEQ ID NO. 94 IGHD5 (ORF)
>IGHD5*01|Canis lupus familiaris_boxer|ORF|D-REGION|
agttctagtagttggggct
SEQ ID NO. 95 IGHD6 (F)
>IGHD6*01|Canis lupus familiaris_boxer|F|D-REGION|
ctaactggggc
Germline J_{H} sequences
SEQ ID NO. 96 IGHJ1 (ORF)
>IGHJ1*01|Canis lupus familiaris_boxer|ORF|J-REGION|
tgacatttactttgacctctggggcccgggcaccctggtcaccatctcctcag
SEQ ID NO. 97 IGHJ2 (F)
>IGHJ2*01|Canis lupus familiaris_boxer|F|J-REGION|
aacatgattacttagacctctggggccagggcaccctggtcaccgtctcctcag
SEQ ID NO. 98 IGHJ3 (F)
>IGHJ3*01|Canis lupus familiaris_boxer|F|J-REGION|
caatgcttttggttactggggccagggcaccctggtcactgtctcctcag
SEQ ID NO. 99 IGHJ4 (F)
>IGHJ4*01|Canis lupus familiaris_boxer|F|J-REGION|
ataattttgactactggggccagggaaccctggtcaccgtctcctcag
SEQ ID NO. 100 IGHJ5 (F)
>IGHJ5*01|Canis lupus familiaris_boxer|F|J-REGION|
acaactggttctactactggggccaagggaccctggtcactgtgtcctcag
SEQ ID NO. 101 IGHJ6 (F)
>IGHJ6*01|Canis lupus familiaris_boxer|F|J-REGION|
attactatggtatggactactggggccatggcacctcactcttcgtgtcctcag

TABLE 2
Canine IGK Sequence Information
Germline Vκ sequences
SEQ ID NO. 102 IGKV2-4 (F)
>IGKV2-4*01|Canis lupus familiaris_boxer|F|V-REGION|
gatattgtcatgacacagacgccaccgtccctgtctgtcagccctagagagacggcctcc
atctcctgcaaggccagtcagagcctcctgcacagtgatggaaacacctatttggattgg
tacctgcaaaagccaggccagtctccacagcttctgatctacttggtttccaaccgcttc
actggcgtgtcagacaggttcagtggcagcgggtcagggacagatttcaccctgagaatc
agcagagtggaggctaacgatactggagtttattactgcgggcaaggtacacagcttcct
cc
SEQ ID NO. 103 IGKV2-5 (F)
>IGKV2-5*01|Canis lupus familiaris_boxer|F|V-REGION|
gatattgtcatgacacagaccccactgtccctgtccgtcagccctggagagccggcctcc
atctcctgcaaggccagtcagagcctcctgcacagtaatgggaacacctatttgtattgg
ttccgacagaagccaggccagtctccacagcgtttgatctataaggtctccaacagagac
cctggggtcccagacaggttcagtggcagcgggtcagggacagatttcaccctgagaatc
agcagagtggaggctgatgatgctggagtttattactgcgggcaaggtatacaagatcct
cc
SEQ ID NO. 104 IGKV2-6 (F)
>IIGKV2-6*01|Canis lupus familiaris_boxer|F|V-REGION|
gatattgtcatgacacagaccccactgtccctgtctgtcagccctggagagactgcctcc
atctcctgcaaggccagtcagagcctcctgcacagtgatggaaacacgtatttgaactgg
ttccgacagaagccaggccagtctccacagcgtttaatctataaggtctccaacagagac
cctggggtcccagacaggttcagtggcagcgggtcagggacagatttcaccctgagaatc
agcagagtggaggctgacgatactggagtttattactgcgggcaaggtatacaagatcct
cc
SEQ ID NO. 105 IGKV2-7 (F)
>IGKV2-7*01|Canis lupus familiaris_boxer|F|V-REGION||
gatattgtcatgacacagaacccactgtccctgtccgtcagccctggagagacggcctcc
atctcctgcaaggccagtcagagcctcctgcacagtaacgggaacacctatttgaattgg
ttccgacagaagccaggccagtctccacagggcctgatctataaggtctccaacagagac
cctggggtcccagacaggttcagtggcagcgggtcagggacagatttcaccctgagaatc
agcagagtggaggctgacgatgctggagtttattactgcatgcaaggtatacaagctcct
cc
SEQ ID NO. 106 IGKV2-8 (F)
>IGKV2-8*01|Canis lupus familiaris_boxer|F|V-REGION|
gatattgtcatgacacagaccccaccgtccctgtccgtcagccctggagagccggcctcc
atctcctgcaaggccagtcagagcctcctgcacagtaacgggaacacctatttgaattgg
ttccgacagaagccaggccagtctccacagggcctgatctatagggtgtccaaccgctcc
actggcgtgtcagacaggttcagtggcagcgggtcagggacagatttcaccctgagaatc
agcagagtggaggctgacgatgctggagtttattactgcgggcaaggtatacaagatcct
cc
SEQ ID NO. 107 IGKV2-9 (F)
>IGKV2-9*01|Canis lupus familiaris_boxer|F|V-REGION|
gatattgtcatgacacagaccccactgtccctgtctgtcagccctggagagactgcctcc
atctcttgcaaggccagtcagagcctcctgcacagtgatggaaacacgtatttgaattgg
ttccgacagaagccaggccagtctccacagcgtttgatctataaggtctccaacagagac
cctggggtcccagacaggttcagtggcagcgggtcagggacagatttcaccctgagaatc
agcagagtggaggctgacgatactggagtttattactgcgggcaagttatacaagatcct
cc
SEQ ID NO. 108 IGKV2-10 (F)
>IGKV2-10*01|Canis lupus familiaris_boxer|F|V-REGION|
gatattgtcatgacacagaccccactgtccctgtccgtcagccctggagagactgcctcc
atctcctgcaaggccagtcagagcctcctgcacagtgatggaaacacgtatttgaattgg
ttccgacagaagccaggccagtctccacagcgtttgatctataaggtctccaacagagac
cctggggtcccagacaggttcagtggcagcgggtcagggacagatttcaccctgagaatc
agcagagtggaggctgacgatactggagtttattactgcatgcaaggtacacagtttcct
cg
SEQ ID NO. 109 IGKV2-11 (F)
>IGKV2-11*01|Canis lupus familiaris_boxer|F|V-REGION|
gatatcgtcatgacacagaccccactgtccctgtccgtcagccctggagagactgcctcc
atctcctgcaaggccagtcagagcctcctgcacagtaacgggaacacctatttgttttgg
ttccgacagaagccaggccagtctccacagcgcctgatcaacttggtttccaacagagac
cctggggtcccacacaggttcagtggcagcgggtcagggacagatttcaccctgagaatc
agcagagtggaggctgacgatgctggagtttattactgcgggcaaggtatacaagctcct
cc
SEQ ID NO. 110 IGKV2S12 (P)
>IGKV2S12*01|Canis lupus familiaris_boxer|P|V-REGION|
gatatcgtgatgacccagaccccattgtccttgcctgtcacccctggagagctagcctca
tcactgtgcaggaggccagtcagagcctcctgcacagtgatggatatatttatttgaatt
ggtactttcagaaatcaggccagtctccatactcttgatctatatgctttacaaccagac
ttctggagtcccaggctggttcattggcagtggatcagggacagatttcaccctgaggat
cagcagggtggaggctgaagatgctggagtttattattgccaacaaactctacaaaatcc
tcc
SEQ ID NO. 111 IGKV2S13 (F)
>IGKV2S13*01|Canis lupus familiaris_boxer|F|V-REGION|
gatatcgtcatgacgcagaccccactgtccctgtctgtcagccctggagagccggcctcc
atctcctgcagggccagtcagagcctcctgcacagtaatgggaacacctatttgtattgg
ttccgacagaagccaggccagtctccacagggcctgatctacttggtttccaaccgtttc
tcttgggtcccagacaggttcagtggcagcgggtcagggacagatttcaccctgagaatc
agcagagtggaggctgacgatgctggagtttattactgcgggcaaaatttacagtttcct
tc
SEQ ID NO. 112 IGKV2S14 (P)
>IGKV2S14*01|Canis lupus familiaris_boxer|P|V-REGION|
gaggttgtgatgatacagaccccactgtccctgtctgtcagccctggagagccggcctcc
atctcctgcagggccagtcagagtctccggcacagtaatggaaacacctatttgtattgg
tacctgcaaaagccaggccagtctccacagcttctgatcgacttggtttccaaccatttc
actggggtgtcagacaggttcagtggcagcgggtctggcacagattttaccctgaggatc
agcagggtggaggctgaggatgttggagtttattactgcatgcaaagtacacatgatcct
cc
SEQ ID NO. 113 IGKV2S15 (P)
>IGKV2S15*01|Canis lupus familiaris_boxer|P|V-REGION|
gatatcatgatgacacagaccccactctccctgcctgccacccctggggaattggctgcc
atcttctgcagggccagagtctcctgcacaataatggaaacacttatttacactggttcc
tgcagacatcaggccaggttccaaggcatctgaaccatttggcttccagctgttactctg
gggtctcagacaggttcagtggcaacgggtcagggacagatttcacactgaaaatcagca
gagtggaggctgaggatgttagtgtttattagtgcctgcaagtacacaaccttccatc
SEQ ID NO. 114 IGKV2S16 (F)
>IGKV2S16*01|Canis lupus familiaris_boxer|F|V-REGION|
gaggccgtgatgacgcagaccccactgtccctggccgtcacccctggagagctggccact
atctcctgcagggccagtcagagtctcctgcgcagtgatggaaaatcctatttgaattgg
tacctgcagaagccaggccagactcctcggccgctgatttatgaggcttccaagcgtttc
tctggggtctcagacaggttcagtggcagcgggtcagggacagatttcacccttaaaatc
agcagggtggaggctgaggatgttggagtttattactgccagcaaagtctacattttcct
cc
SEQ ID NO. 115 IGKV2S18 (P)
>IGKV2S18*01|Canis lupus familiaris_boxer|P|V-REGION|
gatatcgtcatgacacagaccccactgtccgtgtctgtcagccctggagagacggcctcc
atctcctgcagggccagtcagagcctcctgcacagtgatggaaacacctatttggattgg
tacctgcagaagccaggccagattccaaaggacctgatctatagggtgtccaactgcttc
actggggtgtcagacaggttcagtggcagcgggtcagggacagatttcaccctgagaatc
agcagagtggaggctgacaacgctggagtttattactgcatgcaaggtatacaagatcct
cc
SEQ ID NO. 116 IGKV2S19 (F)
>IGKV2S19*01|Canis lupus familiaris_boxer|F|V-REGION|
gatatcgtcatgacacagactccactgtccctgtctgtcagccctggagagacggcctcc
atctcctgcagggccaatcagagcctcctgcacagtaatgggaacacctatttggattgg
tacatgcagaagccaggccagtctccacagggcctgatctatagggtgtccaaccacttc
actggcgtgtcagacaggttcagtggcagcgggtcagggacagatttcaccctgaagatc
agcagagtggaggctgacgatgctggagtttattactgcgggcaaggtacacactctcct
cc
SEQ ID NO. 117 IGKV3-3 (P)
>IGKV3-3*01|Canis lupus familiaris_boxer|P|V-REGION|
gaaatagtcttgacctagtctccagcctccctggctatttcccaaggggacagagtcaac
catcacctatgggaccagcaccagtaaaagctccagcaacttaacctggtaccaacagaa
ctctggagcttcttctaagctccttgtttacagcacagcaagcctggcttctgggatccc
agctggcttcattggcagtggatgtgggaactcttcctctctcacaatcaatggcatgga
ggctgaaggtgctgcctactattactaccagcagtagggtagctatctgct
SEQ ID NO. 118 IGKV3S1 (F)
>IGKV3S1*01|Canis lupus familiaris_boxer|F|V-REGION|
gaaatcgtgatgacacagtctccagcctccctctccttgtctcaggaggaaaaagtcacc
atcacctgccgggccagtcagagtgttagcagctacttagcctggtaccagcaaaaacct
gggcaggctcccaagctcctcatctatggtacatccaacagggccactggtgtcccatcc
cggttcagtggcagtgggtctgggacagacttcagcttcaccatcagcagcctggagcct
gaagatgttgcagtttattactgtcagcagtataatagcggatata
SEQ ID NO. 119 IGKV3S2 (P)
>IGKV3S2*01|Canis lupus familiaris_boxer|P|V-REGION|
gagattgtgccaacctagtctctagccttctaagactccagaagaaaaagtcaccatcag
ctgctgggcagtcagagtgttagcagctacttagcctggtaccagcaaaaacctggacag
gctcccaggctcttcatctatggtgcatccaacagggccactggtgtcccagtccgcttc
agcggcagtgggtgtgggacagatttcaccctcatcagcagcagtctggagtcagtctga
agatgttgcaacatattactgccagcagtataatagctacccacc
SEQ ID NO. 120 IGKV4S1 (F)
>IGKV4S1*01|Canis lupus familiaris_boxer|F|V-REGION|
gaaatcgtgatgacccagtctccaggctctctggctgggtctgcaggagagagcgtctcc
atcaactgcaagtccagccagagtcttctgtacagcttcaaccagaagaactacttagcc
tggtaccagcagaaaccaggagagcgtcctaagctgctcatctacttagcctccagctgg
gcatctggggtccctgcccgattcagcagcagtggatctgggacagatttcaccctcacc
atcaacaacctccaggctgaagatgtgggggattattactgtcagcagcattatagttct
cctcc
SEQ ID NO. 121 IGKV4-1 (ORF)
>IGKV4-1*01|Canis lupus familiaris_boxer|ORF|V-REGION|
gacatcacgatgactcagtgtccaggctccctggctgtgtctccaggtcagcaggtcacc
acgaactgcagggccagtcaaagcgttagtggctacttagcctggtacctgcagaaacca
ggacagcgtcctaagctgctcatctacttagcctccagctgggcatctggggtccctgcc
cgattcagcagcagtggatctgggacagatttcaccctcaccgtcaacaacctcgaggct
gaagatgtgagggattattactgtcagcagcattatagttctcctct
SEQ ID NO. 122 IGKV7-2 (P)
>IGKV7-2*01|Canis lupus familiaris_boxer|P|V-REGION|
gacattatgctgacccagtctccagcctccttgaccatgtgtctccaggagagagggcca
ccatctcttgcagggccagtcagaaagccagtgatatttggggcattacccaccatatta
ccttgtaccaacagaaatcagaacagcatcctaaagtcctgattaatgaagcctccagtt
gggtctggggtcctaggcaggttcagtggctgtgggtctgggactgatttcagcctcaca
attgatcctgtggaggctggcgatgctgtcaactattactgccagcagagtaaggagtct
cctcc
SEQ ID NO. 123 IGKV(II)-1 (P)
>IGKV(II)-1*01|Canis lupus familiaris_boxer|P|V-REGION|
gaaattgcagattgtcaaatggataataccaggatgcggtctctagcctccctgactccc
aggggagagaaccatcattacccataaaataaatcctgatgacataataagtttgcttgg
tatcaatagaaaccaggtgagattcctcgagtcctggtatacgacacttccatccttaca
ggtcccaaactggttcagtggcagtgtctccaagtcagatcttactctcatcatcagcaa
tgtgggcacacctgatgctgctacttattactgttatgagcattcagga
Germline Jκ sequences
SEQ ID NO. 124 IGKJ1 (F)
>GKJ1*01|Canis lupus familiaris_boxer|F|-REGION|
gtggacgttcggagcaggaaccaaggtggagctcaaac
SEQ ID NO. 125 IGKJ2 (ORF)
>IGKJ2*01|Canis lupus familiaris_boxer|ORF|J-REGION|
tttatactttcagccagggaaccaagctggagataaaac
SEQ ID NO. 126 IGKJ3 (F)
>IGKJ3*01|Canis lupus familiaris_boxer|F|J-REGION|
gttcacttttggccaagggaccaaactggagatcaaac
SEQ ID NO. 127 IGKJ4 (F)
>IGKJ4*01|Canis lupus familiaris_boxer|F|J-REGION|
gcttacgttcggccaagggaccaaggtggagatcaaac
SEQ ID NO. 128 IGKJ5 (F)
>IGKJ5*01|Canis lupus familiaris_boxer|F|J-REGION|
gatcacctttggcaaagggacacatctggagattaaac

TABLE 3
Canine Igλ Sequence Information
Germline V_{λ} sequences
SEQ ID NO. 129 IGLV1-35 (P)
>IGLV1-35*01|Canis lupus familiaris_boxer|P|V-REGION|
cagtctgtgctgactcagctggcctcggtgtctggggccctgggccacagggtcagcatc
tcctggactggaagcagctccaacataagggttgattatcctttgagctgataccaacag
ctcccagaatgaagaacgaacccaaactcctcatctatggtaacagcaattggctctcag
gggttccagatccattctctagaggctccaagtctggcacctcaggctccctgaccaact
ctggcctccaggctgaggacgaggctgattgttactgcgcagcgtgggacatggatctca
gtgctc
SEQ ID NO. 130 IGLV1-37 (ORF)
>IGLV1-37*01|Canis lupus familiaris_boxer|ORF|V-REGION|
caatctgtgctgactcagctggcctcagtgtctgggtccttgggccagagggtcaccatc
tcctgctctggaagcacaaatgacattggtattattggtgtgaactggtaccagcagctc
ccagggaaggcccctaaactcctcatatacgataatgagaagcgaccctcaggtatcccc
gatcgattctctggctccaagtctggcaactcaggcaccctgaccatcactgggctccag
gctgaggacgaggctgattattactgccagtccatggatttcagcctcggtggt
SEQ ID NO. 131 IGLV1-41 (ORF)
>IGLV1-41*01|Canis lupus familiaris_boxer|ORF|V-REGION|
cagtctgtgctgactcagccagcctccgtgtctgggtccctgggccagagggtcaccatt
tcctgcactggaagcagctccaacgttggttatagcagtagtgtgggctggtaccagcag
ttcccaggaacaggccccagaaccatcatctattatgatagtagccgaccctcgggggtc
cccgatcgattctctggctccaagtctggcagcacagccaccctgaccatctctgggctc
caggctgaggatgaggctgattattactgctcatcttgggacaacagtctcaaagctcc
SEQ ID NO. 132 IGLV1-44 (F)
>IGLV1-44*01|Canis lupus familiaris_boxer|F|V-REGION|
caggctgtgctgaatcagccggcctcagtgtctggggccctgggccagaaggtcaccatc
tcctgctctggaagcacaaatgacattgatatatttggtgtgagctggtaccaacagctc
ccaggaaaggcccctaaactcctcgtggacagtgatggggatcgaccctcaggggtccct
gacagattttctggctccagctctggcaactcaggcaccctgaccatcactgggctccag
gctgaggacgaggctgattattactgtcagtctgttgattccacgcttggtgctca
SEQ ID NO. 133 IGLV1-45 (P)
>IGLV1-45*01|Canis lupus familiaris_boxer|P|V-REGION|
cagtctgtactgactcaatcagcctcagcgtctgggtccttgggccagagggtctccgtc
tcctgctctagcagcacaaacaacattggtattattggtgtgaagtggtaccagcagatc
ccaagaaaggcccctaaactcctcatatatgataatgagaagagaccctcaggtgtcccc
aattgattctctggctccaagtctggcaacttaggcaccctaaccatcaatgggcttcag
gctgagggcgaggctgattattactgccagtccatggatttcagcctcggtggt
SEQ ID NO. 134 IGLV1-46 (F)
>IGLV1-46*01|Canis lupus familiaris_boxer|F|V-REGION|
cagtctgtgctgactcaaccagcctcagtgtccgggtctctgggccagagggtcaccatc
tcctgcactggaagcagctccaacattggtagagattatgtgggctggtaccaacagctc
ccgggaacacgccccagaaccctcatctatggtaatagtaaccgaccctcgggggtcccc
gatcgattctctggctccaagtcaggcagcacagccaccctgaccatctctgggctccag
gctgaggacgaggctgattattactgctctacatgggacaacagtctcactgttcc
SEQ ID NO. 135 IGLV1-48 (F)
>IGLV1-48*01|Canis lupus familiaris_boxer|F|V-REGION|
cagtctatgctgactcagccagcctcagtgtctgggtccctgggccagaaggtcaccatc
tcctgcactggaagcagctccaacatcggtggtaattatgtgggctggtaccaacagctc
ccaggaataggccctagaaccgtcatctatggtaataattaccgaccttcaggggtcccc
gatcgattctctggctccaagtcaggcagttcagccaccctgaccatctctgggctccag
gctgaggacgaggctgagtattactgctcatcatgggatgatagtctcagaggtca
SEQ ID NO. 136 IGLV1-49 (F)
>IGLV1-49*01|Canis lupus familiaris_boxer|F|V-REGION|
caggctgtgctgactcagccgccctcagtgtctgcggtcctgggacagagggtcaccatc
tcctgcactggaagcagcaccaacattggcagtggttatgatgtacaatggtaccagcag
ctcccaggaaagtcccctaaaactatcatctatggtaatagcaatcgaccctcaggggtc
ccggatcgcttctctggctccaagtcaggcagcacagcctctctgaccatcactgggctc
caggctgaggacgaggctgattattactgccagtcctctgatgacaacctcgatgatca
SEQ ID NO. 137 IGLV1-50 (P)
>IGLV1-50*01|Canis lupus familiaris_boxer|P|V-REGION|
cagtctgtgctgactcagccggcctca...gtgtccgggtctctgggccagagagtcacc
atctcctgcactggaagcagctccaacatc..................gatagaaaatat
gttggctggtaccaacagctc...ccgggaacaggccccagaaccgtcatctatgataat
.....................agtaaccgaccctcgggggtccct...gatcgattctct
ggctccaag......tcaggcagcacagccaccctgaccatctctgggctccaggctgag
gacgaggctgat...tattactgctcaacatacgacagcagtctcagtagtgg
SEQ ID NO. 138 IGLV1-52 (P)
>IGLV1-52*01|Canis lupus familiaris_boxer|P|V-REGION|
cagtctgtgctgactcagccggcctcagtgtctgggtccctgggccagagggtcaccatc
tcctgcactggaagcagctccaacatcagtagatataatgtgaactggtaccaacagctc
ctgggaacaggccccagaaccctcatctatggtagtagtaaccgaccctcgggggtcccc
gattgattctctggctccaagtcaggcagcccagctaccctgaccatctctgggctccag
gctgaggatgaggctgattattactgctcaacatacgacaggggtctcagtgctcg
SEQ ID NO. 139 IGLV1-54 (P)
>IGLV1-54*01|Canis lupus familiaris_boxer|P|V-REGION|
cagcctgtgctgactcagccgccctcagggtctgggggcctgggccagaggttcagcatc
tcctgttctggaagcacaaacaacatcagtgattattatgtgaactggtactaacagctc
ccagggacagcccctaaaaccattatctatttggatgataccagaccccctggggtcccg
gattgattctctgtctccaagtctagcagctcagctaccctgaccatctctgggctccag
gctgaggatgaagctgattattactgctcatcctggggtgatagtctcaatgctcc
SEQ ID NO. 140 IGLV1-55 (F)
>IGLV1-55*01|Canis lupus familiaris_boxer|F|V-REGION|
cagtctgtgctgactcagccggcctcagtgtctgggtccctgggccagaggatcaccatc
tcctgcactggaagcagctccaacattggaggtaataatgtgggttggtaccagcagctc
ccaggaagaggccccagaactgtcatctatagtacaaatagtcgaccctcgggggtgccc
gatcgattctctggctccaagtctggcagcacagccaccctgaccatctctgggctccag
gctgaggatgaggctgattattactgctcaacgtgggatgatagtctcagtgctcc
SEQ ID NO. 141 IGLV1-56 (ORF)
>IGLV1-56*01|Canis lupus familiaris_boxer|ORF|V-REGION|
cggtctgtgctgactcagccgccctcagtgtcgggatctgtgggccagagaatcaccatc
tcccgctctggaagcacaaacagcattggtatacttggtgtgaactggtaccaagagctc
ccaggaaaggcccctaaactcctcgtagatggtactgggaatagaccctcaggggtccct
gaccgattttctggctccaaatctggcaactcaggcactctgaccatcactgggcttcag
cctgaggacgaggctgattattattgtcagtccattgaacccatgcttggtgctcc
SEQ ID NO. 142 IGLV1-57 (F)
>IGLV1-57*01|Canis lupus familiaris_boxer|F|V-REGION|
caggctgtgctgactccgctgccctcagtgtctgcggccctgggacagacggtcaccatc
tcttgtactggaaatagcacccaaatcagcagtggttatgctgtacaatggtaccagcag
ctcccaggaaagtcccctgaaactatcatctatggtgatagcaatcgaccctcgggggtc
ccagatcgattctctggcttcagctctggcaattcagccacactggccatcactgggctc
caggatgaggacgaggctgattattactgccagtccttagatgacaacctcaatggtca
SEQ ID NO. 143 IGLV1-58 (F)
>IGLV1-58*01|Canis lupus familiaris_boxer|F|V-REGION|
cagtctgtgctgactcagccggcctcagtgtctgggtccctgggccagagggtcaccatc
tcctgcactggaagcagctccaacatcggtagatatagtgttggctggttccagcagctc
ccgggaaaaggccccagaaccgtcatctatagtagtagtaaccgaccctcaggggtccct
gatcgattctctggctccaagtcaggcagcacagccaccctgaccatctctgggctccag
gctgaggacgaggctgattattactgctcaacatacgacagcagtctcagtagtag
SEQ ID NO. 144 IGLV1-61 (P)
>IGLV1-61*01|Canis lupus familiaris_boxer|P|V-REGION|
cagtctgtgctgacatagccaccctcagtgtctggggccctgggccagagggtcaccatc
tcctgcactggaagcagctcaagcatgggtagttattatgtgagctggcacaagcagctc
ccaggaacaggccccagaaccatcatgtgttgtaaaaacatcgaccttcgggaatctcca
atcaagtctctggctcccattctggcaacacagccaccctgaccatcactgggctcctgg
ctgaggatgaggctgattattactgttcaacatgggatgacaatctcaatgcacc
SEQ ID NO. 145 IGLV1-63 (P)
>IGLV1-63*01|Canis lupus familiaris_boxer|P|V-REGION|
cagtctgtgctgactcagctgccctcagtgtctggggccctgggccagagggtcaccatc
tcctgctctggaagcagctctaaacttggggcttatgctctgaactagaaccaacaattc
ccaggaacagattccaatttcctcatctatgatgatagtaattgatctttctggatgcct
gattaattctgtggctccacatccagcagttcaggctccctgaccatcactgggctctgg
gatgaggacaaggctgattattactgccagtgccattaccatagcctccgtgct
SEQ ID NO. 146 IGLV1-65 (P)
>IGLV1-65*01|Canis lupus familiaris_boxer|P|V-REGION|
cagtctgtgctgactcagccagcctcagtgtctggatccctgggccaaagggtcaccatc
tcctgcactggaagcacaaacaacatcggtggtgataattatgtgcactggtaccaacag
ctcccaggaaaggcacccagtctcctcatctatggtgatgataacagagaatctggggtc
ccggaacgattctctggctccaagtcaggcagctcagccactctgaccatcactgggctc
catgctgaggacgaggctgatattattgccagtcctacgatgacagcctcaatactca
SEQ ID NO. 147 IGLV1-66 (F)
>IGLV1-66*01|Canis lupus familiaris_boxer|F|V-REGION|
cagtctgtgctgactcagccgccctcagtgtcaggatctgtgggccagagaatcaccatc
tcctgctctggaagcacaaacagcattggtatacttggtgtgaactggtaccaactgctc
tcaggaaaggcccctaaactcctcgtagatggtactggaaatcgaccctcaggggtccct
gaccgattttctggctccaaatctggcaactcaggcactctgaccatcactgggcttcag
cctgaggacgaggctgattattattgtcagtccattgaacccatgcttggtgctcc
SEQ ID NO. 148 IGLV1-67 (F)
>IGLV1-67*01|Canis lupus familiaris_boxer|F|V-REGION|
cagtctgtcctgactcagccggcctcagtgtctggggttctgggccagagggtcaccatc
tcctgcactggaagcagctccaacattggtggaaattatgtgagctggcaccagcaggtc
ccagaaacaggccccagaaacatcatctatgctgataactaccgagcctcgggggtccct
gatcgattctctggctccaagtcaggcagcacagccaccctgaccatctctgtgctccag
gctgaggatgaggctgattattactgctcagtgggggatgatagtctcaaagcacc
SEQ ID NO. 149 IGLV1-68 (P)
>IGLV1-68*01|Canis lupus familiaris_boxer|P|V-REGION|
cagtccatcctgactcagcagccctcagtctctgggtcactgggccagagggtcaccatc
tcttgcactggattccctagcaacaatgattatgatgcaatgaaaattcatacttaagtg
ggctggtaccaacagtccccaggaaagtcacccagtctcctcatttatgatgaaaccaga
aactctggggtccctgatcgattctctggctccagaactggtagctcagcctccctgccc
atctctggactccaggctgaggacaagactgagtattactgctcagcatgggatgatcgt
cttgatgctca
SEQ ID NO. 150 IGLV1-69 (P)
>IGLV1-69*01|Canis lupus familiaris_boxer|P|V-REGION|
cagtctgtgctaactcagccaccctcagtgtcggggtcgctgggccagagggtcaccatc
tcctgctctggaagcacaaacaacatcagtattgttggtgcgagctggtaccaacagctc
ccaggaaaggcccctaaactcctcgtggacagtgatggggatcgaccgtcaggggtccct
gaccgattttctggctctaagtctggcaaatcagccaccctgaccatcactgggcttcag
gctgaggacgaggctgattattactgtatattggtcccacgctttgtgctca
SEQ ID NO. 151 IGLV1-69-1 (P)
>IGLV1-69-1*01|Canis lupus familiaris_boxer|P|V-REGION|
cagtctgtgctgactcagccactgttagggcctggggccctgggcagagggtcaccctct
cctgacctggaagagtcccagtattggtgattatggtatgaaatggtacaagcagcttgc
aaggacagaccccagactcgtcatctatggcaatagcaattgatcctcgggtccccaatc
aattttctggctctggttttggcatcactggctccttgaccacctctgggctccagactg
aaaaataggctgattactagtgcttctccagtgatccaggcctgt
SEQ ID NO. 152 IGLV1-70 (F)
>IGLV1-70*01|Canis lupus familiaris_boxer|F|V-REGION|
cagtctgtgctgactcaaccggcctccgtgtctgggtccctgggccagagagtcaccatc
tcttgcactagaagcagctcgaacgttggctatggcaatgatgtgggatggtaccagcag
ctcccaggaacaggccccagaaccatcatctataataccaatactcgaccctctggggtt
cctgatcgattctctggctccaaatcaggcagcacagccaccctgaccatctctggactc
caggctgaggacgaggctgattattactgctcttcctatgacagcagtctcaatgctca
SEQ ID NO. 153 IGLV1-72 (ORF)
>IGLV1-72*01|Canis lupus familiaris_boxer|ORF|V-REGION|
cagtctgtgctaactcagccggcctcagtgtctggttccctgggtcagagggtcaccatc
tgcactggaagcagctccaacattggtacatatagtgtaggctggtaccaacagctccca
ggatcaggccccagaaccatcatctatggtagtagtaaccgaccgttgggggtccctgat
cgattctctggctccaggtcaggcagcacagccaccctgaccatctctgggctccaggct
gaggacgaagctgattattactgcttcacatacgacagtagtctcaaagctcc
SEQ ID NO. 154 IGLV1-73 (F)
>IGLV1-73*01|Canis lupus familiaris_boxer|F|V-REGION|
cagtctgtgctgaatcagccaccttcagtgtctggatccctgggccagagaatcaccatc
tcctgctctggaagcacgaatgacatcggtatgcttggtgtgaactggtaccaacagctc
ccaggaaatgcccctaaactccttgtagatggtactgggaatcgaccctcaggggtccct
gaccaattttctggctccaaatctggcaattcaggcactctgaccatcactgggctccag
gctgaggacgaggctgattattattgtcagtcctatgatctcacgcttggtgctcc
SEQ ID NO. 155 IGLV1-74 (P)
>IGLV1-74*01|Canis lupus familiaris_boxer|P|V-REGION||
cagtccatgatgactcagccaccctcagtgtctgggtcactgggccagagggtcaccatc
tactgcactggaatccctagcaacactgattatagtggattggaaatttatacttatgtg
agctggtaccaacagtataaggaaaggcacccagtctcctcatctatggggatgataccg
gaaactctgaggtccctgatcaattctctggctccaggtctggtagctcaacctccctga
ccatctctggactccaggctgaggatagtcttaatgctca
SEQ ID NO. 156 IGLV1-75 (F)
>IGLV1-75*01|Canis lupus familiaris_boxer|F|V-REGION|
cagtctgtgctgactcagccggcctcagtgactgggtccctgggccagagggtcaccatc
tcctgcactggaagcagctccaacatcggtggatataatgttggctggttccagcagctc
ccgggaacaggccccagaaccgtcatctatagtagtagtaaccgaccctcgggggtcccg
gatcgattctctggctccaggtcaggcagcacagccaccctgaccatctctgggctccag
gctgaggacgaggctgagtattactgctcaacatgggacagcagtctcaaagctcc
SEQ ID NO. 157 IGLV1-78 (P)
>IGLV1-78*01|Canis lupus familiaris_boxer|P|V-REGION|
cagtctgtgctgactcaaccggcctcagtgtccaggtccctgggccagatagtcaccatc
tcttgcgctggaagcagctccaacatccgtacaaaatatgtgggctggtactaacagctc
ccgagaacaggccccagaaccgtcatctatggtaatagtaactgaccctcgggggtcctc
gatcaattctctggctccaagtcaggcagcatagccaccctgaccatctctgtgctccag
gctgaggacgaggcttattattactgctcaacatatgacagcagtctcagtgctct
SEQ ID NO. 158 IGLV1-79 (P)
>IGLV1-79*01|Canis lupus familiaris_boxer|P|V-REGION|
cagtctgtgctgactcaaccggcctctgtgtctggggccctgggccagaggtcaccatct
cctgcactaggagcagctccaatgttggttatagcagttatgtgggctggtaccagcagc
tcccaggaacaggccccaaaaccatcatctataataccaatactcgaccctctggggttc
ctgatcgattctctggctccaaatcaggcagcacagccacccttaccattgctggactcc
aggctgaggacgaggctgattattactgctcatcctatgacagcagtctcaaagctcc
SEQ ID NO. 159 IGLV1-79-1 (P)
>IGLV1-79-1*01|Canis lupus familiaris_boxer|P|V-REGION|
cagtctatgctgactcaccctggccagaggatcaccctctcctgacctggaagagtccca
gtattggtgattatggtgtgaaatggtacaggcagctagcaagaacagaccccagactcc
tcatttatagcaatagcaatcgatccttgagtccccaatcaattttccgcctctggtttt
gacattactggctccttgaccacctccaggctccagactgaaaaataggctgattactag
tgcttatacagtgatccaggcttgtggggctg
SEQ ID NO. 160 IGLV1-80 (F)
>IGLV1-80*01|Canis lupus familiaris_boxer|F|V-REGION|
cagtctgtgctgactcagccgacctcagtgtcgtggtccctgggccagagggtcacaatc
tcatgctctagaagcacgaataacatcggtattgtcggggcgagctggtaccaacagctc
ccaggaaaggcccctaaactcctcgtggacagtgatggggatcaactgtcaggggtccct
gaccgattttctggctccaagtctggcaactcagccaacctgaccatcactgggctccag
gctgaggacaaggctgattattactgccagtcctttgatcacacgcttggtgctcg
SEQ ID NO. 161 IGLV1-81 (P)
>IGLV1-81*01|Canis lupus familiaris_boxer|P|V-REGION|
cagtctgtgttgagtcagccagcctcagtgtctggggttctgggccagagggtcaccatc
tcctgcactggaagcagctccaacatcggtggaaattacgtgagctggcaccagcaggtc
ccagaaacaggccccagaaacatcatctatgctgataactactgagcctcgggggtccct
gatggattctctggctccaagtaaggcagcacagccaccccgaccatctctgtgctccag
gctgaggatgaggctgattattactgctcagtgggggataatagtctcaaagcacc
SEQ ID NO. 162 IGLV1-82 (F)
>IGLV1-82*01|Canis lupus familiaris_boxer|F|V-REGION|
cagtctgtgctgactcagccagcctcagtgtcggggtccctgggccagagagtcaccatc
tcctgctctggaaggacaaacatcggtaggtttggtgctagctggtaccaacagctccca
ggaaaggcccctaaactcctcgtggacagtgatggggatcgaccgtcaggggtccctgac
cgattttccggctccaagtctggcaactcggccactctgaccatcactggtctccatgct
gaggacgaggctgattattactgtctgtctattggtcccacgcttggtgctca
SEQ ID NO. 163 IGLV1-82-1 (P)
>IGLV1-82-1*01|Canis lupus familiaris_boxer|P|V-REGION|
cagtctgtgctgactcagccactgttagggcctggggccctggccagaggctcactctct
cctgccctggaagagtcccagtattggtgattatgatgtgaagtggtacaggcagctcac
aagaacagaccctagactcctcatccatggtgatagcaattgatcctcgggtccccaatc
acttttctggctctgtttttggcatcactggctgcttgaccacctctgggctccagactg
aaaaataggctgattactagtgcttatccagtgatccag
SEQ ID NO. 164 IGLV1-83 (P)
>IGLV1-83*01|Canis lupus familiaris_boxer|P|V-REGION|
cagtctgtgctgactcaaccggcctctgtgtctggggccctgggccagaggtcaccatct
cctgcactaggagcagctccaatgttggttatagcagttatgtgggctggtaccagcagc
tcccaggaacaggccccaaaaccatcatctataataccaatactcgaccctctggggtcc
ctgatcgattctctggctccaaatcaggcaggacagccacccttaccattgctggactcc
aggctgaggacgaggctgattattactgctcatcctatgacagcagtctcaaagctcc
SEQ ID NO. 165 IGLV1-84 (F)
>IGLV1-84*01|Canis lupus familiaris_boxer|F|V-REGION|
caggctgtgctgactcagccggcctcagtgtctgggtccctgggccagagggtcaccatc
tcctgcactggaagcagctccaatgttggttatggcaattatgtgggctggtaccagcag
ctcccaggaacaggccccagaaccctcatctatggtagtagttaccgaccctcgggggtc
cctgatcgattctctggctccagttcaggcagctcagccacactgaccatctctgggctc
caggctgaggatgaagctgattattactgctcatcctatgacagcagtctcagtggtgg
SEQ ID NO. 166 IGLV1-84-1 (ORF)
>IGLV1-84-1*01|Canis lupus familiaris_boxer|ORF|V-REGION|
cagtctgtgctgactcagccagcctcagcgtctgggtccttgggccagagggtcactgtc
tcctgctctagcagcacaaacaacatcggtattattggtgtgaagtggtaccagcagatc
ccaggaaaggcccataaactcctcatatatgataatgagaagcgaccctcaggtgtcccc
aatcgattctctggctccaagtctggcgacttaagcaccctgaccatcaatgggcttcag
ggtgaggacgaggctgattattattgccagtccatggatttcagcctcggtggtca
SEQ ID NO. 167 IGLV1-86 (ORF)
>IGLV1-86*01|Canis lupus familiaris_boxer|ORF|V-REGION|
cagtctgtgctgactcagccagcctcagtgtctgggtccctgggccagagggtcaccatc
tcctgcactggaatccccagcaacacagattttgatggaatagaatttgatacttctgtg
agctggtaccaacagctcccagaaaagccccctaaaaccatcatctatggtagtactctt
tcattctcgggggtccccgatcgattctctggctccaggtctggcagcacagccaccctg
accatctctgggctccaggctgaggacgaggctgattattactgctcatcctgggatgat
agtctcaaatcata
SEQ ID NO. 168 IGLV1-87 (F)
>IGLV1-87*01|Canis lupus familiaris_boxer|F|V-REGION|
cagtctgtgctgactcagccagcctcagtgtctggatccctgggccaaagggtcaccatc
tcctgcactggaagcacaaacaacatcggtggtgataattatgtgcactggtaccaacag
ctcccaggaaaggcacccagtctcctcatctatggtgatgataacagagaatctggggtc
cctgaacgattctctggctccaagtcaggcagctcagccactctgaccatcactgggctc
caggctgaggacgaggctgattattattgccagtcctacgatgacagcctcaatactca
SEQ ID NO. 169 IGLV1-88 (P)
>IGLV1-88*01|Canis lupus familiaris_boxer|P|V-REGION|
cagtctgtgctgactcagccgccctcagtgtcgggatctgtgggccagagaatcaccatc
tcctgctctggaagcacaaacagctaccaacagctctcaggaaaggcctctaaactcctc
gtagatggtactgggaaccgaccctcaggggtccccgaccgattttctggctccaaatct
ggcaactcaggcactctgaccatcactgggcttgggacgaggctgaggacgaggctgagg
acgaggctgattattattgttagtccactgatctcacgcttggtgctcc
SEQ ID NO. 170 IGLV1-88-2 (P)
>IGLV1-88-2*01|Canis lupus familiaris_boxer|P|V-REGION|
caggccgccctgggcaatgagttcgtgcaggtcaaggctgagacagacctgcagaattca
ggtttgtctgagacacagctcatcagatgtgtgcagtgtgtgtcctggtaccaacggctc
ccatgaatgggtcctaaatccttatctagaaataacatttagatcactttgtggcccgga
tccattctctggctccatgtctggcaactctggcctcatgaacatcactgggctatggtc
tgaagatggagctgctcttcacaggccctcttgggacaaaattcttggggct
SEQ ID NO. 171 IGLV1-88-3 (P)
>IGLV1-88-3*01|Canis lupus familiaris_boxer|P|V-REGION|
cagtccatcctgactcagccgccctcagtctctgggtcactgggccagagggtcaccatc
tcctgcaatggaatccctgacagcaatgattatgatgcatgaaaattcatacttacgtga
gctggtaccaacagttcccaagaaagtcaccagtctcctcatctacgatgataccagaaa
ctctggggaccctgatcaattctctggctccagatctggtaactcagcctccctgcccat
ctctggactccaggctgaggacgaggctgagtattactgctcagcatgggatgatcgtct
tgatgctca
SEQ ID NO. 172 IGLV1-89 (P)
>IGLV1-89*01|Canis lupus familiaris_boxer|P|V-REGION|
cagtctgtactgactcagccggcctcagtgtctgggtccctgggccagagggtcaccatc
tcctgcactggaagcagctccaacatcggtggatattatgtgagctggctctagcagctc
ccgggaacaggccccagaaccatcatctatagtagtagtaaccgaccttcaggggtccct
gatcgattctctggctccaggtcaggcagcacagccaccctgaccatctctgggctccag
gctgaggatgaggctgattattactgttcaacatacgacagcagtctcaaagctcc
SEQ ID NO. 173 IGLV1-89-2 (P)
>IGLV1-89-2*01|Canis lupus familiaris_boxer|P|V-REGION|
cttcctgtgctgacccagccaccctcaaggtctgggggtctggttcagaagatcaccatc
ttctgttctggaagcacaaacaacatgggtgataattatgttaactggtacaaacagctt
ccaggaacggcccctaaaaccatcatctaagtggatcatatcagaccctcaggggtcctg
gagagattctctgtctccaattctggcagctcagccaacctgaccatctctgggctccag
gatgaggactaggctgattattattgctcatcctggcatgatagtctcagtgctcc
SEQ ID NO. 174 IGLV1-91 (P)
>IGLV1-91*01|Canis lupus familiaris_boxer|P|V-REGION|
caggctgtgctgactcagctgccctcagtgtctgcagccctgggacagagggtcaccatc
tgcactggaagcagcaccaacatcggcagtggttattatacactatggtaccagcagctg
caggaaagtcccctaaaactatcatctatggtaatagcaatcgacccttgagggtcccgg
atcgattctctggctccaagtatggcaattcagccacgctgaccatcactgggctccagg
ctgaggacgaggatgattattactgccagtcctctgatgacaacctcgatggtca
SEQ ID NO. 175 IGLV1-92 (F)
>IGLV1-92*01|Canis lupus familiaris_boxer|F|V-REGION|
cagtctgtgctgactcagccggcctcggtgtctgggtccctgggccagagggtcaccatc
tcctgcactggaagcagctccaatgttggttatggcaattatgtgggctggtaccagcag
cttccaggaacaggccccagaaccattatctgttataccaatactcgaccctctggggtt
cctgatcgatactctggctccaagtcaggcagcacagccaccctgaccatctctgggctc
caggctgaagacgagactgattattactgtactacgtgtgacagcagtctcaatgctag
SEQ ID NO. 176 IGLV1-94 (F)
>IGLV1-94*01|Canis lupus familiaris_boxer|F|V-REGION|
cagtctgtgctgactcagcctccctcagtgtccgggttcctgggccagagggtcaccatc
tcctgcactggaagcagctccaacatcggtagaggttatgtgcactggtaccaacagctc
ccaggaacaggccccagaaccctcatctatggtattagtaaccgaccctcaggggtcccc
gatcgattctctggctccaggtcaggcagcacagccactctgacaatctctgggctccag
gctgaggatgaggctgattattactgctcatcctgggacagcagtctcagtgctct
SEQ ID NO. 177 IGLV1-95-1 (P)
>IGLV1-95-1*01|Canis lupus familiaris_boxer|P|V-REGION|
cagtctgtgctgactcagccactgttagggcctgggttcctggccagagggtcaccctct
cctgccctggaagagtctcagttttggtgattatggtgtgaaacggtacaggaagctcgc
atggacagaccccagactcctcatctatggcaatagcaattgattctcgggtccccagtc
tattttctggctctggttttggcatcactggctccttgaccacctccgggctccagactg
aaaaataggctgatttctagtgcttctccagtgatccaggccttt
SEQ ID NO. 178 IGLV1-96 (F)
>IGLV1-96*01|Canis lupus familiaris_boxer|F|V-REGION|
cagtctgcgctgactcaaacggcctccatgtctgggtctctgggccagagggtcaccgtc
tcctgcactggaagcagttccaacgttggttatagaagttatgtgggctggtaccagcag
ctcccaggaacaggccccagaaccatcatctataataccaatactcgaccctctggggtt
cctgatcgattctctggctccatatcaggcagcacagccaccctgactattgctggactc
caggctgaggacgaggctgattattactgctcatcctatgacagcagtctcaaagctcc
SEQ ID NO. 179 IGLV1-97 (P)
>IGLV1-97*01|Canis lupus familiaris_boxer|P|V-REGION|
cagtctgtgctgaatcagctgccttcagtgttaggatccctgggccagagaatcaccatc
tcctgctctggaagcacgaatgacatcggtatgcttggtgtgaactggtaccaagagccg
ccaggaaaggcccctaaactcctcgtagatggtactgggaatcgaccctcagggtccctg
ccgattttctggctccaaatctggcaactcaggcactctgaccatcactgggctccaggc
tgaggacgaggctgattattattgtcagtccactgatctcacgcttggtgctcc
SEQ ID NO. 180 IGLV1-97-4 (F)
>IGLV1-97-4*01|Canis lupus familiaris_boxer|F|V-REGION|
cagtctgtgctgactcagcctccctcagtgttcaggtccctgggccagagggtcactata
tcctgcactggaagcagctccaacgtcggtagaggttatgtgatctggtaccaacagctc
ctgggaacacgcccaagaaccctcatatatggtagtagtaaccaaccctcaggggtcccc
aatcaattctctggctccaggtcaggcagcacagacactctgacaatctctgggttccag
gctgaggatgaggctgattattactgctcatcctgggacagcagtctcagtgctct
SEQ ID NO. 181 IGLV1-98 (P)
>IGLV1-98*01|Canis lupus familiaris_boxer|P|V-REGION|
cagtctgtgctgactcaaccagtctcagtgtctggggccctgtgccagagggtcaccatc
tcctgcactggaaacagctccaacattggttatagcagttgtgtgagctgatatcagcag
ctcccaggaacaggccccagaaccatcatctatagtatgaatactcaaccctctggggtt
cctgatcgattctctggctccaggtcaggcaactcagccaccctaaccatctctgggctc
caggctgaggacaaggctgactattactgctcaacatatgacagcagtctcagtgctca
SEQ ID NO. 182 IGLV1-100 (F)
>IGLV1-100*01|Canis lupus familiaris_boxer|F|V-REGION|
cagtctgtgctgactcagccgacctcagtgtcggggtcccttggccagagggtcaccatc
tcctgctctggaagcacgaacaacatcggtattgttggtgcgagctggtaccaacagctc
ccaggaaaggcccctaaactcctcgtggacagtgatggggatcgaccgtcaggggtccct
gaccggttttccggctccaagtctggcaactcagccaccctgaccatcactgggcttcag
gctgaggacgaggctgattattactgccagtcctttgataccacgcttgatgctca
SEQ ID NO. 183 IGLV1-100-1 (P)
>IGLV1-100-1*01|Canis lupus familiaris_boxer|P|V-REGION|
cagtctgtactgactcagcagccgttagtgcttggggccctggccagagggtcagcttct
cctgccttggaagagtcccagtattggtaattatggtgtgaaatggtacaagcagctcaa
aaggacagaccccagacttctcatctatggcaatagcaattgatcctcgggtccccaatc
aattttctggctctggttttggcatcactggctccttgaccacctatgggctccagactg
aaaaataggctgattactagtgcttttccagtgatccagtcctgaggggc
SEQ ID NO. 184 IGLV1-101 (P)
>IGLV1-101*01|Canis lupus familiaris_boxer|P|V-REGION|
cagtctgtgctgactcaaccggcctccgtgtctggggccttgggccagagggtcaccatc
tcctgcactggaagcagctccaatgttggttatagcagctatgtgggcttgtaccagcag
ctcccaggaacaggcctcaaaaccatcatctataataccaatactcgaccctctggggtt
cctgatcaattctctggctccaaatcaggcagcacagccacctgaccattgctggacttc
aggctgaggacgaggctgattattactgctcatcctatgacagcagtctcaaagctcc
SEQ ID NO. 185 IGLV1-103 (F)
>IGLV1-103*01|Canis lupus familiaris_boxer|F|V-REGION|
caggctgtgctgactcagccaccctctgtgtctgcagccctggggcagagggtcaccatc
tcctgcactggaagtaacaccaacatcggcagtggttatgatgtacaatggtaccagcag
ctcccaggaaagtcccctaaaactatcatttatggtaatagcaatcgaccctcgggggtc
ccggttcgattctctggctccaagtcaggcagcacagccaccctgaccatcactgggatc
caggctgaggatgaggctgattattactgccagtcctatgatgacaacctcgatggtca
SEQ ID NO. 186 IGLV1-104 (P)
>IGLV1-104*01|Canis lupus familiaris_boxer|P|V-REGION|
cagtctgtgctgactcagccagcttcagtgtctgggtccctgggccagaggatcaccatc
tcctgcactaaaagcagctccaacatcggtaggtattatgtgagctgacaacagctccca
ggaacaggccccagaaccgtcatctatgataataataactgaccctcgggggtccctgat
caattttctggctctaaatcaggcagcacagccaccctgaccatctctaggctccaggct
gaggacgatgctgattattactgctcgccatatgccagcagtctcagtgctgg
SEQ ID NO. 187 IGLV1-106 (F)
>IGLV1-106*01|Canis lupus familiaris_boxer|F|V-REGION|
cagtctgtgttgactcaaccggcctcagtgtctgggtccctgggccagagggtcatcatc
tcctgcactggaagcagctccagcattggcagaggttatgtgggctggtaccaacagctc
ccaggaacaggccccagaaccctcatctatggtattagtaacctacccccgggagtcccc
aatagattctctggttcgaggtcaggcagcacagccaccctgaccatcgctgagctccag
gctgaggacgaggctgattattactgctcatcgtgggacagaagtctcagtgctcc
SEQ ID NO. 188 IGLV1-107 (P)
>IGLV1-107*01|Canis lupus familiaris_boxer|P|V-REGION|
caggctgtgctgactcagcccgccctcagtgtctgcggccttgggacagagggtcaccat
ctcctgcactggaagcagcaccaacatcagcagtggttacgttgtacaatggtaccagca
gctcccaggaaagtcccctaaaacaatctatggtactagcaagtgacccttggggatccc
ggttcaattctctggctccaagtcaggcagcacagccaccctgaccatcactggtatcta
ggctgaggacgaggctgattattactgccaatcctatgatgacaacctcgatggtca
SEQ ID NO. 189 IGLV1-110 (P)
>IGLV1-110*01|Canis lupus familiaris_boxer|P|V-REGION|
caggctgtacggaatcaaccgccctcagagtctgcagccctgggacagagagtcaccatc
tcctgcacgggaagcagatccaacattggcagtggttatgctgtacaatggtaccaacgg
ctcacaggaaagtctccttaaaactatcatctatggtaatagcaatcaaccctcgggggt
cctggatcaattctctggctccaagtgaggcagcacagccaccctgaccatcactgggat
ccagtctgaggacgaggctgattattactgccagtcctatgatagaagtctctgtgctca
SEQ ID NO. 190 IGLV1-111 (ORF)
>IGLV1-111*01|Canis lupus familiaris_boxer|ORF|V-REGION|
cagtctgtgctgactcagccggcctcagtgtctgggtccctgggcctgagggtcaccatc
tgctgcactggaagcagctccaacatcagtagttattatgtgggctggtaccaaccactc
gcgggaacaggccccagaactgtcatctatgataatagtaaccgtccctcgggggtccct
gatcaattctctggctccaagtcaggcagcacagccaccctgaccatctctcggctccag
gctgaggacgaggctgattattacggctcatcatatgacagcagtctcaatgctgg
SEQ ID NO. 191 IGLV1-112 (P)
>IGLV1-112*01|Canis lupus familiaris_boxer|P|V-REGION|
cagtctgtgctgactcagccagcctcagtgtctcagtccctgggtcagagggtcaccatc
tcctgtactggaagcagctccaatgttggttataacagttatgtgagctggtaccagcag
ctcccaggaacagtccccagaaccatcatctattataccaatactcgaccctatggggtt
cctgatcgattctctggctccaaatcaggcaactcagccaccctgaccattgctggactc
caggctgaggacgaggctgattattattgctcaacatatgacagcagtctcagtggtgc
SEQ ID NO. 192 IGLV1-113 (P)
>IGLV1-113-1*01|Canis lupus familiaris_boxer|P|V-REGION|
cagtctgtgctgaatcagacgccctcagtgtcggggtccctgggccagagagtcgccatc
tcctgctctggaagcacaaacatcagtaggtttggtgcgagctggtaacaacagctcctg
ggaaaggcttcaaaactcctcctagacagtgatggggatcaaccatcagtggtccctgac
tgattttccggctccaagtctggcaactcaggtgccctgaccatcactgggctccaggct
gaggacgaggctgattattactgccagtcctttgatcccacacttggtgctca
SEQ ID NO. 193 IGLV1-114 (P)
>IGLV1-114*01|Canis lupus familiaris_boxer|P|V-REGION|
caggctttgctgactcagccaccctcagtgtctgaggccctgggacagagggtcaccatc
tcctgcactggaagcagcaccaacatcggcagtggttatgatgtacaatggtaccagcag
ctcccaggaaagtcccctcaaactatcgtatacggtaatagcaattgaccctcgggggtc
ccagatcaattctctggctccaagtctcacaattcagccaccctgaccatcactgggctc
cagactgaggacgaggctgattattactgccagtcctctgatgacaacctcga
SEQ ID NO. 194 IGLV1-115 (P)
>IGLV1-115*01|Canis lupus familiaris_boxer|P|V-REGION|
cagtctgtgctgactcagccagcctcagtgtctgggtccctgggccagagggtcaccatc
tcctgcactggaagcagctccaacatcggtagatatagtgtaggctgataccagcagctc
ccgggaacaggccccagaactgtcatctatggtagtagtagccgaccctcgggggtcccc
gatcgattctctggctccaagtcaggcagcacagccaccctgaccatctcagggctccag
gctgaggacgaggctgattattactgttcaacatacgacagcagtctcaaagctcc
SEQ ID NO. 195 IGLV1-116 (F)
>IGLV1-116*01|Canis lupus familiaris_boxer|F|V-REGION|
cagcctgtgctcactcagccgccctcagtgtctgggttcctgggacagagggtcactatc
tcctgcactggaagcagctccaacatccttggtaattctgtgaactggtaccagcagctc
acaggaagaggccccagaaccgtcatctattatgataacaaccgaccctctggggtccct
gatcaattctctggctccaagtcaggcaactcagccaccctgaccatctctgggctccag
gctgaggacgagactgattattactgctcaacgtgggacagcaggctcagagctcc
SEQ ID NO. 196 IGLV1-118 (P)
GLV1-118*01|Canis lupus familiaris_boxer|P|V-REGION|
cagtctgtgctgactcagccggcctcagtgtctgggtccctgggccagagggtcaccatc
tcctgcactgaaagcagctccaacatcggtggatattatgtgggctggtaccaacagctc
ccaggaacaggccccagaaccatcatctatagtagtagtaaccgaccctcaggggtccct
gattgattctctggctccaggtcaggcagcacagccaccctgaccatctctgggctccag
gctgaggacgaggctgattattactgctctacatgggacagcagtctcaaagctcc
SEQ ID NO. 197 IGLV1-118-2 (P)
>IGLV1-118-2*01|Canis lupus familiaris_boxer|P|V-REGION|
ctgcctgtgctgacccagccgccctcaaggtctgggggtctggttcagaggttcaccatc
ttctgttctggaagcacaaacaacataggtgataattattttaactggtacaaacagctt
ccaggaacggcccctaaaaccatcatctaagtggatcatatcagaccctcaggggtcctg
gagagattctctgtctccaattctggcagctcagccaacctgaccatctctgggctccag
gctgaggactaggctgattattattgctcatcctgggatgatagtctcaatgctcc
SEQ ID NO. 198 IGLV1-122 (P)
>IGLV1-122*01|Canis lupus familiaris_boxer|P|V-REGION|
caggctgtgctgactcagctgccctcagtgtctgcagccctgggacagagggtcaccatc
tgcactggaagcagcaccaacatcggcagtggttattatacactatggtaccagtagctg
caggaaagtcccctaaaactatcatctatggtaatagcaatcgacccttgagggtcccgg
atcgattctctggctccaagtatggcaattcagccacgctgaccatcactgggctccagg
ctgaggacgaggatgattattactgccagtcctctgatgacaacctcgatggtca
SEQ ID NO. 199 IGLV1-123 (P)
>IGLV1-123*01|Canis lupus familiaris_boxer|P|V-REGION|
cagtctgtgctgactcagccggcctcagtgtctgggtccctgggtcagagggtcaccatc
tcctgcactggaagcagctccaacatcggtgaatattatgtgagttggctccagcagctc
ccgggaacacgccccagaaccgtcatctatagtagtagtaaccgaccctcaggggtccct
gatcgattctctggctccaagtcaggtagcatagccaccctatctctgggctccaggctg
aagacgaggctgattattactgtactacgtgggacagcagtctcaatgctgg
SEQ ID NO. 200 IGLV1-125 (F)
>IGLV1-125*01|Canis lupus familiaris_boxer|F|V-REGION|
cagtctgtgctgactcagccggcctcagtgtccgggtccctgggccagagggtcaccatc
tcctgcactggaagcagctccaacatcggtagaggttatgtgggctggtaccaacagctc
ccgggaacaggccccagaaccctcatctatggtaatagtaaccgaccctcaggggtcccc
gatcggttctctggctccaggtcaggcagcacagccaccctgaccatctctgggctccag
gctgaggatgaggctgattattactgctcatcgtgggacagcagtctcagtgctct
SEQ ID NO. 201 IGLV1-127 (P)
>IGLV1-127*01|Canis lupus familiaris_boxer|P|V-REGION|
cagtctgtgctgactcagcctccctcagtgtctgggtccctgggccagaggtcaccgtct
cctgcactggaagctgcttcaacattggtagatatagtgtgagctggctccagcagctcc
cgggaacaggccccagaaccatcatctattatgatcgtagccgaccctcaggggttcccg
atcgattctctggctccaagtcaggcagcacagccaccctgaccatctctgggctccagg
ctgaggacgaggctgattattactgctcatcctatgacagcagtctcaaaggtca
SEQ ID NO. 202 IGLV1-129 (P)
>IGLV1-129*01|Canis lupus familiaris_boxer|P|V-REGION|
cagtctgtgctgactcaaccagtctcagtgtctggggccctgtgccagagggtcaccatc
tcctgcactggaagcagctccaacattggttatagcagctgtgtgagctgatatcagcag
ctcccaggaacaggccccagaaccatcatctatagtatgaatactctaccctctggggtt
cctgatcgattgtctggctccaggtcaggcaactcagccaccctaaccatctctgggctc
caggctgaggacaaggctgactattactgctcaacatatgacagcagtctcaatgctca
SEQ ID NO. 203 IGLV1-130 (P)
>IGLV1-130*01|Canis lupus familiaris_boxer|P|V-REGION|
cagtctgtgctgacccagctggcctcagtgtctgggtccctgggccagagggtcaccatc
acctgcactggaagcagctccaacattggtagtgattatgtgggctggttccaacagctc
ccaggaacaggccctagaaccctcatctaaggcaatagtaaccgaccctcgggggtccct
gatcaattctctggctccaagtctggcagtacagccaccctgaccatctctgggctccag
gctgaggatgatgctgattattactgcacatcatgggatagcagtctcaaggctcc
SEQ ID NO. 204 IGLV1-132 (ORF)
>IGLV1-132*01|Canis lupus familiaris_boxer|ORF|V-REGION|
cagtctgtgctgactcagcctccctcagtgtctgggaccctggggcaaagggtcatcatc
tcctgcactggaatccccagcaacataaatttagaagaattgggaatcgctactaaggtg
aactggtaccaacagctcccaggaaaggcacccagtctcctcatctatgatgatgatagc
agaggttctgggattcctgatcgattctctggctccaagtctggcaactcaggcaccctg
accatcactgggctccaggctgaggatgaggctgattattattgccaatcctatgatgaa
agccttggtgtt
SEQ ID NO. 205 IGLV1-133 (P)
>IGLV1-133*01|Canis lupus familiaris_boxer|P|V-REGION|
cagtctgtgctgactcagcctccctcagtgttcaggtccctgggccagagggtcaccatc
tcctgcactggaagcagctccaacgtcggtagaggttatgtgatctggtaccaaagctcc
tgggaacacgcccaagaaccctcatatatggtagtagtaaccaaccctcaggggtcccca
atcgattctctggctccaggtcaggcagcacagacactctgacaatctctgtgttccagg
ctgaggatgaggctgattattactgctcatcctgggacagcagtctcagtgctct
SEQ ID NO. 206 IGLV1-135 (F)
>IGLV1-135*01|Canis lupus familiaris_boxer|F|V-REGION|
cagtctgtgctgaatcagctgccttcagtgttaggatccctgggccagagaatcaccatc
tcctgctctggaagcacgaatgacatcggtatgcttggtgtgaactggtaccaagagctc
ccaggaaaggcccctaaactcctcgtagatggtactgggaatcgaccctcaggggtccct
gaccgattttctggctccaaatctggcaactcaggcactctgaccatcactgggctccag
gctgaggacgaggctgattattattgtcagtccactgatctcacgcttggtgctcc
SEQ ID NO. 207 IGLV1-136 (F)
>IGLV1-136*01|Canis lupus familiaris_boxer|F|V-REGION|
cagtctgtgctgactcagccggcctcagtgtctgggtccctgggccagagggtcaccatc
tcctgcactggaagcagctccaacatcggtagaggttatgtgggctggtaccagcagctc
ccaggaacaggccccagaaccctcatctatgatagtagtagccgaccctcgggggtccct
gatcgattctctggctccaggtcaggcagcacagcaaccctgaccatctctgggctccag
gctgaggacgaggctgattattactgctcagcatatgacagcagtctcagtggtgg
SEQ ID NO. 208 IGLV1-138 (F)
>IGLV1-138*01|Canis lupus familiaris_boxer|F|V-REGION|
cagtctgtgctgactcagccggcctcagtgtctgggtccctgggccagagggtcaccatc
tcctgcactggaagcagctccaatgttggttatggcaattatgtgggctggtaccagcag
ctcccaggaacaagccccagaaccctcatctatgatagtagtagccgaccctcgggggtc
cctgatcgattctctggctccaggtcaggcagcacagcaaccctgaccatctctgggctc
caggctgaggatgaagccgattattactgctcatcctatgacagcagtctcagtggtgg
SEQ ID NO. 209 IGLV1-139 (F)
>IGLV1-139*01|Canis lupus familiaris_boxer|F|V-REGION|
caggctgtgctgactccgctgccctcagtgtctgcggccctgggacagacggtcaccatc
tcttgtactggaaatagcacccaaatcagcagtggttatgctgtacaatggtaccagcag
ctcccaggaaagtcccctgaaactatcatctatggtgatagcaatcgaccctcgggggtc
ccagatcgattctctggcttcagctctggcaattcagccacactggccatcactgggctc
caggatgaggacgaggctgattattactgccagtccttagatgacaacctcaatggtca
SEQ ID NO. 210 IGLV1-140 (P)
>IGLV1-140*01|Canis lupus familiaris_boxer|P|V-REGION|
cagtctgtgctgactcaaccggcctccgtgtctggggacttgggccagagggtcaccatc
tcctgcactggaagcagctccaattttggttatagcagctatgtgggcttgtaccagcag
ctcccaggaacaggccccagaaccatcatctataataccaatactcgaccctctggggtt
cctgatcgattctctggctccaaatcaggcagcacagccacctgaccattgctggacttc
aagctgaggacgaggctgattattactgctcatcctatgacagcagtctcaaagctcc
SEQ ID NO. 211 IGLV1-140-1 (P)
>IGLV1-140-1*01|Canis lupus familiaris_boxer|P|V-REGION|
cagtctgtactgactcagccgccattagtgcttggggccctggccagagggtcaccttct
cctgccttggaagagtcccagtattggtgattatggtgtgaaatggtacaagcagctcaa
aaggacagaccccagacttctcatctatggcaatagcaattgatcctcgggtccccaatc
aattttctggctctggttttggcatcactggctccttgaccacctatgggctccagactg
aaaaataggctgattactagtgcttctccggtgatccag
SEQ ID NO. 212 IGLV1-141 (F)
>IGLV1-141*01|Canis lupus familiaris_boxer|F|V-REGION|
cagtctgtgctgactcagccgacctcagtgtcggggtcccttggccagagggtcaccatc
tcctgctctggaagcacgaacaacatcggtattgttggtgcgagctggtaccaacagctc
ccaggaaaggcccctaaactcctcgtgtacagtgttggggatcgaccgtcaggggtccct
gaccggttttccggctccaactctggcaactcagccaccctgaccatcactgggcttcag
gctgaggacgaggctgattattactgccagtcctttgataccacgcttggtgctca
SEQ ID NO. 213 IGLV1-143 (P)
>IGLV1-143*01|Canis lupus familiaris_boxer|P|V-REGION|
cagtctgtgctgactcaaccagtctcagtgtctggggccctgtgccagagggtcaccatc
tcctgcactggaagcagctccaacattggttatagcagctgtgtgagctgatatcagcag
ctcccaggaacaggccccagaaccatcatctatagtatgaatactctaccctctggggtt
cctgatcgattgtctggctccaggtcaggcaactcagccaccctaaccatctctgggctc
caggctgaggacaaggctgactattactgctcaacatatgacagcagtctcaatgctca
SEQ ID NO. 214 IGLV1-144 (F)
>IGLV1-144*01|Canis lupus familiaris_boxer|F|V-REGION|
cagtctgtgctgactcagcctccctcagtgttcaggtccctgggccagagggtcaccatc
tcctgcactggaagcagctgcaacgtcggtagaggttatgtgatctggtaccaacagctc
ctgggaacacgcccaagaaccctcatatatggtagtagtaaccaaccctcaggggtcccc
aatcgattctctggctccaggtcaggcagcacagccactctgacaatctctgggttccag
gctgaggatgaggctgattattactgctcatcctgggacagcagtctcagtgctct
SEQ ID NO. 215 IGLV1-146 (P)
>IGLV1-146*01|Canis lupus familiaris_boxer|P|V-REGION|
cagtctgtgctgaatcagctgccttcagtgttaggatccctgggccagagaatcaccatc
tcctgctctggaagcacgaatgacatcggtatgcttggtgtgaactggtaccaagagctc
ccaggaaaggcccctaaactcctcgtagatggtactgggaatcgaccctcaggggtccct
gactgattttctggctccaaatctggcaactcaggcactctgaccatcactgggctccag
gctgaggacgaggctgattattattgtcagtccactgatctcacgcttggtgctcc
SEQ ID NO. 216 IGLV1-147 (F)
>IGLV1-147*01|Canis lupus familiaris_boxer|F|V-REGION|
cagtctgtgctgactcagccggcctcagtgtctgggtccctgggccagagggtcaccatc
tcctgcactggaagcagctccaacatcggtagaggttatgtgggctggtaccagcagctc
ccaggaacaggccccagaaccctcatctatgataatagtaaccgaccctcgggggtccct
gatcgattctctggctccaagtcaggcagcacagccaccctgaccatctctgggctccag
gctgaggacgaggctgattattactgctcaacatacgacagcagtctcagtggtgg
SEQ ID NO. 217 IGLV1-149 (F)
>IGLV1-149*01|Canis lupus familiaris_boxer|F|V-REGION|
cagtctgtgctgactcagccggcctcagtgtctgggtccctgggccagagggtcaccatc
tcctgcactggaagcagctccaatgttggttatggcaattatgtgggctggtaccagcag
ctcccaggaacaggccccagaaccctcatctatcgtagtagtagccgaccctcgggggtc
cctgatcgattctctggctccaggtcaggcagcacagcaaccctgaccatctctgggctc
caggctgaggatgaagccgattattactgctcatcctatgacagcagtctcagtggtgg
SEQ ID NO. 218 IGLV1-150 (F)
>IGLV1-150*01|Canis lupus familiaris_boxer|F|V-REGION|
caggctgtgctgactccgctgccctcagtgtctgcggccctgggacagacggtcaccatc
tcttgtactggaaatagcacccaaatcggcagtggttatgctgtacaatggtaccagcag
ctcccaggaaagtcccctgaaactatcatctatggtgatagcaatcgaccctcgggggtc
ccagatcgattctctggcttcagctctggcaattcagccacactggccatcactgggctc
caggatgaggacgaggctgattattactgccagtccttagatgacaacctcgatggtca
SEQ ID NO. 219 IGLV1-151 (F)
>IGLV1-151*01|Canis lupus familiaris_boxer|F|V-REGION|
cagtctgcgctgactcaaacggcctccatgtctgggtctctgggccagagggtcaccgtc
tcctgcactggaagcagttccaacgttggttatagaagttatgtgggctggtaccagcag
ctcccaggaacaggccccagaaccatcatctataataccaatactcgaccctctggggtt
cctgatcgattctctggctccatatcaggcagcacagccaccctgactattgctggactc
caggctgaggacgaggctgattattactgctcatcctatgacagcagtctcaaagctcc
SEQ ID NO. 220 IGLV1-151-1 (P)
>IGLV1-151-1*01|Canis lupus familiaris_boxer|P|V-REGION|
cagtctgtgctgactcagccactgttagggcctgggttcctggccagagggtcaccctct
cctgccctggaagagtctcagttttggtgattatggtgtgaaacggtacaggaagctcgc
atggacagaccccagactcctcatctatggcaatagcaattgattctcgggtccccagtc
tattttctggctctggttttggcatcactggctccttgaccacctccgggctccagactg
aaaaataggctgatttctagtgcttc
SEQ ID NO. 221 IGLV1-152 (P)
>IGLV1-152*01|Canis lupus familiaris_boxer|P|V-REGION|
caatctgtgctgatccagccggcctcagtgtcgggatccctgggccagagagtcaccatc
tcctgctctggaaggacaaacaacatcggtaggtttggtgcgagctggtaccaacagctc
ccaggaaaggcccctaaactcctcgtggacagtgatggggattgaccgtcaggggtccct
gaccggttttccggctccaggtctggcagctcagccaccctgaccatcactggggtccag
gctgaggatgaggctgattattactgccagtcctttgatcccacgcttggtgctca
SEQ ID NO. 222 IGLV1-154 (P)
>IGLV1-154*01|Canis lupus familiaris_boxer|P|V-REGION|
cagtctgtgctgactcaaccgtcctcagtgtccgggtccctgggccagagggtcactgtc
ccctgcactggaagcagctccaacattggtagatatagtgtgagctggctatatctgctg
gctccagcagctcccgggaacaggccccagaaccatcatctattatgattgtagccgacc
ctcaggggttcccgatcgattctctggctccaagtcaggcagcacagccaccctgaccat
ctctgggctccaggctgaggacgaggctgattattactgctcatcctatgacagcagtct
caaaggtca
SEQ ID NO. 223 IGLV1-155 (F)
>IGLV1-155*01|Canis lupus familiaris_boxer|F|V-REGION|
cagtctgtgctgactcagcctccctcagtgtccgggttcctgggccagagggtcaccatc
tcctgcactggaagcagctccaacatcggtagaggttatgtgcactggtaccaacagctc
ccaggaacaggccccagaaccctcatctatggtattagtaaccgaccctcaggggtcccc
gatcgattctctggctccaggtcaggcagcacagccactctgacaatctctgggctccag
gctgaggatgaggctgattattactgctcatcctgggacagcagtctcagtgctct
SEQ ID NO. 224 IGLV1-157 (F)
>IGLV1-157*01|Canis lupus familiaris_boxer|F|V-REGION|
cagtctgtgctgactcagccggcctcagtgtctgggtccctgggccagagggtcaccatc
tcctgcactggaagcagctccaacatcggtagaggttatgtgggctggtaccagcagctc
ccaggaacaggccccagaaccctcatctatgataatagtaaccgaccctcgggggtccct
gatcgattctctggctccaagtcaggcagcacagccaccctgaccatctctgggctccag
gctgaggacgaggctgattattactgctcaacatacgacagcagtctcagtggtgg
SEQ ID NO. 225 IGLV1-158 (F)
>IGLV1-158*01|Canis lupus familiaris_boxer|F|V-REGION|
cagtctgtgctgaatcagctgccttcagtgttaggatccctgggccagagaatcaccatc
tcctgctctggaagcacgaatgacatcggtatgcttggtgtgaactggtaccaagagctc
ccaggaaaggcccctaaactcctcgtagatggtactgggaatcgaccctcaggggtccct
gaccgattttctggctccaaatctggcaactcaggcactctgaccatcactgggctccag
gctgaggacgaggctgattattattgtcagtccactgatctcacgcttggtgctcc
SEQ ID NO. 226 IGLV1-159 (F)
>IGLV1-159*01|Canis lupus familiaris_boxer|F|V-REGION|
cagtctgtgctgactcagcctccctcagtgttcaggtccctgggccagagggtcaccatc
tcctgcactggaagcagctgcaacgtcggtagaggttatgtgatctggtaccaacagctc
ctgggaacacgcccaagaaccctcatatatggtagtagtaaccaaccctcaggggtcccc
aatcgattctctggctccaggtcaggcagcacagccactctgacaatctctgggttccag
gctgaggatgaggctgattattactgctcatcctgggacagcagtctcagtgctct
SEQ ID NO. 227 IGLV1-160 (P)
>IGLV1-160*01|Canis lupus familiaris_boxer|P|V-REGION|
cagtctgtgctgactcaaccagtctcagtgtctggggccctgtgccagagggtcaccatc
tcctgcactggaagcagctccaacattggttatagcagctgtgtgagctgatatcagcag
ctcccaggaacaggccccagaaccatcatctatagtatgaatactctaccctctggggtt
cctgatcgattgtctggctccaggtcaggcaactcagccaccctaaccatctctgggctc
caggctgaggacaaggctgactattactgctcaacatatgacagcagtctcaatgctca
SEQ ID NO. 228 IGLV1-161 (P)
>IGLV1-161-1*01|Canis lupus familiaris_boxer|P|V-REGION|
caaggtcagctgccctgaggacagagtccatgacaggtcagggcagaaacagggactctg
aatccagctctgagtcaggacacatcaggagtgtccaatatgtgtcctgctaccaacagc
tccatgagtgggcagtcaaatcctcatgtattatgatggcttgaccttctgtggaccctg
gtccattctctgcctccatgtctggcagctctggctctctggccattgctgggctgagcc
aggaggatgaggtcatgcttcactgcccctccagtgacagcatttcaaggat
SEQ ID NO. 229 IGLV1-162 (F)
>IGLV1-162*01|Canis lupus familiaris_boxer|F|V-REGION|
cagtctgtgctgactcagccgacctcagtgtcggggtcccttggccagagggtcaccatc
tcctgctctggaagcacgaacaacatcggtattgttggtgcgagctggtaccaacagctc
ccaggaaaggcccctaaactcctcgtgtacagtgatggggatcgaccgtcaggggtccct
gaccggttttccggctccaactctggcaactcagacaccctgaccatcactgggcttcag
gctgaggacgaggctgattattactgccagtcctttgataccacgcttgatgctca
SEQ ID NO. 230 IGLV2-31 (F)
>IGLV2-31*01|Canis lupus familiaris_boxer|F|V-REGION|
cagtctgccctgactcaaccttcctcggtgtctgggactttgggccagactgtcaccatc
tcctgtgatggaagcagcagtaacattggcagtagtaattatatcgaatggtaccaacag
ttcccaggcacctcccccaaactcctgatttactataccaataatcggccatcagggatc
cctgctcgcttctctggctccaagtctgggaacacggcctccttgaccatctctgggctc
caggctgaagatgaggctgattattactgcagcgcatatactggtagtaatactttc
SEQ ID NO. 231 IGLV2-31-1 (P)
>IGLV2-31-1*01|Canis lupus familiaris_boxer|P|V-REGION|
cagtctaacctaattgagcccccctttttgtccaggattctaggatggactgtcactgtc
tcctgtgttttaagcagctgtgacatcaggagtgataatgaaatatcctggtaccaatag
cacccgagcatgactcagaaattcctgatttactataccagttcttgggcatcagatatc
cctgattgctttcctggctcccagtctggaaacatggcctgtctgaccatttccaggctc
caggctaatgatgacgctgattatcattgttacttatatgatggtagtggcgctttt
SEQ ID NO. 232 IGLV2-32 (P)
>IGLV2-32*01|Canis lupus familiaris_boxer|P|V-REGION|
cagtctgccctgactcagcctccctcgatgtctgggacactgggacagaccatcatcatt
tcctgtactggaagcggcagtgacattgggaggtatagttatgtctcctggtaccaagag
ctcccaagcacgtcccccacactcctgatttatggtaccaataatcggccattagagatc
cctgctcgcttctctggctccaagtctggaaacacagcccccatgaccatctctgggctt
caggctgaagatgaggctaattattactgttgctcatatacaaccagtggcacaca
SEQ ID NO. 233 IGLV2-32-1 (P)
>IGLV2-32-1*01|Canis lupus familiaris_boxer|P|V-REGION|
cagtctgccttgacccaacctccctttgtgtctgggactttgagacaaactgtcacatct
cttgcaatggaagcagcagccacactggaacttataaccctacctctggcaccagcaatg
tctggaaaggcccccacactccagatagatgctgtgagttctttgccttcagggcttcca
gctctgtcctcaggctctgagtctagcaacacagcctccagtccatttttggactgcacc
ctgaggacaaggctgattattactgattgtccagggacagccagag
SEQ ID NO. 234 IGLV3-1 (P)
>IGLV3-1*01|Canis lupus familiaris_boxer|P|V-REGION|
gccaacaagctgactcaatccctgtttatgtcagtggccctgggacagatggccaggatc
acctgtgggagagacaactctggaagaaaaagtgctcactggtaccagcagaagccaagc
caggctcccgtgatgcttatcgatgatgattgcttccagccctcaggattctctgagcaa
ttctcaggcactaactcggggaacacagccaccctgaccattagtgggcccccagcgagg
acgcggctattactgtgccaccagccatggcagttggagcacct
SEQ ID NO. 235 IGLV3-1-1 (P)
>IGLV3-1-1*01|Canis lupus familiaris_boxer|P|V-REGION|
tccaatgtactgacacagccacccttggtgtcagtgaacctgggacagaaggccagcctc
acctgtggaagaaacagcattgaagataaatatgtttcatggtcccagcaggagccaggc
caggcccccatgctggtcatctattatagtacacaagaaaccctgagcgattttctgcct
ccagctctagctcggggtacatgatcaccctgaccaacagtggggcctaggacaaggacg
aggatggctattactgtcagtcctatgacagtagtggtactcct
SEQ ID NO. 236 IGLV3-2 (F)
>IGLV3-2*01|Canis lupus familiaris_boxer|F|V-REGION|
tcctatgtgctgactcagtcaccctcagtgtcagtgaccctgggacagacggccagcatc
acctgtaggggaaacagcattggaaggaaagatgttcattggtaccagcagaagccgggc
caagcccccctgctgattatctataatgataacagccagccctcagggatccctgagcga
ttctctgggaccaactcagggagcacggccaccctgaccatcagtgaggcccaaaccaac
gatgaggctgactattactgccaggtgtgggaaagtagcgctgatgct
SEQ ID NO. 237 IGLV3-3 (F)
>IGLV3-3*01|Canis lupus familiaris_boxer|F|V-REGION|
tcctatgtgctgacacagctgccatccaaaaatgtgaccctgaagcagccggcccacatc
acctgtgggggagacaacattggaagtaaaagtgttcactggtaccagcagaagctgggc
caggcccctgtactgattatctattatgatagcagcaggccgacagggatccctgagcga
ttctccggcgccaactcggggaacacggccaccctgaccatcagcggggccctggccgag
gacgaggctgactattactgccaggtgtgggacagcagtgctaaggct
SEQ ID NO. 238 IGLV3-4 (F)
>IGLV3-4*01|Canis lupus familiaris_boxer|F|V-REGION|
tccactgggttgaatcaggctccctccatgttggtggccctgggacagatggaaacaatc
acctgctccggagatatcttagggaaaagatatgcatattggtaccagcataagccaagc
caagcccctgtgctcctaatcaataaaaataatgagcgggcttctgggatccctcactgg
ttctctggttccaactcgggcaacatggccaccctgaccatcagtggggcccgggctgag
gacgaggctgactattactgccagtcctatgacagcagtggaaatgct
SEQ ID NO. 239 IGLV3-7 (P)
>IGLV3-7*01|Canis lupus familiaris_boxer|P|V-REGION|
tcctatgtgctgactctgctgctatcagtgaccgtgaacctgggacagaccaccagcatc
acctgtggtggagacagcattggagggagaactgtttactggtaccagcagaagcctggc
cagcgccccctgctgattatctataatgatagcaattgaccctcagggatccctgcctga
ttctctggctccaactcagggaacagggcctccctaaccatcattggggcctgggcctaa
gacgagtctgagtattacggagaggtgtgggacagcagtgctaaggct
SEQ ID NO. 240 IGLV3-7-1 (P)
>IGLV3-7-1*01|Canis lupus familiaris_boxer|P|V-REGION|
tcctatatgctgactcagcagccattggcaagtgtaaacctcagccagtgggccagcacc
acctgtggtggagataacattggagaaaaaaccgtccaatggaaccagcagaagcctggc
taagctcccattacggctatctataaaggtagtgatctgccctcagggatccctgagcaa
ttccctggccccaatttggggaacggggcctccctgaacatcagcggggctaagccgacg
acgaggctattactgccagtcagcagacattagtggtaaggct
SEQ ID NO. 241 IGLV3-8 (F)
>IGLV3-8*01|Canis lupus familiaris_boxer|F|V-REGION|
tcctatgtgctgacacagctgccatccgtgagtgtgaccctgaggcagacggcccgcatc
acctgtgggggagacagcattggaagtaaaagtgtttactggtaccagcagaagctgggc
caggcccctgtactgattatctatagagatagcaacaggccgacagggatccctgagcga
ttctctggcgccaactcggggaacacggccaccctgaccatcagcggggccctggccgag
gacgaggctgactattactgccaggtgtgggacagcagtactaaggct
SEQ ID NO. 242 IGLV3-9 (P)
>IGLV3-9*01|Canis lupus familiaris_boxer|P|V-REGION|
tccactgggttgaatcaggctccctccgtgttgctggcactgggacagatggcaacaatc
acctgatccagagatgtctttgggaaaaatatgcatattggtaccagcagaagccaagcc
aagcccctgtgctcctaatcaataaaaataatgagcaggattctgggatccctgaccggt
tctctggctccaactcgggcaacacggccaccctgaccatcagtggggcccgggccgagg
acgaggctgactattactgccagtcctatgacagcagtggaaatgtt
SEQ ID NO. 243 IGLV3-11 (F)
>IGLV3-11*01|Canis lupus familiaris_boxer|F|V-REGION|
tcctatgtgctgtctcagccgccatcagcgactgtgactctgaggcagacggcccgcctc
acctgtgggggagacagcattggaagtaaaagtgttgaatggtaccagcagaagccgggc
cagccccccgtgctcattatctatggtgatagcagcaggccgtcagggatccctgagcga
ttctccggcgccaactcggggaacacggccaccctgaccatcagcggggccctggccgag
gacgaggctgactattactgccaggtgtgggacagcagtactaaggct
SEQ ID NO. 244 IGLV3-13 (P)
>IGLV3-13*01|Canis lupus familiaris_boxer|P|V-REGION|
tcctatgtactgactcagctgccatcagtgactgtgaacctgggacagaccaccagcatc
acctgtggtggagacagcattggagggagaactgtttactggtaccagcagaagcctggc
cagcgccccctgctgattatctataatgatagcaattggccctcagagatccctgcctga
ttctctggctccaactcagggaacagggcctccctaaccatcattggggcctgggcctaa
gatgagtctgagtattacggagaggtgtgggacagcagtgctaaggct
SEQ ID NO. 245 IGLV3-13-1 (P)
>IGLV3-13-1*01|Canis lupus familiaris_boxer|P|V-REGION|
tcctatatgctgactcagcagccattggcaagtgtaaacctcagccagtgggccagcacc
acctgtggtggagataacattggagagaaaactgtccaatggaaccagcagaagcctggc
taagctctcattatggctatctataaaggtagtgatctaccctcagggatccctgagcaa
ttccctggccccaactcgggtcggggcctccctgaacatcagcggggctacgccgacgac
taggctattactgccagtcagcagacattagtggtaaggct
SEQ ID NO. 246 IGLV3-14 (F)
>IGLV3-14*01|Canis lupus familiaris_boxer|F|V-REGION|
tcctatgtgctgacacagctgccatccatgagtgtgaccctgaggcagacggcccgcatc
acctgtgagggagacagcattggaagtaaaagagtttactggtaccagcagaagctgggc
caggtccctgtactgattatctatgatgatagcagcaggccgtcagggatccctgagcga
ttctccggcgccaactcggggaacacagccaccctgaccatcagcggggccctggccgag
gacgaggctgactattactgccaggtgtgggacagcagtactaaggct
SEQ ID NO. 247 IGLV3-15 (P)
>IGLV3-15*01|Canis lupus familiaris_boxer|P|V-REGION|
tccactgggttgaatcaggctccctccgtgttggtggccctgggacagatggaaacaatc
acctgctcgagagatgtcttagggaaaagatatgcatataggtaccagcataagccaagc
caagcccctgtgctcctaatcaataaaaataatgagcaggattctgggatccctgaccgg
ttctctggctccaactcgggcaacacggccaccctgaccatcagtggggcccgggctgag
gacgaggctgagtattactgccagtcctatgacagcagtggaaatgtt
SEQ ID NO. 248 IGLV3-18 (P)
>IGLV3-18*01|Canis lupus familiaris_boxer|P|V-REGION|
tcctatgtgctgacacagctgccatccgtgaatgtgacccagaggcagacggcccgcatc
acctgtgggggagacagcattggaagtaaaagtgtttactggtaccagcagaagctgggc
caggcccctgttgattatctatagagacagcaacaggccgacagggatccctgagcgatt
ctctggcgccaacacggggaacatggccaccctgactatcagcggggccctggccgtgga
cgaggctgactattactgccaggtgtgggacagcagtgctaaggct
SEQ ID NO. 249 IGLV3-19 (ORF)
>IGLV3-19*01|Canis lupus familiaris_boxer|ORF|V-REGION|
tcccctgggctgaatcagcctccctccgtgttggtggccctgggacagatggcaacaaac
acctgctccggagatgtcttagggaaaagatatgcatattggtaccagcataagccaagc
caagcccctgtgctcctaatcaataaaaataatgagctgggttctgggatccctgaccga
ttctctggctccaactcgggcaacacggccaccctgaccatcagtggggcccgggccgag
gacgaggctgactattactgccagtcctatgacagcagtggaaatgct
SEQ ID NO. 250 IGLV3-21 (F)
>IGLV3-21*01|Canis lupus familiaris_boxer|F|V-REGION|
tcctatgagctgactcagccaccatccgtgaatgtgaccctgagggagacggcccacatc
acctgtgggggagacagcattggaagtaaatatgttcaatggatccagcagaatccaggc
caggcccccgtggtgattatctataaagatagcaacaggccgacagggatccctgagcga
ttctctggcgccaactcagggaacacggctaccctgaccatcagtggggccctggccgaa
gacgaggctgactattactgccaggtgggggacagtggtactaaggct
SEQ ID NO. 251 IGLV3-23 (P)
>IGLV3-23*01|Canis lupus familiaris_boxer|P|V-REGION|
tcctatgtactgactcagctgccatcagtgactgtgaacctgggacagaccaccagcatc
acctgtggtggagacagcattggagggagaactgtttactggtaccagcagaagcctggc
cagcgccccctgctgattatctataatgatagcaattggccctcagagatccctgcctga
ttctctggctccaactcagggaacagggcctccctaaccatcattggggcctgggcctaa
gacgagtctgagtattacggagaggtgtgggacagcagtgctaaggct
SEQ ID NO. 252 IGLV3-23-1 (P)
>IGLV3-23-1*01|Canis lupus familiaris_boxer|P|V-REGION|
tcctatatgctgactcagcagccattggcaagtgtaaacctcagccagtgggccagcacc
acctgtggtggagataacattggagaaaaaactgtccaatggaaccagcagaagcctggc
taagctcccattacggctatctataaaggtagtgatctgccctcagggattcctgagcaa
ttccctggccccaactcgggaaacggggcctccctgaacatcagcggggctaagccgacg
actaggctattactgccagtcagcagacattagtggtaaggct
SEQ ID NO. 253 IGLV3-24 (F)
>IGLV3-24*01|Canis lupus familiaris_boxer|F|V-REGION|
tcctatgtgctgacacagctgccatccgtgagtgtgaccctgaggcagacggcccgcatc
acctgtgggggagacagcattggaagtaaaaatgtttactggtaccagcagaagctgggc
caggcccctgtactgattatctatgatgatagcagcaggccgtcagggatccctgagcga
ttctccggcgccaactcggggaacacggccaccctgaccatcagcggggccctggccgag
gatgaggctgactattactgccaggtgtgggacagcagtactaagcct
SEQ ID NO. 254 IGLV3-25 (ORF)
>IGLV3-25*01|Canis lupus familiaris_boxer|ORF|V-REGION|
tccactgggttgaatcaggcttcctccgtgttggtggccctgggacagatggaaacaatc
acctgctcgagagatgtcttagggaaaagatatgcatataggtaccagcataagccaagc
caagcccctgtgctcctaatcaataaaaataatgagcaggattctgggatccctgaccgg
ttctctggctccaactcgggcaacacggccaccctgaccatcagtggggcccgggctgag
gacgaggctgagtattactgccagtcctatgacagcagtggaaatgtt
SEQ ID NO. 255 IGLV3-26 (F)
>IGLV3-26*01|Canis lupus familiaris_boxer|F|V-REGION|
tcctatgtgctgacacagctgccatccgtgaatgtgaccctgaggcagccggcccacatc
acctgtgggggagacagcattggaagtaaaagtgttcactggtaccaacagaagctgggc
caggcccctgtactgattatctatggtgatagcaacaggccgtcagggatccctgagcga
ttctctggtgacaactcggggaacacggccaccctgaccatcagtggggccctggccgag
gacgaggcttactattactgccaggtgtgggacagcagtgctcaggct
SEQ ID NO. 256 IGLV3-27 (F)
>IGLV3-27*01|Canis lupus familiaris_boxer|F|V-REGION|
tccagtgtgctgactcagcctccttcagtatcagtgtctctgggacagacagcaaccatc
tcctgctctggagagagtctgagtaaatattatgcacaatggttccagcagaaggcaggc
caagtccctgtgttggtcatatataaggacactgagcggccctctgggatccctgaccga
ttctccggctccagttcagggaacacacacaccctgaccatcagcggggctcgggccgag
gacgaggctgactattactgcgagtcagaagtcagtactggtactgct
SEQ ID NO. 257 IGLV3-28 (F)
>IGLV3-28*01|Canis lupus familiaris_boxer|F|V-REGION|
tcctatgtgttgactcagctgccttcagtgtcagtgaacctgggaaagacagccagcatc
acctgtgagggaaataacataggagataaatatgcttattggtaccagcagaagcctggc
caggcccccgtgctgattatttatgaggatagcaagcggccctcagggatccctgagcga
ttctctggctccaactcggggaacacggccaccctgaccatcagcggggccagggccgag
gatgaggctgactattactgtcaggtgtgggacaacagtgctaaggct
SEQ ID NO. 258 IGLV3-29 (F)
>IGLV3-29*01|Canis lupus familiaris_boxer|F|V-REGION|
tccagtgtgctgactcagcctccctcggtgtcagtgtccctgggacagacggcgaccatc
acctgctctggagagagtctgagcagatactatgcacaatggtatcagcagaagccaggc
caagcccccatgacagtcatatatggggacagagagcgaccctcagggatccctgaccga
ttctccagctccagttcagagaacacacacaccttgacaatcagtggagcccaggctgag
gatgaggctgaatattactgtgagatatgggacgccagtgctgatgat
SEQ ID NO. 259 IGLV3-30 (F)
>IGLV3-30*01|Canis lupus familiaris_boxer|F|V-REGION|
tcctacgtggtgacccagccaccctcagtgtcagtgaacctgggacagacggccagcatc
acctgtgggggagacaacattgcaagcacatatgtttcctggcagcagcagaagtcgggt
caagcccctgtgacgattatctatcgtgatagcaaccggccctcagggatccctgagcga
ttctctggctccaactcggggaacacggccaccctgaccatcagcagggcccaggccgag
gatgaggctgactattactgccaggtgtggaagagtggtaataaggct
SEQ ID NO. 260 IGLV4-5 (F)
>IGLV4-5*01|Canis lupus familiaris_boxer|F|V-REGION|
ttgcccgtgctgacccagcctacaaatgcatctgcctccctggaagagtcggtcaagctg
acctgcactttgagcagtgagcacagcaattacattgttcagtggtatcaacaacaacca
gggaaggcccctcggtatctgatgtatgtcaggagtgatggaagctacaaaaggggggac
gggatccccagtcgcttctcaggctccagctctggggctgaccgctatttaaccatctcc
aacatcaagtctgaagatgaggatgactattattactgtggtgcagactatacaatcagt
ggccaatacggttaagc
SEQ ID NO. 261 IGLV4-6 (P)
>IGLV4-6*01|Canis lupus familiaris_boxer|P|V-REGION|
ttgcccgtgctgacccagcctccaagtgcatctgcctccctggaagcctcggtcaagctc
acatgcactctgagcagtgagcacagcagttactatatttactggtatgaacaacaacaa
ccagggaaggcccctcggtatctgatgagggttaacagtgatggaagccacagcaggggg
gacgggatccccagtcgcttctcaggctccagctctggggctgaccgctatttaaccatc
tccaacatccagtctgaggatgaggcagattattactgtggtgcacccgctggtagcagt
agc
SEQ ID NO. 262 IGLV4-10 (F)
>IGLV4-10*01|Canis lupus familiaris_boxer|F|V-REGION|
ttgcccgtgctgacccagcctacaaatgcatctgcctccctggaagagtcggtcaagctg
acctgcactttgagcagtgagcacagcaattacattgttcattggtatcaacaacaacca
gggaaggcccctcggtatctgatgtatgtcaggagtgatggaagctacaaaaggggggac
gggatccccagtcgcttctcaggctccagctctggggctgaccgctatttaaccatctcc
aacatcaagtctgaagatgaggatgactattattactgtggtgcagactatacaatcagt
ggccaatacggttaagc
SEQ ID NO. 263 IGLV4-12 (P)
>IGLV4-12*01|Canis lupus familiaris_boxer|P|V-REGION|
ttgcccgtgctgacccagcctccaagtgcatctgcctccctggaagcctcggtcaagctc
acatgcactctgagcagtgagcacagcagttactatatttactggtatcaacaacaacca
gggaaggcccctcggtatctgatgaaggttaacagtgatggaagccacagcaggggggac
gggatccccagtcgcttctcaggctccagctctggggctgaccgctatttaaccatctcc
aacatccagtctgaggatgaggcaggttattactatggtgtacccctggtagcagtagc
SEQ ID NO. 264 IGLV4-16 (ORF)
>IGLV4-16*01|Canis lupus familiaris_boxer|ORF|V-REGION|
ttgcccatgctgacccagcctacaaatgcatctgcctccctggaagagtcggtcaagctc
acatgcactttgagcagtgagcacagcaattacattgttcaatggtatcaacaacaacca
gggaaggcccctcggtatctgatgcatgtcaggagtgatggaagctacaacaggggggac
gggatccccagtcgcttctcaggctccagctctggggctgaccgctatttaaccatctcc
aacatcaagtctgaagatgaggatgactattattacagtggtgcatactatacaatcagt
ggccaatacggttaagc
SEQ ID NO. 265 IGLV4-17 (P)
>IGLV4-17*01|Canis lupus familiaris_boxer|P|V-REGION|
ttgcccatgctgacccagcctccaagtgcatctgcctccctggaagcctcggtcaagctc
acatgcactctgagcagtgagcaaagcagttactatatttactggtatcaacaacaacaa
ccagggaaggcccctcggtatctgatgaaggttaacagtgatggaagccacagcagggcg
tcgggatccccagtcgcttctcaggctccagctctggggctgaccgctatttaaccatct
ccaacatccagtctgaggatgaggcagattattactgtggtgtacccactggtagcagta
gc
SEQ ID NO. 266 IGLV4-20 (ORF)
>IGLV4-20*01|Canis lupus familiaris_boxer|ORF|V-REGION|
ttgcccatgctgaccgagcctacaaatgcatctgcctccctggaagagtcagtcaagctc
acctgcactttgagcagtgagcacagcaattacattgttcgatggtatcaacaacaacca
gggaaggcccctcggtatctgatgtatgtcaggagtgatggaagctacaacaggggggac
gggatccccagtcgcttttcaggctccagctctggggctgaccgctatttaaccatctcc
aacatcaagtctgaagatgaggctgagtattattacggtggtgcagactataaaatcagt
gaccaatatggttaaga
SEQ ID NO. 267 IGLV4-22 (F)
>IGLV4-22*01|Canis lupus familiaris_boxer|F|V-REGION|
ttgcccgtgctgacccagcctccaagtgcatctgcctgcctggaaacctcggtcaagctc
acatgcactctgagcagtgagcacagcagttactatatttactggtatcaacaacaacaa
ccagggaaggcccctcggtatctgatgaaggttaacagtgatggaagccacagcaggggg
gacgggatccccagtcgcttctcaggctccagctctggggctgaccgctatttaaccatc
tccaacatccagtctgaagatgaggcagattattactgtggtgtacccgctggtagcagt
agc
SEQ ID NO. 268 IGLV5-34 (P)
>IGLV5-34*01|Canis lupus familiaris_boxer|P|V-REGION|
caggctgtgctgacccagccgccctccctctctgcatccctgggatcaacagccagactc
acctgcaccctgagcagtggcttcagtgttggcagctactacatatactggtaccagtag
aagccagggagccctccccggtatctcctgtactaactactactcaagtacacagctggg
ccccggggtccccagccatttctctggatccaaagacaactcggccaatgcagggctcct
gctcacctctgggctgcagcctgaggacgaggctgactactactgtgctacaggttattg
ggatgggagcaactatgcttacc
SEQ ID NO. 269 IGLV5-38 (P)
>IGLV5-38*01|Canis lupus familiaris_boxer|P|V-REGION|
cagcctgtgctgacccagccgccctccctctctgcatccctgggaacagcggccagaaat
acctgcactctgagcagtgacctcagtgttggcagctgtgctataagctgatcccagcag
aagccagggagccctccctggtatctcctgaactactaaacacacccatgcaagcaccag
gactcacatctgtagccgcttctctggatttgaggatgcctctgccagtgcagggctctg
ctcatctctggaggctgaccatcactgtgctaagatcatggcagtgggggcagctagtgt
taca
SEQ ID NO. 270 IGLV5-38-1 (P)
>IGLV5-38-1*01|Canis lupus familiaris_boxer|P|V-REGION|
cagcctgtgctgacccagccgccgtcctctctgcatccctgggaacaacagccagactca
cctgcaccctgagcagtggcttcaatatgtggggctaccatatattctggtaccagcaga
agccagggagccctccccggtatctgctgaacttctactcagataagcaccagggctcca
aggacacctcggccaatgcagggatcctgctcatctctgggctccagcctgaggacgagg
ctgactactactgtaaaatctggtacagtggtctggt
SEQ ID NO. 271 IGLV5-40-1 (P)
>IGLV5-40-1*01|Canis lupus familiaris_boxer|P|V-REGION|
cagcctctgctacccagccacccccttctctgcgtctccaggtactacagccagacccac
ctgcaccctgagcagtggcaacagtgttggcagctgttccttataacggctcccacaaag
acagagggccctccctggtatctgctgaggttcccctctaatagacaccatgtctctgga
tccacacataccttggccaatgcagggctcctgctcat
SEQ ID NO. 272 IGLV5-42 (P)
>IGLV5-42*01|Canis lupus familiaris_boxer|P|V-REGION|
cagcctgtgctgaccaagtgccctctctttctgcatctcctggaacaacagtcagactca
cttgcacctggagcagtggctccagcactggcagctactatatacactggttccagagcc
acagagccagagccacagagctctccctggtatctcctgtactactactcagactcagat
aagcaccagggctctggggttctcagctctgtctcctgatccaaggatgcctcagttatt
ggagggctctctcatctctgggctgcagcctgaggattagactgaccttcactgtctaat
cagaaacaataatgcttct
SEQ ID NO. 273 IGLV5-47 (P)
>IGLV5-47*01|Canis lupus familiaris_boxer|P|V-REGION|
cagcctgtgctgacccagctgccctccctctctgcataccggggaacaaactccagatgt
acctacaccctgagcagtgtcgccaactactaaacatacttctcaaagagaatacagggc
accttccacagtacatcctgtactactactcagactcaagtgcatgattgggatttgggg
tcccaggcacttctctggatccaaagatgcctcagccaatgcagggatcctgctgatctc
tgggctgcagccagaggacaagtctgactgtcactgtgctacagatcatggcagtgggag
cagcttccgatact
SEQ ID NO. 274 IGLV5-47-1 (P)
>IGLV5-47-1*01|Canis lupus familiaris_boxer|P|V-REGION|
cagccagggctgacccagccacactccctctctgcatatcagggagaaacagccacacat
acctgcaccctgagcggtggcttcagtgttggcagctgccatatatactggatccagaag
aagccagagagccctccctgatgtctcctgaactactactaagactcagataaggcctcg
acgtccccagccctactctgaatccaaagacaccttgcccaaggtgggaatcctgctcat
ctctgggctgcagccggaggacaaggctgtctcttactgtataatatggcacagtggttc
tggtcacagggaca
SEQ ID NO. 275 IGLV5-48-1 (P)
>IGLV5-48-1*01|Canis lupus familiaris_boxer|P|V-REGION|
caccctgtgctgacccagctgccctccctctctgcatccctgggaacaacagccagactc
atgtgcaccctgagcagtggctgcagtggtggccatacgctggttccagcagccaggagg
cctcctgagtacctgctgatggtctactgagactcaccagggccccggtggccccagccg
cttctctggctccaaggacacctcggccaatgcagggctcctgctcatctctaggctgca
gcctgaggacgaggctgactgtcactgtgttacagaccatggcagtgggagcagctcccg
aaactca
SEQ ID NO. 276 IGLV5-49-1 (P)
>IGLV5-49-1*01|Canis lupus familiaris_boxer|P|V-REGION|
cagccagggctggcccagcttccccccacctccctctgcatctccaggaacaacagccag
actcacatgaaccatgagcagtggcttcatcgttggcgctgctacatatactggttccaa
cagaagccagggagcaccgccccagtatctcctgaggttctactcagactcagataagca
ctagggctcaacgaccccagccctgttctggatctgaagacacctccgccgaagcagggc
ctctgctcatctctgggctgcagcgtgaggacaaggctgactcttatgggacaatctggc
acagtggtcctggtcacagggacaca
SEQ ID NO. 277 IGLV5-51 (P)
>IGLV5-51*01|Canis lupus familiaris_boxer|P|V-REGION|
cagcctgtgctgacccagctgccctccctttctgcatccctgggaacaacagccagactc
acatgcaccctgagcagcggctgcagcggtggccacacattggttccagcagccaggagg
cctcctgagtacctgctgatggtctactgagactcaccagggccccggtgttgccagcct
cttctctggctccaaggacacctcggccaatgcaggactcctgctcatctctgggctgca
gcctgaggatgaggctgactgtcactgtgctacagaccatggcagtgggagcagctccgg
atact
SEQ ID NO. 278 IGLV5-53 (P)
>IGLV5-53*01|Canis lupus familiaris_boxer|P|V-REGION|
cagcctgtgctgacccagctgccctccctttctgcatccctgagaacaacagccagactc
acctgcaccctgagcagtggctgcagtggtggccatatgctggttccagcagccaggaag
cctcctgagtatctgctgacggtcttctgagactcaccagggccccgaggtccccagcct
cttctctggctccaaggacacctcagccaatgcaggactcctgctcatctctgggctgca
gcctgaggatgaggctgactgtcactgtgctacagaccatggcagtgggagcagctcccg
atact
SEQ ID NO. 279 IGLV5-53-1 (P)
>IGLV5-53-1*01|Canis lupus familiaris_boxer|P|V-REGION|
caccctgggctgacccagtcgtcctccctctctgcatccctgggaacaacagccagactc
acctgcaccctgagcagtggcttcagaaatgacaggtatgtaataagttggttccagcag
aaatcagggagcccttcctggtgtctcctgtattattactcgaactcaagtacacatttg
ggctctgaggttcccagctgcttctctggatccaagacaaggccacacccacactgagta
gacccctctctgggtgggtctagagctccagctccacctgaggctgatgcacaattgcag
SEQ ID NO. 280 IGLV5-57-1 (P)
>IGLV5-57-1*01|Canis lupus familiaris_boxer|P|V-REGION|
cagccagggctggcccagctgccctccctctctgcatctccaggaacaacagccagactc
acatgaaccatgagcagtggcttcattgttggtggctgctacatatactggttccaacag
aagccagggagcatgccccccagtatctcctgaggttctactcagactcagataagcacc
aggtctcaacatccccagcccggctctggatctgaagacactcagccgaagcagggcctc
tgctcatctctgggctgcagcatgaggacaaggctgactcttactgtacaatctggcaca
gtggtcctggtcacagggaca
SEQ ID NO. 281 IGLV5-58-1 (P)
>IGLV5-58-1*01|Canis lupus familiaris_boxer|P|V-REGION|
cagcctgtgctgacccattgccctccctctctgcatcctgggaaataacaaccagactca
cctgcactctgagcagcggctgcagcggtggccatacagtggttccagcagcaaggaagc
ctcctgagtacctgctgacgttctactgagactcaccagggctctagggtccccagccac
ttctctggtttcaaggacaccacggccaatgcagggcact
SEQ ID NO. 282 IGLV5-59 (P)
>IGLV5-59*01|Canis lupus familiaris_boxer|P|V-REGION|
cagcctgtgctgacccagtcgccctccctctcggcatctttggaacaacagtcagactca
cctgtaccctgatcagtggctccagtgttggcagctattacatcaactggttccagaaga
agccacggagccctccccagtatctcctgtactactacttagactcagataagcaccagg
gctctggggtccccagctgcttctcctgatccaaggatgcctcagtcattggaggacacc
ctcatctctgaactgcagcctgaggactagactgaccttcgctgtctaatcagaaacaat
aatgcttct
SEQ ID NO. 283 IGLV5-62 (P)
>IGLV5-62*01|Canis lupus familiaris_boxer|P|V-REGION|
cagcctgtgctgacccagcctccctctctctctgcatctctgggaacaatagccagacaa
acatgcagcctgagcaggggctacagtatggggacttatgtcatacgctggttccagcag
tagcaagaaactctcctgagtatctgctgaggttatactgagcctcagcaggtctctggg
gaccccagctgagtctttagatccaagatgcctcagccaattcagggctcctgcttatct
ctgtgctgcagcctgaggacaagggttactattactgttctgtacatcatggaattgtga
gcagctatacttacc
SEQ ID NO. 284 IGLV5-64 (F)
>IGLV5-64*01|Canis lupus familiaris_boxer|F|V-REGION|
cagcttgtggtgacccagccgccctccctctctgcatccctgggatcatccgccagactc
acctgcaccctgagcagtggcttcagtgttggcagttattctgtaacttggttccagcag
aagccagggagccctctctggtacctcctgtactaccactcagactcagataagcaccag
ggctccagggtccccagccgcttctctggatccaaggacacctcggccaatgcagggctc
ctgctcatctctgggctgcagcctgaggatgaggctgactactactgtgcctccgctcat
ggcagtgggagcaactaccattact
SEQ ID NO. 285 IGLV5-67-1 (P)
>IGLV5-67-1*01|Canis lupus familiaris_boxer|P|V-REGION|
cagccagtgctgacccagctgccctccttctctgtatctctgggaacaacagtcagactc
acctgcaccctgagcagtgttggcagctactaaacatccttttcaaggagaaaccaagga
gccccccaccccggtatctcctatactactattcagactcagataaaccccaggtctctg
gggtccccagccacttctctgcatccaaagactcctaggccaatgcagggctcctgctcg
cctctgggctgcagcctgaggacgaggctgactatcactgtgctataaatcatgacagtg
ggagtagttcctgatact
SEQ ID NO. 286 IGLV5-70-1 (P)
>IGLV5-70-1*01|Canis lupus familiaris_boxer|P|V-REGION|
cagcctttggtgacccagcgccctccctctctgcatctcctgaaacaacagtcagactca
catgcaccctgagcagtggccccagtgctggcagctactacatacactggttccagtgga
agccacggtgcccgccccggtatctcctgtactactactcagactcagatgagcaccagg
gctctggggtccccagccgcttctcctgatccaaggatgcctcagccagggcagggctcc
ctcatctctgggctacagtctgaggtctacactgaccttcactgtctaatcggaaacaat
aatgtttct
SEQ ID NO. 287 IGLV5-72-1 (P)
>IGLV5-72-1*01|Canis lupus familiaris_boxer|P|V-REGION|
cagcctgtgctgacccagcgacctccctctctgcatccctgggaacaacagccagactca
cctgcaccctgagcagcggctgaagcggtggccatacgctggttccagcagccaggaagc
ctcctgagtacctgctgatggtctactgagactcaccaggctatggggtccccagcatct
tctctggctccaaggacacctcggccaatgcagggctcctgctcatctctgggctgcagc
ctgaggtcgaggctgactgtcactgtgctacagaccatggcagtgggagcagctcccgat
act
SEQ ID NO. 288 IGLV5-76 (P)
>IGLV5-76*01|Canis lupus familiaris_boxer|P|V-REGION|
cagcctgtgctgacccagtcgccctccctctcagcatctttggaacaacagtcagactca
cctgtaccctgatcagtggctccagtgttggcagctattacatcaactggttccagaaga
agccacggagccctccccagtatctcctatactactacttagactcagataagcaccagg
gctctggggtccccagctgcttctcctgatccaaggatgcctcagtcattggagggcacc
ctcatctctgagctgcagcctgaggactagactgaccttcgctgtctaatcggaaacaat
aatgcttct
SEQ ID NO. 289 IGLV5-77 (P)
>IGLV5-77*01|Canis lupus familiaris_boxer|P|V-REGION|
cagcctgtgctgacccagccaccctccctctctgcatccccgggaacaacagccagactc
acctgcaccctgagcagtggcttcagtgttggtgactatgacatgtactggtaccagaag
aagccaggaagcccccaccccgggatctcctgtactactactcagactcatataaacacc
agggctccggggtctccagcagcttctctggatccaaggatacctcagccaatacagggc
tcctgctcatctctgggccacagcctgaggacgaggctgactactactgtgctacagatc
atggcagtgagagcaggtactcttacc
SEQ ID NO. 290 IGLV5-77-1 (P)
>IGLV5-77-1*01|Canis lupus familiaris_boxer|P|V-REGION|
cagcctctgctacccagcacccccttcgctgcgtttccaggtactacagccagaatcacc
tgcaccctgagcaggggcatcagtgttgggagctgttccttataacggctcccgcagagg
cagggagccctgcctggtatctgctgaggttcccctctaatagacaccacatctctggat
ccaaagaaacctcggccaatgcagggctcctgctcattgttgtgctgccacctgacaact
agtctatcagtggtggttgaggactaggactattactgggatgctttggttt
SEQ ID NO. 291 IGLV5-78-1 (P)
>IGLV5-78-1*01|Canis lupus familiaris_boxer|P|V-REGION|
cagcctttgctgatccagcgccctccctctctgcatctcctggaacaacagtcagactca
cctgcacccagagcagtggcccctgtgttggcagctactacatacactggttccagtgga
agccatggagccctccctggtatcttctgtactactaatcagactcagatgagcaccagg
gctctggggtccccagccgcttctcctgatccaaggatgcctcagccagagcagggctcc
ctcatctctggactgcagcctgaggactagactgaccttcactgtctaatcagaaacaat
aatgttt
SEQ ID NO. 292 IGLV5-83-1 (P)
>IGLV5-83-1*01|Canis lupus familiaris_boxer|P|V-REGION|
tgcaggtccctgtcccagcctttgccctccctctttgcatctcctggaagaacagtcaga
tccacctgcacccagagcagtggcccctgtgttggcagctactacatacaccggttccag
tggaagccacggagccgtctccatatctcctgtactactactcagactcagatgagcacc
agagctctggagtccccaactgcttctcctgatccaaggatgcctcagggaaggcagggc
tccctcatctctgggctacaggctgaggacaagactgacctttactgtctaatccaaaac
aataatgtttct
SEQ ID NO. 293 IGLV5-85 (F)
>IGLV5-85*01|Canis lupus familiaris_boxer|F|V-REGION|
cagcctgtgctgacccagccaccctccctctctgcatccctgggatcaacagccagaccc
acctgcaccctgagcagtggcttcagtgttggaagctaccatatactctggttccagcag
aagtcagagagccctccccggtatctcctgaggttctactcagattctaatgaacaccag
ggtcccggggtccccagccgcttctctggatccaaggacacctcaacctatgcagggctc
ttgctcatctctgggctgcagcctgaggacgaggctgactactactgtgctacagaccat
ggcagtgggagcagctacacttacc
SEQ ID NO. 294 IGLV5-86-1 (P)
>IGLV5-86-1*01|Canis lupus familiaris_boxer|P|V-REGION|
cagcctttgctgacccagcgccctccctctctgcatctcctggaacaaaagtcagactca
cctgcatccagagcagtggatccagcgttggcagctactacatacactggttccagtaga
agccatggagccctccccagtatctcctgtactactacttagactcagataagcactagg
cctatggggaacccagatccttcccctgatccaaggatgcctcagtcaatgcagggtcaa
agagaggggattatttagagtggacaattggggcctttggccaggag
SEQ ID NO. 295 IGLV5-88-1 (P)
>IGLV5-88-1*01|Canis lupus familiaris_boxer|P|V-REGION|
cagccagtgcagacccagctgccctccttctctgtacctctgggaacaacagccagactc
acctgcaccctgagcagtgttggcggccagtaaacatccttttcaaggagaaaccaagga
gccccccagtctctcctgtactattacccagactcagataaaccccaggtctctggggtc
cccagccacttctctgaatccaaagactcctaggccaatgcagggctcctgctcgcctct
gggctgcagcctgaggacgaggctgactatcactgtgctgtaaatcatgacagtgggagc
agctccggatact
SEQ ID NO. 296 IGLV5-89-1 (P)
>IGLV5-89-1*01|Canis lupus familiaris_boxer|P|V-REGION|
caggctgtggtgacccagcttccttctctgcatccctgggaacaacagccagactcacat
gcaccctgagctgtggcttcagtattgatagatatgctataaactggttccagcagaagg
cagagagccttccctggtacctactgtgctattactggtactcaagtacacagttgggct
tcagcgtccccagctgcatctctggatccaagacaaggccacattcacaaacgagtagac
ccatctctggttgggtctagagctccagccccacctgagactgatgcacaattgcagc
SEQ ID NO. 297 IGLV5-92-2 (P)
>IGLV5-92-2*01|Canis lupus familiaris_boxer|P|V-REGION|
cagcctgtatagacccagtcaccctccctttctgcatctttggaacaacagtcagagtca
cctgtaccctgagcagtggctccagtgttggcagctactacatatactggttccaggaga
agccatggagcaatccccggtatctcctgtactactcaggctcagatgagcaccagggct
ctgggatccgtagctgcttctcctgatacaatgatgcctcagccaaggcagagctcccta
atctctgggctgcagcctgaggactatactgaccttcactgtctaatcagaaacaataat
cctttt
SEQ ID NO. 298 IGLV5-94-1 (P)
>IGLV5-94-1*01|Canis lupus familiaris_boxer|P|V-REGION|
tagcctgtgctgacccagcgccctcccactctgcatccctgggaacaacagccagactca
cctgcgccctgagcagcggctgcagcagtgaccatacgctggttccagcagccagaaggc
ctcctgagtacctgctgacggtctactgagactcaccagcgccccggggtcctcagcctc
ttctctggctccaaggacacctcggccaatgcagggcactcagatgg
SEQ ID NO. 299 IGLV5-95 (P)
>IGLV5-95*01|Canis lupus familiaris_boxer|P|V-REGION|
cagcctgtgatgacccagctgtcctccctctctgcatccctggaaacaacaaccagacac
acctgcaccctgagcagtggcttcagaaataacagctgtgtaataagttgattccagcag
aagtcagggagccctccctggtgtctcctgtactattactcagactcaagtatacatttg
ggctctgaggttcccagctgcttctctggatccaagacaaggccacacccacactgagta
gacccatccctgggtgggtctagagctccagccccactggaggctgatgcacaattgcag
c
SEQ ID NO. 300 IGLV5-96-1 (P)
>IGLV5-96-1*01|Canis lupus familiaris_boxer|P|V-REGION|
caacctttgcggacccagcgcactccctctgcatctcctggaacaacagttagactcatc
tgcacccagagcagtggccccagtgttggcagctactacaaacactggttccagcagaag
ccacggagccctccccggtacttcctgtactacttctcagactcagatgagcaccagggc
tctggggaccgcagccacttctcctgatccaaggatgactcaggaaaggcagggctccct
catctctgggctacagcctgaggactagactgaccttcactgtctaatcagaaacaataa
tgcttct
SEQ ID NO. 301 IGLV5-97-1 (P)
>IGLV5-97-1*01|Canis lupus familiaris_boxer|P|V-REGION|
ttaaaaccaaccaaaccaaaccaaaccaaaacaaaacaaaacaaaataacagccagattc
acctgctccctgagcagtggcttcagtgttggtggctataacacactggtaccagcagaa
gccagggagccctccctgttacctcctgtactactactcagaatcagataaacaccatgg
ctccgggatcaccagctgcttccctggccctatggacacctcggccaatgcagggctcct
gctcatctcagggctgcagcctgaggacgaggctgactactactgcggtatactccacag
cagtgggagcagctactcttacc
SEQ ID NO. 302 IGLV5-97-2 (P)
>IGLV5-97-2*01|Canis lupus familiaris_boxer|P|V-REGION|
caggctgtgaggacacactcctccttcctctctgcacctttgggatcatcaaccagactc
acctgcatccttcccagggcctgaatgttggcaggtactgaacatactggacaaggagaa
tcaaggagacatcaggagttccctcagatccagataagtgccagggcacggggttctcag
ccacttctatggatctaatgatgcctcaggcaatgcaggtctcctgctcatgtctgggct
gcagcctgaggacgaggctgactatgactatgctgcacattgtggggtgggagcagctcc
cgatact
SEQ ID NO. 303 IGLV5-97-3 (P)
>IGLV5-97-3*01|Canis lupus familiaris_boxer|P|V-REGION|
cagcctgtgctgacccagccgccctccctctctgcatccctgggaacaacagccagactc
acctgcaccctgagcagcagctgcagcggtggccatatgctggttccagcatgcaagagg
cctcctgagtacctgctgatggtctactgagactcaccagggccctggggtccccagcct
cttctctggctccaaggaagcctcggccaatgcagggctcctgctcatctctgggctgca
gcctgagaatgaggctgactgtcactgtgctacagaccatggcagtgggaacagctccca
atact
SEQ ID NO. 304 IGLV5-101-1 (P)
>IGLV5-101-1*01|Canis lupus familiaris_boxer|P|V-REGION|
cagcctttgctgacccagcgtcctccctctctgcatctcctggaacaacagtcagactca
catgtaccctgagcagtggccccggtgctggcagctactacacacactggttccagcaga
ggccacagagtcctccccggtatctcctgtactactactcagactcagatgatctccagg
gctccgggttccccagccactcctcctgatccaaggatgcctcagccagggcagggctcc
catctctggggtacagcctgaggactacactgaccttcactgtctaatcggaaacaataa
tgtttct
SEQ ID NO. 305 IGLV5-103-1 (P)
>IGLV5-103-1*01|Canis lupus familiaris_boxer|P|V-REGION|
cagccagggctggcccagctgccccccacctccctctgcatctccaggaacaacagccag
actcacatgaaccatgagcagtggcttcattgttggcagctgctacatatactggttcca
acagaagccagggagcccccctcccccaatatctcttgaggttgtattcagaatcagata
aacaccagggctcaatgtccccagccctgctctggatctgaagacacctccgccgaagca
gggcctctgctcatctctgggctgcagcgtgaggacaaggctgactcttactgtacaatc
tgg
SEQ ID NO. 306 IGLV5-105 (P)
>IGLV5-105*01|Canis lupus familiaris_boxer|P|V-REGION|
cagcctgtgctgacccagccgccctccctctctgcatccctgggaacaacagccagactc
acctgcaccatgagcagcagctacagtggtggccatacactggttccagcagccaggagg
cctcctgagtacctgctgatggtctactgagatttaccagggccccggggtccccagccg
cttctctggctccaaggacatctcggccaatgcagggctcctgctcatctctgggctgta
gcctgaggacgaggctgactgtcactgtgctacagaacatggcagcgggagcagctccca
atact
SEQ ID NO. 307 IGLV5-105-1 (P)
>IGLV5-105-1*01|Canis lupus familiaris_boxer|P|V-REGION|
ctgcctctgctacccagccaccgccttctctgcatctccaggtactacagccagacccac
ctgcaccctgaacagtggcatcagtattcgcagctgttccttataatggctcccgcaaag
gcagggagccctgcctggtatctgctaaggttgtactctaataaataccatggctctagg
gtcccaagccacatctctggatccaaagaaacctc
SEQ ID NO. 308 IGLV5-106-1 (P)
>IGLV5-106-1*01|Canis lupus familiaris_boxer|P|V-REGION|
cagcctttgctgacccagcgtcctccctctctgcatctcctggaacaacagtcagactca
cctgtatccagagcagtggccccagtgttggcagctactacatacaccggttccagcgga
aaccacggagccctcccctgtatctcctgtactactactcagactcagataagcactagg
cctacagggtccccagctgcttctcctgatccatggatgcctcagccagtgcagtgctcc
ctcatctctgggctacagcctgaggactagactgaccttcactgtctaatcggaaacaat
aatgcttct
SEQ ID NO. 309 IGLV5-109 (F)
>IGLV5-109*01|Canis lupus familiaris_boxer|F|V-REGION|
cagcttgtgctgacccagccgccctccctctctgcatccctgggatcaacaaccagactc
acctgcaccctgagcagtggcttcagtgttggtggctatagcatatactggcaccagcag
aagccagggagcactccctggtacctcctgtactactactcaagtacagagttgggacct
ggggtccccagctgcttctctggatccaaagacacctcagccaatgtagggctcctgctc
atctcagggctgcagcctgaggatgagactgactactactgtgctataggtcacggcagt
gggagcagctacacttacc
SEQ ID NO. 310 IGLV5-110-1 (P)
>IGLV5-110-1*01|Canis lupus familiaris_boxer|P|V-REGION|
cagccagggctggcccagctgcccccccacctccctctgcatctccaggaataacagcca
gactcacatgaaccatgagcagtggcttcattgttggccgctgctacatatactgattcc
aacagaagccaaggagcccccgctccaccagtatctcctgatattctactcagactcaga
taagcaccagggctcaacgtccccagccctgctctgaatctgaagacacctccgcgaagc
agggcttctgctcatctctgggctcagcgtgaggacaaggctgactcttactgtacaatc
tgg
SEQ ID NO. 311 IGLV5-111-1 (P)
>IGLV5-111-1*01|Canis lupus familiaris_boxer|P|V-REGION|
tagcctgtgctgacccagtgctctccctctctgcatccctgggaacaacagccagactcc
cctgcaccctgagcagcggctgcagcggtgtccatacgcaggttccagcagccaggaggc
ctcctgaatacctgctgatggtctacggtgactcaccagggccccggggtccccagccgc
ttctctggctccgaggacacctcggccaatgcagggctcctgctcatctctgggctgcag
cctgaggacaagactgactgtcactgtgctacagaccatggcagtaggagcagttcccaa
tact
SEQ ID NO. 312 IGLV5-111-2 (P)
>IGLV5-111-2*01|Canis lupus familiaris_boxer|P|V-REGION|
cagcctgtgctgacccagctgcccttcctctctgcatccctggagacaacaagcagatgt
acctacacccagagcggtgtcggcagctactacacatactcatcaaggacaatccaggga
gacctccctggtatttcctgtactactactcagactcaactacatggttgggatttggtg
tccccaaccacttctctgtatccaaagatgcctcagccaatgcagggctcctgctcatct
ctgggctgcagccagaggacaaggatgactgtcactgtgctgcattcagatcatggcagt
gggagcagctcccgatact
SEQ ID NO. 313 IGLV5-113-2 (P)
>IGLV5-113-2*01|Canis lupus familiaris_boxer|P|V-REGION|
cagcctttgctgatccagtgccctccctctctgcatctcctggaacaagagtcagactca
cctgcacccagagcagtggccccagggttggcagctactacatacactggttgcagcgga
aaccacggagccctcctcagtatctcctgtactactactcagaatcagatgagcaccagg
gctctggggtccccagccacttctcctgatccaaggatgcctcaggcaaggcagggctcc
ctcatccctgggctacagcctgagggctagactgaccttcactgtctaatccgaaacaat
aatgtttct
SEQ ID NO. 314 IGLV5-114-1 (P)
>IGLV5-114-1*01|Canis lupus familiaris_boxer|P|V-REGION|
cagccagggctggcccagctgccctccctctctgcatctccaggaacaacagccagactc
acatgaaccatgaacagtggcttcattcttggcggctgatacatatacttgttccaacag
aaaccagggaacccccgctccccgtattgcctgaggttctactcagactcagataagcac
cagggctcaacatccccagccctgctctggatctgaagacacctcaactgaagcagggcc
tctgctcatctctggatgtccagcgtgaggacaaggttgattcttactgtacaatctggc
acagtggtcctggt
SEQ ID NO. 315 IGLV5-115-1 (P)
>IGLV5-115-1*01|Canis lupus familiaris_boxer|P|V-REGION|
cagcctctgctgacccagccaccctccctctctgcatccctgggaacaagacccagagtc
acctgcaccctgagcaacaactgcagtggtggccatacgctggttccagcagccaggaag
cctcctgaatacctattgatggtttactgagacttaccagggcccccggggccccagctg
cttctctggctccaaggacaccttggccaatgcaggactcctgctcatctctgggctgta
gcctgaggatgaggctgactgtcactgtgctacagaccatggcagtgggagcagctcccg
atact
SEQ ID NO. 316 IGLV5-118-1 (P)
>IGLV5-118-1*01|Canis lupus familiaris_boxer|P|V-REGION|
caggctgtggtgacccagcttccttctctgcatccctgggaacaacagccagattcacat
gcaccctgagctatggcttcagtattgatagatatgttataagctggttccagcagaagg
cagagagccttccctggtacctactgtactattactgatactcaagtacacagttgggct
tcggcattcccagctgcgtctctggatccaagacaaggccacattcacaaatgagtagac
ccatctctggttgggtctagagctccagccccacctgagactgatgcacaattgcagcca
cattgtcttgatatcggaaa
SEQ ID NO. 317 IGLV5-124-1 (P)
>IGLV5-124-1*01|Canis lupus familiaris_boxer|P|V-REGION|
cagcctgtatagacccagtcaccctccctttctgcatctttggaacaacagtcagactca
cctgtaccctgagcagtggctccagtgttggcagctactacatatactggttccaggaga
agccatggagcaatccccggtatctcctgtactattcaggctcagatgagcaccagggct
ctgggatccctagctgcttctcctgatccaaggatgcctcagccaaggcagagctccctc
atctctgggctgcagcctgaggactagactgaccttcactgtctaatcagaaacaataat
gcttct
SEQ ID NO. 318 IGLV5-125-1 (P)
>IGLV5-125-1*01|Canis lupus familiaris_boxer|P|V-REGION|
cagcctgtgctgacccagcgccctcccactctgcatccctgggaacaacagccagactca
cctgcaccctgagcagcggctgcagcggtggccatatgctggttccagcagccagaaggc
ctcctgagtacctgctgacggtctactgagactcaccagggcccctgggtcctcagcctc
ttctctgactccaaagacacctcggccaatgcagggcactcagatggctgtgaagttcat
acaacagggtcctcatgggggctcatggtaccacttcacgttt
SEQ ID NO. 319 IGLV5-126 (P)
>IGLV5-126*01|Canis lupus familiaris_boxer|P|V-REGION|
cagcctgtgatgacccagctgtcctccctctcagcatccctggaaacaacaacaagactc
acctgaaccctgagcagtggcttcagaaatgacagatgtgtaataagttggttccagcag
aagtcagggagccctccctggtgtctcctgtactattactcggactcaagtacacatttg
ggctctgaggttcccagctgcttctctggatccaagacaaggccacacccacactgagta
gacccatccccgggtgggtctagagctccagccccactggaggctgatgcacaattgcag
c
SEQ ID NO. 320 IGLV5-128-1 (P)
>IGLV5-128-1*01|Canis lupus familiaris_boxer|P|V-REGION|
caacctttgcggacccagcgccctccctctctgcatctcctggaacaacagttagactca
tctgcacccagagcagtggccccagtgttggcagctactacaaacactggttccagcaga
agccacggagccctccccggtacctcctgtactactactcagactcagatgagcaccagg
gctctggggaccacagccacttctcctgatccaaggatgcctcaggaaaggcagggctcc
ctcatctctgggctacagcctgaggactagactgaccttcactgtctaatcagaaacaat
aatgcttct
SEQ ID NO. 321 IGLV5-129-1 (P)
>IGLV5-129-1*01|Canis lupus familiaris_boxer|P|V-REGION|
cagcctgtgctgaccagctgccctctctgcatccctgggaacaacaggcagatgtactta
caccctgagcagttttggcagctactacacatactcgtcaaggagaatacagggagacct
ccctggtatttcctgtactactactcagactcaactacatggttgggatttggggtcccc
aaccacttctctggatccaaagatgcctcagccaatgcagggctcctgctcatctctggg
ctgcagccagaggacaaggatgactgtcactgtgctgcatacatatcaaggcagtggaag
cagctcccaatact
SEQ ID NO. 322 IGLV5-129-2 (P)
>IGLV5-129-2*01|Canis lupus familiaris_boxer|P|V-REGION|
ctgcctgtgctgacccagtgccctccctctctgcatccctgggaacaacagccagactca
cctgcaccctgagcagtggctgcagcggtggccatatgctggttccagcagccaggaggc
ctcctaagtacctgctgatggtctactgagactcatcacggtcctggggtccctagcctc
ttctctggctccaaggacacctcggccaatgcagggctcctgctcatctctgggctgcag
cctgaggacgaggctgactgtcattgtgctacagaccatggcagtgggagcagctcctga
tact
SEQ ID NO. 323 IGLV5-131 (F)
>IGLV5-131*01|Canis lupus familiaris_boxer|F|V-REGION|
cagcctgtgctgacccagccaccctccctctctgcatccctgggaacaacagccagactc
acctgcaccctgagcagtggcttcagtgttggtgactatgacatgtactggtaccagcag
aagccagggagccctccccgggatctcctgtactactactcggactcatataaaaaccag
ggctctggggtctccaaaagcttctctggatccaaggatacctcagccaatgcagggctc
ctgctcatctctgggctgcagcctgaggacgaggctgactactactgtgctacagatcat
ggcagtgagagcagctactcttacc
SEQ ID NO. 324 IGLV5-132-1 (P)
>IGLV5-132-1*01|Canis lupus familiaris_boxer|P|V-REGION|
cagcctgtatagacccagtcaccctccctttctgcatctttggaacaacagtcagactca
cctgtaccctgagcagtggctccagtgttggcagctactacatatactggttccaggaga
agccatggagcaatccccggtatctcctgtactactcaggctcagatgagcaccagggct
ctgggatccctagctgtttctcctgatccaaggatgcctcagccaaggcagagctccctc
atctctgggctgcagcctgaggactatactgaccttcactgtctaatcagaaacaataat
gcttct
SEQ ID NO. 325 IGLV5-134 (P)
>IGLV5-134*01|Canis lupus familiaris_boxer|P|V-REGION|
cagcctgtgctgacccagccgccctccctctctgcatccctgggaacaacagccagactc
acctgcaccatgagcagcagctgcagcggtggccatatgctggtaccagcatgcaagagg
cctcctgagtacctgctgatggtctactgagactcaccagggccctggggtccccagcct
cttctctggctccaaggacaccttggccaatgcagggctcctgctcatctctgggctgca
gcctgagaatgaggctgactgtcactgtgctacagaccatggcagtgggaacagctccca
atact
SEQ ID NO. 326 IGLV5-134-1 (P)
>IGLV5-134-1*01|Canis lupus familiaris_boxer|P|V-REGION|
taaaaccaaaccaaaccaaaccaaaccaaaacaaaacaaaacaaaataacagccagattc
acctgctccctgagcagtggcttcagtgttggtggctataacacactggtaccagcagaa
gccagggagccctccctgttacctcctgtactactactcagaatcagataaacaccatgg
ctccgggatcaccagctgcttccctggccctatggacacctcggccaatgcagggctcct
gctcatccttgggctgcagcctgaggacgaggctgactactactgcggtatactccacag
cagtgggagcagctactcttacc
SEQ ID NO. 327 IGLV5-135-1 (P)
>IGLV5-135-1*01|Canis lupus familiaris_boxer|P|V-REGION|
aagcctgtgctgacccagcgccctccctctctgcatccctgggaacaacagccagactca
cctgcaccctgagcagcggctggagtggtggctataggctggttccagcagccaggaagc
ctcctgagtacctgctgatggtctactgagactcaccaggctatggggtccccagcatct
tctctggctccaaggaagcctcggccaatgcagggctcctgctcatctctggcctgcagc
ctgaggtcgaggctgactgtcactgtgctacagaccatggcagtgggagcagctcccgat
ac
SEQ ID NO. 328 IGLV5-137-1 (P)
>IGLV5-137-1*01|Canis lupus familiaris_boxer|P|V-REGION|
cagcctgtgctaacccagtcgctctccctcttgacatctttggaacaacagtcagactca
cctgtaccgtgaacagtggctccagtgttggcagctattacatcaactggttccagtata
agccatggagctctccctagtatcacctgtactactacttagactcagataagcaccagg
gctctggggtccccagctgcttctcctgatccaaggatgcctcagtcattggagggcacc
ctcatctctgggctgcagcctgaggactagactgaccttcacgtctaatcagaaacaata
atgcttct
SEQ ID NO. 329 IGLV5-137-2 (P)
>IGLV5-137-2*01|Canis lupus familiaris_boxer|P|V-REGION|
ctgcctgtgctgacccagccgccctccctctctgcatccctgggatcaacagccagactc
acctgcacactgagcagtggctgcagcggtggccatatgctggttccagcagccaggagg
cctcctgtgtacctgctgatggtctactgagactcaccagggccccagtgtccccagcca
ctactctggtttcaaagacacctcggccaatgcaggtcactcagatagctgcgaaattca
tacaacaagggtcctcatggggactcatgggcaccccttcagattttcctgcctgcatga
acag
SEQ ID NO. 330 IGLV5-138-1 (P)
>IGLV5-138-1*01|Canis lupus familiaris_boxer|P|V-REGION|
cagggatggcccagctgttcccccacctccctctgcatctccaggaacaacagccagact
cacatgaaccatgagcagtggcttcattgttggcggctgctacatatactggttccaaca
gaagccagggagtccccttccccccatatctcctgagtttctactcagactcagataagc
accagggctcaaaatccccagccctgttctggatctgaagacacctcagccaaagcagcg
cctctgctcatctctgggctgcagggtgaggataagaatgactcttactctacaatctgg
SEQ ID NO. 331 IGLV5-139-1 (P)
>IGLV5-139-1*01|Canis lupus familiaris_boxer|P|V-REGION|
caacctttgcggacccagtgccctccctctctgcatctcctggaacaacagttagactca
tctgcacccagagcagtggccccagtgttggcagctactacaaacactggttccagcaga
agccacggagccctccccagtacctcctgtactacttctcagactcagatgagcaccagg
gctctggggactgcagccacttcccctgatccaaggatgcctcaggaaagcagggctccc
tcatctctgggctacagcctgaggactagactgaccttcactgtctaatcagaaacaata
atgcttcttacagt
SEQ ID NO. 332 IGLV5-145 (P)
>IGLV5-145*01|Canis lupus familiaris_boxer|P|V-REGION|
cagcctgtgctgacccagccgccctccctctctgcatccctgggaacaacattcagactc
acctgcaccctgagcagcagctgcagcggtggccatatgctggttccagcatgcaagagg
cctcctgagtacctactgatggtctactgagactcaccagggccctggggtccccagcct
cttctccggctccaaggacaccttggccaatgcagggctcctgctcatctctgggctgca
gcctgagaatgaggctgactgtcactgtgctacagaccatggcagtgggaacagctccca
atact
SEQ ID NO. 333 IGLV5-145-1 (P)
>IGLV5-145-1*01|Canis lupus familiaris_boxer|P|V-REGION|
caggctgtgacgacacactcctccttcctctctgcacctttgggatcatcaaccagactc
acctgcatccttcccagggcctgaatgttggcaggtactgaacatactggacaaggagaa
tcaaggaggcatcaggagttccctcagatccagataagtgccagggcacggggttctcag
ccacttctatggatctaatgatgcctcaggcaatgcaggtctcctgctcatgtctgggct
gcagcctgaggacgaggctgactatgactatgctgcacattgtggggtgggagcagctcc
cgatact
SEQ ID NO. 334 IGLV5-146-1 (P)
>IGLV5-146-1*01|Canis lupus familiaris_boxer|P|V-REGION|
aagcctgtgctgacccagcgccctttctctctgcatccctgggaacaacagccagactca
cctgcaccctgagcagcggctggagtggtggctataggctggttccagcagccaggaagc
ctcctgagtacctgctgatggtctactgagactcaccaggctatggggtccccagcatat
tctctggctccaaggaagcctcggccaatgcagggctcctgctcatctctgggctgcagc
ctgaggtcgaggctgactgtcactgtgctacagaccatggcagtgggagcagctcccgat
act
SEQ ID NO. 335 IGLV5-148 (P)
>IGLV5-148*01|Canis lupus familiaris_boxer|P|V-REGION|
cagactgtggtcaccaaggatccatcactctcagtgtttccaggagggacagtcacattc
acatgtggcctcagctctgggtcagtctttacaagtaactaccccagctggtaccagcag
acccatggccgggctcctcacatgcttatctacagcacaagcagctgcccccccggggtc
cctgatcgcttctctggatccatctctgggaacaaagttgccctcaccatcacaggagcc
cagcctgaggatgagactattattgttcactgcgtatgggtagtacattta
SEQ ID NO. 336 IGLV5-148-1 (P)
>IGLV5-148-1*01|Canis lupus familiaris_boxer|P|V-REGION|
cagcctgtgctaacccagtcgccctccctcttgacatctttggaacaacagtcagactca
cctgtaccgtgaacagtggctccagtattggcagctattacatcaactggttccaggaga
agccatggagctctccctggtatcacctatactacttcttagactcagataagcaccagg
gctctggggtccccagctgcttctcctgatccaaggatgcctcagtcattggagggcacc
ctcatctctgggctgcagcctgaggactagactgaccttcactgtctaatcagaaacaat
aatgcttct
SEQ ID NO. 337 IGLV5-148-2 (P)
>IGLV5-148-2*01|Canis lupus familiaris_boxer|P|V-REGION|
ctgcctgtgctgacccagccgccctccctctctgcatccctgggatcaacagccagactc
acctgcacactgagcagtggctgcagcggtagccatatgctggttccagcagccaggagg
cctcctgggtacctgctgatggtctactgagactcaccagggccccagtgtccccagcca
ctactctggatgcaaagacacctcggccaatgcaggt
SEQ ID NO. 338 IGLV5-149-1 (P)
>IGLV5-149-1*01|Canis lupus familiaris_boxer|P|V-REGION|
cagggatggcccagctgttcccccacctccctctgcatctccaggaacaacagccagact
cacatgaaccatgagcagtggcttcattgttggcggctgctacatatactggttccaaca
gaagccagggagtccccttccccccatatctcctgagtttctactcagactcagataagc
accagggctcaaaatccccagccctgttctggatctgaagacacctcagccaaagcagcg
cctctgctcatctctgggctgcagggtgaggataagaatgactcttactctacaatctgg
SEQ ID NO. 339 IGLV5-150-2 (P)
>IGLV5-150-2*01|Canis lupus familiaris_boxer|P|V-REGION|
caacctttgcggacccagcgcactccctctgcatctcctggaacaacagttagactcatc
tgcacccagagcagtggccccagtgttggcagctactacaaacactggttccagcagaag
ccacggagccctccccggtacttcctgtactacttctcagactcagatgagcaccagggc
tctggggaccgcagccacttctcctgatccaaggatgactcaggaaaggcagggctccct
catctctgggctacagcctgaggactagactgaccttcactgtctaatcagaaacaataa
tgcttct
SEQ ID NO. 340 IGLV5-154-1 (P)
>IGLV5-154-1*01|Canis lupus familiaris_boxer|P|V-REGION|
cagcctgtgatgacccagctgtcctccctctctgcatccctggaaacaacaaccagacac
acctgcaccctgagcagtggcttcagaaataacagctgtgtaataagttgattccagcag
aagtcagggagccctccctggtgtctcctgtactattactcagactcaagtatacatttg
ggctctgaggttcccagctgcttctctggatccaagacaaggccacacccacactgagta
gacccatccctgggtgggtctagagctccagccccactggaggctgatgcacaattgcag
c
SEQ ID NO. 341 IGLV5-155-1 (P)
>IGLV5-155-1*01|Canis lupus familiaris_boxer|P|V-REGION|
cagcctgtgctaacccagtcgctctccctcttgacatctttggaacaacagtcagactca
cctgtaccgtgaacagtggctccagtgttggcagctattacatcaactggttccagtata
agccatggagctctccctagtatcacctgtactactacttagactcagataagcaccagg
gctctggggtccccagctgcttctcctgatccaaggatgcctcagtcattggagggcacc
ctcatctcggggctgcagcctgaggactagactgaccttcactgtctaatcagaaacaat
aatgcttctaacagtga
SEQ ID NO. 342 IGLV5-157-1 (P)
>IGLV5-157-1*01|Canis lupus familiaris_boxer|P|V-REGION||
cccagcgccctttctctctgcatccctgggaacaacagccagactcacctgcaccctgag
cagcggctagagtggtggctataggctggttccagcagccaggaagcctcctgagtacct
gctgatggtctactgagactcaccaggctatggggtccccagcatcttctctggctccaa
ggacacctcggccaatgcagggctcctgctcatctctgggctgcagcctgaggtcgaggc
tgactgtcactgtgctacagaccatggcagtgggagcagctcccgata
SEQ ID NO. 343 IGLV5-158-1 (P)
>IGLV5-158-1*01|Canis lupus familiaris_boxer|P|V-REGION|
ataacagccagattcacctgctccctgagcagtggcttcagtgttggtggctataacaca
ctggtaccagcagaagccagggagccctccctgttacctcctgtactactactcagaatc
agataaacaccatggctccgggatcaccagctgcttccctggccctatggacacctcggc
caatgcagggctcctgctcatctcagggctgcagcctgaggacgaggctgactactactg
cggtatactccacagcagtgggagcagctactcttacc
SEQ ID NO. 344 IGLV5-158-2 (P)
>IGLV5-158-2*01|Canis lupus familiaris_boxer|P|V-REGION|
caggctgtgacgacacactcctccttcctctctgcacctttgggatcatcaaccagactc
acctgcatccttcccagggcctgaatgttggcaggtactgaacatactggacaaggagaa
tcaaggaggcatcaggagttccctcagatccagataagtgccagggcacggggttctcag
ccacttctatggatctaatgatgcctcaggcaatgcaggtttcctgctcatgtctgggct
gcagcctgaggacgaggctgactatgactatgctgcacattgtggggtgggagcagctcc
cgatact
SEQ ID NO. 345 IGLV5-158-3 (P)
>IGLV5-158-3*01|Canis lupus familiaris_boxer|P|V-REGION|
cagcctgtgctgacccagccgccctccctctctgcatccctgggaacaacattcagactc
acctgcaccctgagcagcagctgcagcggtggccatatgctggttccagcatgcaagagg
cctcctgagtacctactgatggtctactgagactcaccagggccctggggtccccagcct
cttctctggctccaaggacaccttggccaatgcagggctcctgctcatctctgggctgca
gcctgagaatgaggctgactgtcactgtgctacagaccatggcagtgggaacagctccca
atact
SEQ ID NO. 346 IGLV7-32-2 (P)
>IGLV7-32-2*01|Canis lupus familiaris_boxer|P|V-REGION|
caggctgtggtgactccagagcccttctgaccatccccaggagtgacagtcacttttacc
tgtgactccagcactggagagtcattaatagtgactatccacgttagttccagcagaagc
ctagacaaactcgcaccacacacacaacaaacactcacggactcccacccagttctcagg
ctccctccaggctcaaaactgccctcacctttttggggtcccagcctgagaaagaaggtg
agtactaccatatgctggtctatcttggttcttgg
SEQ ID NO. 347 IGLV7-33 (P)
>IGLV7-33*01|Canis lupus familiaris_boxer|P|V-REGION||
caggctgtggtgactcaggaaccctcactgaccgtgtccctggagggacagtcactctca
cctgtgcctccagcactggcgaggtcaccaatggacactatccatactggttccagcaga
agcctggccaagtccccaggacattgatttataatacacacataatactcctggacccct
acccggttctcaggctgcctctttgggggcaaagctgccttgaccatcacaggggcccag
cccgaggatgaagctgaggactactgctggctagtatatatggtaatagg
SEQ ID NO. 348 IGLV7-36-1 (P)
>IGLV7-36-1*01|Canis lupus familiaris_boxer|P|V-REGION|
caggctgtggtgattcaggaatcctcactaacagtgcccccaggaggaacactctcacct
gtgcctcgaacactggcacagtcaccaatgtcagtatccttactggtttcagcagaaccc
tagtcaagtccccagggcattgacttaggatacaagcaataaacacttctggatccctac
caagctttcagtttccctccttggatgtaaaactcccctgaccttctctggttccctagc
ctgaggccaaggctgattaccactggtgggtactcatagtggtgctgca
SEQ ID NO. 349 IGLV7-38-2 (P)
>IGLV7-38-2*01|Canis lupus familiaris_boxer|P|V-REGION|
caggtcatggtgactcaggagccttcatggccatgtccccaggagggacagtcactctca
cctatgcctccagcacaggacactatccatactggatccaagaaaatattggccaagtca
gggccatttatttataataaaaacaacaaatactgatttctcatgctcccttcttgggag
caaatctgacatgaccatctcctagtgcccagcctgaggacgaggatgagtacccatggg
ggctacactatagtggtgctggg
SEQ ID NO. 350 IGLV7-43-1 (P)
>IGLV7-43-1*01|Canis lupus familiaris_boxer|P|V-REGION|
cagattgtggtgactcaggagccttcatggtcgtgtccccaggagggacagtcactctca
ctatgcctccagcacagaacactatccatactggatccaggaaaatattggccaagtcta
gagcatttatttataaaagaaacaataaatactgatttctaggctcccttcttgggaata
aatctgacttgaccatctgctagtgcgcagcctgaggacgaggctgagtacccctagggg
ttacac
SEQ ID NO. 351 IGLV7-44-1 (P)
>IGLV7-44-1*01|Canis lupus familiaris_boxer|P|V-REGION|
caggctgtgatgactcaggagtcctcactaacagtgtccccaggagggacattcactctc
acctgtgcctccagccactggcatagtaacaatgctcagtatccttcctggttttaccag
aagcctggccaagttcccagggcattgatttaggatacaagcaatgaaaattcctggacc
cccaccaagtgctcaggttccctttgtggagcaatattctcctgaccctctacagtgcct
tggtgagaacatagctgagtggcactggtggctgcttttattgtgatgctgggtgc
SEQ ID NO. 352 IGLV7-84-2 (P)
>IGLV7-84-2*01|Canis lupus familiaris_boxer|P|V-REGION|
caggctgtgatgactcaagagtcctcactaacagtgtccccaggagggacattcactctc
acctgcgcctccagctactggcatagtaacaatgctcagtatccttactggttttagcag
aatcctggccaagtccccagggcattgatttaggatacaagcaatgaacacacctggacc
cccaccatgtgctcaggttccctttgtggagcaatattctcctgaccctctacagtgcct
tggtgagaacatagctgagtggcactggtggctgcttttattgtgatg
SEQ ID NO. 353 IGLV7-90-2 (P)
>IGLV7-90-2*01|Canis lupus familiaris_boxer|P|V-REGION|
cagactgtggtggcataggagccttcatggccatatccccaggagggacagtcactctca
cctatccctccagcacaggacactatctatactggatctagtagcatactggccaagtct
aggtcatttatttataataaaaacaataaatactcatagacctccactcatttctcaggc
tcccatcttgggggcaaatctgactggattgtcccctagtgcccagcctgaggatgaggc
tgagtaccgctggggctacactatggtggtgtggg
SEQ ID NO. 354 IGLV7-120-1 (P)
>IGLV7-120-1*01|Canis lupus familiaris_boxer|P|V-REGION|
cagactgtggtggcataggagccttcatggccatatccccaggagggacagtcactctca
cctatccctccagcacaggacactatctatactggatctagtagcatactggccaagtct
aggtcatttatttataataaaaacaataaatactcatagacctccactcatttctcaggc
tcccatcttgggggcaaatctgactggattgtcccctagtgcccagcctgaggatgaggc
tgagtaccgctggggctacactatggtggtgtggg
SEQ ID NO. 355 IGLV8-36 (F)
>IGLV8-36*01|Canis lupus familiaris_boxer|F|V-REGION|
cagactgtggtgacccaggagccatcactctcagtgtctctgggagggacagtcaccctc
acatgtggcctcagctccgggtcagtctctacaagtaactaccccaactggtcccagcag
accccagggcaggctcctcgcacgattatctacaacacaaacagccgcccctctggggtc
cctaatcgcttcactggatccatctctgggaacaaagccgccctcaccatcacaggagcc
cagcctgaggacgaggctgactactactgtgctctgggattaagtagtagtagtagtta
SEQ ID NO. 356 IGLV8-39 (F)
>IGLV8-39*01|Canis lupus familiaris_boxer|F|V-REGION|
cagactgtggtaacccaggagccatcactctcagtgtctccaggagggacagtcacactc
acatgtggcctcagctctgggtcagtctctacaagtaaccaccctagctggtaccagcag
acccaagggaaggctcctcgcatgcttatctacaacacaaacaaccgcccctctgggatc
cctaattgcttctctggatccatctctgggaacaaagcctccctcaccatcacaggagcc
cagcctgaggacgagactgactattactgtttattgtatatgggtagtaacattta
SEQ ID NO. 357 I GLV8-40 (P)
>IGLV8-40*01|Canis lupus familiaris_boxer|P|V-REGION|
cagattgtggtgacccaggagccatcactctaagtttctccaggagggacagtcacactc
acatgtggcctcagctctgggtcagtccctacaagtaactaccccagctggtttcagcag
accccaggccgggctcctagaacagttatctacaacacaaacagctgcccctctggggtc
cctaatcgcttcactggatccatctctggcaacaaagccgccctcaccatcacaagagcc
cagcctgaggatgaggctgactcctgctgtgctgaatatcaaagcagtgggagcagctac
acttacc
SEQ ID NO. 358 IGLV8-43 (P)
>IGLV8-43*01|Canis lupus familiaris_boxer|P|V-REGION|
cagactgtggtaacccaggaaccatcactctcagtgtctccatgagggacagtcacactc
acatgtggcctcagctctgggtcagtctctacaagtaactaccccaactggtaccagcag
acccaaggccgggctcctcacagggttatctacaacacaaacaaccgcccctctggggtc
cctgatcgcttctctggatccatctctgggaacaaagccgccctcaccatcacagctgcc
cagcctgaggacgaggctgactattactgttcattgtatatgggtagtaacatttg
SEQ ID NO. 359 IGLV8-60 (P)
>IGLV8-60*01|Canis lupus familiaris_boxer|P|V-REGION|
cagactgtgatcacccaagatacatcactctcagtgtctccaggagggacagtcacactc
acatgtggcctcagctctgggtcagtctctacaagtaactaccccagctggtaccagcag
acccaaggccgggatcctcgcatgcttatctacagcacaaacagccacccctctggggtc
cctaattgcttcactagatccatctctgggaagaaagctgccctcaccatcacaggagcc
cagcctgaggatgagactattattgttcactaaatatgggtagtacatgta
SEQ ID NO. 360 IGLV8-71 (P)
>IGLV8-71*01|Canis lupus familiaris_boxer|P|V-REGION|
cagattgtggtgacccaggacccatcactgtcagtgtctagaggagggacagtcacactc
acttgtggcctcagctctgggtcagtcactacaataaataccccagctggtcccagcaga
ccccagggcaggctcctcgcatgattatctatgacacaaacagccgcccctctggggtcc
ctgatcgcttctctggatccatctgtgggaacaaagctgccctcaccatcacaggagccc
atcctgaggatgagactgactactactgtggtatacaacatggcagtgggagcagcctca
cttacc
SEQ ID NO. 361 IGLV8-74-1 (ORF)
>IGLV8-74-1*01|Canis lupus familiaris_boxer|ORF|V-REGION|
cagattgtggtgacccaggagccatcactgtcagtgtctccaggaggaacagttacactc
acatgtggcctaagctctgggtcagtcactataagtaactaccctgattggtaccagcag
actccaggcaggtctcctcgcatgcttatctacaacacaaacaaccgcccctctggggtc
cctaatcacttctctggatccatctctgggaacaaagccgccctcaccatcacaggagcc
cagcctgaggatgaggcttactactactgtgctgtgtatcaaggcagtgggagcagctac
acttacc
SEQ ID NO. 362 IGLV8-76-1 (P)
>IGLV8-76-1*01|Canis lupus familiaris_boxer|P|V-REGION|
cagactgtggtcacccaggatccatcactctcagtgtctccaggaggaacagtcacactc
acatgtggcctcagctctgggtcagtctctacaagtaactaccccggctggtaccagcag
acccaagtgaaagctccttgcatgcttatctacagcacaaacagctacccctctggggtt
cctaattgcttcactggatccatctctgggaagaaagctgccctcaccatcacaggagac
cagcctgaggatgagactattattgttcactgcatatgggtagtacactta
SEQ ID NO. 363 IGLV8-88-4 (P)
>IGLV8-88-4*01|Canis lupus familiaris_boxer|P|V-REGION|
cagactgtggtggctcaggagtcatcagtctcagtgtctccaggagggacagtcacactc
acttgtggcctcagctctgggtcagtgactacaagtaactaccacagctggtaccagcgg
acccaaggccggtctcctcacatgcttatctatgacacaagcagccgtccttctgaggtc
ctgatcgcttccctggttccatctctgggaacaaagctgccctcactgtcagaggagccc
agcctgaggacgaggctgactactactgtggcatgcatgatgtcagtgggaggaattaca
attacc
SEQ ID NO. 364 IGLV8-89-3 (P)
>IGLV8-89-3*01|Canis lupus familiaris_boxer|P|V-REGION|
cagattgtggtggccaggaggcattgttgtcagtgtctccaggagggagagtcacactca
cttgtggcctcagctctgggtcagtcactacaagtaactaccccaactggttccagcaga
ccccagggcgggctcctggcacgattatctacagcacaaaagactgcccctctggggtcc
ctgactgcttctctagatccatctctgggaacaaagccgccctcaccatcacaggagccc
agtctgaggacgaggctattactgttttacacgacatggtagtgggagctgctacactta
cc
SEQ ID NO. 365 IGLV8-90 (P)
>IGLV8-90*01|Canis lupus familiaris_boxer|P|V-REGION|
cagactgtggtaacccaggagccatcactctcagtgtctccaggagggacagtcacactc
acttgtggcctcagctctgggtcagtctctacaggtaacaaacctggctggtaccagcac
accccaggccaggctcctcgcaggattatctatgacacaagcagccgcccttctggggtc
cctgatcgcttctctggatccatctctgagaacaaaactgccctcaccatcacagaagcc
caacctgaggatgaggctgactacatcatatatgagtggtggtgctta
SEQ ID NO. 366 IGLV8-90-1 (P)
>IGLV8-90-1*01|Canis lupus familiaris_boxer|P|V-REGION|
cagattgtggtgacccaggaggcatcgttgttagtgtctcctggagggatagtcacactc
acttgtggcctcagctctggatcaatcactacaagtaactaccccaactggctccagcag
accccagggcgggctcctcgcagatgatctatggcacaaaaagccgcccctctggggtcc
ctgatcgcttctgtagatccatctctgggaacaaagccgccctcaccatcacaggagccc
agtctgaggatgaggctgactattactgttttacacgacatggcagtgggagcagctaca
attac
SEQ ID NO. 367 IGLV8-90-3 (P)
>IGLV8-90-3*01|Canis lupus familiaris_boxer|P|V-REGION|
cagactgtggtgacccaggagtcatcagtctcagtgtctccaggaggaacagtcacactc
ccttgtggcctcagctctgggtcactgactacaagtaacactacaccagctggtaccagc
agacccaaggccagtctcctcgcatgcttgtctatgacacaagcagctgtccctctgagg
ttcctgatcacttctctggatccatttctgggaacaaagccaccctcaccatcacaggag
cccagcctgaggacgaggctgactactactgtggcatgcatgatgtcagtgggagcagct
aaaattacc
SEQ ID NO. 368 IGLV8-90-4 (P)
>IGLV8-90-4*01|Canis lupus familiaris_boxer|P|V-REGION|
catattttggtgactcaggagccatcactgtcagtgtctccatgagggacagtcacactc
acttgtggcctcagctctgggtcagtcactacaagtaactaccccaggtataccagcaga
acccaggcaaggctcctagcacagttatctacaacaaaaacagctgcccctctggggtcc
atggtcgattctctggatccatctctggaagcaaagccgccttcacaatcacaggagccc
agcctgaggttgaggctgactactactgtgttacagaacatggctcctcacatgggaaca
gcctcactcac
SEQ ID NO. 369 IGLV8-92-1 (P)
>IGLV8-92-1*01|Canis lupus familiaris_boxer|P|V-REGION|
cagactgtggtcacccaggatccgtcactctcagtgtctccaggagggacagtcacattc
acatgtggcctcagctctgggtaagtctctacaagaaactaccccagctggtaccagcag
acccaaggccaggctccttgcatgcttatctacagcacaagcagacacccttctggggtc
cctgatcgcttctctggatccatctctgggaacaaagtcgccctcaccatcacaggagcc
cagcctgaggataagactattattgttcactgcatatgggtagtacattta
SEQ ID NO. 370 IGLV8-93 (F)
>IGLV8-93*01|Canis lupus familiaris_boxer|F|V-REGION|
cagactgtggtaacccaggagccatcactctcagtgtctccaggagggacagtcacactc
acatgtggcctcagctctgggtcagtctctacaagtaattaccctggctggtaccagcag
acccaaggccgggctcctcgcacgattatctacaacacaagcagccgcccctctggggtc
cctaatcgcttctctggatccatctctggaaacaaagccgccctcaccatcacaggagcc
cagcccgaggatgaggctgactattactgttccttgtatacgggtagttacactga
SEQ ID NO. 371 IGLV8-99 (F)
>IGLV8-99*01|Canis lupus familiaris_boxer|F|V-REGION|
cagactgtggtcacccagaagccatcactctcagtgtctccaggagggacagtcacactc
atatgtggcttcagctctgggtcagtctctacaagtaattaccctggctggtaccagcag
acccaaggccgggcttctcgcacaattatctacagcacaagcagccgcccctctggggtc
cctaatcgcttccctggatccatctctgggaacaaagccgccctcaccatcacaggagcc
cagcctgaggacgaggctgactattactgttccttgtatatgggtagttacactga
SEQ ID NO. 372 IGLV8-102 (ORF)
>IGLV8-102*01|Canis lupus familiaris_boxer|ORF|V-REGION|
cagattgtagtgacccaggaaccatcactgtctccaggagggacagtcctactcacttgt
ggcctcagctctgggtcagtcactacaagtaactactccagctggtaccagcagacccca
gggcgggctcctcgcacgattatctacaacactaacagccacccctctggagtccctgat
cgcttctctggatccatctctgggaacaaagcggcgctcaccatcacaggagcccagcct
gaggacgaggctgactactactgtgttacagaacatggtagtgggagcagcttcacttac
SEQ ID NO. 373 IGLV8-108 (F)
>IGLV8-108*01|Canis lupus familiaris_boxer|F|V-REGION|
cagactgtggtgactcaggagtcatcagtctcagtgtctccaggagggacagtcacactc
acgtgtgacctcagctctgggtcagtgactacaagtaacaaccccagctggtaccagcag
acccaaggccgatctcctcgcatgcttatctatgacacaagcagctgtccctcggaggtc
cctgatcgcttctctggatccatttctgggaacacagctgccctcaccatcacaggagcc
cagcctgaggacaaggctgactactactgtagtatgcatgatgtcagtgggagcagctac
aattacc
SEQ ID NO. 374 IGLV8-113 (P)
>IGLV8-113*01|Canis lupus familiaris_boxer|P|V-REGION|
cagactgtggtcacccaggagccatcactctcagtgtctccaggagggacagtcacactc
acatgtggcctcagttctgggtcagtcactataagtaactaccccagctggtcccagcag
accccagggcaggctcctcacacaataatctacaggacaaacagctgaccctctggggtc
cctgatcgcttctctggatccatctctgggaacaacgccgccctcagcatcacagtcgcc
cagcctgaggacgaggctgactattactgttcattgtatatgggtagtaacattta
SEQ ID NO. 375 IGLV8-113-3 (P)
>IGLV8-113-3*01|Canis lupus familiaris_boxer|P|V-REGION|
cagattgtggtgacccaggagccatcactctcagtgtctagaggagggacagtcacactc
acttgtggcctcagctctgagtcaatcactacaactaccccagctgatcccagcagaccc
cagggcaggctcctcacacaattatctatgacaaaaacagccgcccctctggggtccctg
atcacttctcaggatccatctgtgggaacaaagccaccctcaccatcacaggaacccagc
ctgaggacaaggctgactactactgtggtatccaacatggcagtaggaggagcctcatta
acc
SEQ ID NO. 376 IGLV8-117 (P)
>IGLV8-117*01|Canis lupus familiaris_boxer|P|V-REGION|
cagactgttgtgactcaggagtcatcagtctcagtgtctccaggagggacagtaacactc
acgtgtagcctcagctctgggtcagtgactacaagtaagtactccagctggaccagtaga
cccaaggccgatctcctcgcatgcttatctatgacacaagcagccgtccctctgaggtcc
ctgatcgcttctctggatccatctccgggaacaaagctgccctcaccatcacaggagccc
agcctgaggacgaggctgactactactgtggtatgcatgatgtcagtgggaggagttaca
attacc
SEQ ID NO. 377 IGLV8-118-3 (P)
>IGLV8-118-3*01|Canis lupus familiaris_boxer|P|V-REGION|
cagattgtggtggccaggaggcattgttgtcagtgtcctctggagggagagtcacactca
cttgtggcctcagctctgggtcagtcactacaagtaactaccccaactggttccagcaga
ccccagggcgggctcctggcacgattatgtacagcacaaaagactgcccctctggggtcc
ctgattgcttctctagatccatctctgggaacaaagccgccctcaccatcacaggagccc
agtctgaggacgaggttattactgttttacacgacatggtagtgggagctgctacactta
cc
SEQ ID NO. 378 IGLV8-119 (P)
>IGLV8-119*01|Canis lupus familiaris_boxer|P|V-REGION|
cagactgtggtaacccaggagccatcactctcagtgtctccaggagggacagtcacactc
acttgtggcctcagctctgggtcagtctctacaggtaacaaacctggctggtaccagcac
accccaggccaggctcctcgcaggattatctatgacacaagcagccgcccttctggggtc
cctgatcgcttctctggatccatctctgagaacaaagctgccctcaccatcacagaagcc
cagcctgaggatgaggctgcctaccactgttcgctgtatatgagtggtggtgctta
SEQ ID NO. 379 IGLV8-120 (P)
>IGLV8-120*01|Canis lupus familiaris_boxer|P|V-REGION|
cagattgtggtgacccaggaggcatcgttgtcagtgtctcctggagggatagtcacactc
acttgtggcctcagctctggatcaatcactacaagtaactaccccaactggttccagcag
accccagggcgggctcctcgcagatgatctatggcacaaaaagccgcccctctggggtcc
ctgatcgcttctgtagatccatctctgggaacaaagccgccctcaccatcacaggagccc
agtctgaggatgaggctgactattactgttttacacgacatggcagtgggagcagctaca
attacc
SEQ ID NO. 380 IGLV8-121 (P)
>IGLV8-121*01|Canis lupus familiaris_boxer|P|V-REGION|
cagactgtggtgacccaggagtcatcagtctcagtgtctccagtcggaacagtcacactc
acttgtggcctcagctctgggtcactgactacaagtaactacaccagctggtaccagcag
acccaaggccagtctcctcgcatgcttgtctatgacacaagcagctgtccctctgaagtt
cctgatcacttctctggatccatttctgggaacaaagccgccctcaccatcacaggagcc
cagcctgaggacgaggctgactactactgtggtatgcatgatgtcagtgggagcagctaa
aattacc
SEQ ID NO. 381 IGLV8-121-1 (P)
>IGLV8-121-1*01|Canis lupus familiaris_boxer|P|V-REGION|
catattttggtgactcaggagccatcactgtcagtgtctccatgagggacagtcacactc
acttgtggcctcagctctgggtcagtcactacaagtaactaccccaggtataccagcaga
acccaggcaaggctcctagcacagttatctacaacaaaaacagctgcccctctggggtcc
atggtcgattctctggatccatctctggaagcaaagccgccttcacaatcacaggagccc
agcctgaggttgaggctgactactactgtgttacagaacatggctcct
SEQ ID NO. 382 IGLV8-124 (P)
>IGLV8-124*01|Canis lupus familiaris_boxer|P|V-REGION|
cagactgtggtcaaccaggatccgtcactctcagtgtctccaggagggacagtcacattc
acatgtggcctcagctctgggtaagtctctgcaagaaactaccccagctggtaccagcag
acccaaggccaggctccttgcatgcttatctacagcacaagcagccgcccttctggggtc
cctgatcgcttctctggatccatctctgggaacaaagtcgccctcaccatcacaggagcc
cagcctgaggatgagactattattgttcactgcatatgggtagtacattta
SEQ ID NO. 383 IGLV8-128 (F)
>IGLV8-128*01|Canis lupus familiaris_boxer|F|V-REGION|
cagactgtggtaacccaggagccatcactctcagtgtctccaggagggacagtcacactc
acatgtggcctcagctctgggtcagtctctacaagtaattaccctggctggtaccagcag
accctaggccgggctcctcgcacgattatctacagaacaagcagccgcccctctggggtc
cctaatcgcttctctggatccatctctgggaacaaagccgccctcaccatcacaggagcc
cagcctgaggacgaggctgactattactgttccttgtatatgggtagttacactga
SEQ ID NO. 384 IGLV8-137 (P)
>IGLV8-137*01|Canis lupus familiaris_boxer|P|V-REGION|
cagactgtggtcaccaaggatccatcactctcagtgtctccaggagggacagtcacattc
acatgtggcctcagctctgggtcagtctttacaagtaactaccccagctggtaccagcag
acccatggccgggctcctcgcatgcttatctacagcacaaggagctgcccccccggggtc
cctgatcgcttctctggatccatctctgggaacaaagttgccctcaccatcacaggagcc
cagcctgaggatgagactattattgttcactgtgtatgggtagtacattta
SEQ ID NO. 385 IGLV8-142 (F)
>IGLV8-142*01|Canis lupus familiaris_boxer|F|V-REGION|
cagactgtggtcacccagaagccatcactctcagtgtctccaggagggacagtcacactc
atatgtggcctcagctctgggtcagtctctacaagtaattaccctggctggtaccagcag
acccaaggccgggcttctcgcacaattatctacagcacaagcagccgcccctctggggtc
cctaatcgcttcactggatccatctctgggaacaaagccgccctcaccatcacaggagcc
cagcctgaggacgaggctgactattactgttccttgtatatgggtagttacactga
SEQ ID NO. 386 IGLV8-150-1 (ORF)
>IGLV8-150-1*01|Canis lupus familiaris_boxer|ORF|V-REGION|
cagattgtggtgacccaggaaccatcactgtcagtgtctccaggagggacactcacactc
acttgtggcctcagctctgggtcagtcactacaagtaactaccccagctggtaccagcag
accccaggccaggctcctagcacagttatctacaacacaaacagccgcccctctggtgtc
cctgatcacttctctggatccgtctctgggaacaaagccgccctcatcatcacaggagcc
cagcctgaggacgaggctgatgactactctgttgcagaacatgtcagtgggagcagcttc
acttacc
SEQ ID NO. 387 IGLV8-153 (F)
>IGLV8-153*01|Canis lupus familiaris_boxer|F|V-REGION|
cagactgtggtaacccaggagccatcactctcagtgtctccaggagggacagtcacactc
acatgtggcctcagctctgggtcagtctctacaagtaattaccctggctggtaccagcag
acccaaggccgggctcctcgcacgattatctacaacacaagcagccgcccctctggggtc
cctaatcgcttctctggatccatctctggaaacaaagccgccctcaccatcacaggagcc
cagcccgaggatgaggctgactattactgttccttgtatacgggtagttacactga
SEQ ID NO. 388 IGLV8-156 (P)
>IGLV8-156*01|Canis lupus familiaris_boxer|P|V-REGION|
cagactgtggtcaccaaggatccatcactctcagtgtttccaggagggacagtcacattc
acatgtggcctcagctctgggtcagtctttacaagtaactaccccagctggtaccagcag
acccatggccgggctcctcgcatgcttatctacagcacaagcagctgcccccccggggtc
cctgatcgcttctctggatccatctctgggaacaaagttgccctcaccatcacaggagcc
cagcctgaggatgagactattattgttcactgtgtatgggtagtacattta
SEQ ID NO. 389 IGLV8-161 (F)
>IGLV8-161*01|Canis lupus familiaris_boxer|F|V-REGION|
cagactgtggtcacccagaagccatcactctcagtgtctccaggagggacagtcacactc
atatgtggcctcagctctgggtcagtctctacaagtaattaccctggctggtaccagcag
acccaaggccgggcttctcgcacaattatctacagcacaagcagccgcccctctggggtc
cctaatcgcttccctggatccatctctgggaacaaagccgccctcatcatcacaggagcc
cagcctgaggacgaggctgactattactgttccttgtatatgggtagttacactga
Germline J_{λ} sequences
SEQ ID NO. 390 IGLJ1 (F)
>IGLJ1*01|Canis lupus familiaris_boxer|F|J-REGION|
ttgggtattcggtgaagggacccagctgaccgtcctcg
SEQ ID NO. 391 IGLJ2 (F)
>IGLJ2*01|Canis lupus familiaris_boxer|F|J-REGION|
tatggtattcggcagagggacccagctgaccatcctcg
SEQ ID NO. 392 IGLJ3 (F)
>IGLJ3*01|Canis lupus familiaris_boxer|F|J-REGION|
tagtgtgttcggcggaggcacccatctgaccgtcctcg
SEQ ID NO. 393 IGLJ4 (F)
>IGLJ4*01|Canis lupus familiaris_boxer|F|J-REGION||
ttacgtgttcggctcaggaacccaactgaccgtccttg
SEQ ID NO. 394 IGLJ5 (F)
>IGLJ5*01|Canis lupus familiaris_boxer|F|J-REGION|
tattgtgttcggcggaggcacccatctgaccgtcctcg
SEQ ID NO. 395 IGLJ6 (F)
>IGLJ6*01|Canis lupus familiaris_boxer|F|J-REGION|
tggtgtgttcggcggaggcacccacctgaccgtcctcg
SEQ ID NO. 396 IGLJ7 (F)
>IGLJ7*01|Canis lupus familiaris_boxer|F|J-REGION|
tgctgtgttcggcggaggcacccacctgaccgtcctcg
SEQ ID NO. 397 IGLJ8 (F)
>IGLJ8*01|Canis lupus familiaris_boxer|F|J-REGION|
tgctgtgttcggcggaggcacccacctgaccgtcctcg
SEQ ID NO. 398 IGLJ9 (F)
>IGLJ9*01|Canis lupus familiaris_boxer|F|J-REGION|
ttacgtgttcggctcaggaacccaactgaccgtccttg

TABLE 4
Canine constant region genes

IGHC sequences
Functionality is shown between brackets, [F] and [P], when the
accession number (underlined) refers to rearranged genomic DNA
or cDNA and the corresponding germline gene has not yet been
isolated.
IGHA (F)
SEQ ID NO. 399
>IGHA*01|Canis lupus familiaris_boxer|F|CH1|
nagtccaaaaccagccccagtgtgttcccgctgagcctctgccaccaggagtcagaaggg
tacgtggtcatcggctgcctggtgcagggattcttcccaccggagcctgtgaacgtgacc
tggaatgccggcaaggacagcacatctgtcaagaacttcccccccatgaaggctgctacc
ggaagcctatacaccatgagcagccagttgaccctgccagccgcccagtgccctgatgac
tcgtctgtgaaatgccaagtgcagcatgcttccagccccagcaaggcagtgtctgtgccc
tgcaaa
SEQ ID NO. 400
>IGHA*01|Canis lupus familiaris_boxer|F|H-CH2|
gataactgtcatccgtgtcctcatccaagtccctcgtgcaatgagccccgcctgtcacta
cagaagccagccctcgaggatctgcttttaggctccaatgccagcctcacatgcacactg
agtggcctgaaagaccccaagggtgccaccttcacctggaacccctccaaagggaaggaa
cccatccagaagaatcctgagcgtgactcctgtggctgctacagtgtgtccagtgtccta
ccaggctgtgctgatccatggaaccatggggacaccttctcctgcacagccacccaccct
gaatccaagagcccgatcactgtcagcatcaccaaaaccaca
SEQ ID NO. 401
>>IGHA*01|Canis lupus familiaris_boxer|F|CH3-CHS|
gagcacatcccgccccaggtccacctgctgccgccgccgtcggaagagctggccctcaat
gagctggtgacactgacgtgcttggtgaggggcttcaaaccaaaagatgtgctcgtacga
tggctgcaagggacccaggagctaccccaagagaagtacttgacctgggagcccctgaag
gagcctgaccagaccaacatgtttgccgtgaccagcatgctgagggtgacagccgaagac
tggaagcagggggagaagttctcctgcatggtgggccacgaggctctgcccatgtccttc
acccagaagaccatcgaccgcctggcgggtaaacccacccacgtcaacgtgtctgtggtc
atggcagaggtggacggcatctgctac
SEQ ID NO. 402
>>IGHA*01|Canis lupus familiaris_boxer|F|M|
gactcacagtgtcttgcaggttaccgggagccacttccctggctggtgctggacctgtcg
caggaggacctggaggaggatgccccaggagccagcctgtggcccactaccgtcaccctt
ctcaccctcttcctactgagtctcttctacagcacagcactgactgtgacaagcgtgcgg
ggcccaactgacagcagagagggcccccagtac
IGHD (ORF)
SEQ ID NO. 403
>>|IGHD*01|Canis lupus familiaris_boxer|ORF|CH1|
gaatcgtcacttctgctccccttggtctcaggatgtaaggtcccaaaaaatggtgaggac
ataaccctggcctgcttggcaaaaggacccttcctagattctgtgcgggtcacgacaggc
ccagagtcacaggcccagatggaaaagaccacactgaagatgctaaagataccggaccac
actcaggtgtctctcctgtccaccccctggaaaccaggcctgcactactgcgaagccatc
aggaaagataacaaagagaagctgaagaaagccatccactggcca
SEQ ID NO. 404
>>IGHD*01|Canis lupus familiaris_boxer|ORF|H1|
gcatcctgggaaactgctatctccctgttgactcatgcgccatcccgaccccaggaccac
acccaagcccccagcatggccagggtctca
SEQ ID NO. 405
>>IGHD*01|Canis lupus familiaris_boxer|ORF|H2|
gtgcctcccaccagccacacccagacgcaagcccaggagccaggatgcccagtggacacc
atcctcaga
SEQ ID NO. 406
>>IGHD*01|Canis lupus familiaris_boxer|ORF|CH2|
gagtgttggaaccacacccaccctcccagcctctacatgctgcgccctcccctgcgggga
ccatggctccagggagaagctgctttcacctgcctggtggtgggagatgaccttcagaag
gcccacctgtcctgggaggtagccggggcgccccccagcgaggctgtggaggagaggcca
ctgcaggagcatgagaatggctcccagagctggagcagccgcctggtcttgcccatatcc
ctgtgggcctcaggagccaacatcacctgcacgctgagcctccccagcatgccttcccag
gtggtgtccgcagcagccagagagcat
SEQ ID NO. 407
>>IGHD*01|Canis lupus familiaris_boxer|ORF|CH3|
gctgccagagcacccagcagcctcaatgtccatgccctgaccatgcccagagcagcctcc
tggttcctgtgcgaggtgtccggcttctcaccccctgacatcctcctcacctggatcaag
gaccagattgaggtggacccttcttggttcgccactgcaccccccatggcccagccgggc
agtggcacgttccagacctggagtctcctgcgtgtcctcgctccccagggccctcacccg
cccacctacacgtgtgtagtcaggcacgaggcctcccggaagctgctcaacaccagctgg
agcctggacagt
SEQ ID NO. 408
>>IGHD*01|Canis lupus familiaris_boxer|ORF|M1|
ggtctgaccatgacccccccagcccctcagagccacgacgagagcagcggggactccatg
gatctggaagatgccagcggactgtggcccacgttcgctgccctcttcgtcctcactctg
ctctacagcggcttcgtcaccttcctcaaa
SEQ ID NO. 409
>>IGHD*01|Canis lupus familiaris_boxer|ORF|M2|
gtgaag
IGHE (F)
SEQ ID NO. 410
>IGHE*01|Canis lupus familiaris_boxer|F|CH1|
nccaccagccaggacctgtctgtgttccccttggcctcctgctgtaaagacaacatcgcc
agtacctctgttacactgggctgtctggtcaccggctatctccccatgtcgacaactgtg
acctgggacacggggtctctaaataagaatgtcacgaccttccccaccaccttccacgag
acctacggcctccacagcatcgtcagccaggtgaccgcctcgggcgagtgggccaaacag
aggttcacctgcagcgtggctcacgctgagtccaccgccatcaacaagaccttcagt
SEQ ID NO. 411
>IGHE*01|Canis lupus familiaris_boxer|F|CH2|
gcatgtgccttaaacttcattccgcctaccgtgaagctcttccactcctcctgcaacccc
gtcggtgatacccacaccaccatccagctcctgtgcctcatctctggctacgtcccaggt
gacatggaggtcatctggctggtggatgggcaaaaggctacaaacatattcccatacact
gcacccggcacaaaggagggcaacgtgacctctacccacagcgagctcaacatcacccag
ggcgagtgggtatcccaaaaaacctacacctgccaggtcacctatcaaggctttaccttt
aaagatgaggctcgcaagtgctca
SEQ ID NO. 412
>IGHE*01|Canis lupus familiaris_boxer|F|CH3|
gagtccgacccccgaggcgtgagcagctacctgagcccacccagcccccttgacctgtat
gtccacaaggcgcccaagatcacctgcctggtagtggacctggccaccatggaaggcatg
aacctgacctggtaccgggagagcaaagaacccgtgaacccgggccctttgaacaagaag
gatcacttcaatgggacgatcacagtcacgtctaccctgccagtgaacaccaatgactgg
atcgagggcgagacctactattgcagggtgacccacccgcacctgcccaaggacatcgtg
cgctccattgccaaggcccct
SEQ ID NO. 413
>IGHE*01|Canis lupus familiaris_boxer|F|CH4-CHS|
ggcaagcgtgcccccccggatgtgtacttgttcctgccaccggaggaggagcaggggacc
aaggacagagtcaccctcacgtgcctgatccagaacttcttccccgcggacatttcagtg
caatggctgcgaaacgacagccccatccagacagaccagtacaccaccacggggccccac
aaggtctcgggctccaggcctgccttcttcatcttcagccgcctggaggttagccgggtg
gactgggagcagaaaaacaaattcacctgccaagtggtgcatgaggcgctgtccggctct
aggatcctccagaaatgggtgtccaaaacccccggtaaa
SEQ ID NO. 414
>IGHE*01|Canis lupus familiaris_boxer|F|M1|
gagctccaggagctgtgcgcggatgccactgagagtgaggagctggacgagctgtgggcc
agcctgctcatcttcatcaccctcttcctgctcagcgtgagctacggcgccaccagcacc
ctcttcaag
SEQ ID NO. 415
>IGHE*01|Canis lupus familiaris_boxer|F|M2|
gtgaagtgggtactcgccaccgtcctgcaggagaagccacaggccgcccaagactacgcc
aacatcgtgcggccggcacag
IGHG1 [F]
SEQ ID NO. 416
>AF354264|IGHG1*01|Canis lupus familiaris|(F)|CH1||
gcctccaccacggccccctcggttttcccactggcccccagctgcgggtccacttccggc
tccacggtggccctggcctgcctggtgtcaggctacttccccgagcctgtaactgtgtcc
tggaattccggctccttgaccagcggtgtgcacaccttcccgtccgtcctgcagtcctca
gggcttcactccctcagcagcatggtgacagtgccctccagcaggtggcccagcgagacc
ttcacctgcaacgtggtccacccagccagcaacactaaagtagacaagcca
SEQ ID NO. 417
>IGHG1*01|Canis lupus familiaris|(F)|H|
Gtgttcaatgaatgcagatgcactgatacacccccatgccca
SEQ ID NO. 418
>IGHG1*01|Canis lupus familiaris|(F)|CH2|
gtccctgaacctctgggagggccttcggtcctcatctttcccccgaaacccaaggacatc
ctcaggattacccgaacacccgaggtcacctgtgtggtgttagatctgggccgtgaggac
cctgaggtgcagatcagctggttcgtggatggtaaggaggtgcacacagccaagacccag
tctcgtgagcagcagttcaacggcacctaccgtgtggtcagcgtcctccccattgagcac
caggactggctcacagggaaggagttcaagtgcagagtcaaccacatagacctcccgtct
cccatcgagaggaccatctctaaggccaga
SEQ ID NO. 419
>IGHG1*01|Canis lupus familiaris|(F)|CH3-CHS|
gggagggcccataagcccagtgtgtatgtcctgccgccatccccaaaggagttgtcatcc
agtgacacagtcagcatcacctgcctgataaaagacttctacccacctgacattgatgtg
gagtggcagagcaatggacagcaggagcccgagaggaagcaccgcatgaccccgccccag
ctggacgaggacgggtcctacttcctgtacagcaagctctctgtggacaagagccgctgg
cagcagggagaccccttcacatgtgcggtgatgcatgaaactctacagaaccactacaca
gatctatccctctcccattctccgggtaaa
IGHG2 (F)
SEQ ID NO. 420
>IGHG2*01|Canis lupus familiaris_boxer|F|CH1|
ncctccaccacggccccctcggttttcccactggcccccagctgcgggtccacttccggc
tccacggtggccctggcctgcctggtgtcaggctacttccccgagcctgtaactgtgtcc
tggaattccggctccttgaccagcggtgtgcacaccttcccgtccgtcctgcagtcctca
gggctctactccctcagcagcatggtgacagtgccctccagcaggtggcccagcgagacc
ttcacctgcaacgtggcccacccggccagcaaaactaaagtagacaagcca
SEQ ID NO. 421
>|IGHG2*01|Canis lupus familiaris_boxer|F|H|
Gtgcccaaaagagaaaatggaagagttcctcgcccacctgattgtcccaaatgccca
SEQ ID NO. 422
>IGHG2*01|Canis lupus familiaris_boxer|F|CH2|
gcccctgaaatgctgggagggccttcggtcttcatctttcccccgaaacccaaggacacc
ctcttgattgcccgaacacctgaggtcacatgtgtggtggtggatctggacccagaagac
cctgaggtgcagatcagctggttcgtggacggtaagcagatgcaaacagccaagactcag
cctcgtgaggagcagttcaatggcacctaccgtgtggtcagtgtcctccccattgggcac
caggactggctcaaggggaagcagttcacgtgcaaagtcaacaacaaagccctcccatcc
ccgatcgagaggaccatctccaaggccaga
SEQ ID NO. 423
>IGHG2*01|Canis lupus familiaris_boxer|F|CH3-CHS|
gggcaggcccatcaacccagtgtgtatgtcctgccgccatcccgggaggagttgagcaag
aacacagtcagcttgacatgcctgatcaaagacttcttcccacctgacattgatgtggag
tggcagagcaatggacagcaggagcctgagagcaagtaccgcacgaccccgccccagctg
gacgaggacgggtcctacttcctgtacagcaagctctctgtggacaagagccgctggcag
cggggagacaccttcatatgtgcggtgatgcatgaagctctacacaaccactacacacag
aaatccctctcccattctccgggtaaa
SEQ ID NO. 424
>IGHG2*01|Canis lupus familiaris_boxer|F|M1|
gagctgatcctggatgacagctgtgctgaggaccaggacggggagctggacgggctgtgg
accaccatctccatcttcatcaccctcttcctgctcagcgtgtgctacagcgccactgtc
accctcttcaag
SEQ ID NO. 425
>|IGHG2*01|Canis lupus familiaris_boxer|F|M2|
gtgaagtggatcttctcatcagtggtggagctgaagcgcacgattgtccccgactacagg
aatatgatcgggcagggggcc
IGHG3 [F]
SEQ ID NO. 426
>AF354266|IGHG3*01|Canis lupus familiaris|(F)|CH1|
gcctccaccacggccccctcggttttcccactggcccccagctgtgggtcccaatccggc
tccacggtggccctggcctgcctggtgtcaggctacatccccgagcctgtaactgtgtcc
tggaattccgtctccttgaccagcggtgtgcacaccttcccgtccgtcctgcagtcctca
gggctctactccctcagcagcatggtgacagtgccctccagcaggtggcccagcgagacc
ttcacctgcaatgtggcccacccggccaccaacactaaagtagacaagcca
SEQ ID NO. 427
>IGHG3*01|Canis lupus familiaris|(F)|H|
Gtggccaaagaatgcgagtgcaagtgtaactgtaacaactgcccatgccca
SEQ ID NO. 428
>IGHG3*01|Canis lupus familiaris|(F)|CH2|
ggttgtggcctgctgggagggccttcggtcttcatctttcccccaaaacccaaggacatc
ctcgtgactgcccggacacccacagtcacttgtgtggtggtggatctggacccagaaaac
cctgaggtgcagatcagctggttcgtggatagtaagcaggtgcaaacagccaacacgcag
cctcgtgaggagcagtccaatggcacctaccgtgtggtcagtgtcctccccattgggcac
caggactggctttcagggaagcagttcaagtgcaaagtcaacaacaaagccctcccatcc
cccattgaggagatcatctccaagacccca
SEQ ID NO. 429
>IGHG3*01|Canis lupus familiaris|(F)|CH3-CHS|
gggcaggcccatcagcctaatgtgtatgtcctgccgccatcgcgggatgagatgagcaag
aatacggtcaccctgacctgtctggtcaaagacttcttcccacctgagattgatgtggag
tggcagagcaatggacagcaggagcctgagagcaagtaccgcatgaccccgccccagctg
gatgaagatgggtcctacttcctatacagcaagctctccgtggacaagagccgctggcag
cggggagacaccttcatatgtgcggtgatgcatgaagctctacacaaccactacacacag
atatccctctcccattctccgggtaaa
IGHG4 [F]
SEQ ID NO. 430
>AF354267|IGHG4*01|Canis lupus familiaris|(F)|CH1|
gcctccaccacggccccctcggttttcccactggcccccagctgcgggtccacttccggc
tccacggtggccctggcctgcctggtgtcaggctacttccccgagcctgtaactgtgtcc
tggaattccggctccttgaccagcggtgtgcacaccttcccgtccgtcctgcagtcctca
gggctctactccctcagcagcacggtgacagtgccctccagcaggtggcccagcgagacc
ttcacctgcaacgtggtccacccggccagcaacactaaagtagacaagcca
SEQ ID NO. 431
>IGHG4*01|Canis lupus familiaris|(F)|H|
Gtgcccaaagagtccacctgcaagtgtatatccccatgccca
SEQ ID NO. 432
>IGHG4*01|Canis lupus familiaris|(F)|CH2|
gtccctgaatcactgggagggccttcggtcttcatctttcccccgaaacccaaggacatc
ctcaggattacccgaacacccgagatcacctgtgtggtgttagatctgggccgtgaggac
cctgaggtgcagatcagctggttcgtggatggtaaggaggtgcacacagccaagacgcag
cctcgtgagcagcagttcaacagcacctaccgtgtggtcagcgtcctccccattgagcac
caggactggctcaccggaaaggagttcaagtgcagagtcaaccacataggcctcccgtcc
cccatcgagaggactatctccaaagccaga
SEQ ID NO. 433
>IGHG4*01|Canis lupus familiaris|(F)|CH3-CHS|
gggcaagcccatcagcccagtgtgtatgtcctgccaccatccccaaaggagttgtcatcc
agtgacacggtcaccctgacctgcctgatcaaagacttcttcccacctgagattgatgtg
gagtggcagagcaatggacagccggagcccgagagcaagtaccacacgactgcgccccag
ctggacgaggacgggtcctacttcctgtacagcaagctctctgtggacaagagccgctgg
cagcagggagacaccttcacatgtgcggtgatgcatgaagctctacagaaccactacaca
gatctatccctctcccattctccgggtaaa
IGHM (F)
SEQ ID NO. 434
>IGHM*01|Canis lupus familiaris_boxer|F|CH1|
nagagtccatcccctccaaacctcttccccctcatcacctgtgagaactccctgtccgat
gagaccctcgtggccatgggctgcctggcccgggacttcctgcctggctccatcaccttc
tcctggaagtacgagaacctcagtgcaatcaacaaccaggacattaagaccttcccttca
gttctgagagagggcaagtatgtggcgacctctcaggtgttcctgccctccgtggacatc
atccagggttcagacgagtacatcacatgcaacgtcaagcactccaatggtgacaaatct
gtgaacgtgcccatcaca
SEQ ID NO. 435
>IGHM*01|Canis lupus familiaris_boxer|F|CH2|
gggcctgtaccaacgtctcccaacgtgactgtcttcatcccaccccgcgacgccttctct
ggcaatggccagcgcaagtcccagctcatctgccaggctgcaggtttcagccccaagcag
atttccgtgtcttggttccgtgatggaaagcagattgagtctggcttcaacacagggaag
gcagaggccgaggagaaagagcatgggcctgtgacctacagcatcctcagcatgctgacc
atcaccgagagtgcctggctcagccagagcgtgttcacctgccacgtggagcacaatggg
atcatcttccagaagaacgtgtcctccatgtgcacctcc
SEQ ID NO. 436
>IGHM*01|Canis lupus familiaris_boxer|F|CH3|
aatacacccgttggcatcagcatcttcaccatccccccctcctttgccagcatcttcaac
accaagtcagccaagctgtcctgcctggtcactgacctggccacttatgacagcctgacc
atctcctggacccgtcagaatggcgaggctctgaaaacccacaccaacatctctgagagc
catcccaacaacaccttcagtgccatgggggaagccactgtctgcgtggaggaatgggag
tcaggcgagcagttcacctgcacagtgacccacacagatctgccctcaccgctgaagaag
accatctccaggcccaag
SEQ ID NO. 437
>IGHM*01|Canis lupus familiaris_boxer|F|CH4-CHS|
gatgtcaacaagcacatgccttctgtctacgtcctgcccccgagccgggagcagctgagc
ctgcgggaatcggcctcactcacctgcctggtgaaaggcttctcacccccagatgtgttc
gtgcagtggctgcagaagggccagcccgtgccccctgacagctacgtgaccagcgccccg
atgcccgagccccaagcccccggcctctactttgtccacagcatcctgaccgtgagtgag
gaggactggaatgccggggagacctacacctgtgttgtaggccatgaggccctgccccat
gtggtgaccgagaggagcgtggacaagtccaccggtaaacccaccttgtacaacgtgtcc
ctggtcttatctgacacagccagcacctgctac
SEQ ID NO. 438
>IGHM*01|Canis lupus familiaris_boxer|F|M1|
gggggggaggtgagtgccgaggaggaaggcttcgagaacctgaataccatggcatccacc
ttcatcgtcctcttcctcctcagtgtcttctacagcaccacagtcactctgttcaag
SEQ ID NO. 439
>IGHM*01|Canis lupus familiaris_boxer|F|M2|
gtgaaa
IGKC sequences
IGKC (F)
SEQ ID NO. 440
>IGKC*01|Canis lupus familiaris_boxer|F|C-REGION|
cggaatgatgcccagccagccgtctatttgttccaaccatctccagaccagttacacaca
ggaagtgcctctgttgtgtgcttgctgaatagcttctaccccaaagacatcaatgtcaag
tggaaagtggatggtgtcatccaagacacaggcatccaggaaagtgtcacagagcaggac
aaggacagtacctacagcctcagcagcaccctgacgatgtccagtactgagtacctaagt
catgagttgtactcctgtgagatcactcacaagagcctgccctccaccctcatcaagagc
ttccaaaggagcgagtgtcagagagtggac
IGLC sequences
[F], Functionality defined for the available sequence of
the gene (partial gene in 3′ because of gaps in the sequence)
SEQ ID NO. 441 IGLC1 (F)
>IGLC1*01|Canis lupus familiaris_boxer|F|C-REGION|
ggtcagcccaagtcctcccccttggtcacactcttcccgccctcctctgaggagctcggc
gccaacaaggctaccctggtgtgcctcatcagcgacttctaccccagtggcctgaaagtg
gcttggaaggcagatggcagcaccatcatccagggcgtggaaaccaccaagccctccaag
cagagcaacaacaagtacacggccagcagctacctgagcctgacgcctgacaagtggaaa
tctcacagcagcttcagctgcctggtcacgcaccaggggagcaccgtggagaagaaggtg
gcccctgcagagtgctct
SEQ ID NO. 442 IGLC2 (F)
>IGLC2*01|Canis lupus familiaris_boxer|F|C-REGION|
ggtcagcccaaggcctccccctcagtcacactcttcccaccctcctctgaggagctcggc
gccaacaaggccaccctggtgtgcctcatcagcgacttctaccccagcggcgtgacggtg
gcctggaaggcagacggcagccccggcatccagggcgtggagaccaccaagccctccaag
cagagcaacaacaagtacgcggccagcagctacctgagcctgacgcctgacaagtggaaa
tctcacagcagcttcagctgcctggtcacgcatgaggggagcaccgtggagaagaaggtg
gcccccgcagagtgctct
SEQ ID NO. 443 IGLC3 (F)
>IGLC3*01|Canis lupus familiaris_boxer|F|C-REGION|
ggtcagcccaaggcctccccctcggtcacactcttcccgccctcctctgaggagctcggc
gccaacaaggccaccctggtgtgcctcatcagcgacttctaccccagtggcgtgacggtg
gcctggaaggcagacggcagccccgtcacccagggcgtggagaccaccaagccctccaag
cagagcaacaacaagtacgcggccagcagctacctgagcctgacgcctgacaagtggaaa
tctcacagcagcttcagctgcctggtcacacacgaggggagcaccgtggagaagaaggtg
gcccccgcagagtgctct
SEQ ID NO. 444 IGLC4 [F]
>IGLC4*01|Canis lupus familiaris_boxer|F|C-REGION|
ggtcagcccaaggcctccccctcggtcacactcttcccgccctcctctgaggagctcggc
gccaacaaggccaccctggtgtgcctcatcagcgacttctaccccagcggtgtgacggtg
gcctggaaggcagacggcagccccgtcacccagggcgtggagaccaccaagccctccaag
cagagcaacaacaagtacgcggccagcagctacctgagcctgacgcctgacaagtggaaa
tctcacagcagcttcagctgcctggtcacacacgaggggagcactgtgg
SEQ ID NO. 445 IGLC5 (F)
>IGLC5*01|Canis lupus familiaris_boxer|F|C-REGION|
ggtcagcccaaggcctccccttcggtcacactcttcccgccctcctctgaggagcttggc
gccaacaaggccaccctggtgtgcctcatcagcgacttctaccccagcggcgtgacagtg
gcctggaaggcagacggcagccccatcacccagggtgtggagaccaccaagccctccaag
cagagcaacaacaagtacgcggccagcagctacctgagcctgacgcctgacaagtggaaa
tctcacagcagcttcagctgcctggtcacgcacgaggggagcaccgtggagaagaaggtg
gcccccgcagagtgctct
SEQ ID NO. 446 IGLC6 (F)
>IGLC6*01|Canis lupus familiaris_boxer|F|C-REGION|
ggtcagcccaaggcctccccctcggtcacactcttcccgccctcctctgaggagctcggc
gccaacaaggccaccctggtgtgcctcatcagcgacttctaccccagcggtgtgacggtg
gcctggaaggcagacggcagccccgtcacccagggcgtggagaccaccaagccctccaag
cagagcaacaacaagtacgcggccagcagctacctgagcctgacgcctgacaagtggaaa
tctcacagcagcttcagctgcctggtcacgcacgaggggagcaccgtggagaagaaggtg
gcccccgcagagtgctct
SEQ ID NO. 447 IGLC7 (F)
>IGLC7*01|Canis lupus familiaris_boxer|F|C-REGION|
ggtcagcccaaggcctccccctcggtcacactcttcccgccctcctctgaggagctcggc
gccaacaaggccaccctggtgtgcctcatcagcgacttctaccccagcggcgtgacggtg
gcctggaaggcagacggcagccccgtcacccagggcgtggagaccaccaagccctccaag
cagagcaacaacaagtacgcggccagcagctacctgagcctgacgcctgacaagtggaaa
tctcacagcagcttcagctgcctggtcacgcacgaggggagcaccgtggagaagaaggtg
gcccccgcagagtgctct
SEQ ID NO. 448 IGLC8 (F)
>IGLC8*01|Canis lupus familiaris_boxer|F|C-REGION|
ggtcagcccaaggcctccccctcggtcacactcttcccgccctcctctgaggagctcggc
gccaacaaggccaccctggtgtgcctcatcagcgacttctaccccagcggcgtgacggtg
gcctggaaggcagacggcagccccgtcacccagggcgtggagaccaccaagccctccaag
cagagcaacaacaagtacgcggccagcagctacctgagcctgacgcctgacaagtggaaa
tctcacagcagcttcagctgcctggtcacgcacgaggggagcaccgtggagaagaaggtg
gcccccgcagagtgctct
SEQ ID NO. 449 IGLC9 (F)
>IGLC9*01|Canis lupus familiaris_boxer|F|C-REGION|
ggtcagcccaaggcctccccctcggtcacactcttcccgccctcctctgaggagctcggc
gccaacaaggccaccctggtgtgcctcatcagcgacttctaccccagcggcgtgacggtg
gcctggaaggcagacggcagccccatcacccagggcgtggagaccaccaagccctccaag
cagagcaacaacaagtacgcggccagcagctacctgagcctgacgcctgacaagtggaaa
tctcacagcagcttcagctgcctggtcacgcacgaggggagcactgtggagaagaaggtg
gcccccgcagagtgctct
// End of canine Ig sequences

TABLE 5
PCR Primers

| | |
|---|---|
| SEQ ID NO. 450 | 1F: ACATAATACACTGAAATGGAGCCC |
| SEQ ID NO. 451 | IR: GTCCTTGGTCAACGTGAGGG |
| SEQ ID NO. 452 | 2F: CATAATACACTGAAATGGAGCCCT |
| SEQ ID NO. 453 | 2R: GCAACAGTGGTAGGTCGCTT |

TABLE 6
Miscellaneous sequence data

Pre-DJ
This is a 21609 bp fragment upstream of the Ighd-5
DH gene. The pre-DJ sequence can be found in Mus
musculus strain C57BL/6J chromosome 12, Assembly:
GRCm38.p4, Annotation release 106, Sequence ID:
NC_000078.6
The entire sequence lies between the two 100 bp
sequences shown below:
Upstream of the Ighd-5 DH gene segment,
corresponding to positions 113526905-113527004 in
NC_000078.6:
ATTTCTGTACCTGATCTATGTCAATATCTGTACCATGGCTCTAGCAGAGAT
GAAATATGAGACAGTCTGATGTCATGTGGCCATGCCTGGTCCAGACTTG
(SEQ ID NO. 454)
2 kb upstream of the ADAM6A gene corresponding to
positions 113548415-113548514 in NC_000078.6:
GTCAATCAGCAGAAATCCATCATACATGAGACAAAGTTATAATCAAGAAAT
GTTGCCCATAGGAAACAGAGGATATCTCTAGCACTCAGAGACTGAGCAC
(SEQ ID NO. 455)
ADAM6A
ADAM6A (a disintegrin and metallopeptidase domain
6A) is a gene involved in male fertility. The
ADAM6A sequence can be found in Mus musculus strain
C57BL/6J chromosome 12, Assembly: GRCm38.p4,
Annotation release 106, Sequence ID: NC_000078.6 at
position 113543908-113546414.
ADAM6A sequence ID: OTTMUSG00000051592 (VEGA)

TABLE 7
Chimeric canine/mouse Ig gene sequences

IGK Version A
Sequence upstream of mouse Igkv 1-133 (SEQ ID NO: 456)
GCATTGAATAAACCAGTATAAACAAGCAAGCAAAGATAGATAGATAGATAGATAGATAGATAGATAGATAC
ATAGATAGATAGATAGATAGATAGATGATAGATAGATAGATAGATAGATAGATTTTTACGTATAATACAAT
AAAAACATTCATTGTCCCTCTATTGGTGACTACTCAAGGAAAAAAATGTTCATATGCAAGAAAAAATGTTA
TCATTACCAGATGATCCAGCAATCTAGCAATATATATATTGTTTATTCACAAAACATGAATGAACCTTTTA
AGAAGCTGTTACAGTGTAAAAATTAAGTTAAATCACTGAAGAACATATACTGTGTGATTTCATTCAAATGA
AATTTGAGAAGTAAATATATATGTATATATATATATATGTAAAAAATATAAGTCTGAACTACAAAAATTCA
ATTTGTTTGATATGTAAGAATAAGAAAAATTGACCCCCAAAATTTGTTAATAATTAGGTATGTGTATTTTT
ATGAATATATAAGTATAATAATGCTTATAGTATACACTATTCTGAATCACATTTATTCCCTAAGTGTGTTC
CCTTGATTATAATTAAAAGTATATTTTTTAAATACAGAGTCAGAGTACAGTCAATAAGGCGAAAATATAGT
TGAATGATTTGCTTCAGCTTTTGTAATGTACTAGAGATTGTGAGTACAAAGTCTCAGAGCTCATTTTATCC
CTGACAATAACCAGCTCTGTGCTTCAAGTACATTTCCATCTTTCTCTGAAATTTAGTCTTATATAGATAGA
CAAAATTTAAGTAAATTTCAAACTACACAGAACAACTAAGTTGTTGTTTCATATTGATAATGGATTTGAAC
TGCATTAACAGAACTTTAACATCCTGCTTATTCTCCCTTCAGCCATCATATTTTGCTTTATTATTTTCACT
TTTTGAGTTATTTTTCACATTCAGAAAGCTCACATAATTGTCACTTCTTTGTATACTGGTATACAGACCAG
AACATTTGCATATTGTTCCCTGGGGAGGTCTTTGCCCTGTTGGCCTGAGATAAAACCTCAAGTGTCCTCTT
GCCTCCACTGATCACTCTCCTATGTTTATTTCCTCAAA
Canine exon 1 (leader) from LOC475754 (SEQ ID NO. 457):
atgaggttcccttctcagctcctggggctgctgatgctctggatcc
Canine intron 1 from LOC475754 (SEQ ID NO. 458)
Caggtaaggacagggcggagatgaggaaagacatgggggcgtggatggtgagctcccctggtgctgtttct
ctccctgtgtattctgtgcatgggacagattgccctccaacagggggaatttaatttttagactgtgagaa
ttaagaagaatataaaatatttgatgaacagtactttagtgagatgctaaagaagaaagaagtcactctgt
cttgctatcttgggttttccatgataattgaatagatttaaaatataaatcaaaatcaaaatatgatttag
cctaaaatatacaaaacccaaaatgattgaaatgtcttatactgtttctaacacaacttgtacttatctct
cattattttaggatccagtggg
Canine 5′ part of exon 2 (leader) from LOC475754 (SEQ ID NO. 459)
aggatccagtggg
Canine Vκ from LOC475754 (SEQ ID NO. 460)
Gatattgtcatgacacagaccccactgtccctgtctgtcagccctggagagactgcctccatctcctgcaa
ggccagtcagagcctcctgcacagtgatggaaacacgtatttgaactggttccgacagaagccaggccagt
ctccacagcgtttaatctataaggtctccaacagagaccctggggtcccagacaggttcagtggcagcggg
tcagggacagatttcaccctgagaatcagcagagtggaggctgacgatactggagtttattactgcgggca
aggtatacaagat
Mouse RSS heptamer (SEQ ID NO: 461)
CACAGTG
Mouse sequence downstream of RSS heptamer (SEQ ID NO. 462)
ATACAGACTCTATCAAAAACTTCCTTGCCTGGGGCAGCCCAGCTGACAATGTGCAATCTGAAGAGGAGCAG
AGAGCATCTTGTGTCTGTGTGAGAAGGAGGGGCTGGGATACATGAGTAATTCTTTGCAGCTGTGAGCTCTG
IGK version B
Sequence upstream of mouse Igkv 1-133 (SEQ ID NO. 463)
GCATTGAATAAACCAGTATAAACAAGCAAGCAAAGATAGATAGATAGATAGATAGATAGATAGATAGATAC
ATAGATAGATAGATAGATAGATAGATGATAGATAGATAGATAGATAGATAGATTTTTACGTATAATACAAT
AAAAACATTCATTGTCCCTCTATTGGTGACTACTCAAGGAAAAAAATGTTCATATGCAAGAAAAAATGTTA
TCATTACCAGATGATCCAGCAATCTAGCAATATATATATTGTTTATTCACAAAACATGAATGAACCTTTTA
AGAAGCTGTTACAGTGTAAAAATTAAGTTAAATCACTGAAGAACATATACTGTGTGATTTCATTCAAATGA
AATTTGAGAAGTAAATATATATGTATATATATATATATGTAAAAAATATAAGTCTGAACTACAAAAATTCA
ATTTGTTTGATATGTAAGAATAAGAAAAATTGACCCCCAAAATTTGTTAATAATTAGGTATGTGTATTTTT
ATGAATATATAAGTATAATAATGCTTATAGTATACACTATTCTGAATCACATTTATTCCCTAAGTGTGTTC
CCTTGATTATAATTAAAAGTATATTTTTTAAATACAGAGTCAGAGTACAGTCAATAAGGCGAAAATATAGT
TGAATGATTTGCTTCAGCTTTTGTAATGTACTAGAGATTGTGAGTACAAAGTCTCAGAGCTCATTTTATCC
CTGACAATAACCAGCTCTGTGCTTCAAGTACATTTCCATCTTTCTCTGAAATTTAGTCTTATATAGATAGA
CAAAATTTAAGTAAATTTCAAACTACACAGAACAACTAAGTTGTTGTTTCATATTGATAATGGATTTGAAC
TGCATTAACAGAACTTTAACATCCTGCTTATTCTCCCTTCAGCCATCATATTTTGCTTTATTATTTTCACT
TTTTGAGTTATTTTTCACATTCAGAAAGCTCACATAATTGTCACTTCTTTGTATACTGGTATACAGACCAG
AACATTTGCATATTGTTCCCTGGGGAGGTCTTTGCCCTGTTGGCCTGAGATAAAACCTCAAGTGTCCTCTT
GCCTCCACTGATCACTCTCCTATGTTTATTTCCTCAAA
Mouse IGKV 1-133 exon 1 (leader) (SEQ ID NO. 464)
ATGATGAGTCCTGCCCAGTTCCTGTTTCTGTTAGTGCTCTGGATTCAGG
Mouse IGKV 1-133 intron 1 (SEQ ID NO. 465)
GTAAGGAGTTTTGGAATGTGAGGGATGAGAATGGGGATGGAGGGTGATCTCTGGATGCCTATGTGTGCTGT
TTATTTGTGGTGGGGCAGGTCATATCTTCCAGAATGTGAGGTTTTGTTACATCCTAATGAGATATTCCACA
TGGAACAGTATCTGTACTAAGATCAGTATTCTGACATAGATTGGATGGAGTGGTATAGACTCCATCTATAA
TGGATGATGTTTAGAAACTTCAACACTTGTTTTATGACAAAGCATTTGATATATAATATTTTTAAATCTGA
AAAACTGCTAGGATCTTACTTGAAAGGAATAGCATAAAAGATTTCACAAAGGTTGCTCAGGATCTTTGCAC
ATGATTTTCCACTATTGTATTGTAATTTCAG
Mouse IGKV 1-133 5′ part of exon 2 (leader) (SEQ ID NO. 466)
AAACCAACGGT
Canine Vκ from LOC475754 (SEQ ID NO. 467)
Gatattgtcatgacacagaccccactgtccctgtctgtcagccctggagagactgcctccatctcctgcaa
ggccagtcagagcctcctgcacagtgatggaaacacgtatttgaactggttccgacagaagccaggccagt
ctccacagcgtttaatctataaggtctccaacagagaccctggggtcccagacaggttcagtggcagcggg
tcagggacagatttcaccctgagaatcagcagagtggaggctgacgatactggagtttattactgcgggca
aggtatacaagat
Mouse RSS heptamer (SEQ ID NO: 468)
CACAGTG
Mouse sequence downstream of RSS heptamer (SEQ ID NO. 469)
ATACAGACTCTATCAAAAACTTCCTTGCCTGGGGCAGCCCAGCTGACAATGTGCAATCTGAAGAGGAGCAG
AGAGCATCTTGTGTCTGTGTGAGAAGGAGGGGCTGGGATACATGAGTAATTCTTTGCAGCTGTGAGCTCTG

1. A transgenic rodent or rodent cell comprising a genome comprising an engineered partly canine immunoglobulin light chain locus comprising canine immunoglobulin λ light chain variable region gene segments, wherein the engineered immunoglobulin locus is capable of expressing immunoglobulin comprising canine variable domains and wherein the transgenic rodent produces more, or is more likely to produce, immunoglobulin comprising λ light chain than immunoglobulin comprising κ light chain.

2. The transgenic rodent according to claim 1, wherein more λ light chain producing cells than κ light chain producing cells are likely to be isolated from said rodent.

3. The transgenic rodent according to claim 1, wherein the transgenic rodent produces at least about 25%, 30%, 35%, 40%, 45%, 50%, 55%, 60%, 65%, 70%, 75%, 80%, 85%, 90% or 95% and up to about 100% immunoglobulin comprising λ light chain.

4. The transgenic rodent cell according to claim 1, wherein the transgenic rodent cell, or its progeny, has at least about a 25%, 30%, 35%, 40%, 45%, 50%, 55%, 60%, 65%, 70%, 75%, 80%, 85%, 90%, or 95% and up to about 100%, probability of producing immunoglobulin comprising λ light chain.

5. The transgenic rodent or rodent cell according to claim 1, wherein the engineered immunoglobulin locus comprises canine V_{λ} and J_{λ} gene segment coding sequences embedded in rodent non-coding regulatory or scaffold sequences of a rodent immunoglobulin λ light chain variable region gene locus.

6. The transgenic rodent or rodent cell according to claim 1, wherein the engineered immunoglobulin locus comprises canine V_{λ} and J_{λ} gene segment coding sequences embedded in rodent non-coding regulatory or scaffold sequences of a rodent immunoglobulin κ light chain variable region gene locus.

7. The transgenic rodent or rodent cell according to claim 6, wherein the engineered immunoglobulin variable region locus comprises one or more canine V_{λ} gene segment coding sequences and one or more J-C units wherein each J-C unit comprises a canine J_{λ} gene segment coding sequence and a rodent λ constant region coding sequence.

8. The transgenic rodent or rodent cell according to claim 7, wherein the rodent λ constant region coding sequence comprises a rodent C_{λ1}, C_{λ2}, C_{λ3} coding sequence, or a combination thereof.

9. The transgenic rodent or rodent cell according to claim 7, wherein the J-C units comprise canine J_{λ} gene segment coding sequences and rodent λ constant region coding sequences embedded in non-coding regulatory or scaffold sequences of a rodent immunoglobulin κ light chain locus.

10. The transgenic rodent or rodent cell according to claim 6, wherein the engineered immunoglobulin locus comprises a rodent immunoglobulin κ locus in which one or more rodent V_{κ} gene segment coding sequences and one or more rodent J_{κ} gene segment coding sequences have been deleted and replaced by one or more canine V_{λ} gene segment coding sequences and one or more J_{λ} gene segment coding sequences, respectively, and in which rodent C_{κ} coding sequences in the locus have been replaced by rodent C_{λ1}, C_{λ2}, C_{λ3} coding sequence, or a combination thereof.

11. The transgenic rodent or rodent cell according to claim 1 wherein:
(A) an endogenous rodent immunoglobulin κ light chain locus is deleted, inactivated, or made nonfunctional one or more of:
i. deleting or mutating all endogenous rodent V_{κ} gene segment coding sequences;
ii. deleting or mutating all endogenous rodent J_{κ} gene segment coding sequences;
iii. deleting or mutating all endogenous rodent C_{κ} coding sequence;
iv. deleting or mutating a 5′ splice site and adjacent polypyrimidine tract of a rodent C_{κ} coding sequence;
v. deleting, mutating, or disrupting an endogenous intronic κ enhancer (iE_{κ}) and 3′ enhancer sequence; or
(B) an endogenous rodent immunoglobulin λ light chain variable domain is suppressed or inactivated by one or more of:
i. deleting or mutating all endogenous rodent V_{λ} gene segments
ii. deleting or mutating all endogenous rodent J_{λ} gene segments; and
iii. deleting or mutating all endogenous rodent C_{λ} coding sequences.

12. The transgenic rodent or rodent cell according to claim 1, wherein the engineered immunoglobulin locus expresses immunoglobulin light chains comprising a canine λ variable domain and rodent λ constant domain.

13. The transgenic rodent or rodent cell according claim 1, wherein the genome of the transgenic rodent or rodent cell comprises an engineered immunoglobulin locus comprising canine V_{κ} and J_{κ} gene segment coding sequences embedded in rodent non-coding regulatory or scaffold sequences of the rodent immunoglobulin κ light chain variable region gene locus.

14. The transgenic rodent or rodent cell according to claim 13, wherein the canine V_{κ} and J_{κ} coding sequences are inserted upstream of a rodent immunoglobulin κ light chain constant region coding sequence.

15. The transgenic rodent or rodent cell according to claim 1, wherein the genome of the transgenic rodent or rodent cell comprises an engineered immunoglobulin locus comprising canine V_{κ} and J_{κ} gene segment coding sequences embedded in rodent non-coding regulatory or scaffold sequences of the rodent immunoglobulin λ light chain variable region gene locus.

16. The transgenic rodent or rodent cell according to claim 15, comprising a rodent immunoglobulin κ light chain constant region coding sequence inserted downstream of the canine V_{κ} and J_{κ} gene segment coding sequences.

17. The transgenic rodent or rodent cell according to claim 16, wherein the rodent immunoglobulin κ light chain constant region is inserted upstream of an endogenous rodent C_{λ2} coding sequence.

18. The transgenic rodent or rodent cell according to claim 15, wherein expression of an endogenous rodent immunoglobulin λ light chain variable domain is suppressed or inactivated by one or more of:
a. deleting or mutating all endogenous rodent V_{λ} gene segment coding sequences.
b. deleting or mutating all endogenous rodent J_{λ} gene segment coding sequences; and
c. deleting or mutating all endogenous C_{λ} coding sequences or splice sites.

19. The transgenic rodent or rodent cell according to claim 1 wherein the engineered canine immunoglobulin light chain locus comprises a rodent intronic κ enhancer (iE_{κ}) and 3′E_{κ} regulatory sequences.

20. The transgenic rodent or rodent cell according to claim 1, wherein the transgenic rodent or rodent cell comprises an engineered partly canine immunoglobulin heavy chain locus comprising canine immunoglobulin heavy chain variable region gene coding sequences and non-coding regulatory or scaffold sequences of the rodent immunoglobulin heavy chain locus.

21. The transgenic rodent or rodent cell according to claim 20, wherein the engineered canine immunoglobulin heavy chain locus comprises canine V_{H}, D and J_{H} gene segments comprising V_{H}, D or J_{H} coding sequences embedded in non-coding regulatory or scaffold sequences of the rodent immunoglobulin heavy chain locus.

22. The transgenic rodent or rodent cell according to claim 21, wherein the heavy chain scaffold sequences are interspersed by functional ADAM6A genes, ADAM6B genes, or a combination thereof.

23. The transgenic rodent or rodent cell according to claim 1, wherein the rodent regulatory or scaffold sequences comprise enhancer, promoters, splice sites, introns, recombination signal sequences, or combinations thereof.

24. The transgenic rodent or rodent cell according to claim 1, wherein an endogenous rodent immunoglobulin locus has been deleted and replaced with the engineered partly canine immunoglobulin locus.

25. The transgenic rodent or rodent cell according to claim 1, wherein the rodent is a mouse or a rat.

26. The transgenic rodent or rodent cell according to claim 1, wherein the rodent cell is a mouse or rat embryonic stem (ES) cell, or mouse or rat cell of an early stage embryo.

27. A cell of B lymphocyte lineage obtained from the transgenic rodent of claim 1, wherein the engineered immunoglobulin locus expresses a chimeric immunoglobulin heavy chain or light chain comprising a canine variable region and a rodent immunoglobulin constant region.

28. A hybridoma cell or immortalized cell line derived from a cell of B lymphocyte lineage according to claim 27.

29. Antibodies or antigen binding portions thereof produced by the cell of claim 27.

30. A nucleic acid sequence of a V_{H}, D, or J_{H}, or a V_{L} or J_{L} gene segment coding sequence derived from an immunoglobulin produced by the cell of claim 27.
